(19) United States
(12) Patent Application Publication
(10) Pub. No.: US 2026/0258083 A1
(43) Pub. Date: Sep. 3, 2026

(54) T Cell Engager Masking Molecules

(71) Applicant: AMGEN INC., Thousand Oaks, CA (US)

(72) Inventors: Kip Conner, Thousand Oaks, CA (US); Tim Acker, Thousand Oaks, CA (US); Dan Rock, Thousand Oaks, CA (US); Aye Myat Thinn, Thousand Oaks, CA (US); Aiko Umeda, Thousand Oaks, CA (US)

(21) Appl. No.: 19/489,667

(22) PCT Filed: Jun. 14, 2024
(86) PCT No.: PCT/US2024/034204

§ 371 Date: Dec. 3, 2025

Related U.S. Application Data
(60) Provisional application No. 63/508,265, filed on Jun. 14, 2023.
(60) Provisional application No. 63/647,855, filed on May 15, 2024.

(51) Int. Cl. C07K 7/08; A61K 38/00; A61K 39/00; A61K 47/60; A61P 37/06; C07K 16/28; C07K 16/30

(52) U.S. Cl. CPC C07K 7/08; A61K 47/60; A61P 37/06; C07K 16/2809; C07K 16/3069; A61K 38/00; A61K 2039/505; C07K 2317/31; C07K 2317/34; C07K 2317/524; C07K 2317/526; C07K 2317/53; C07K 2317/55; C07K 2317/565; C07K 2317/622; C07K 2317/92; C07K 2319/31; C07K 2319/70

(57) ABSTRACT

The present invention relates to T cell engager masking molecules, and methods related thereto, that reduce the severity of cytokine release syndrome, wherein said molecules have (i.) a binding peptide which binds to a T-cell engaging paratope of a bispecific T cell engager molecule (TCE), (ii.) a linker, and (iii.) a half-life extending polymer.

### TECHNICAL FIELD

This invention relates to aspects of biotechnology and medicine, in particular, to preventing and/or reducing the severity of cytokine release.

### BACKGROUND

The advent of redirecting T cell cytotoxicity in the field of immunotherapy has provided an abundantly increasing number of therapeutic approaches over the last years. Besides genetically modified T cells with chimeric antigen receptors (CAR), various bispecific T cell engagers (TCE) have successfully entered the market and are under development to treat various conditions including cancer. Exemplary bispecific molecules are recombinant protein constructs made from two flexibly linked antibody derived binding domains. One binding domain of TCEs is typically specific for a selected tumor-associated surface antigen on target cells; the second binding domain is specific for CD3, a subunit of the T cell receptor (TCR) complex on T cells.

However, T cell redirection on-target activity, like CAR T cells, is naturally associated with the risk of leading to a strong release of pro-inflammatory cytokines which may induce a cytokine release syndrome (CRS). CRS is considered a major dose-limiting clinical toxicity associated with T cell-directing immunotherapy that limits a patient's ability to achieve efficacious TCE doses quickly and safely (Shimabukuro-Vornhagen, A. at al., (2018) Cytokine release syndrome. Journal for immunotherapy of cancer 6, 56). While the relationship between TCE exposure and CRS is still under investigation, the etiology of CRS may be derived from the following: (i.) clinical CRS is predominantly associated with first cycle TCE therapy; (Shimabukuro-Vornhagen, A., et al., supra). (ii.) cytokine release tends to decrease with repeat TCE treatment cycles; (Chen, X. et al., (2019) A Modeling Framework to Characterize Cytokine Release upon T-Cell-Engaging Bispecific Antibody Treatment: Methodology and Opportunities. 12, 600-608); and (iii.) TCE regimens that include step-dosing can lessen CRS incidence, but the success of this dosing strategy can be dependent on the specific target/indication (Jacobs, K. et al., (2017) Lead-in Dose Optimization to Mitigate Cytokine Release Syndrome in AML and MDS Patients Treated with Flotetuzumab, a CD123×CD3 Dart® Molecule for T-Cell Redirected Therapy. Blood 130, 3856-3856).

Even if CRS may present as a transient condition, and even if it is most frequently associated with the first dose or cycle of treatment, a higher degree CRS requires intensive care, may lead to the aborting of the T cell-redirecting therapy, and in some cases may lead to the death of the patient if not managed properly. Clinical strategies to address CRS during immunotherapy are known and include: (a) altering dosing regimens, such as lengthy step dosing or extended intravenous (eIV) administration protocols; (b) prophylactic treatment with corticosteroids (e.g. dexamethasone); and (c) intervention therapy with targeted anti-cytokine antibody drugs (e.g., anti-IL-6R, tocilizumab). Other strategies include the use of target cell pre-depletion approaches and/or target cell masking antibodies, the engineering of CD3 binders of T cell engagers to reduce first infusion cytokine release, or the use of small molecule kinase inhibitors to prevent cytokine release following treatment with T cell engaging therapies (see G Leclercy et al. “Novel strategies for the mitigation of cytokine release syndrome induced by T cell engaging therapies with a focus on the use of kinase inhibitors”, ONCOIMMUNOLOGY 2022, VOL. 11, NO. 1). On a molecular design level, masking of TCEs, such as AMX-818, ANX007 and other clinical candidates, is currently under investigation to mitigate high potency and on-target, off-tumor toxicities. However, such approaches typically rely on protease-mediated mask cleavage to elicit TCE activity. Accordingly, one or more protease cleavage sites have to be introduced into the linker which covalently links the masking moiety and the drug molecule to be masked. These one or more cleavage sites are cleaved by proteases which may be frequently dysregulated in tumors but have a significant chance to be not dysregulated or overexpressed in the tumor which is actually targeted in each and every case. Also, a covalently linked masking moiety requires significant molecule engineering of the specific drug molecule which may impact efficacy and/or stability properties of the original un-masked drug molecule. Also, engineering efforts have to be repeated for tailored masking for each drug molecule.

However, despite the advances in managing CRS and current engineering approaches, known mitigation efforts may reduce or eliminate entirely the efficacy of the immunotherapeutic treatment. Also, targeted approaches are typically not sufficient to prevent the strong and rapid onset of a cytokine storm that can occur after CAR T cell treatment or by molecules engineered with high avidity binding to the tumor-associated antigen and/or toward the, e.g., CD3ε of the TCR. Hence, there is a strong need for an improved versatile CRS mitigation strategy. There exists a need for CRS mitigation strategies that are applicable for a variety of TCE, e.g. by transient inhibition of the broad spectrum of cytokines and chemokines in order to more efficiently resolve CRS symptoms, as observed, e.g., with glucocorticoids.

### SUMMARY

Surprisingly, a TCE masking molecule comprising a binding peptide which is functionalized by a polymer for increased half-life and which is typically derived from the CD3 T-cell co-receptor, e.g., from the endogenous N-terminal sequence of CD3ε T cell co-receptor, when combined, e.g. co-administered, with the TCE can effectively moderate the pharmacokinetic (PK) exposure profile of the TCE. characterized by gradual onset (tₘₐₓ) of an attenuated maximal exposure (Cₘₐₓ) of the TCE.

Hence, in one aspect, this disclosure provides a
T cell engager (TCE) masking molecule, the molecule comprising
(i.) at least one binding peptide which binds to a T-cell engaging paratope of a T-cell engager (TCE) molecule, wherein the T-cell engaging paratope is an anti-CD3 paratope directed against an epitope located within CD3δ (SEQ ID NO: 256), CD3ε (SEQ ID NO: 257) or CD3δ and CD3ε,
(ii.) at least one linker, wherein the linker is covalently linked to the C-terminus of the peptide; and
(iii.) at least one half-life extending polymer of preferably at least 2 kDa, wherein the polymer is covalently linked to the linker.

According to said aspect, it is also envisaged that the binding peptide binds to an anti-CD3 paratope of a TCE, wherein said anti-CD3 paratope binds to an epitope comprising at least one residue selected from CD3ε (SEQ ID NO: 257): K73 and S83; and CD3δ (SEQ ID NO: 256) K82 and C93, wherein the epitope preferably comprises the region of CD3ε defined by K73, N74, 175, G76, S77, D78, E79, D80, H81, L82, and S83, and wherein the epitope comprises the region of CD3δ defined by K82, E83, S84, T85, V86, Q87, V88, H89, Y90, R91, M92, and C93.

In another aspect of this disclosure, A TCE masking molecule comprising:
(i) a binding peptide that binds to an anti-CD3 epsilon (CD3ε) paratope of a CD3ε binding domain of a TCE, wherein the anti-CD3ε paratope is directed against an epitope located within CD3ε, wherein CD3ε comprises the amino acid sequence of SEQ ID NO: 257;
(ii) a linker covalently linked to the C-terminus of the binding peptide; and
(iii) a half-life extending polymer covalently linked to the linker.

According to said aspect, it is also envisaged that the anti-CD3ε paratope is directed to a CD3ε epitope consisting of the amino acid sequence of SEQ ID NO: 258 or consisting of a shorter N-terminal sequence of SEQ ID NO: 258, preferably the amino acid sequence of SEQ ID NO: 259 or QDGNEE or QDGNEEM or QDGNEEMG.

According to said aspect, it is also envisaged that the binding peptide comprises 5, 6, 7, 8, 9, 10, 11, 12, 13, 14, 15, 16, 17 or 18 amino acids, preferably 5, 6, 7, 8, 9 or 10 amino acids, more preferably 10 amino acids.

According to said aspect, it is also envisaged that the binding peptide binds to an anti-CD3ε paratope and comprises at the N-terminus at least the sequence of X1DGX2E (SEQ ID NO: 260), wherein X1 is selected from Q, pyroglutamic acid (pE) and S, and wherein X2 is selected from N, E, S, T, V, and I.

According to said aspect, it is also envisaged that the binding peptide binds to an anti-CD3ε paratope and comprises at the N-terminus at least the sequence of X1X2X3X4EX5 (SEQ ID NO: 392), wherein X1 is Q, pyroglutamic acid (pE), or S, and wherein X2 is D, H or N, and wherein X3 is G, F or Y, and wherein X4 is N, E, S, T, V, or I, and wherein X5 is E, L, P, or W.

According to said aspect, it is also envisaged that the binding peptide binds to an anti-CD3ε paratope and comprises at the N-terminus at least the sequence of X1DGX2EE (SEQ ID NO: 261), wherein X1 is Q, pE, or S, and wherein X2 is N, E, S, T, V, or I.

According to said aspect, it is also envisaged that the binding peptide binds to an anti-CD3ε paratope and comprises at the N-terminus at least the sequence of XJX2X3X4EX5X6X7 (SEQ ID NO: 393), wherein X1 is Q, pyroglutamic acid (pE), or S, and wherein X2 is D, H or N. and wherein X3 is G, F or Y, and wherein X4 is N, E, S, T, V, or I, and wherein X5 is E, L, P, or W, and wherein X6 is A, C, D, E, F, G, H, I, K, L, M, N, P, Q, R, S, T, V, W, or Y, and wherein X7 is A, C, D, E, F, G, H, I, K, L, M, N, P, Q, R, S, T, V, W, or Y, preferably pEX2X3X4EEX6X7, even more preferably pEDGX4EEX6X7.

According to said aspect, it is also envisaged that the binding peptide binds to an anti-CD3ε paratope and comprises at the N-terminus at least the sequence of pEX1X2X3EX4LK (SEQ ID NO: 396), wherein X1 is D, H or N, and wherein X2 is G, F or Y. and wherein X3 is N, E, S, T, V, or I, and wherein X4 is E, L, P, or W.

According to said aspect, it is also envisaged that the anti-CD3ε binding peptide comprises a palindrome.

According to said aspect, it is also envisaged that the palindrome has a K at the position between the mirrored amino acids.

According to said aspect, it is also envisaged that the binding peptide binds to an anti-CD3ε paratope and comprises at the N-terminus at least the sequence of any one of SEQ ID NO: 263 to 284, 286 to 293, 295 to 303, 308 to 338, 385 to 388, 397 to 419 and 431 or any of 263 to 284, 286 to 293, 295 to 338, 385 to 388, 397 to 419 and 431.

According to said aspect, it is also envisaged that the binding peptide at its C-terminus further comprises the amino acids G and C or G and K..

According to said aspect, it is also envisaged that the binding peptide binds to an anti-CD3ε paratope and comprises the sequence of pEX1X2X3EX4X5X6GX7 (SEQ ID NO: 433), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, and wherein X4 is E, L, P, or W, and wherein X5 is A, C, D, E, F, G, H, I, K, L, M, N, P, Q, R, S, T, V, W, or Y, and wherein X6 is A, C, D, E, F, G, H, I, K, L, M, N, P, Q, R, S, T, V, W, or Y, and wherein X7 is C or K, preferably pEX1X2X3EEX5X6GX7, more preferably pEDGX3EEX5X6GX7.

According to said aspect, it is also envisaged that the binding peptide binds to an anti-CD3ε paratope and comprises the sequence of pEX1X2X3EELKGX4 (SEQ ID NO: 434), wherein X1 is D, H or N, and wherein X2 is G, F or Y, preferably G or F, wherein X3 is N, H, Q, R, E, S, T, V, C, D, F, K L, M, W, Y or I, preferably N, E, H, Q or R, most preferably N or E, and wherein X4 is C or K.

According to said aspect, it is also envisaged that the binding peptide binds to an anti-CD3ε paratope and comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably E, N, H, Q or R, most preferably N or E, and wherein X3 is C or K.

According to said aspect, it is also envisaged that the binding peptide binds to an anti-CD3ε paratope and comprises the sequence of pEDGX1EELKGX2 (SEQ ID NO: 436), wherein X1 is N, H, Q. R, E, S, T, V, C, D, F, K L, M, W, Y or I, preferably N, E, H, Q or R, most preferably N or E, and wherein X2 is C or K.

According to said aspect, it is also envisaged that the binding peptide binds to an anti-CD3ε paratope and comprises the sequence of SEQ ID NO: 297, 300, 303, 304, 305, 306, 307, 384 or 389.

According to said aspect, it is also envisaged that the linker is selected from the group consisting of (i.) disulfide linkers comprising disulfide (R—S—S—R′) wherein R is a half-life extending polymer and R′ is a binding peptide;, (ii.) free thiol containing linear or branched PEGs, OPSS derivatives, maleimides, nor-bornenes, acrylimides, acrylates, vinyl sulfones, and amines and (iii.) carboxyl linkers comprising amines, amides, and epsilon derivatized acetyl bromides.

According to said aspect, it is also envisaged that the linker is selected from the following moieties:

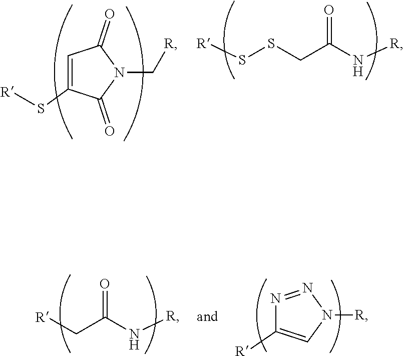

wherein R is a half-life extending polymer and R′ is a CD3ε masking peptide.

According to said aspect, it is also envisaged that the linker is selected from a maleimide-thiosuccinimide linker having the formula

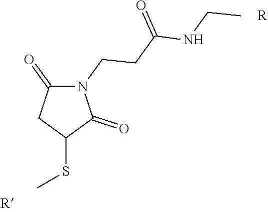

when the C-terminal amino acid of the binding peptide is K, and an acetamie—thioether having the formula

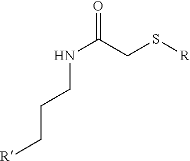

when the C-terminal amino acid of the binding peptide is C, and a disulfide having the formula R′—S—S—R when the C-terminal amino acid of the binding peptide is C, wherein R in the context of a linker herein is always a half-life extending polymer and R′ in the context of a liker herein is always a binding peptide.

According to said aspect, it is also envisaged that the half-life extending polymer is selected from mono-methoxy polyethylene glycol (mPEG), linear, 2-arm, 4-arm, 8-arm polyethylene glycol (PEG).: PLGA; peptide acrylate, polyglycerols, polyoxazolines, polyvinylpyrrolidone, polyacrylamides, poly(N-acryloylmorpholine), poly(N,N-dimethylacrylamide), poly(2-hydroxypropylmethacrylamide), polysarcosine, poly(2-hydroxyethylmethacrylamide), hyaluronic acids, sialic acid, poly[(organo)phosphazenes], and heparin.

According to said aspect, it is also envisaged that the half-life extending polymer is a PEG selected from the following moieties:

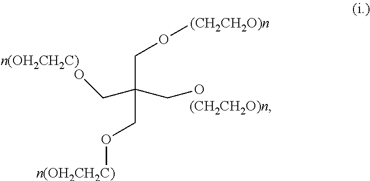

wherein n equals an integer of about 20 to about 200, preferably 20 to 169, more preferably about 20, 30, 40, 50, 60, 70, 80, 90 100, 103, 110 or 113,

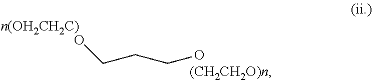

wherein n equals an integer of about 40 to about 400, preferably about 40 to about 208 or about 57 to about 338, more preferably about 50, 57, 60, 70, 80, 90, 100, 110, 120, 130, 140, 150, 160, 170, 180, 190, 200, 210, 220, 230 or about 225, and
(iii.) (CH₂CH₂O)n, wherein n equals an integer of about 80 to about 700, preferably about 80 to about 675, more preferably about 80, 83, 90, 100, 110, 113, 120, 130, 140, 150, 160, 166, 170, 180, 190, 200, 400, 410, 417, 420, 430, 440, 450, 460 or about 470.

According to said aspect, it is also envisaged that the half-life extending polymer is a non-branched linear, or a branched 2-arm, 4-arm, or 8-arm PEG, preferably linear or 4-arm, with a molecular weight of about 2 kDa to about 60 kDa, preferably about 4 kDa to about 30 kDa or 10 kDa to about 30 kDa, or more preferably about 5 or about 20 kDa.

According to said aspect, it is also envisaged that the half-life extending polymer is a branched polymer, preferably a, 2-arm, 4-arm and/or 8-arm PEG with am molecular weight of about 2 to 60 kDa to which more than one binding peptide is linked via a linker each, preferably 2, 4 or 8 binding peptides to one half-life extending polymer.

According to said aspect, it is also envisaged that the binding peptide comprises the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, most preferably N or E, and
(a.) wherein X4 is C; a linker chosen from maleimide-thiosuccinimide linker having the formula

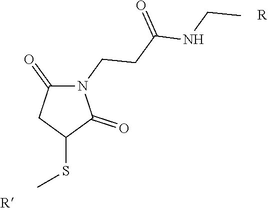

and a disulfide having the formula R′—S—S—R; and a half-life extending polymer that is a PEG of the following formula

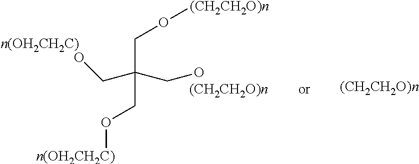

having a molecular weight of about 5 to 20 kDa, preferably about 5 kDa or about 10 kDa or about 20 kDa; or (b.) and wherein X4 is K; an acetamide-thioether having the formula

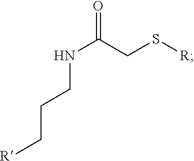

and a half-life extending polymer that is a PEG of the following formula

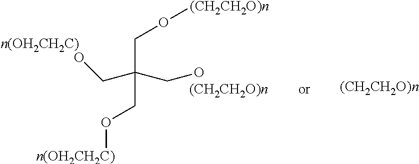

having a molecular weight of about 5 to 20 kDa, preferably about 5 kDa or about 10 kDa or about 20 kDa.

According to said aspect, it is also envisaged that the binding peptide comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably E, N, H, Q or R, most preferably N or E, and
(a.) wherein X3 is C; a linker chosen from maleimide-thiosuccinimide linker having the formula

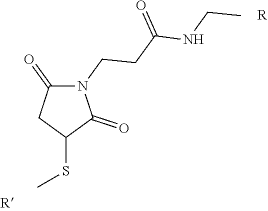

and a disulfide having the formula R′—S—S—R; and a half-life extending polymer that is a PEG of the following formula

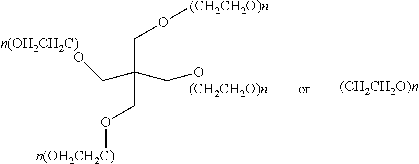

having a molecular weight of about 5 to 20 kDa, preferably about 5 kDa or about 10 kDa or about 20 kDa; or (b.) and wherein X3 is K; an acetamide-thioether having the formula

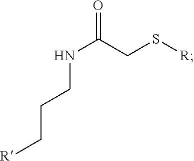

and a half-life extending polymer that is a PEG of the following formula

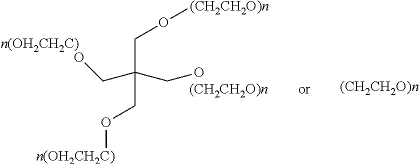

having a molecular weight of about 5 kDa to about 20 kDa, preferably about 5 kDa or about 10 kDa, or about 20 kDa, wherein R′ stands for the binding peptide and R stands for the half-life extending PEG polymer.

According to said aspect, it is also envisaged that comprising the sequence of pEDGX1EELKGX2 (SEQ ID NO: 436), wherein X1 is N or E, and wherein (a.) X2 is C; a linker chosen from maleimide-thiosuccinimide linker having the formula

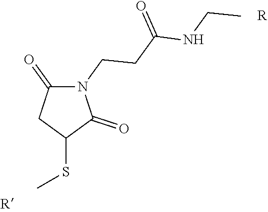

and a disulfide having the formula R′—S—S—R; and a half-life extending polymer that is a PEG of the following formula

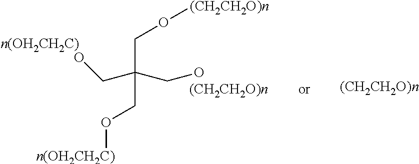

having a molecular weight of about 5 kDa or about 20 kDa; or (b) the sequence of pEDGX1EELKGX2 (SEQ ID NO: 436), wherein X1 is N or E. and wherein X2 is K; an acetamide-thioether having the formula

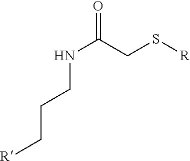

and a half-life extending polymer that is a PEG of the following formula

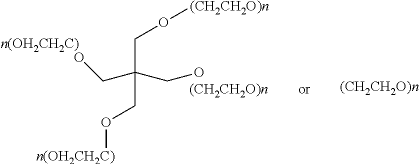

having a molecular weight of about 5 kDa to about 20 kDa, preferably about 5 kDa or about 10 kDa or about 20 kDa, R′ stands for the binding peptide and R stands for the half-life extending PEG polymer.

According to said aspect, it is also envisaged that the masking molecule comprises a T cell engager masking molecule comprising:
(i.) a binding peptide binds to an anti-CD3ε paratope and comprises the amino acid sequence of any one of SEQ ID NO: 263 to 284, 286 to 293, 295 to 303, 304 to 307, 308 to 338, 385 to 388, 397 to 419 and 431.;
(ii.) a linker, wherein the linker is covalently linked to the C-terminus of the binding peptide; and
(iii.) at least one half-life extending polymer of preferably at least 2 kDa, wherein the polymer is covalently linked to the linker.

According to said aspect, it is also envisaged that the masking molecule comprises a T cell engager masking molecule comprising:
(a.) the amino acid sequence of SEQ ID NO: 304, 306, 384 or 389, and
a linker chosen from maleimide-thiosuccinimide linker having the formula

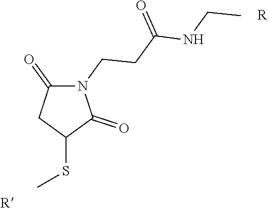

and a disulfide having the formula R′—S—S—R;
   and a half-life extending polymer that is a PEG of the following formula

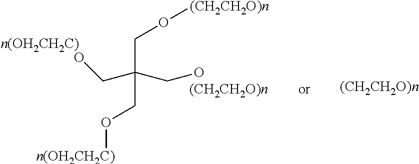

having a molecular weight of about 5 kDa to about 20 kDa, preferably about 5 kDa or about 10 kDa or about 20 kDa or
(b) the amino acid sequence of SEQ ID 305 or 307;

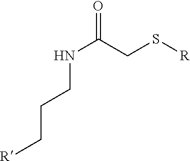

an acetamide-thioether having the formula
   and a half-life extending polymer that is a PEG of the following formula

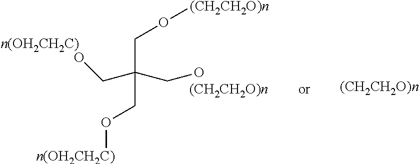

having a molecular weight of about 5 kDa to about 20 kDa, preferably about 5 kDa or about 10 kDa or about 20 kDa, wherein R′ stands for the binding peptide and R stands for the half-life extending PEG polymer.

According to said aspect, it is also envisaged that the masking molecule comprises a T cell engager masking molecule comprising:
(i.) a binding peptide having SEQ ID NO: 304;
(ii.) maleimide-thiosuccinimide linker having the formula

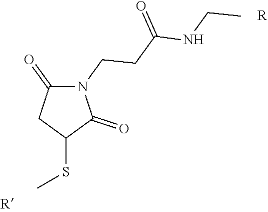

or an disulfide R′—S—S—R
covalently linked to the C-terminus of the binding peptide; and
(iii.) a half-life extending polymer of the following formula

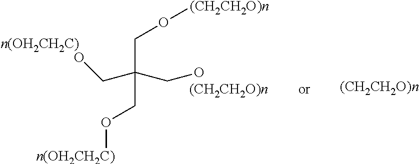

having a molecular weight of 5 kDa or 20 Da.

According to said aspect, it is also envisaged that the masking molecule comprises a T cell engager masking molecule comprising:
(i.) a binding peptide having SEQ ID 305;
(ii.) an acetamides-thioether having the formula

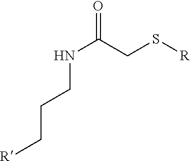

covalently linked to the C-terminus of the binding peptide; and
(iii.) a half-life extending polymer of the following formula

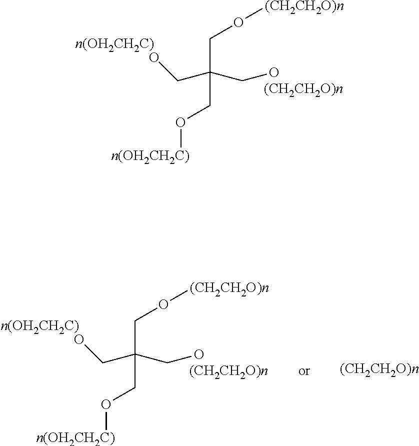

having a molecular weight of 5 kDa or 20 Da.

According to said aspect, it is also envisaged that the masking molecule comprises a T cell engager masking molecule comprising:
(i.) a binding peptide having SEQ ID 306;
(ii.) a maleimide-thiosuccinimide linker having the formula

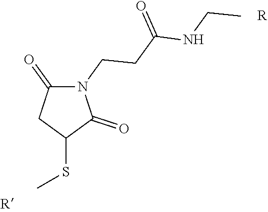

or a disulfide R′—S—S—R
covalently linked to the C-terminus of the binding peptide; and
(iii.) a half-life extending polymer of the following formula

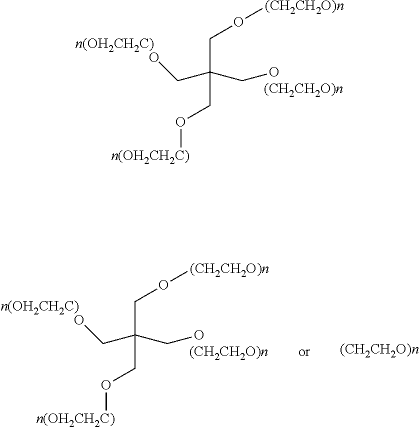

having a molecular weight of 5 kDa or 20 Da.

According to said aspect, it is also envisaged that the masking molecule comprises a T cell engager masking molecule comprising:
(i.) a binding peptide having SEQ ID 307;
(ii.) a acetamides-thioether having the formula

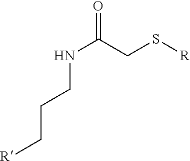

covalently linked to the C-terminus of the binding peptide; and
(iii.) a half-life extending polymer of the following formula

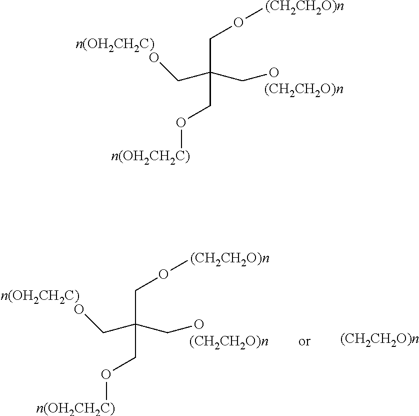

having a molecular weight of 5 kDa or 20 Da.

According to said aspect, it is also envisaged that the TCE masking molecule comprises any of the combinations (a.) to (k.) of a binding peptide, a linker and a half-life extending polymer, wherein R′ stands for the binding peptide and R stands for the half-life extending polymer in following table 1:

TABLE 1
Composition of exemplary TCE masking molecules

| | Peptide seq | Linker | Polymer |
|---|---|---|---|
| (a.) | pEDGNEELKGC | Thiosuccinimide | PLGA of any of about 5, |
| | (SEQ ID NO 304) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (b.) | pEDGNEELKGC | Thiosuccinimide | PLGA of any of about 5, |
| | (SEQ ID NO 304) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (c.) | pEDGEEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (d.) | pEDGNEELKGK | Thioether | PLGA of any of about 5, |
| | (SEQ ID NO 305) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (e.) | pEDGEEELKGK | Thioether | PLGA of any of about 5, |
| | (SEQ ID NO 307) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (f.) | pEDGNEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (g.) | pEDGEEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (h.) | pEDGNEELKGK | Thioether | PLGA of any of about 5, 10, 20, |
| | (SEQ ID NO 305) | | 30, 40, or 50 kDa or PEG linear |
| | | | of any of 5, 10 or 20 kDa; or |
| | | | PEG 4-arm |
| | | | of any or 5, 10 or 20 kDa |
| (i.) | pEDGEEELKGK | Thioether | PLGA of any of about 5, 10, 20, |
| | (SEQ ID NO 307) | | 30, 40, or 50 kDa or PEG linear |
| | | | of any of 5, 10 or 20 kDa; or |
| | | | PEG 4-arm |
| | | | of any or 5, 10 or 20 kDa |
| (j.) | pEDGNEELKGK | Thioether | PLGA of any of about 5, 10, 20, |
| | (SEQ ID NO 305) | | 30, 40, or 50 kDa or PEG linear |
| | | | of any of 5, 10 or 20 kDa; or |
| | | | PEG 4-arm |
| | | | of any or 5, 10 or 20 kDa |
| (k.) | pEDGEEELKGK | Thioether | PLGA of any of about 5, 10, 20, |
| | (SEQ ID NO 307) | | 30, 40, or 50 kDa or PEG linear |
| | | | of any of 5, 10 or 20 kDa; or |
| | | | PEG 4-arm |
| | | | of any or 5, 10 or 20 kDa |

According to said aspect, it is also envisaged that the TCE masking molecule comprises any of the combinations (a.) to (k.) of a binding peptide, a linker and a half-life extending PEG polymer, wherein R′ stands for the binding peptide and R stands for the half-life extending PEG polymer.

TABLE 2
Composition of exemplary TCE masking molecules with structural formula

| | Peptide seq | Linker | PEG configuration |
|---|---|---|---|
| (a.) | pEDGNEELKGC (SEQ ID NO 304) | 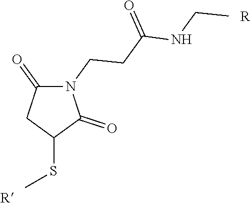 | 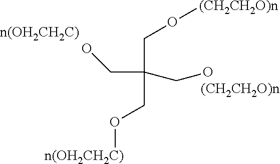 4 arm, 20 kDa |
| | | Thiosuccinimide | |
| (b.) | pEDGNEELKGC (SEQ ID NO 304) | 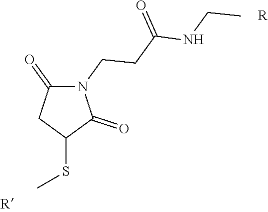 | 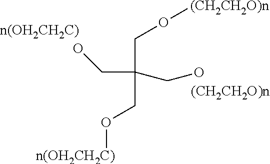 4 arm, 20 kDa |
| | | Thiosuccinimide | |
| (c.) | pEDGEEELKGC (SEQ ID NO 306) | 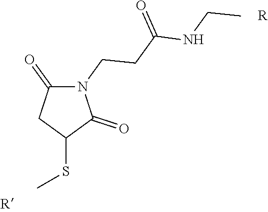 | 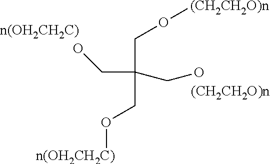 4 arm, 20 kDa |
| | | Thiosuccinimide | |
| (d.) | pEDGNEELKGK (SEQ ID NO 305) | 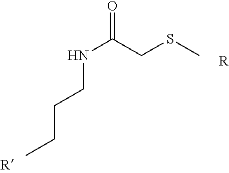 | 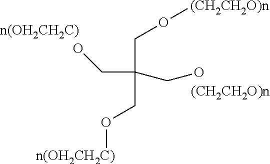 |
| | | Thioether | 4 arm, 20 kDa |
| (e.) | pEDGEEELKGK (SEQ ID NO 307) | 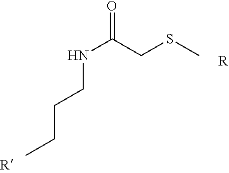 | 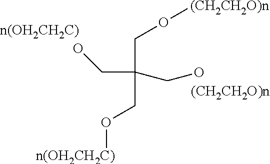 |
| | | Thioether | 4 arm, 20 kDa |
| (f.) | pEDGNEELKGC (SEQ ID NO 306) | 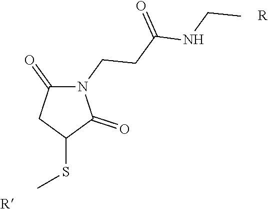 | 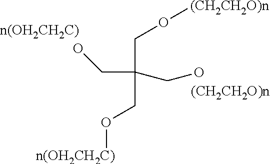 4 arm, 5 kDa |
| | | Thiosuccinimide | |
| (g.) | pEDGEEELKGC (SEQ ID NO 306) | 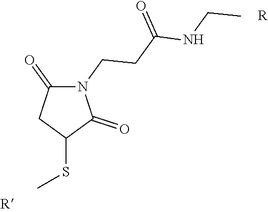 | 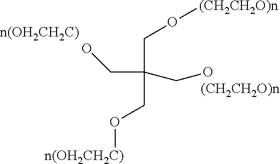 4 arm, 5 kDa |
| | | Thiosuccinimide | |
| (h.) | pEDGNEELKGK (SEQ ID NO 305) | 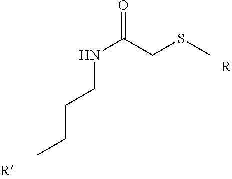 | (CH₂CH₂O)n Linear, 20 kDa |
| | | Thioether | |
| (i.) | pEDGEEELKGK (SEQ ID NO 307) | 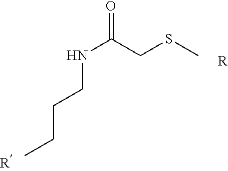 | (CH₂CH₂O)n Linear, 20 kDa |
| | | Thioether | |
| (j.) | pEDGNEELKGK (SEQ ID NO 305) | 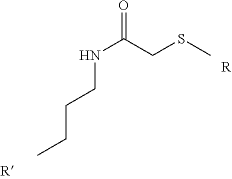 | (CH₂CH₂O)n Linear, 10 kDa |
| | | Thioether | |
| (k.) | pEDGEEELKGK (SEQ ID NO 307) | 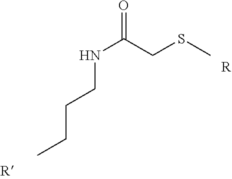 | (CH₂CH₂O)n Linear, 10 kDa |
| | | Thioether | |

According to said aspect, it is also envisaged that the TCE masking molecule has a half-life significantly shorter half-life than the TCE of about 1 to about 48 hours, preferably about 1.5 to about 24 hours, more preferably about 1.5 to about 4 hours or about 2 hours.

According to said aspect, the binding peptide binds to an anti-CD3ε paratope of a CD3ε binding domain of a TCE with an affinity in terms of a Kd value of typically less than 10 nM or less than 5 nM or less than 0.5 nM or about 0.1 nM to 5 nM. Preferably, this can be achieved if the amino acid sequence of the binding peptide is, e.g., pEX1GX2EELKGX3 (SEQ ID NO: 435), wherein X1 is D, H or N, and wherein X2 is N.

According to said aspect, the binding peptide binds to an anti-CD3ε paratope of a CD3ε binding domain of aTCE with an affinity of typically about 10 to 100 nM if the amino acid sequence of the binding peptide is pEX1GX2EELKGX3 (SEQ ID NO: 435), wherein X1 is D, H or N, and wherein X2 is E.

According to another aspect of the invention, it is also envisaged to provide a method for reducing the severity of cytokine release or cytokine release syndrome in a human subject undergoing treatment with a TCE, the method comprising administering to the human subject an effective dose of a T cell engager masking molecule, wherein the TCE masking molecule comprises:
(i.) a binding peptide that binds an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii.) a linker, wherein the linker is covalently linked to the C-terminus of the binding peptide; and
(iii.) at least one half-life extending polymer of preferably at least 2 kDa, wherein the polymer is covalently linked to the linker.

According to said aspect the T cell engager masking molecule comprises:
a binding peptide which binds to an anti-CD3ε paratope and comprises the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, and (a.) wherein X4 is C; a linker chosen from maleimide-thiosuccinimide linker having the formula

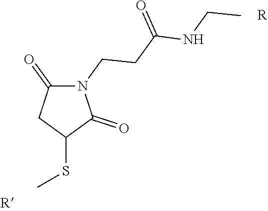

and a disulfide having the formula R′—S—S—R; and a half-life extending polymer that is a PEG of the following formula

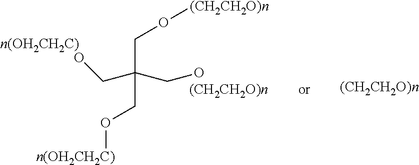

having a molecular weight of about 5 to 20 kDa, preferably about 5 kDa or about 10 kDa or about 20 kDa; or (b.) wherein X4 is K; an acetamide-thioether having the formula

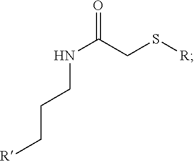

and a half-life extending polymer that is a PEG of the following formula

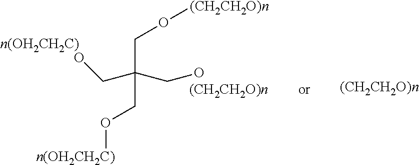

having a molecular weight of about 5 to 20 kDa, preferably about 5 kDa or about 10 kDa or about 20 kDa, wherein R′ stands for the binding peptide and R stands for the half-life extending PEG polymer.

According to said aspect the T cell engager masking molecule comprises:
a binding peptide which binds to an anti-CD3ε paratope and comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably N, H, Q or R, and (a.) wherein X3 is C; a linker chosen from maleimide-thiosuccinimide linker having the formula

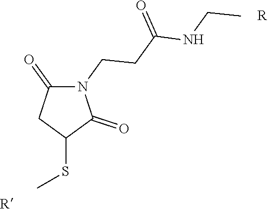

and a disulfide having the formula R′—S—S—R; and a half-life extending polymer that is a PEG of the following formula

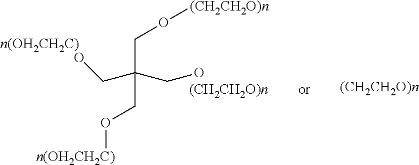

having a molecular weight of about 5 to 20 kDa, preferably about 5 kDa or about 10 kDa or about 20 kDa; or (b.) and wherein X3 is K; an acetamide-thioether having the formula

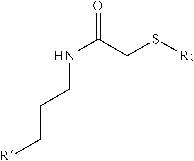

and a half-life extending polymer that is a PEG of the following formula

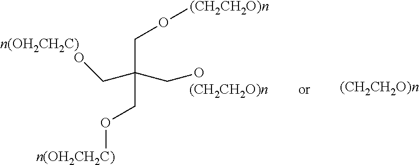

having a molecular weight of about 5 to 20 kDa, preferably about 5 kDa or about 10 kDa or about 20 kDa, wherein R′ stands for the binding peptide and R stands for the half-life extending PEG polymer.

According to said aspect the T cell engager masking molecule comprises:
a binding peptide which binds to an anti-CD3ε paratope and comprises the sequence of pEDGX1EELKGX2 (SEQ ID NO: 436), wherein X1 is D, H or N, and wherein X1 is N, H, Q, R, E, S, T, V, C, D, F, K L, M, W, Y or I, preferably N, E, H, Q or R, most preferably N or E, and (a.) wherein X2 is C; a linker chosen from maleimide-thiosuccinimide linker having the formula

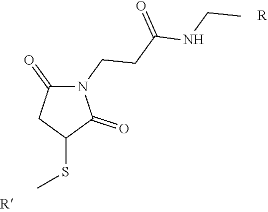

and a disulfide having the formula R′—S—S—R; and a half-life extending polymer that is a PEG of the following formula

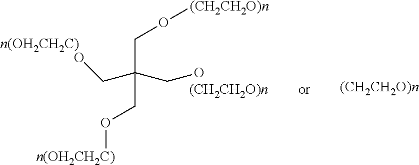

having a molecular weight of about 5 to 20 kDa, preferably about 5 kDa or about 10 kDa or about 20 kDa; or (b.) and wherein X2 is K; an acetamide-thioether having the formula

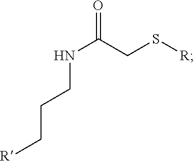

and a half-life extending polymer that is a PEG of the following formula

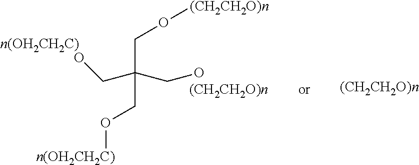

having a molecular weight of about 5 to 20 kDa, preferably about 5 kDa or about 10 kDa or about 20 kDa, wherein R′ stands for the binding peptide and R stands for the half-life extending PEG polymer.

According to said aspect the T cell engager masking molecule comprises:
(a) the amino acid sequence of SEQ ID NO: 304, 306, 384 or 389, and
a linker chosen from maleimide-thiosuccinimide linker having the formula

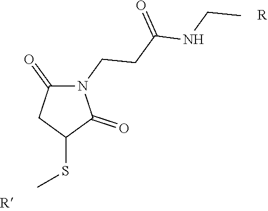

and a disulfide having the formula R′—S—S—R;
and a half-life extending polymer that is a PEG of the following formula

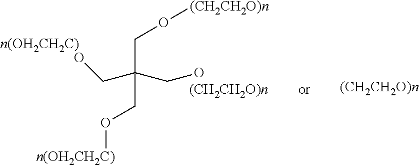

having a molecular weight of about 5 kDa to about 20 kDa, preferably about 5 kDa or about 10 kDa or about 20 kDa; or
(b) the amino acid sequence of SEQ ID 305 or 307;
an acetamide-thioether having the formula

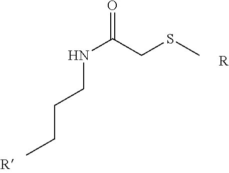

and a half-life extending polymer that is a PEG of the following formula

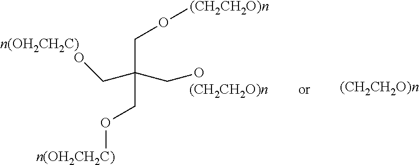

having a molecular weight of about 5 kDa to about 20 kDa, preferably about 5 kDa or about 10 kDa or about 20 kDa, wherein R′ stands for the binding peptide and R stands for the half-life extending PEG polymer.

According to said aspect, it is also envisaged that the method of reducing the severity of cytokine release in a human subject undergoing preferably treatment with a TCE comprises use or administration of the TCE masking molecule comprises any of the combinations (a.) to (k.) of a binding peptide, a linker and a half-life extending polymer, wherein R′ stands for the binding peptide and R stands for the half-life extending polymer:

| | Peptide seq | Linker | Polymer |
|---|---|---|---|
| (a.) | pEDGNEELKGC | Thiosuccinimide | PLGA of any of about 5, |
| | (SEQ ID NO 304) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (b.) | pEDGNEELKGC | Thiosuccinimide | PLGA of any of about 5, |
| | (SEQ ID NO 304) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (c.) | pEDGEEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (d.) | pEDGNEELKGK | Thioether | PLGA of any of about 5, |
| | (SEQ ID NO 305) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (e.) | pEDGEEELKGK | Thioether | PLGA of any of about 5, |
| | (SEQ ID NO 307) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (f.) | pEDGNEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (g.) | pEDGEEELKGC | Thiosuccinimide | PLGA of any of about 5, |
| | (SEQ ID NO 306) | | 10, 20, 30, 40, or 50 kDa or |
| | | | PEG linear of any of 5, 10 or |
| | | | 20 kDa; or PEG 4-arm |
| | | | of any or 5, 10 or 20 kDa |
| (h.) | pEDGNEELKGK | Thioether | PLGA of any of about 5, 10, 20, |
| | (SEQ ID NO 305) | | 30, 40, or 50 kDa or PEG linear |
| | | | of any of 5, 10 or 20 kDa; or |
| | | | PEG 4-arm |
| | | | of any or 5, 10 or 20 kDa |
| (i.) | pEDGEEELKGK | Thioether | PLGA of any of about 5, 10, 20, |
| | (SEQ ID NO 307) | | 30, 40, or 50 kDa or PEG linear |
| | | | of any of 5, 10 or 20 kDa; or |
| | | | PEG 4-arm |
| | | | of any or 5, 10 or 20 kDa |
| (j.) | pEDGNEELKGK | Thioether | PLGA of any of about 5, 10, 20, |
| | (SEQ ID NO 305) | | 30, 40, or 50 kDa or PEG linear |
| | | | of any of 5, 10 or 20 kDa; or |
| | | | PEG 4-arm |
| | | | of any or 5, 10 or 20 kDa |
| (k.) | pEDGEEELKGK | Thioether | PLGA of any of about 5, 10, 20, |
| | (SEQ ID NO 307) | | 30, 40, or 50 kDa or PEG linear |
| | | | of any of 5, 10 or 20 kDa; or |
| | | | PEG 4-arm |
| | | | of any or 5, 10 or 20 kDa |

According to said aspect, the method of reducing the severity of cytokine release in a human subject undergoing preferably treatment with a TCE comprises use or administration of any of the combinations (a.) to (k.) of a binding peptide, a linker and a half-life extending PEG polymer, wherein R′ stands for the binding peptide and R stands for the half-life extending PEG polymer:

| | Peptide seq | Linker | PEG configuration |
|---|---|---|---|
| (a.) | pEDGNEELKGC (SEQ ID NO 304) | 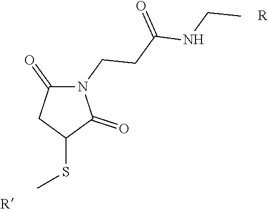 | 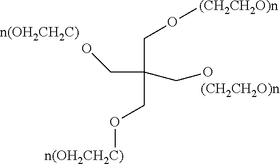 4 arm, 20 kDa |
| | | Thiosuccinimide | |
| (b.) | pEDGNEELKGC (SEQ ID NO 304) | 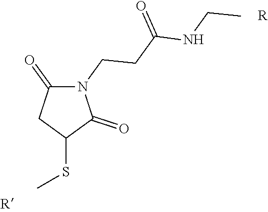 | 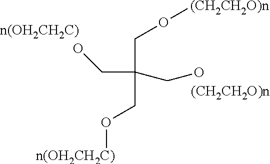 4 arm, 20 kDa |
| | | Thiosuccinimide | |
| (c.) | pEDGEEELKGC (SEQ ID NO 306) | 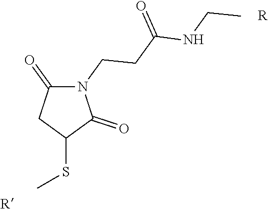 | 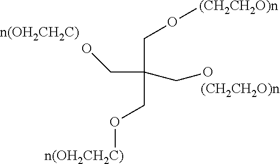 4 arm, 20 kDa |
| | | Thiosuccinimide | |
| (d.) | pEDGNEELKGK (SEQ ID NO 305) | 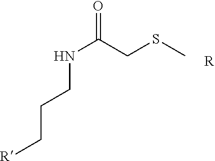 | 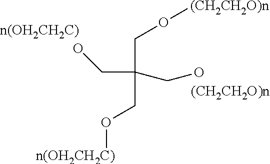 |
| | | Thioether | 4 arm, 20 kDA |
| (e.) | pEDGEEELKGK (SEQ ID NO 307) | 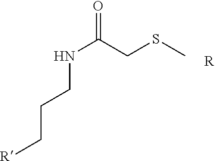 | 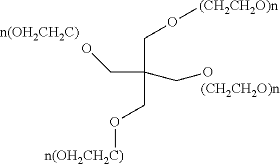 |
| | | Thioether | 4 arm, 20 kDA |
| (f.) | pEDGNEELKGC (SEQ ID NO 306) | 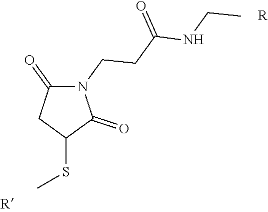 | 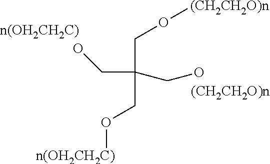 4 arm, 5 kDa |
| | | Thiosuccinimide | |
| (g.) | pEDGEEELKGC (SEQ ID NO 306) | 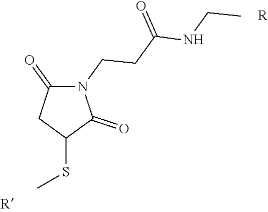 | 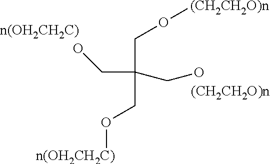 4 arm, 5 kDa |
| | | Thiosuccinimide | |
| (h.) | pEDGNEELKGK (SEQ ID NO 305) | 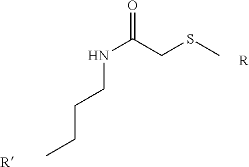 | (CH₂CH₂O)n Linear, 20 kDa |
| | | Thioether | |
| (i.) | pEDGEEELKGK (SEQ ID NO 307) | 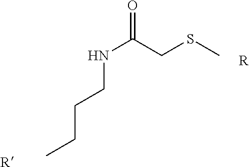 | (CH₂CH₂O)n Linear, 20 kDa |
| | | Thioether | |
| (j.) | pEDGNEELKGK (SEQ ID NO 305) | 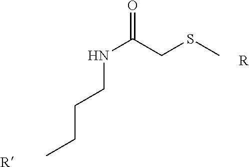 | (CH₂CH₂O]n Linear, 10 kDa |
| | | Thioether | |
| (k.) | pEDGEEELKGK (SEQ ID NO 307) | 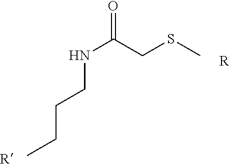 | (CH₂CH₂O]n Linear, 10 kDa |
| | | Thioether | |

According to said aspect the immunotherapy comprises administering a T cell engager.

According to said aspect the TCE masking molecule is administered before, during or after administration of a TCE.

According to said aspect, it is also envisaged that the molar ratio of masking molecule to TCE is in the range of 1000:1 to 10:1 or 250:1 to 10:1, preferably 100:1 to 25:1.

According to said aspect, it is also envisaged that the TCE is selected from
(ii.) A TCE comprising at least three domains in an amino to carboxyl order, wherein:
(a.) a first domain binds to a target cell surface antigen, which is preferably a tumor antigen;
(b.) a second domain binds to an extracellular epitope of the human and/or the Macaca CD3 chain, preferably CD3ε; and
(c.) a third domain comprises two polypeptide monomers, each comprising a hinge, a CH2 and a CH3 domain, wherein said two polypeptide monomers are fused to each other via a peptide linker, wherein said third domain comprises in an amino to carboxyl order:
   hinge-CH2-CH3-linker-hinge-CH2-CH3;
(ii.) A TCE comprising
(a.) a first binding domain which binds to a first target cell surface antigen (e.g. TAA1),
(b.) a second binding domain, which binds to an extracellular epitope of the CD3ε,
(c.) a spacer, wherein said spacer is a single chain fragment crystallizable (scFc), human serum albumin (HSA), programmed death receptor 1 (PD1), or a hetero fragment crystallizable (hetero FC);
(d.) a third binding domain, which binds to a second target cell surface antigen (e.g. TAA2), and
(e.) a fourth binding domain, which binds to an extracellular epitope of CD3ε,
wherein the first binding domain binds to the first target cell surface antigen and the third binding domain binds to the second target cell surface antigen simultaneously, wherein the first target cell surface antigen and the second target cell surface antigen are on the same target cell,
wherein the TCE is a single polypeptide chain, wherein the first target cell surface antigen and the second target cell surface antigen are not identical, and
wherein the first binding domain and the second binding domain form a first bispecific entity and the third and the fourth binding domain form a second bispecific entity, and
   wherein the spacer entity is positioned between the first and the second bispecific entities;
   and
(iii.) an IgG-based full length bispecific antibody; or
(iv.) a heterodimeric antibody.

According to said aspect, it is also envisaged that the TCE under [55](i.) or (ii.) is a single chain molecule.

According to said aspect, it is also envisaged that a glycosylation site at Kabat position 314 of the CH2 domains in the third domain of the bispecific antigen-binding molecule is removed by a N314X substitution, wherein X is any amino acid excluding Q.

According to said aspect, it is also envisaged that each of said polypeptide monomers of the third domain has an amino acid sequence that is at least about 80, 85, 90, 95 or 100% identical to a sequence selected from the group consisting of: SEQ ID NOs: 437 to 444, or has an amino acid sequence selected from the group consisting of SEQ ID NOs: 437 to 444.

According to said aspect, it is also envisaged that the CH2 domain comprises an intra domain cysteine disulfide bridge.

According to said aspect, it is also envisaged that the tumor antigen is selected from the group consisting of CDH19, CDH3, MSLN, DLL3, FLT3, EGFRvIII, BCMA, PSMA, CD33, CD19, CD20, CLDN18.2, CLDN 6, MUC17, EpCAM, STEAP1 and CD70.

According to said aspect, it is also envisaged that the antibody construct comprises in an amino to carboxyl order:
(a) the first domain;
(b) a peptide linker having an amino acid sequence selected from the group consisting of SEQ ID NOs: 187-189;
(c) the second domain;
(d) a peptide linker having an amino acid sequence selected from the group consisting of SEQ ID NOs: 187, 188, 189, 195, 196, 197 and 198;
(e) the first polypeptide monomer of the third domain having an amino acid sequence of any of SEQ ID NOs 437 to 444;
(f) a peptide linker having an amino acid sequence selected from the group consisting of SEQ ID NOs: 191, 192, 193 and 194; and
(g) the second polypeptide monomer of the third domain having an amino acid sequence of any of SEQ ID NOs 437 to 444.

According to said aspect, it is also envisaged that the first binding domain of the construct comprises a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 1, CDR-L2 as depicted in SEQ ID NO: 2 and CDR-L3 as depicted in SEQ ID NO: 3,
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36,
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47,
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58,
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70,
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88,
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99,
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111,
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120,
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131,
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142,
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157,
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172,
(n) CDR-H1 as depicted in SEQ ID NO: 203, CDR-H2 as depicted in SEQ ID NO: 204, CDR-H3 as depicted in SEQ ID NO: 205, CDR-L1 as depicted in SEQ ID NO: 206, CDR-L2 as depicted in SEQ ID NO: 207 and CDR-L3 as depicted in SEQ ID NO: 208;
(o) CDR-H1 as depicted in SEQ ID NO: 214, CDR-H2 as depicted in SEQ ID NO: 215, CDR-H3 as depicted in SEQ ID NO: 216, CDR-L1 as depicted in SEQ ID NO: 217, CDR-L2 as depicted in SEQ ID NO: 218 and CDR-L3 as depicted in SEQ ID NO: 219;
(p) CDR-H1 as depicted in SEQ ID NO: 226, CDR-H2 as depicted in SEQ ID NO: 227, CDR-H3 as depicted in SEQ ID NO: 228, CDR-L1 as depicted in SEQ ID NO: 229, CDR-L2 as depicted in SEQ ID NO: 230 and CDR-L3 as depicted in SEQ ID NO: 231;
(q) CDR-H1 as depicted in SEQ ID NO: 238, CDR-H2 as depicted in SEQ ID NO: 239, CDR-H3 as depicted in SEQ ID NO: 240, CDR-L1 as depicted in SEQ ID NO: 241, CDR-L2 as depicted in SEQ ID NO: 242 and CDR-L3 as depicted in SEQ ID NO: 243, and
(r) CDR-H1 as depicted in SEQ ID NO: 420, CDR-H2 as depicted in SEQ ID NO: 421, CDR-H3 as depicted in SEQ ID NO: 422, CDR-L1 as depicted in SEQ ID NO: 423, CDR-L2 as depicted in SEQ ID NO: 424 and CDR-L3 as depicted in SEQ ID NO: 425.

According to said aspect, it is also envisaged that the bispecific antigen-binding molecule has the SEQ ID NO: 17, 52, 63, 81, 93, 104, 114, 125, 136, 147, 162, 177, 213, 226, 238, 248, 255 or 430, preferably 104 or 255.

According to said aspect, it is also envisaged that the bispecific antigen-binding molecule is a heterodimeric antibody comprising:
a) a first monomer comprising a first heavy chain comprising:
   1) a first variable heavy domain;
   2) a first constant heavy chain comprising a first CH1 domain and a first Fc domain;
   3) a scFv that binds human CD3 and comprises a scFv variable light domain, an scFv linker and a scFv variable heavy domain; wherein said scFv is covalently attached between the C-terminus of said CH1 domain and the Nterminus of said first Fc domain using domain linker(s);
b) a second monomer comprising a second heavy chain comprising a second variable heavy domain and a second constant heavy chain comprising a second Fc domain; and
c) a common light chain comprising a variable light domain and a constant light domain;
wherein said first variable heavy domain and said variable light domain bind human STEAP1, said second variable heavy domain and said variable light domain bind human STEAP1, and wherein
a) the first monomer comprises the sequence of SEQ ID NO: 375, the second monomer comprises the sequence of SEQ ID NO: 374, and the common light chain comprises the sequence of SEQ ID NO: 373;
b) the first monomer comprises the sequence of SEQ ID NO: 380, the second monomer comprises the sequence of SEQ ID NO: 376, and the common light chain comprises the sequence of SEQ ID NO: 373; or
c) the first monomer comprises the sequence of SEQ ID NO: 379, the second monomer comprises the sequence of SEQ ID NO: 378, and the common light chain comprises the sequence of SEQ ID NO: 377.

According to said aspect the CD3ε binding domain of a TCE has the sequence of SEQ ID NO 26, 381, 382 or 383.

According to another aspect, a TCE masking molecule is provided for use in reducing the severity of cytokine release in a human subject undergoing treatment with a TCE, wherein the TCE masking molecule comprises:
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii) a linker covalently linked to the C-terminus of the binding peptide; and
(iii) a half-life extending polymer covalently linked to the linker.

According to another aspect, a method of attenuating the in vivo exposure of a TCE, wherein the administration is preferably via the i.v. or s.c. route, the method comprising the steps of
(a) providing a TCE masking molecule, comprising
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257,
(ii) a linker covalently linked to the C-terminus of the binding peptide; and
(iii) a half-life extending polymer covalently linked to the linker, wherein the binding peptide binds to an anti-CD3ε paratope with a Kd which is at least 1.5-fold lower than the Kd for the amino acid sequence of SEQ ID NO: 262 binding to an anti-CD3ε paratope, preferably, at least 2-fold lower, more preferably 3-fold lower, and
(b) administering the TCE masking molecule at a molar excess of about 10 to about 1000, or about 25 to about 250 with respect to the TCE. According to said aspect, the TCE masking molecule is administered before, during or after the administration of the TCE.

According to another aspect, a method for reducing the severity of cytokine release in a human subject undergoing immunotherapy is envisaged, the method comprising administering to the human subject an effective dose of a T cell engager masking molecule before, concurrently with, or after immunotherapy, wherein the T cell engager masking molecule comprises:
(i.) a binding peptide having SEQ ID selected from the group consisting of SEQ ID NOs 263 to 284, 286 to 293, 295 to 338, 385 to 388, 397 to 419 and 431;
(ii.) a linker, wherein the linker is covalently linked to the C-terminus of the binding peptide; and
(iii.) at least one half-life extending polymer of preferably at least 2 kDa, wherein the polymer is covalently linked to the linker.

According to another aspect, a method is also envisaged comprising administering to the subject in need of such treatment an effective dosage of the TCE masking molecule as disclosed herein or a pharmaceutically acceptable salt thereof, in combination with a therapeutically effective amount of a T cell engager in a molar ration preferably in the range of 1000:1 to 10:1 or 250:1 to 10:1 (masking molecule:TCE). TCE masking molecule is administered before, during or after the administration of the TCE.

According to another aspect, a TCE masking molecule is envisaged for use in the attenuation of in vivo exposure of a TCE after administration of the TCE molecule, wherein the administration is preferably via the i.v. or s.c. route, the method comprising the steps of
(a) providing a TCE masking molecule, comprising
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii) a linker covalently linked to the C-terminus of the binding peptide; and
(iii) a half-life extending polymer covalently linked to the linker,
wherein the binding peptide binds to an anti-CD3ε paratope with a Kd which is at least 1.5-fold lower than the Kd for the amino acid sequence of SEQ ID NO: 262 binding to an anti-CD3ε paratope, preferably, at least 2-fold lower, more preferably 3-fold lower, and
(b) administering the TCE masking molecule before, during or after the administration of the TCE at a molar excess of about 10 to about 1000, or about 25 to about 250 with respect to the TCE.

### BRIEF DESCRIPTION OF THE DRAWINGS

FIG. 1: FIG. 1 shows the general pharmacokinetic and pharmacodynamic relationship between TCE exposure and cytokine release. (Left) General PK profile for TCE administered by IV bolus and the relationship to cytokine release. The depiction here shows a TCE dosed to maintain minimal plasma concentrations above a pre-defined exposure target (EC90). This scenario often leads to patient overexposure that results in acute cytokine release at early times post-dose, which can induce runaway CRS. (Right) Alternative scenario where the active TCE is administered more gradually to reduce maximum exposure (Cmax) but maintaining TCE coverage above a desired effective concentration to ensure efficacy, but with mitigated cytokine stimulation.

FIG. 2: FIG. 2 shows exemplary in silico PK modeling highlighting the sensitivity of active, or ‘free’ TCE exposure to co-administration with masking molecule CD3ε PepPOL. (Left) In silico PK model schematic defining the relationship between plasma (Cp) and tissue (Cp) ‘free’ TCE concentrations, unbound CD3ε PepPOL (TI), and the binding kinetics driving TCE-(CD3ε PepPOL) complexation. Note, the elimination rate (kel,D) of TCE and TCE-(CD3ε PepPOL) is assumed to be identical. The elimination rate of CD3ε PepPOL, kel,T, is assumed >>than kel,D. (Right) Simulations (non-human primate PK parameters of CD20 HLE-BiTE) of TCE dosed by IV bolus (10 ug/kg) alone (black), or in combination with CD3ε PepPOL: equimolar dosing, KD=1 nM (orange), equimolar dosing, KD=0.1 nM (blue, solid); or 1000-fold molar excess dosing, KD=0.1 nM (blue dashed). Note the impact of both affinity and overage on both Cmax and tmax over the first ~2 days post dose, with very little perturbation of exposure thereafter.

FIG. 3: FIG. 3 shows transient association of exemplary masking molecule CD3ε PepPOL with exemplary TCE molecule with a half-life extending scFc moiety.

FIG. 4: FIG. 4 shows exemplary plasma concentration over time in C57BL/6 mice of a binding peptide only vs. masking molecule with half-life extending polymer (SEQ ID NO: 297).

FIG. 5: FIG. 5 shows PK impact of exemplary polymer on masking molecule in mice.

FIG. 6: FIG. 6 shows PK impact of maleamide linker in exemplary masking molecules.

FIG. 7: FIG. 7 shows TCE-mediated B-cell cytolytic effect.

FIG. 8: FIG. 8 shows reduction in cytokine release with CD3ε PepPol co-administration at >1000× Peptide:TCE overage.

FIG. 9: FIG. 9 shows co-administration of muCD19 TCE+CD3ε-PepPOL, v1 co-administration reduces cytokine release as a function of CD3ε-PepPOL, v1 dose.

FIG. 10: FIG. 10 shows co-administration of muCD19 TCE+CD3ε-PepPol, v1 reduces cytokine release with minimal impact on PD response in the huCD3ε KI model.

FIG. 11: FIG. 11 shows Co-administration of CD3ε-PepPOL, v1 reduces cytokine release with retention of anti-tumor activity in preclinical MC38-muCD19 tumor model. (A) Serum cytokine INF-y; (B) serum cytokine release TNF-α, (C) Target cell depletion in spleen and (D) in blood; (E) Tumor growth inhibition. Note: Statistical analysis performed using 1 way ANOVA for panels (A) and (B) and Repeated Measures 2way ANOVA for panel (C); GraphPad Prism v 9.5.1.

FIG. 12: FIG. 12 shows Co-administration of CD20 TCE+CD3ε-PepPOL, v1 uncouples cytokine release from PD response (CD20+ B cell lysis) in cynomolgus monkeys.

FIG. 13: FIG. 13 shows clinical chemistry parameters upon Co-administration of CD20 TCE+high affinity CD3ε-PepPOL (pGlu, 20 kDa).

FIG. 14: FIG. 14 shows Co-administration of CD20 TCE+high affinity CD3ε-PepPOL (pGlu, 20 kDa) reduces cytokine release without impacting PD effect in cynomolgus monkeys.

FIG. 15: FIG. 15 shows inhibition of cytotoxicity of heterodimeric STEAP1×CD3 TCE (comprising CD3e binder SEQ ID NO 381), CLDN6×CD3 TCE (comprising CD3e binder I2E, SEQ ID NO 383) and DLL3×CD3 TCE (comprising CD33 binder I2C, SEQ ID NO 26) by masking CD3e. CD3e pepPEG with high affinity against CD3e binder showed higher inhibition activity against TCE whereas peptides with intermediate affinity showed lower inhibition activity. All the peptides with high affinity showed similar potency with respect to each other. Masking molecules identified as 79674-1 (20 kDA 4-arm PEG and thiosuccinimide linker) and 79675-1 (5 kDa 4-arm PEG and thiosuccinimide linker) showed the highest inhibition activity in vitro.

FIG. 16 shows inhibition of cytokine release shows representative TCE masking molecules' inhibition of cytokine release induced by heterodimeric STEAP1×CD3ε TCE

FIG. 17 shows inhibition of cytokine release shows representative TCE masking molecules' inhibition of cytokine release induced by DLL3×CD3ε TCE.

FIG. 18 shows inhibition of cytokine release shows representative TCE masking molecules' inhibition of cytokine release induced by CLDN6×CD3ε TCE.

FIG. 19 shows affinity of representative binding peptides and one negative example on FLT3×CD3 TCE and STEAP1×CD3 TCE.

FIG. 20 shows inhibition of cytotoxicity on CDH3×MSLN dual targeting TCE

FIG. 21 shows inhibition of cytokine release on CDH3×MSN dual targeting TCE

### DETAILED DESCRIPTION

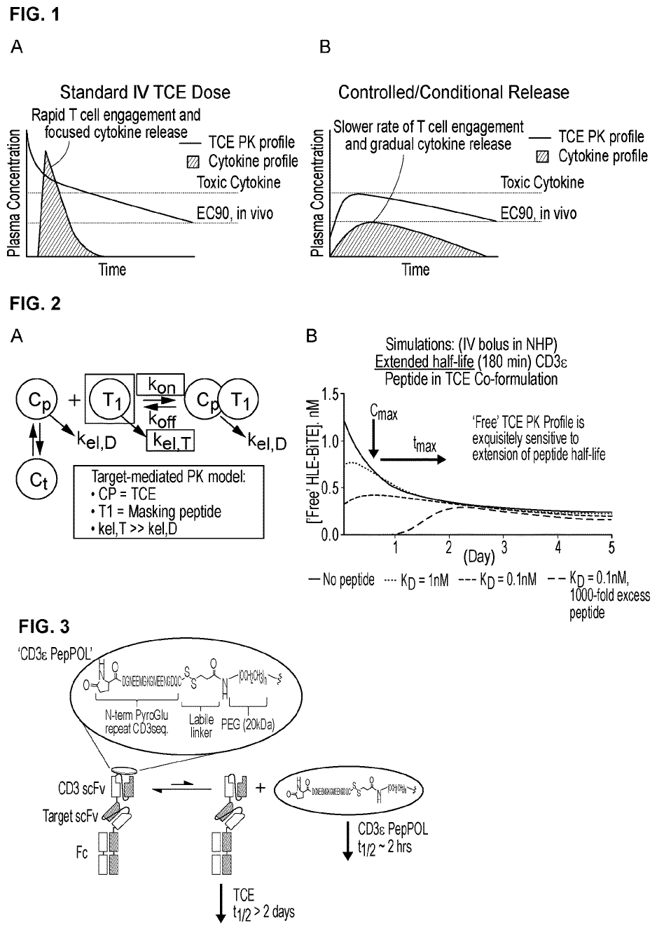

Without being limiting by theory, it is believed that pro-inflammatory cytokine release such as in CRS is a phenomenon rooted in sub-optimal TCE pharmacokinetics in the context of high TCE therapeutic potency. Here, it was surprisingly found that a comparable degree of control over CRS incidence can be obtained during standard IV bolus administration of TCE when combined with a masking molecule which transiently binds to a T-cell engaging paratope of a TCE and thereby transiently masks the TCE. The general principle of underlying pharmacokinetic and pharmacodynamic relationship between TCE exposure-non controlled and controlled, e.g., by a masking molecule- and cytokine release is depicted in FIG. 1. The masking molecule of the invention typically comprises a peptide which binds to the T-cell engaging paratope of the TCE, a linker and a half-life extending polymer. Preferred paratopes of CD3 binding domains of TCEs comprise those which bind to CD36, CD3ε, or both. However, in the context of this disclosure, CD3ε. The masking molecule may be abbreviated as CD3 PepPOL in the following. The abbreviation CD3ε PepPOL refers to a masking molecule which binds to a CD3ε binding paratope of a TCE. By combining CD3 PepPOL with TCE in defined molar proportion, a gradual onset PK profile of ‘free’ or ‘active’ TCE can be achieved without the inconveniences associated with multiple dosing steps or long IV infusion cycles requiring in-patient drug administration.

Some of the benefits of the present disclosure are (i.) to lessen the frequency of CRS incidence at any TCE dose level, (ii.) to aid clinical dosing schema by allowing for a higher initial dose of the TCE, and (iii.) to reduce the number of dose fractionation steps of the TCE required to achieve an efficacious dose thereby reducing therapeutic cycle time.

Advantageously, the masking molecules CD3 PepPOL if combined, e.g. administered before, during or after administration of a TCE, can achieve a more favorable TCE exposure profile than TCE administration without masking molecule, e.g. IV bolus or s.c. administration of TCE monotherapy. Without wanting to be bound by theory, this is due to a combination of high affinity/specificity binding of the CD3 PepPOL to the TCE anti-CD3ε engager moiety and the relatively short-lived CD3 PepPOL PK profile. Without wanting to be bound be theory, because the peptide binding moiety of CD3 PepPOL is derived from the respective CD3 domain such as CD3δ domain, CD3ε domain or a combination thereof of the T cell receptor (TCR) complex, it binds the anti-CD3 T cell engager moiety antagonistically with moderate to high affinity to prevent T cell engagement. Thus, as long as significant CD3 PepPOL remains in circulation relative to blood concentration of TCE, engagement with the immune system is typically prevented. Importantly, because CD3 PepPOL is typically cleared from the plasma quicker than the TCE, competitive blockade of TCE on T cells is transient, with the duration defined by CD3 PepPOL clearance (CL) and volume of distribution (V), as well as the amount of molar excess CD3 PepPOL administered. For illustration, when TCE exposure is monitored from the perspective of active, or ‘free’ therapeutic exposure (i.e., TCE exhibiting a full binding compliment to tumor antigen and TCR), a PK profile characterized by an attenuated maximum concentration (Cmax) with delayed time of maximal exposure (tmax) is anticipated. The general concept is illustrated via modeling and simulation in FIG. 2.

In general, binding peptides of the present disclosure are preferred which have a higher affinity in terms of a lower Kd value, preferably equilibrium Kd value, for the CD3ε paratope of the TCE to be masked than the physiological CD3ε epitope (e.g. a sequence comprising SEQ ID NO: 262, QDGNEEMG). Typically, Kd values of preferred binding peptides are at least 1.5 lower than, e.g., reference epitope QDGNEEMG (SEQ ID NO: 262), preferably at least 2-fold lower, more preferably 3-fold lower or even about 5-fold, about 10-fold or about 100-fold lower. For example, SEQ ID NO. 262 shows a binding affinity in terms of a Kd of 35.8 nM for a FLT3×CD3ε TCE (SEQ ID NO 93), see Example 1. Particularly preferred are binding peptides of, for example, any of SEQ ID NO: 300, 304 to 307, 384 and 389. A binding peptide with high affinity will typically allow a lower molar ratio of masing molecule:TCE and thus, a more economic employment of the masking molecule. Also p

Binding peptides are preferred which provide affinity gains, in particular by the substitution of N-terminal amino acid Q by pE. Affinity can further be fine-tuned, in particular, by the amino acid in position 4 in N to C orientation of a binding peptide according to the present invention. The amino acid N is typically associated with higher affinity and the amino acid E with slightly attenuated, i.e. intermediate affinity. Intermediate affinity can be of advantage to facilitate a relatively short time of masking the TCE. An important problem solved by the present invention is to mitigate cytokine release triggered by the TCE therapy. As explained herein and without wanting to be bound by theory, said cytokine release may be associated with initial high systemic exposure of TCE, e.g. in the blood plasma. Accordingly, it is sufficient to mask the TCE transiently to mitigate cytokine release on the onset of the TCE therapy. The masking time is typically driven by the affinity to the CD3ε paratope and the stability of the TCE masking molecule. In view thereof, a binding peptide with a half-life of less than about 24 hours, preferably about 12, 10, 8, 6, 4 or 2 hours is advantageous to mitigate initial high systemic exposure of TCE but do not reduce TCE exposure longer than required for initial cytokine release mitigation in order to ensure TCE on target activity. TCEs typically comprising a half-life extending domain such as an Fc-based domain typically have a half-life of more than 24 hours, preferably 2 days or more. Half-live of the binding peptide alone would be only a few minutes as shown in the Examples, which would be too short to effectively mitigate the initial TCE-mediated cytokine release. Half-life is typically understood herein as half-life in serum.

In general, all polymers disclosed herein are suitable to be linked to a binding peptide as disclosed herein via a linker as disclosed herein in order to increase the half-life of the binding peptide. Peptides of proven clinical safety and regulatory approval are preferred. In this regard PEG and PLGA are preferred. Interestingly, the branching of the polymer can provide additional benefits for TCEs having more than one CD3ε domain. For example, a dual targeting CDH3×MSLN molecule with two CD3ε domains such as SEQ ID NO 255 may be more efficiently masked by a masking molecule comprising a 4-arm PEG than a linear PEG.

In the context of this disclosure, a binding peptide which binds to an anti-CD3ε paratope of a CD3ε binding domain of a TCE with an affinity indicated as Kd of about 0.05 to about 10 nM, typically 0.1 pM to 10 nM or 500 pM to 5 nM is considered of “high” affinity, and a binding peptide which binds to an anti-CD3ε paratope of a CD3ε binding domain of a TCE with an affinity of about 10 to 150, preferably 10 to 100 mM is considered of “high” affinity. The exact value may vary with the respective TCE. However, the

Binding peptides are preferred which comprise (i.) a pE at the N-terminus to preferably provide the affinity gain with respect to the physiologic epitope comprising QDGNEEM to which the anti-CD3ε paratope of a CD3ε binder of a TCE binds, (ii.) an E or N is position 4 in N to C orientation to preferably fine tune high or intermediate affinity, (iii.) an E in position 5 in E to N orientation and (iv.) a C or K at the N-terminus to preferably provide for efficient linker connection. For example, with respect to a 10mer binding peptide such as pEX1X2X3EX4X5X6GX7 (SEQ ID NO: 433), wherein X1 is D, H or N, preferably D, and wherein X2 is G, F or Y, preferably G, and wherein X3 is N, H, Q, R, E, S, T, V, C, D, F, K L, M, W, Y or I, preferably N, or E, and wherein X4 is E, L, P, or W, preferably E, and wherein X5 can be any proteogenic amino acid, i.e. A, C, D, E, F, G, H, I, K, L, M, N, P, Q, R, S, T, V, W, or Y, and wherein X5 can be any proteogenic amino acid, i.e. A, C, D, E, F, G, H, I, K, L, M, N, P, Q, R, S, T, V, W, or Y, and wherein X7 is C or K, preferably pEX1X2X3EEX5X6GX7, more preferably pEDGX3EEX5X6GX7.

In general, with respect to the anti-CD3ε paratope binding peptides of this disclosure, the following findings were made, see Table 3:

Table 3: Exemplary c octameric (8mer) CD3ε peptide binding sequence structure activity relationships. The peptides were screened for binding in solution using a solution competition SPR assay format

The term “binding peptide” in the context of the present disclosure refers to a chain of about 5 to about 20 amino acids, preferably about 6 to about 12 amino acids, e.g. 10 amino acids, which binds to the anti-CD3ε paratope of the CD3ε binding domain of a TCE. The amino acid chain may comprise proteogenic and non-proteogenic amino acids. A preferred non-proteogenic amino acid is pyroglutamic acid (pE) also known as 5-oxoproline.

The term “CD3” refers to “cluster of differentiation 3” which is a Tcell co-receptor that is involved in activating, e.g., cytotoxic CD8+ T cells. A co-receptor is generally known as a cell surface receptor that binds a signaling molecule in addition to a primary receptor in order to facilitate ligand recognition and initiate biological processes. CD3-epsilon, together with CD3-gamma, -delta and -zeta, and the T-cell receptor alpha/beta and gamma/delta heterodimers, forms the T cell receptor-CD3 complex. This complex plays an important role in coupling antigen recognition to several intracellular signal-transduction pathways.

The term “TCE masking molecule” refers to a molecule comprising an anti-CD3 paratope binding peptide, preferably an anti-CD3ε paratope binding peptide, a suitable linker and a suitable polymer which extends the half-life of the TCE masking molecule with respect to the binding peptide alone. The TCE masking molecule may interchangeably also be referred to herein as CD3 peptide polymer (CD3 pepPOL) or more specifically as CD3 pepPEG.

The term “antibody product” refers to “secreted protein” or “secreted recombinant protein” and means a protein (e.g., a recombinant protein) that originally contained at least one secretion signal sequence when it is translated within a mammalian cell, and through, at least in part, enzymatic cleavage of the secretion signal sequence in the mammalian cell, is secreted at least partially into the extracellular space (e.g., a liquid culture medium). Skilled practitioners will appreciate that a “secreted” protein need not dissociate entirely from the cell to be considered a secreted protein.

The term “bispecific antibody” refers to full-length bispecific antibodies such as IgG-based antibodies. In contrast, bispecific antibody fragments are not full-length antibodies but parts thereof with a designated function, wherein both are colloquially referred to herein as bispecific antigen-binding molecules.

The term “T cell engager” refers to a bispecific antigen-binding molecule which comprises at least one binding domain which binds to an antigen or target (e.g. the target cell surface antigen, preferably a tumor associated antigen (TAA)), and the second binding domain binds to another antigen or target, in the present context CD3, preferably CD3ε. If not indicated otherwise, any reference in this disclosure to CD3 is meant to refer to CD3ε. CD3ε may also be written as CD3e and is intended to refer to the same subject matter. A TCE is understood to be an “antigen-binding molecule” which refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. An antigen-binding molecule is hence binds to its specific target or antigen. Furthermore, the binding domain of an antigen-binding molecule according to the invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to an specific antibody competing with the epitope of the defined antibody. The antibodies on which the constructs according to the invention are based include for example monoclonal, recombinant, chimeric, deimmunized, humanized and human antibodies.

The binding domain of an antigen-binding molecule according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antigen-binding molecules according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272.

Also within the definition of “binding domain” or “domain which binds” are fragments of full-length antibodies, such as VH, VHH, VL, (s)dAb, Fv, Fd, Fab, Fab′, F(ab′)2 or “r IgG” (“half antibody”). Antigen-binding molecules according to the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

Furthermore, the definition of the term “antigen-binding molecule” includes monovalent, bivalent and polyvalent/multivalent constructs and, thus, bispecific constructs, specifically binding to only two antigenic structure, as well as polyspecific/multispecific constructs, which specifically bind more than two antigenic structures, e.g. three, four or more, through distinct binding domains. Moreover, the definition of the term “antigen-binding molecule” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The term “bispecific” as used herein refers to an antigen-binding molecule which is “at least bispecific”, i.e., it comprises at least a first binding domain and a second binding domain, wherein the first binding domain binds to one antigen or target (e.g. the target cell surface antigen), and the second binding domain binds to another antigen or target. In the context of this disclosure, one of the two binding domains of a bispecific antigen-binding molecule binds to CD3, preferably CD3ε, more preferably and extracellular epitope of CD3ε. Because a bispecific antigen-binding molecule has defined above can engage T cells, the term bispecific antigen-binding molecule is used herein interchangeably with T cell engager (TCE) or TCE molecule. A TCE having two CD3ε binding domains in addition to at least one target binding domain is also considered bispecific. However, a TCE which comprises at least two target binding domains in addition to at least one CD3ε binding domain is understood to be dual targeting in addition to being bispecific. Accordingly, antigen-binding molecules according to the invention comprise specificities for at least two different antigens or targets. For example, the first domain does preferably not bind to an extracellular epitope of CD3ε of one or more of the species as described herein. The term “target cell surface antigen” refers to an antigenic structure expressed by a cell and which is present at the cell surface such that it is accessible for an antigen-binding molecule as described herein. It may be a protein, preferably the extracellular portion of a protein, or a carbohydrate structure, preferably a carbohydrate structure of a protein, such as a glycoprotein. It is preferably a tumor antigen. The term “bispecific antigen-binding molecule” of the invention also encompasses multispecific antigen-binding molecules such as trispecific antigen-binding molecules, the latter ones including three binding domains, or constructs having more than three (e.g. four, five . . . ) specificities.

Given that the antigen-binding molecules according to the invention are (at least) bispecific, they do not occur naturally and they are markedly different from naturally occurring products. A “bispecific” antigen-binding molecule or immunoglobulin is hence an artificial hybrid antibody or immunoglobulin having at least two distinct binding sides with different specificities. Bispecific antigen-binding molecules can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains (VH/VL) of the antigen-binding molecule of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antigen-binding molecule of the invention are linked with each other. The peptide linkers can also be used to fuse the third domain to the other domains of the antigen-binding molecule of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antigen-binding molecule of the invention.

The antigen-binding molecules of the present invention are preferably “in vitro generated antigen-binding molecules”. This term refers to an antigen-binding molecule according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antigen-binding molecule as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™ Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antigen-binding molecules include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antigen-binding molecules, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antigen-binding molecules involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antigen-binding molecules of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antigen-binding molecule, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. MoI. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064. [129]“Humanized” antibodies, antigen-binding molecules, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antigen-binding molecule” and “human binding domain” includes antibodies, antigen-binding molecules and binding domains having antibody regions such as variable and constant regions or domains which correspond substantially to human germline immunoglobulin sequences known in the art, including, for example, those described by Kabat et al. (1991) (loc. cit.). The human antibodies, antigen-binding molecules or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antigen-binding molecules or binding domains can have at least one, two, three, four, five, or more positions replaced with an amino acid residue that is not encoded by the human germline immunoglobulin sequence. The definition of human antibodies, antigen-binding molecules and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antigen-binding molecules of the invention are “isolated” or “substantially pure” antigen-binding molecules. “Isolated” or “substantially pure”, when used to describe the antigen-binding molecules disclosed herein, means an antigen-binding molecule that has been identified, separated and/or recovered from a component of its production environment. Preferably, the antigen-binding molecule is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antigen-binding molecules may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antigen-binding molecule in a wide variety of organisms and/or host cells that are known in the art. In preferred embodiments, the antigen-binding molecule will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antigen-binding molecule will be prepared by at least one purification step.

The term “binding domain” characterizes in connection with the present invention a domain which (specifically) binds to/interacts with/recognizes a given target epitope or a given target side on the target molecules (antigens), e.g. CD33 and CD3, respectively. The structure and function of the first binding domain (recognizing e.g. CD33), and preferably also the structure and/or function of the second binding domain (recognizing e.g. CD3), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domain which binds to the target cell surface antigen and/or the binding domain which binds to CD3ε is/are human binding domains. Antibodies and antigen-binding molecules comprising at least one human binding domain avoid some of the problems associated with antibodies or antigen-binding molecules that possess non-human such as rodent (e.g. murine, rat, hamster or rabbit) variable and/or constant regions. The presence of such rodent derived proteins can lead to the rapid clearance of the antibodies or antigen-binding molecules or can lead to the generation of an immune response against the antibody or antigen-binding molecule by a patient. In order to avoid the use of rodent derived antibodies or antigen-binding molecules, human or fully human antibodies/antigen-binding molecules can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antigen-binding molecules are expected to minimize the immunogenic and allergic responses intrinsic to mouse or mouse-derivatized mAbs and thus to increase the efficacy and safety of the administered antibodies/antigen-binding molecules. The use of fully human antibodies or antigen-binding molecules can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antigen-binding molecules comprising a human binding domain against the target cell surface antigen and a human binding domain against CD3ε in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), here: target cell surface antigen and CD3ε, respectively.

The term “epitope” refers to a side on an antigen to which a binding domain, such as an antibody or immunoglobulin, or a derivative, fragment or variant of an antibody or an immunoglobulin, binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 or 5 or 27 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

A method for epitope mapping is described in the following: When a region (a contiguous amino acid stretch) in the human target cell surface antigen protein is exchanged/replaced with its corresponding region of a non-human and non-primate target cell surface antigen (e.g., mouse target cell surface antigen, but others like chicken, rat, hamster, rabbit etc. might also be conceivable), a decrease in the binding of the binding domain is expected to occur, unless the binding domain is cross-reactive for the non-human, non-primate target cell surface antigen used. Said decrease is preferably at least 10%, 20%, 30%, 40%, or 50%; more preferably at least 60%, 70%, or 80%, and most preferably 90%, 95% or even 100% in comparison to the binding to the respective region in the human target cell surface antigen protein, whereby binding to the respective region in the human target cell surface antigen protein is set to be 100%. It is envisaged that the aforementioned human target cell surface antigen/non-human target cell surface antigen chimeras are expressed in CHO cells. It is also envisaged that the human target cell surface antigen/non-human target cell surface antigen chimeras are fused with a transmembrane domain and/or cytoplasmic domain of a different membrane-bound protein such as EpCAM.

In an alternative or additional method for epitope mapping, several truncated versions of the human target cell surface antigen extracellular domain can be generated in order to determine a specific region that is recognized by a binding domain. In these truncated versions, the different extracellular target cell surface antigen domains/sub-domains or regions are stepwise deleted, starting from the N-terminus. It is envisaged that the truncated target cell surface antigen versions may be expressed in CHO cells. It is also envisaged that the truncated target cell surface antigen versions may be fused with a transmembrane domain and/or cytoplasmic domain of a different membrane-bound protein such as EpCAM. It is also envisaged that the truncated target cell surface antigen versions may encompass a signal peptide domain at their N-terminus, for example a signal peptide derived from mouse IgG heavy chain signal peptide. It is furthermore envisaged that the truncated target cell surface antigen versions may encompass a v5 domain at their N-terminus (following the signal peptide) which allows verifying their correct expression on the cell surface. A decrease or a loss of binding is expected to occur with those truncated target cell surface antigen versions which do not encompass any more the target cell surface antigen region that is recognized by the binding domain. The decrease of binding is preferably at least 10%, 20%, 30%, 40%, 50%; more preferably at least 60%, 70%, 80%, and most preferably 90%, 95% or even 100%, whereby binding to the entire human target cell surface antigen protein (or its extracellular region or domain) is set to be 100.

A further method to determine the contribution of a specific residue of a target cell surface antigen to the recognition by an antigen-binding molecule or binding domain is alanine scanning (see e.g. Morrison K L & Weiss G A. Cur Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is a mature technology which has been used for a long period of time.

The interaction between the binding domain and the epitope or the region comprising the epitope implies that a binding domain exhibits appreciable affinity for the epitope/the region comprising the epitope on a particular protein or antigen (here: target cell surface antigen and CD3, respectively) and, generally, does not exhibit significant reactivity with proteins or antigens other than the target cell surface antigen or CD3. “Appreciable affinity” includes binding with an affinity of about 106 M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the target cell surface antigen or CD3. Preferably, a binding domain of the invention does not essentially or substantially bind to proteins or antigens other than the target cell surface antigen or CD3 (i.e., the first binding domain is preferably not capable of binding to proteins other than the target cell surface antigen and the second binding domain is not capable of binding to proteins other than CD3). It is an envisaged characteristic of the antigen-binding molecules according to the present invention to have superior affinity characteristics in comparison to other HLE formats. Such a superior affinity, in consequence, suggests a prolonged half-life in vivo. The longer half-life of the antigen-binding molecules according to the present invention may reduce the duration and frequency of administration which typically contributes to improved patient compliance. This is of particular importance as the antigen-binding molecules of the present invention are particularly beneficial for highly weakened or even multimorbide cancer patients.

The term “paratope” is understood as an antigen-binding site, i.e. the part of an antibody or an antibody-derived molecule such as a TCE which recognizes and binds to an antigen. The paratope is generally understood as a small region within the Fv. Each paratope comprises six complementarity-determining regions (CDRs), three derived from each of the light and heavy chains. In the context of the present invention, a CD3 paratope or more preferred a CD3ε paratope facilitates the binding of a TCE molecule to the CD3 receptor on a T cell. The CD3 T-cell co-receptor is the antigen, more preferably the CD3ε chain thereof, and a paratope of the TCE is the part of the CD3 binding domain which binds and makes contact with the epitope of said antigen.

The term “anti-CD3ε paratope” is understood as the CD3ε-binding site within the CD3ε binding domain of a TCE which binds to the corresponding epitope on CD3ε. The anti-CD3ε paratope is the site to which the binding peptide of the TCE masking molecule binds in order to preferably transiently mask the TCE.

The term “does not essentially/substantially bind” or “is not capable of binding” means that a binding domain of the present invention does not bind a protein or antigen other than the target cell surface antigen or CD3, i.e., does not show reactivity of more than 30%, preferably not more than 20%, more preferably not more than 10%, particularly preferably not more than 9%, 8%, 7%, 6% or 5% with proteins or antigens other than the target cell surface antigen or CD3, whereby binding to the target cell surface antigen or CD3, respectively, is set to be 100%.

Binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a f-sheet configuration, connected by three hypervariable regions, which form loops connecting, and in some cases forming part of, the p-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al., Nature, 1989, 342: 877; Martin and Thornton, J. MoI. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antigen-binding molecules, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 101 different antibody molecules (Immunoglobulin Genes, 2^{nd} ed., eds. Jonio et al., Academic Press, San Diego, CA, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “Fc portion” or “Fc monomer” means in connection with this invention a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain. In a preferred aspect of this definition, an Fc monomer can be a polypeptide constant region comprising a portion of the Ig-Fc hinge region, a CH2 region and a CH3 region, wherein the hinge region is amino terminal to the CH2 domain. It is envisaged that the hinge region of the present invention promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of an IgG₁ Fc region, an IgG₂ Fc region, an IgG₃ Fc region, an IgG₄ Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g. to comprise residues D231 (of the hinge domain—corresponding to D234 in Table 4 below)) to P476, respectively L476 (for IgG₄) of the carboxyl-terminus of the CH3 domain, wherein the numbering is according to Kabat. The two Fc portions or Fc monomers, which are fused to each other via a peptide linker define the third domain of the antigen-binding molecule of the invention, which may also be defined as scFc domain.

In some embodiments, it is envisaged that a scFc domain as disclosed herein, respectively the Fc monomers fused to each other are comprised only in the third domain of the antigen-binding molecule. An IgG hinge region can be identified by analogy using the Kabat numbering as set forth in Table 4. In line with the above, it is envisaged that a hinge domain/region may comprise the amino acid residues corresponding to the IgG₁ sequence stretch of D234 to P243 according to the Kabat numbering. It is likewise envisaged that a hinge domain/region of may comprise or consist of the IgG1 hinge sequence DKTHTCPPCP (SEQ ID NO: 182) (corresponding to the stretch D234 to P243 as shown in Table 4 below—variations of said sequence are also envisaged provided that the hinge region still promotes dimerization). In a preferred embodiment, the glycosylation site at Kabat position 314 of the CH2 domains in the third domain of the antigen-binding molecule is removed by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

It is also envisaged that the third domain of the antigen-binding molecule comprises or consists in an amino to carboxyl order: DKTHTCPPCP (SEQ ID NO: 182) (i.e. hinge) —CH2-CH3-linker-DKTHTCPPCP (SEQ ID NO: 182) (i.e. hinge) —CH2-CH3. The peptide linker of the aforementioned antigen-binding molecule is in a preferred embodiment characterized by the amino acid sequence Gly-Gly-Gly-Gly-Ser, i.e. Gly₄Ser (SEQ ID NO: 187), or polymers thereof, i.e. (Gly₄Ser)x, where x is an integer of 5 or greater (e.g. 5, 6, 7, 8 etc. or greater), 6 being preferred ((Gly₄Ser)₆). Said construct may further comprise the aforementioned substitutions N314X, preferably N314G, and/or the further substitutions V321C and R309C. In a preferred embodiment of the antigen-binding molecules as defined herein before, it is envisaged that the second domain binds to an extracellular epitope of the human and/or the Macaca CD3ε chain.

In further embodiments, the hinge domain/region comprises or consists of the IgG2 subtype hinge sequence ERKCCVECPPCP (SEQ ID NO: 183), the IgG3 subtype hinge sequence ELKTPLDTTHTCPRCP (SEQ ID NO: 184) or ELKTPLGDTTHTCPRCP (SEQ ID NO: 185), and/or the IgG4 subtype hinge sequence ESKYGPPCPSCP (SEQ ID NO: 186). The IgG1 subtype hinge sequence may be the following one EPKSCDKTHTCPPCP (as shown in Table 4 and SEQ ID NO: 183). These core hinge regions are thus also envisaged in the context of the present invention.

The location and sequence of the IgG CH2 and IgG CD3 domain can be identified by analogy using the Kabat numbering as set forth in Table 5:

In one embodiment of the invention the emphasized bold amino acid residues in the CH3 domain of the first or both Fc monomers are deleted.

The peptide linker, by whom the polypeptide monomers (“Fc portion” or “Fc monomer”) of the third domain are fused to each other, preferably comprises at least 25 amino acid residues (25, 26, 27, 28, 29, 30 etc.). More preferably, this peptide linker comprises at least 30 amino acid residues (30, 31, 32, 33, 34, 35 etc.). It is also preferred that the linker comprises up to 40 amino acid residues, more preferably up to 35 amino acid residues, most preferably exactly 30 amino acid residues. A preferred embodiment of such peptide linker is characterized by the amino acid sequence Gly-Gly-Gly-Gly-Ser, i.e. Gly₄Ser (SEQ ID NO: 187), or polymers thereof, i.e. (Gly₄Ser)x, where x is an integer of 5 or greater (e.g. 6, 7 or 8). Preferably the integer is 6 or 7, more preferably the integer is 6.

In the event that a linker is used to fuse the first domain to the second domain, or the first or second domain to the third domain, this linker is preferably of a length and sequence sufficient to ensure that each of the first and second domains can, independently from one another, retain their differential binding specificities. For peptide linkers which connect the at least two binding domains (or two variable domains) in the antigen-binding molecule of the invention, those peptide linkers are preferred which comprise only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A preferred embodiment of the peptide linker for a fusion the first and the second domain is depicted in SEQ ID NO: 1. A preferred linker embodiment of the peptide linker for a fusion the second and the third domain is a (Gly)₄-linker, respectively G₄-linker.

A particularly preferred “single” amino acid in the context of one of the above described “peptide linker” is Gly. Accordingly, said peptide linker may consist of the single amino acid Gly. In a preferred embodiment of the invention a peptide linker is characterized by the amino acid sequence Gly-Gly-Gly-Gly-Ser, i.e. Gly₄Ser (SEQ ID NO: 187), or polymers thereof, i.e. (Gly₄Ser)x, where x is an integer of 1 or greater (e.g. 2 or 3). Preferred linkers are depicted in SEQ ID Nos: 1 to 12. The characteristics of said peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which furthermore do not promote any secondary structures are preferred. The linkage of said domains to each other can be provided, e.g., by genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

In a preferred embodiment of the antigen-binding molecule, the first and second domain form an antigen-binding molecule in a format selected from the group consisting of (scFv)₂, scFv-single domain mAb, diabody and oligomers of any of the those formats

According to a particularly preferred embodiment, and as documented in the appended examples, the first and the second domain of the antigen-binding molecule of the invention is a “bispecific single chain antigen-binding molecule”, more preferably a bispecific “single chain Fv” (scFv). Although the two domains of the Fv fragment, VL and VH, are coded for by separate genes, they can be joined, using recombinant methods, by a synthetic linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A single-chain variable fragment (scFv) is hence a fusion protein of the variable region of the heavy chain (VH) and of the light chain (VL) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and introduction of the linker.

Bispecific single chain antigen-binding molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Bruhl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. Cit. and Little (2009), loc. Cit.) can be adapted to produce single chain antigen-binding molecules specifically recognizing (an) elected target(s).

Bivalent (also called divalent) or bispecific single-chain variable fragments (bi-scFvs or di-scFvs having the format (scFv)₂ can be engineered by linking two scFv molecules (e.g. with linkers as described hereinbefore). If these two scFv molecules have the same binding specificity, the resulting (scFv)₂ molecule will preferably be called bivalent (i.e. it has two valences for the same target epitope). If the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

Either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal antigen-binding molecule composed of (at least) two single domain monoclonal antibodies, which are individually selected from the group comprising V_{H}, V_{L}, V_{H}H and V_{NAR}. The linker is preferably in the form of a peptide linker. Similarly, an “scFv-single domain mAb” is a monoclonal antigen-binding molecule composed of at least one single domain antibody as described above and one scFv molecule as described above. Again, the linker is preferably in the form of a peptide linker.

Whether or not an antigen-binding molecule competes for binding with another given antigen-binding molecule can be measured in a competition assay such as a competitive ELISA or a cell-based competition assay. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added and any additional binding is determined. Possible means for the read-out includes flow cytometry.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ((zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3ε gene which resides on chromosome 11. The most preferred epitope of CD3 epsilon is comprised within amino acid residues 1-27 of the human CD3 epsilon extracellular domain. It is envisaged that antigen-binding molecules according to the present invention typically and advantageously show less unspecific T cell activation, which is not desired in specific immunotherapy. This translates to a reduced risk of side effects.

The redirected lysis of target cells via the recruitment of T cells by a multispecific, at least bispecific, antigen-binding molecule involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by antigen-binding molecules of the invention can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque target cell surface antigen which is bound by the first domain, the effector cells should also be of macaque origin such as a macaque T cell line, e.g. 4119LnPx. The target cells should express (at least the extracellular domain of) the target cell surface antigen, e.g. human or macaque target cell surface antigen. Target cells can be a cell line (such as CHO) which is stably or transiently transfected with target cell surface antigen, e.g. human or macaque target cell surface antigen. Alternatively, the target cells can be a target cell surface antigen positive natural expresser cell line. Usually EC₅₀ values are expected to be lower with target cell lines expressing higher levels of target cell surface antigen on the cell surface. The effector to target cell (E:T) ratio is usually about 10:1, but can also vary. Cytotoxic activity of target cell surface antigen×CD3 bispecific antigen-binding molecules can be measured in a ⁵¹Cr-release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

The cytotoxic activity mediated by target cell surface antigen×CD3 bispecific antigen-binding molecules of the present invention is preferably measured in a cell-based cytotoxicity assay. It may also be measured in a ⁵¹Cr-release assay. It is represented by the EC₅₀ value, which corresponds to the half maximal effective concentration (concentration of the antigen-binding molecule which induces a cytotoxic response halfway between the baseline and maximum). Preferably, the EC₅₀ value of the target cell surface antigen×CD3 bispecific antigen-binding molecules is ≤5000 pM or ≤4000 pM, more preferably ≤3000 pM or ≤2000 pM, even more preferably ≤1000 pM or ≤500 pM, even more preferably ≤400 pM or ≤300 pM, even more preferably ≤200 pM, even more preferably ≤100 pM, even more preferably ≤50 pM, even more preferably ≤20 pM or ≤10 pM, and most preferably ≤5 pM.

The above given EC₅₀ values can be measured in different assays. The skilled person is aware that an EC₅₀ value can be expected to be lower when stimulated/enriched CD8⁺ T cells are used as effector cells, compared with unstimulated PBMC. It can furthermore be expected that the EC₅₀ values are lower when the target cells express a high number of the target cell surface antigen compared with a low target expression rat. For example, when stimulated/enriched human CD8⁺ T cells are used as effector cells (and either target cell surface antigen transfected cells such as CHO cells or target cell surface antigen positive human cell lines are used as target cells), the EC₅₀ value of the target cell surface antigen×CD3 bispecific antigen-binding molecule is preferably ≤1000 pM, more preferably ≤500 pM, even more preferably ≤250 pM, even more preferably ≤100 pM, even more preferably ≤50 pM, even more preferably ≤10 pM, and most preferably ≤5 pM. When human PBMCs are used as effector cells, the EC₅₀ value of the target cell surface antigen×CD3 bispecific antigen-binding molecule is preferably ≤5000 pM or 4000 pM (in particular when the target cells are target cell surface antigen positive human cell lines), more preferably ≤2000 pM (in particular when the target cells are target cell surface antigen transfected cells such as CHO cells), more preferably ≤1000 pM or ≤500 pM, even more preferably ≤200 pM, even more preferably ≤150 pM, even more preferably ≤100 pM, and most preferably ≤50 pM, or lower. When a macaque T cell line such as LnPx4119 is used as effector cells, and a macaque target cell surface antigen transfected cell line such as CHO cells is used as target cell line, the EC₅₀ value of the target cell surface antigen×CD3 bispecific antigen-binding molecule is preferably ≤2000 pM or ≤1500 pM, more preferably ≤1000 pM or ≤500 pM, even more preferably ≤300 pM or ≤250 pM, even more preferably ≤100 pM, and most preferably ≤50 pM.

Preferably, the target cell surface antigen×CD3 bispecific antigen-binding molecules of the present invention do not induce/mediate lysis or do not essentially induce/mediate lysis of target cell surface antigen negative cells such as CHO cells. The term “do not induce lysis”, “do not essentially induce lysis”, “do not mediate lysis” or “do not essentially mediate lysis” means that an antigen-binding molecule of the present invention does not induce or mediate lysis of more than 30%, preferably not more than 20%, more preferably not more than 10%, particularly preferably not more than 9%, 8%, 7%, 6% or 5% of target cell surface antigen negative cells, whereby lysis of a target cell surface antigen positive human cell line is set to be 100%. This usually applies for concentrations of the antigen-binding molecule of up to 500 nM. The skilled person knows how to measure cell lysis without further ado. Moreover, the present specification teaches specific instructions how to measure cell lysis.

The difference in cytotoxic activity between the monomeric and the dimeric isoform of individual target cell surface antigen×CD3 bispecific antigen-binding molecules is referred to as “potency gap”. This potency gap can e.g. be calculated as ratio between EC₅₀ values of the molecule's monomeric and dimeric form. Potency gaps of the target cell surface antigen×CD3 bispecific antigen-binding molecules of the present invention are preferably ≤5, more preferably ≤4, even more preferably ≤3, even more preferably ≤2 and most preferably ≤1.

The first and/or the second (or any further) binding domain(s) of the antigen-binding molecule of the invention is/are preferably cross-species specific for members of the mammalian order of primates. Cross-species specific CD3 binding domains are, for example, described in WO 2008/119567. According to one embodiment, the first and/or second binding domain, in addition to binding to human target cell surface antigen and human CD3, respectively, will also bind to target cell surface antigen/CD3 of primates including (but not limited to) new world primates (such as Callithrix jacchus, Saguinus Oedipus or Saimiri sciureus), old world primates (such baboons and macaques), gibbons, and non-human homininae.

In one embodiment of the antigen-binding molecule of the invention the first domain binds to human target cell surface antigen and further binds to macaque target cell surface antigen, such as target cell surface antigen of Macaca fascicularis, and more preferably, to macaque target cell surface antigen expressed on the surface macaque cells. The affinity of the first binding domain for macaque target cell surface antigen is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM or even ≤0.01 nM.

Preferably the affinity gap of the antigen-binding molecules according to the invention for binding macaque target cell surface antigen versus human target cell surface antigen [ma target cell surface antigen:hu target cell surface antigen](as determined e.g. by BiaCore or by Scatchard analysis) is <100, preferably <20, more preferably <15, further preferably <10, even more preferably <8, more preferably <6 and most preferably <2. Preferred ranges for the affinity gap of the antigen-binding molecules according to the invention for binding macaque target cell surface antigen versus human target cell surface antigen are between 0.1 and 20, more preferably between 0.2 and 10, even more preferably between 0.3 and 6, even more preferably between 0.5 and 3 or between 0.5 and 2.5, and most preferably between 0.5 and 2 or between 0.6 and 2.

The second (binding) domain of the antigen-binding molecule binds to human CD3 epsilon and/or to Macaca CD3 epsilon. In a preferred embodiment, the second domain further binds to Callithrix jacchus, Saguinus Oedipus or Saimiri sciureus CD3 epsilon. Callithrix jacchus and Saguinus oedipus are both new world primate belonging to the family of Callitrichidae, while Saimiri sciureus is a new world primate belonging to the family of Cebidae.

It is preferred for the antigen-binding molecule that the second domain which binds to an extracellular epitope of the human and/or the Macaca CD3 comprises a VL region comprising CDR-L1, CDR-L2 and CDR-L3 selected from:
(a) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(b) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567; and
(c) CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567.

In an also preferred embodiment of the antigen-binding molecule, the second domain which binds to an extracellular epitope of the human and/or the Macaca CD3 epsilon chain comprises a VH region comprising CDR-H 1, CDR-H2 and CDR-H3 selected from:
(a) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(b) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
I CDR-H1 as depicted in SEQ ID NO: 48 of WO 2008/119567, CDR-H2 as depicted in SEQ ID NO: 49 of WO 2008/119567 and CDR-H3 as depicted in SEQ ID NO: 50 of WO 2008/119567;
(d) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
I CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(f) CDR-H1 as depicted in SEQ ID NO: 102 of WO 2008/119567, CDR-H2 as depicted in SEQ ID NO: 103 of WO 2008/119567 and CDR-H3 as depicted in SEQ ID NO: 104 of WO 2008/119567;
(g) CDR-H1 as depicted in SEQ ID NO: 120 of WO 2008/119567, CDR-H2 as depicted in SEQ ID NO: 121 of WO 2008/119567 and CDR-H3 as depicted in SEQ ID NO: 122 of WO 2008/119567;
(h) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(i) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567; and
(j) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567.

In a preferred embodiment of the antigen-binding molecule the above described three groups of VL CDRs are combined with the above described ten groups of VH CDRs within the second binding domain to form (30) groups, each comprising CDR-L 1-3 and CDR-H 1-3.

It is preferred for the antigen-binding molecule that the second domain which binds to CD3 comprises a VL region selected from the group consisting of a VL region as depicted in SEQ ID NO: 25, 346, or 354; or as depicted o SEQ ID NO 17, 21, 35, 39, 53, 57, 71, 75, 89, 93, 107, 111, 125, 129, 143, 147, 161, 165, 179 or 183 of WO 2008/119567 or as depicted in SEQ ID NO: 200.

It is also preferred that the second domain which binds to CD3 comprises a VH region selected from the group consisting of a VH region as depicted in SEQ ID NO: 25, 346, or 354; or as depicted o SEQ ID NO 15, 19, 33, 37, 51, 55, 69, 73, 87, 91, 105, 109, 123, 127, 141, 145, 159, 163, 177 or 181 of WO 2008/119567 or as depicted in SEQ ID NO: 201.

More preferably, the antigen-binding molecule is characterized by a second domain which binds to CD3ε comprising a VL region and a VH region selected from the group consisting of:
(a) a VL region as depicted in SEQ ID NO: 17 or 21 of WO 2008/119567 and a VH region as depicted in SEQ ID NO: 15 or 19 of WO 2008/119567;
(b) a VL region as depicted in SEQ ID NO: 35 or 39 of WO 2008/119567 and a VH region as depicted in SEQ ID NO: 33 or 37 of WO 2008/119567;
I a VL region as depicted in SEQ ID NO: 53 or 57 of WO 2008/119567 and a VH region as depicted in SEQ ID NO: 51 or 55 of WO 2008/119567;
(d) a VL region as depicted in SEQ ID NO: 71 or 75 of WO 2008/119567 and a VH region as depicted in SEQ ID NO: 69 or 73 of WO 2008/119567;
I a VL region as depicted in SEQ ID NO: 89 or 93 of WO 2008/119567 and a VH region as depicted in SEQ ID NO: 87 or 91 of WO 2008/119567;
(f) a VL region as depicted in SEQ ID NO: 107 or 111 of WO 2008/119567 and a VH region as depicted in SEQ ID NO: 105 or 109 of WO 2008/119567;
(g) a VL region as depicted in SEQ ID NO: 125 or 129 of WO 2008/119567 and a VH region as depicted in SEQ ID NO: 123 or 127 of WO 2008/119567;
(h) a VL region as depicted in SEQ ID NO: 143 or 147 of WO 2008/119567 and a VH region as depicted in SEQ ID NO: 141 or 145 of WO 2008/119567;
(i) a VL region as depicted in SEQ ID NO: 161 or 165 of WO 2008/119567 and a VH region as depicted in SEQ ID NO: 159 or 163 of WO 2008/119567; and
(j) a VL region as depicted in SEQ ID NO: 179 or 183 of WO 2008/119567 and a VH region as depicted in SEQ ID NO: 177 or 181 of WO 2008/119567.

Also preferred in connection with the antigen-binding molecule is a second domain which binds to CD3 comprising a VL region as depicted in SEQ ID NO: 200 and a VH region as depicted in SEQ ID NO: 201.

According to a preferred embodiment of the antigen-binding molecule, the first and/or the second domain have the following format: The pairs of VH regions and VL regions are in the format of a single chain antibody (scFv). The VH and VL regions are arranged in the order VH-VL or VL-VH. It is preferred that the VH-region is positioned N-terminally of a linker sequence, and the VL-region is positioned C-terminally of the linker sequence.

A preferred embodiment of the above described TCE is characterized by a domain which binds to CD3ε comprising an amino acid sequence selected from the group consisting of SEQ ID Nos: 23, 25, 41, 43, 59, 61, 77, 79, 95, 97, 113, 115, 131, 133, 149, 151, 167, 169, 185 or 187 of WO 2008/119567 or comprising a sequence having 90, 95, 99 or 100% sequence identity with SEQ ID NO: 26, 381, 382 or 383.

Covalent modifications of the antigen-binding molecules are also included within the scope of this invention, and are generally, but not always, done post-translationally. For example, several types of covalent modifications of the antigen-binding molecule are introduced into the molecule by reacting specific amino acid residues of the antigen-binding molecule with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the antigen-binding molecules to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antigen-binding molecules included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antigen-binding molecule is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an antigen-binding molecule is preferably altered through changes at the DNA level, particularly by mutating the DNA encoding the polypeptide at preselected bases such that codons are generated that will translate into the desired amino acids.

Another means of increasing the number of carbohydrate moieties on the antigen-binding molecule is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, I aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antigen-binding molecule may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the antigen-binding molecule are also contemplated herein. For example, another type of covalent modification of the antigen-binding molecule comprises linking the antigen-binding molecule to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen-binding molecule, e.g. in order to facilitate the addition of polymers such as PEG.

In some embodiments, the covalent modification of the antigen-binding molecules of the invention comprises the addition of one or more labels. The labelling group may be coupled to the antigen-binding molecule via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sides for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8^{th} Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

The antigen-binding molecule of the invention may also comprise additional domains, which are e.g. helpful in the isolation of the molecule or relate to an adapted pharmacokinetic profile of the molecule. Domains helpful for the isolation of an antigen-binding molecule may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antigen-binding molecules characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antigen-binding molecule, preferably it is located at the C-terminus. Most preferably, a hexa-histidine tag (HHHHHH) (SEQ ID NO: 199) is linked via peptide bond to the C-terminus of the antigen-binding molecule according to the invention. Additionally, a conjugate system of PLGA-PEG-PLGA may be combined with a poly-histidine tag for sustained release application and improved pharmacokinetic profile.

Amino acid sequence modifications of the antigen-binding molecules described herein are also contemplated. For example, it may be desirable to improve the binding affinity and/or other biological properties of the antigen-binding molecule. Amino acid sequence variants of the antigen-binding molecules are prepared by introducing appropriate nucleotide changes into the antigen-binding molecules nucleic acid, or by peptide synthesis. All of the below described amino acid sequence modifications should result in an antigen-binding molecule which still retains the desired biological activity (binding to the target cell surface antigen and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Giu or E); glycine (Giy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Giu); a positively charged sidechain (e.g., Arg, His, Lys); or an uncharged polar side chain (e.g., Asn, Cys, Gin, Giy, His, Met, Phe, Ser, Thr, Trp, and Tyr).

Amino acid modifications include, for example, deletions from, and/or insertions into, and/or substitutions of, residues within the amino acid sequences of the antigen-binding molecules. Any combination of deletion, insertion, and substitution is made to arrive at the final construct, provided that the final construct possesses the desired characteristics. The amino acid changes also may alter post-translational processes of the antigen-binding molecules, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antigen-binding molecule include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antigen-binding molecule of the invention. An insertional variant of the antigen-binding molecule of the invention includes the fusion to the N-terminus or to the C-terminus of the antigen-binding molecule of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antigen-binding molecules that are preferred locations for mutagenesis is called “alanine scanning mutagenesis” as described by Cunningham and Wells in Science, 244: 1081-1085 (1989). Here, a residue or group of target residues within the antigen-binding molecule is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antigen-binding molecule variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as the target cell surface antigen or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antigen-binding molecule may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antigen-binding molecule retains its capability to bind to the target cell surface antigen via the first domain and to CD3, respectively CD3 epsilon, via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened for a desired characteristic.

TABLE 7
Amino acid substitutions

| Original | Exemplary Substitutions | Preferred Substitutions |
|---|---|---|
| Ala (A) | val, leu, ile | Val |
| Arg I | lys, gln, asn | Lys |
| Asn (N) | gln, his, asp, lys, arg | Gln |
| Asp (D) | glu, asn | Glu |
| Cys I | ser, ala | Ser |
| Gln (Q) | asn, glu | Asn |
| Glu I | asp, gln | Asp |
| Gly (G) | Ala | Ala |
| His (H) | asn, gln, lys, arg | Arg |
| Ile (I) | leu, val, met, ala, phe | Leu |
| Leu (L) | norleucine, ile, val, met, ala | Ile |
| Lys (K) | arg, gln, asn | Arg |
| Met (M) | leu, phe, ile | Leu |
| Phe (F) | leu, val, ile, ala, tyr | Tyr |
| Pro (P) | Ala | Ala |
| Ser (S) | Thr | Thr |
| Thr (T) | Ser | Ser |
| Trp (W) | tyr, phe | Tyr |
| Tyr (Y) | trp, phe, thr, ser | Phe |
| Val (V) | ile, leu, met, phe, ala | Leu |

Substantial modifications in the biological properties of the antigen-binding molecule are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, Ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antigen-binding molecule may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antigen-binding molecule. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antigen-binding molecules according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antigen-binding molecules leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antigen-binding molecules. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the bispecific antigen-binding molecules exhibit high monomer yields under standard research scale conditions, e.g., in a standard two-step purification process. Preferably the monomer yield of the antigen-binding molecules according to the invention is ≥0.25 mg/L supernatant, more preferably ≥0.5 mg/L, even more preferably ≥1 mg/L, and most preferably ≥3 mg/L supernatant.

Likewise, the yield of the dimeric antigen-binding molecule isoforms and hence the monomer percentage (i.e., monomer:(monomer+dimer)) of the antigen-binding molecules can be determined. The productivity of monomeric and dimeric antigen-binding molecules and the calculated monomer percentage can e.g. be obtained in the SEC purification step of culture supernatant from standardized research-scale production in roller bottles. In one embodiment, the monomer percentage of the antigen-binding molecules is ≥80%, more preferably ≥85%, even more preferably ≥90%, and most preferably ≥95%.

In one embodiment, the antigen-binding molecules have a preferred plasma stability (ratio of EC50 with plasma to EC50 w/o plasma) of ≤5 or ≤4, more preferably ≤3.5 or ≤3, even more preferably ≤2.5 or ≤2, and most preferably ≤1.5 or ≤1. The plasma stability of an antigen-binding molecule can be tested by incubation of the construct in human plasma at 37° C. for 24 hours followed by EC50 determination in a “chromium release cytotoxicity assay. The effector cells in the cytotoxicity assay can be stimulated enriched human CD8 positive T cells. Target cells can e.g. be CHO cells transfected with the human target cell surface antigen. The effector to target cell (E:T) ratio can be chosen as 10:1. The human plasma pool used for this purpose is derived from the blood of healthy donors collected by EDTA coated syringes. Cellular components are removed by centrifugation and the upper plasma phase is collected and subsequently pooled. As control, antigen-binding molecules are diluted immediately prior to the cytotoxicity assay in RPMI-1640 medium. The plasma stability is calculated as ratio of EC50 (after plasma incubation) to EC50 (control).

It is furthermore preferred that the monomer to dimer conversion of antigen-binding molecules of the invention is low. The conversion can be measured under different conditions and analyzed by high performance size exclusion chromatography. For example, incubation of the monomeric isoforms of the antigen-binding molecules can be carried out for 7 days at 37° C. and concentrations of e.g. 100 μg/ml or 250 μg/ml in an incubator. Under these conditions, it is preferred that the antigen-binding molecules of the invention show a dimer percentage that is ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is also preferred that the bispecific antigen-binding molecules present with very low dimer conversion after a number of freeze/thaw cycles. For example, the antigen-binding molecule monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antigen-binding molecule, which had been converted into dimeric antigen-binding molecule. Preferably the dimer percentages of the bispecific antigen-binding molecules are ≤5%, more preferably ≤4%, even more preferably ≤3%, even more preferably ≤2.5%, even more preferably ≤2%, even more preferably ≤1.5%, and most preferably ≤1% or even ≤0.5%, for example after three freeze/thaw cycles.

The bispecific antigen-binding molecules preferably show a favorable thermostability with aggregation temperatures ≥45° C. or ≥50° C., more preferably ≥52° C. or ≥54° C., even more preferably ≥56° C. or ≥57° C., and most preferably ≥58° C. or ≥59° C. The thermostability parameter can be determined in terms of antibody aggregation temperature as follows: Antibody solution at a concentration 250 μg/ml is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by Differential Scanning Calorimetry (DSC) to determine intrinsic biophysical protein stabilities of the antigen-binding molecules. These experiments are performed using a MicroCal LLC (Northampton, MA, U.S.A) VP-DSC device. The energy uptake of a sample containing an antigen-binding molecule is recorded from 20° C. to 90° C. compared to a sample containing only the formulation buffer. The antigen-binding molecules are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

The target cell surface antigen×CD3 bispecific antigen-binding molecules of the invention are also envisaged to have a turbidity (as measured by OD340 after concentration of purified monomeric antigen-binding molecule to 2.5 mg/ml and over night incubation) of ≤0.2, preferably of ≤0.15, more preferably of ≤0.12, even more preferably of ≤0.1, and most preferably of ≤0.08.

It is furthermore envisaged that the bispecific antigen-binding molecules exhibit therapeutic efficacy or anti-tumor activity. This can e.g. be assessed in a study as disclosed in the following example of an advanced stage human tumor xenograft model:

The skilled person knows how to modify or adapt certain parameters of this study, such as the number of injected tumor cells, the site of injection, the number of transplanted human T cells, the amount of bispecific antigen-binding molecules to be administered, and the timelines, while still arriving at a meaningful and reproducible result. Preferably, the tumor growth inhibition T/C [%] is ≤70 or ≤60, more preferably ≤50 or ≤40, even more preferably ≤30 or ≤20 and most preferably ≤10 or ≤5 or even ≤2.5.

In a preferred embodiment of the antigen-binding molecule the antigen-binding molecule is a single chain antigen-binding molecule.

Also in a preferred embodiment of the antigen-binding molecule said third domain comprises in an amino to carboxyl order:

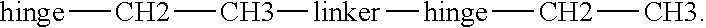

Also in one embodiment of the invention the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the antigen-binding molecule of the invention that the cysteines forming the cysteine disulfide bridge in the mature antigen-binding molecule are introduced into the amino acid sequence of the CH2 domain corresponding to 309 and 321 (Kabat numbering).

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

It is assumed that the preferred features of the antigen-binding molecule of the invention compared e.g. to the bispecific heteroFc antigen-binding molecule known in the art (FIG. 1b) may be inter alia related to the introduction of the above described modifications in the CH2 domain. Thus, it is preferred for the construct of the invention that the CH2 domains in the third domain of the antigen-binding molecule of the invention comprise the intra domain cysteine disulfide bridge at Kabat positions 309 and 321 and/or the glycosylation site at Kabat position 314 is removed by a N314X substitution as above, preferably by a N314G substitution.

In a further preferred embodiment of the invention the CH2 domains in the third domain of the antigen-binding molecule of the invention comprise the intra domain cysteine disulfide bridge at Kabat positions 309 and 321 and the glycosylation site at Kabat position 314 is removed by a N314G substitution.

In one embodiment the invention provides an antigen-binding molecule, wherein:
(182) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains where described herein above and comprise e.g. Fv fragments, scFv fragments or Fab fragments described herein above. Alternatively either one or both of those binding domains may comprise only a single variable domain. Examples for such single domain binding domains where described herein above and comprise e.g. nanobodies or single variable domain antibodies comprising merely one variable domain, which might be VHH, VH or VL, that specifically bind an antigen or epitope independently of other V regions or domains.

In a preferred embodiment of the antigen-binding molecule of the invention first and second domain are fused to the third domain via a peptide linker. Preferred peptide linker have been described herein above and are characterized by the amino acid sequence Gly-Gly-Gly-Gly-Ser, i.e. Gly₄Ser (SEQ ID NO: 187), or polymers thereof, i.e. (Gly₄Ser)x, where x is an integer of 1 or greater (e.g. 2 or 3). A particularly preferred linker for the fusion of the first and second domain to the third domain is depicted in SEQ ID Nos: 1.

In a preferred embodiment the antigen-binding molecule of the invention is characterized to comprise in an amino to carboxyl order:
(a) the first domain;
(b) a peptide linker having an amino acid sequence selected from the group consisting of SEQ ID Nos: 187-189;
I the second domain;
(d) a peptide linker having an amino acid sequence selected from the group consisting of SEQ ID NO: 187, 188, 189, 195, 196, 197 and 198;
I the first polypeptide monomer of the third domain;
(f) a peptide linker having an amino acid sequence selected from the group consisting of SEQ ID Nos: 191, 192, 193 and 194; and
(g) the second polypeptide monomer of the third domain.

In one aspect of the invention the target cell surface antigen bound by the first domain is a tumor antigen, an antigen specific for an immunological disorder or a viral antigen. The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Non-limiting examples of tumor antigens as used herein are CDH19, MSLN, DLL3, FLT3, EGFRvIII, CD33, CD19, MUC17, CLDN18.2, CDH3, CD70, BCMA and PSMA.

Further target cell surface antigens specific for an immunological disorder in the context comprise, for example, TL1A and TNF-alpha. Said targets are preferably addressed by a bispecific antigen-binding molecule, which is preferably a full length antibody. In a very preferred embodiment, an antibody is a hetero IgG antibody.

In a preferred embodiment of the antigen-binding molecule the tumor antigen, preferably tumor antigen, is selected from the group consisting of CDH19, MSLN, DLL3, FLT3, EGFRvIII, CD33, CD19, MUC17, CLDN18.2, CDH3, CD70, CLDN6, STEAP 1, BCMA and PSMA.

In one aspect the antigen-binding molecule comprises in an amino to carboxyl order:
(a) the first domain having 80, 90, 95, 99 or 100% sequence identity with an amino acid sequence selected from the group consisting of SEQ ID Nos: 7, 8, 17, 27, 28, 37, 38, 39, 40, 41, 48, 49, 50, 51, 52, 59, 60, 61, 62, 63, 64, 71, 72, 73, 74, 75, 76, 77, 78, 79, 80, 81, 89, 90, 91, 92, 93, 100, 101, 102, 103, 104, 113, 114, 121, 122, 123, 124, 125, 131, 132, 133, 134, 135, 136, 143, 144, 145, 146, 147, 148, 149, 150, 151, 158, 159, 160, 161, 162, 163, 164, 165, 166, 173, 174, 175, 176, 177, 178, 179, 180, 181, 223, 235 and 246,
(b) a peptide linker having an amino acid sequence selected from the group consisting of SEQ ID Nos: 187-189;
€ the second domain having an amino acid sequence selected from the group consisting of SEQ ID Nos: SEQ ID Nos: 23, 25, 41, 43, 59, 61, 77, 79, 95, 97, 113, 115, 131, 133, 149, 151, 167, 169, 185 or 187 of WO 2008/119567 or having 80, 90, 95, 99 or 100% sequence identity with any of SEQ ID NOs: 26, 382 and 383;
(d) a peptide linker having an amino acid sequence selected from the group consisting of SEQ ID Nos: 187, 188, 189, 195, 196, 197 and 198;
(e) the first polypeptide monomer of the third domain having 80, 90, 95, 99 or 100% sequence identity with a polypeptide sequence selected from the group consisting of SEQ ID Nos: 437 to 444;
(f) a peptide linker having an amino acid sequence selected from the group consisting of SEQ ID Nos: 191, 192, 193 and 194; and
(g) the second polypeptide monomer of the third domain having 80, 90, 95, 99 or 100% sequence identity with a polypeptide sequence selected from the group consisting of SEQ ID Nos: 437 to 444.

In one aspect, the TCE molecule, which is understood herein to be a bispecific antigen-binding molecule, is characterized by having an amino acid sequence selected from the group consisting of and being directed to the respective target cell surface antigen:

| | | |
|---|---|---|
| (a) | SEQ ID Nos: 27, 28, 37 to 41; | CD33 |
| (b) | SEQ ID Nos: each of 48 to 52; | EGFRvIII |
| (c) | SEQ ID Nos: each of 59 to 64; | MSLN |
| (d) | SEQ ID Nos: each of 71 to 82 | CDH19 |
| (e) | SEQ ID Nos: each of 100 to 104 | DLL3 |
| (f) | SEQ ID Nos: 7, 8, 17, 113 | CD19 |
| | and 114 | |
| (g) | SEQ ID Nos: each of 89 to 93 | FLT3 |
| (h) | SEQ ID Nos: each of 121 to 125 | CDH3 |
| (i) | SEQ ID Nos: each of 132 to 136 | BCMA |
| (j) | SEQ ID Nos: each of 143 to | PSMA |
| | 151, 158 to 166 and 173 to 181 | |
| (k) | SEQ ID NO 213 | MUC17 |
| (l) | SEQ ID NOs: each of 225 | CLDN18.2 and |
| | and 237 | |
| (m) | SEQ ID No: 248 | CD70 |
| (n) | SEQ ID NO: 255 | CDH3 and MSLN |
| (o) | SEQ ID NOs: 373-375 | STEAP1 (each molecule |
| | | comprising 3 chains, |
| | | respectively); 373, 376 and |
| | | 380; and 377-379 |
| (p) | SEQ ID NO: 430 | CLDN6. |

The disclosure further provides a polynucleotide/nucleic acid molecule encoding an antigen-binding molecule of the invention. A polynucleotide is a biopolymer composed of 13 or more nucleotide monomers covalently bonded in a chain. DNA (such as cDNA) and RNA (such as mRNA) are examples of polynucleotides with distinct biological function. Nucleotides are organic molecules that serve as the monomers or subunits of nucleic acid molecules like DNA or RNA. The nucleic acid molecule or polynucleotide can be double stranded and single stranded, linear and circular. It is preferably comprised in a vector which is preferably comprised in a host cell. Said host cell is. e.g. after transformation or transfection with the vector or the polynucleotide, capable of expressing the antigen-binding molecule. For that purpose the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code. While the genetic code determines the protein sequence for a given coding region, other genomic regions can influence when and where these proteins are produced.

Furthermore, the disclosure provides a vector comprising a polynucleotide/nucleic acid molecule. A vector is a nucleic acid molecule used as a vehicle to transfer (foreign) genetic material into a cell. The term “vector” encompasses—but is not restricted to—plasmids, viruses, cosmids and artificial chromosomes. In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Modem vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding side. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding side is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density.

Moreover, the disclosure provides a host cell transformed or transfected with the polynucleotide/nucleic acid molecule or with the vector. As used herein, the terms “host cell” or “recipient cell” are intended to include any individual cell or cell culture that can be or has/have been recipients of vectors, exogenous nucleic acid molecules, and polynucleotides encoding the antigen-binding molecule disclosed herein; and/or recipients of the antigen-binding molecule itself. The introduction of the respective material into the cell is carried out by way of transformation, transfection and the like. The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

The antigen-binding molecule can be produced in bacteria. After expression, the antigen-binding molecule is isolated from the E. coli cell paste in a soluble fraction and can be purified through, e.g., affinity chromatography and/or size exclusion. Final purification can be carried out similar to the process for purifying antibody expressed e.g., in CHO cells.

In addition to prokaryotes, eukaryotic microbes such as filamentous fungi or yeast are suitable cloning or expression hosts for the antigen-binding molecule. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antigen-binding molecule are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa calfornica NPV and the Bm-5 strain of Bombyx mori NPV, and such viruses may be used as the virus herein, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)); baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells (Mather et al., Annals N. Y Acad. Sci. (1982) 383: 44-68); MRC 5 cells; FS4 cells; and a human hepatoma line (Hep G2).

In a further embodiment the disclosure provides a process for the production of an antigen-binding molecule, said process comprising culturing a host cell under conditions allowing the expression of the antigen-binding molecule and recovering the produced antigen-binding molecule from the culture.

As used herein, the term “culturing” refers to the in vitro maintenance, differentiation, growth, proliferation and/or propagation of cells under suitable conditions in a medium. The term “expression” includes any step involved in the production of an antigen-binding molecule including, but not limited to, transcription, post-transcriptional modification, translation, post-translational modification, and secretion.

When using recombinant techniques, the antigen-binding molecule can be produced intracellularly, in the periplasmic space, or directly secreted into the medium. If the antigen-binding molecule is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antigen-binding molecule prepared from the host cells can be recovered or purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromato-focusing SDS-PAGE, and ammonium sulfate precipitation are also available depending on the antibody to be recovered. Where the antigen-binding molecule comprises a CH3 domain, the Bakerbond ABX resin (.T. Baker, Phillipsburg, NJ) is useful for purification.

Affinity chromatography is a preferred purification technique. The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose.

Moreover, the disclosure provides a pharmaceutical composition comprising an antigen-binding molecule or an antigen-binding molecule produced according to the process disclosed herein. It is preferred for the pharmaceutical composition that the homogeneity of the antigen-binding molecule is ≥80%, more preferably ≥81%, ≥82%, ≥83%, ≥84%, or ≥85%, further preferably ≥86%, ≥87%, ≥88%, ≥89%, or ≥90%, still further preferably, ≥91%, ≥92%, ≥93%, ≥94%, or ≥95% and most preferably ≥96%, ≥97%, ≥98% or ≥99%.

As used herein, the term “pharmaceutical composition” relates to a composition which is suitable for administration to a patient, preferably a human patient. The particularly preferred pharmaceutical composition comprises one or a plurality of the antigen-binding molecule(s) on, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions include, but are not limited to, liquid, frozen, and lyophilized compositions.

The compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antigen-binding molecule and further one or more excipients such as those illustratively described in this section and elsewhere herein. Excipients can be used in this regard for a wide variety of purposes, such as adjusting physical, chemical, or biological properties of formulations, such as adjustment of viscosity, and or processes to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A.R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH; examples of buffers are borate, bicarbonate, Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycin, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

Different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

It is envisaged that the composition might comprise, in addition to the polypeptide defined herein, further biologically active agents, depending on the intended use of the composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art. It is also envisaged that the antigen-binding molecule is applied in a co-therapy, i.e., in combination with another anti-cancer medicament.

In certain embodiments, compositions may influence the physical state, stability, rate of in vivo release and rate of in vivo clearance of the antigen-binding molecule. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In certain embodiments, the antigen-binding molecule of the compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the antigen-binding molecule may be formulated as a lyophilizate using appropriate excipients such as sucrose.

When parenteral administration is contemplated, the therapeutic compositions for use herein may be provided in the form of a pyrogen-free, parenterally acceptable aqueous solution comprising the desired antigen-binding molecule of the invention in a pharmaceutically acceptable vehicle. A particularly suitable vehicle for parenteral injection is sterile distilled water in which the antigen-binding molecule is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen-binding molecule.

The antigen-binding molecule may also be entrapped in microcapsules prepared, for example, by coacervation techniques or by interfacial polymerization (for example, hydroxymethylcellulose or gelatin-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16^{th} edition, Oslo, A., Ed., (1980).

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilisation and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Another aspect includes self-buffering antigen-binding molecule formulations, which can be used as pharmaceutical compositions, as described in international patent application WO 06138181A2 (PCT/US2006/022599). A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions”, Pharm Biotechnol. 13: 159-75 (2002), see particularly the parts pertinent to excipients and processes of the same for self-buffering protein formulations, especially as to protein pharmaceutical products and processes for veterinary and/or human medical uses.

Salts may be used in accordance with certain embodiments to, for example, adjust the ionic strength and/or the isotonicity of a formulation and/or to improve the solubility and/or physical stability of a protein or other ingredient of a composition. As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes commonly are used at high concentrations (e.g., ≥1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in the antigen-binding molecule formulations in accordance with various embodiments as bulking agents, stabilizers, and antioxidants, as well as other standard uses. Lysine, proline, serine, and alanine can be used for stabilizing proteins in a formulation. Glycine is useful in lyophilisation to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations. Among polyols useful in select embodiments is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use herein. In addition, sugars that form such reactive species, such as sucrose, which is hydrolyzed to fructose and glucose under acidic conditions, and consequently engenders glycation, also is not among preferred polyols in this regard. PEG is useful to stabilize proteins and as a cryoprotectant and can be used in this regard.

Embodiments of the antigen-binding molecule formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product. Surfactants routinely are used to prevent, minimize, or reduce surface adsorption. Useful surfactants in this regard include polysorbate 20, polysorbate 80, other fatty acid esters of sorbitan polyethoxylates, and poloxamer 188. Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of the antigen-binding molecule of the invention formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard. Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins. Magnesium ions (10-120 mM) can be used to inhibit isomerization of aspartic acid to isoaspartic acid. Ca⁺² ions (up to 100 mM) can increase the stability of human deoxyribonuclease. Mg⁺², Mn⁺², and Zn⁺², however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the antigen-binding molecule of the invention formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized-dual chamber cartridge, Pharmacia & Upjohn) contain phenol while Somatrope (Eli Lilly) is formulated with m-cresol.

As might be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (about 18 to 24 months). An important point to note is that preservative effectiveness should be demonstrated in the final formulation containing the active drug and all excipient components.

The antigen-binding molecules disclosed herein may also be formulated as liposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antigen-binding molecule are prepared by methods known in the art, such as described in Epstein et al., Proc. Natl. Acad. Sci. USA, 82: 3688 (1985); Hwang et al., Proc. Natl Acad. Sci. USA, 77: 4030 (1980); U.S. Pat. Nos. 4,485,045 and 4,544,545; and WO 97/38731. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antigen-binding molecule of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81 (19) 1484 (1989).

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Homing S J, Coiffier B, Shipp M A, Fisher R I, Connors J M, Lister T A, Vose J, Grillo-Lopez A, Hagenbeek A, Cabanillas F, Klippensten D, Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 Apr; 17(4):1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above. It is an envisaged characteristic of the antigen-binding molecules of the present invention provided with the specific FC modality that they comprise, for example, differences in pharmacokinetic behavior. A half-life extended targeting antigen-binding molecule according to the present invention preferably shows a surprisingly increased residence time in vivo in comparison to “canonical” non-HLE versions of said antigen-binding molecule.

“Half-life” means the time where 50% of an administered drug are eliminated through biological processes, e.g. metabolism, excretion, etc. By “hepatic first-pass metabolism” is meant the propensity of a drug to be metabolized upon first contact with the liver, i.e. during its first pass through the liver. “Volume of distribution” means the degree of retention of a drug throughout the various compartments of the body, like e.g. intracellular and extracellular spaces, tissues and organs, etc. and the distribution of the drug within these compartments. “Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates, more onset and/or Cmax for a given amount of drug administered. “Bioavailability” means the amount of a drug in the blood compartment. “Lag time” means the time delay between the administration of the drug and its detection and measurability in blood or plasma. “Tmax” is the time after which maximal blood concentration of the drug is reached, and “Cmax” is the blood concentration maximally obtained with a given drug. The time to reach a blood or tissue concentration of the drug which is required for its biological effect is influenced by all parameters. Pharmacokinetic parameters of bispecific antigen-binding molecules exhibiting cross-species specificity, which may be determined in preclinical animal testing in non-chimpanzee primates as outlined above, are also set forth e.g. in the publication by Schlereth et al. (Cancer Immunol. Immunother. 20 (2005), 1-12).

In a preferred aspect of the invention the pharmaceutical composition is stable for at least four weeks at about −20° C. As apparent from the appended examples the quality of an antigen-binding molecule of the invention vs. the quality of corresponding state of the art antigen-binding molecules may be tested using different systems. Those tests are understood to be in line with the “ICH Harmonised Tripartite Guideline: Stability Testing of Biotechnological Biological Products Q5C and Specifications: Test procedures and Acceptance Criteria for Biotech Biotechnological Biological Products Q6B” and, thus are elected to provide a stability-indicating profile that provides certainty that changes in the identity, purity and potency of the product are detected. It is well accepted that the term purity is a relative term. Due to the effect of glycosylation, deamidation, or other heterogeneities, the absolute purity of a biotechnological/biological product should be typically assessed by more than one method and the purity value derived is method-dependent. For the purpose of stability testing, tests for purity should focus on methods for determination of degradation products.

For the assessment of the quality of a pharmaceutical composition comprising an antigen-binding molecule of the invention may be analyzed e.g. by analyzing the content of soluble aggregates in a solution (HMWS per size exclusion). It is preferred that stability for at least four weeks at about −20° C. is characterized by a content of less than about 5% HMWS, more preferably of less than 2.5% HMWS, even more preferably by less than 1.5% HMWS.

Other examples for the assessment of the stability of an antigen-binding molecule in form of a pharmaceutical composition are provided in the appended examples 4-12. In those examples embodiments of antigen-binding molecules are tested with respect to different stress conditions in different pharmaceutical formulations and the results compared with other half-life extending (HLE) formats of bispecific T cell engaging antigen-binding molecule known from the art. In general, it is envisaged that antigen-binding molecules provided with the specific FC modality according to the present invention are typically more stable over a broad range of stress conditions such as temperature and light stress, both compared to antigen-binding molecules provided with different HLE formats and without any HLE format (e.g. “canonical” antigen-binding molecules). Said temperature stability may relate both to decreased (below room temperature including freezing) and increased (above room temperature including temperatures up to or above body temperature) temperature. As the person skilled in the art will acknowledge, such improved stability with regard to stress, which is hardly avoidable in clinical practice, makes the antigen-binding molecule safer because less degradation products will occur in clinical practice. In consequence, said increased stability means increased safety.

One embodiment provides the antigen-binding molecule of the invention or the antigen-binding molecule produced according to the process of the invention for use in the prevention, treatment or amelioration of a proliferative disease, a tumorous disease, a viral disease or an immunological disorder.

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as specified herein below, by the administration of an antigen-binding molecule according to the invention to a subject in need thereof. Such an improvement may also be seen as a slowing or stopping of the progression of the tumor or cancer or metastatic cancer of the patient. The term “prevention” as used herein means the avoidance of the occurrence or re-occurrence of a patient having a tumor or cancer or a metastatic cancer as specified herein below, by the administration of an antigen-binding molecule according to the invention to a subject in need thereof.

The term “disease” refers to any condition that would benefit from treatment with the antigen-binding molecule or the pharmaceutic composition described herein. This includes chronic and acute disorders or diseases including those pathological conditions that predispose the mammal to the disease in question.

A “neoplasm” is an abnormal growth of tissue, usually but not always forming a mass. When also forming a mass, it is commonly referred to as a “tumor”. Neoplasms or tumors or can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metatstatic cancer” encompasses metastases to other tissues or organs than the one of the original tumor. Lymphomas and leukemias are lymphoid neoplasms. For the purposes of the present invention, they are also encompassed by the terms “tumor” or “cancer”.

The term “viral disease” describes diseases, which are the result of a viral infection of a subject.

The term “immunological disorder” as used herein describes in line with the common definition of this term immunological disorders such as autoimmune diseases, hypersensitivities, immune deficiencies.

In one embodiment the invention provides a method for the treatment or amelioration of a proliferative disease, a tumorous disease, a viral disease or an immunological disorder, comprising the step of administering to a subject in need thereof the antigen-binding molecule of the invention, or produced according to the process of the invention.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The molecules of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, ophthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the antigen-binding molecule of this invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the antigen-binding molecule of the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the antigen-binding molecules of the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilisation.

The compositions of the present invention can be administered to the subject at a suitable dose which can be determined e.g. by dose escalating studies by administration of increasing doses of the antigen-binding molecule of the invention exhibiting cross-species specificity described herein to non-chimpanzee primates, for instance macaques. As set forth above, the antigen-binding molecule of the invention exhibiting cross-species specificity described herein can be advantageously used in identical form in preclinical testing in non-chimpanzee primates and as drug in humans.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered antigen-binding molecule, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system.

A therapeutic effective amount of an antigen-binding molecule of the invention preferably results in a decrease in severity of disease symptoms, an increase in frequency or duration of disease symptom-free periods or a prevention of impairment or disability due to the disease affliction. For treating target cell antigen-expressing tumors, a therapeutically effective amount of the antigen-binding molecule of the invention, e.g. an anti-target cell antigen/anti-CD3 antigen-binding molecule, preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy.

The pharmaceutical composition can be administered as a sole therapeutic or in combination with additional therapies such as anti-cancer therapies as needed, e.g. other proteinaceous and non-proteinaceous drugs. These drugs may be administered simultaneously with the composition comprising the antigen-binding molecule of the invention as defined herein or separately before or after administration of said antigen-binding molecule in timely defined intervals and doses.

The term “effective and non-toxic dose” as used herein refers to a tolerable dose of an inventive antigen-binding molecule which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

Finally, the invention provides a kit comprising an antigen-binding molecule of the invention or produced according to the process of the invention, a pharmaceutical composition of the invention, a polynucleotide of the invention, a vector of the invention and/or a host cell of the invention.

In the context of the present invention, the term “kit” means two or more components—one of which corresponding to the antigen-binding molecule, the pharmaceutical composition, the vector or the host cell of the invention—packaged together in a container, recipient or otherwise. A kit can hence be described as a set of products and/or utensils that are sufficient to achieve a certain goal, which can be marketed as a single unit.

The kit may comprise one or more recipients (such as vials, ampoules, containers, syringes, bottles, bags) of any appropriate shape, size and material (preferably waterproof, e.g. plastic or glass) containing the antigen-binding molecule or the pharmaceutical composition of the present invention in an appropriate dosage for administration (see above). The kit may additionally contain directions for use (e.g. in the form of a leaflet or instruction manual), means for administering the antigen-binding molecule of the present invention such as a syringe, pump, infuser or the like, means for reconstituting the antigen-binding molecule of the invention and/or means for diluting the antigen-binding molecule of the invention.

The invention also provides kits for a single-dose administration unit. The kit of the invention may also contain a first recipient comprising a dried/lyophilized antigen-binding molecule and a second recipient comprising an aqueous formulation. In certain embodiments of this invention, kits containing single-chambered and multi-chambered pre-filled syringes (e.g., liquid syringes and lyosyringes) are provided.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### Example 1: Binding Peptide Modification, Binding Peptide to Polymer Conjugation and Characterization

Materials and methods for all experiments: Peptide—Carrier conjugation:

### Disulfide Conjugation Reaction:

### Reagents:

Reaction Buffer: PBS 100 mM Phosphate, 250 mM NaCl, pH 6.5, filtered (Endotoxin Free)

Cell Culture Water

15 mL Falcon Tube

Amicon filtration centrifuge tubes

### Procedure:

Dissolve OPSS or Maleimide containing PEG to 4 mM in Reaction Buffer. Dissolve binding peptide to 5 mM in Cell Culture Water or Reaction Buffer. Reactions were carried out with between 62.5 uM and 125 uM PEG in a total volume of 7200 ul. Add PEG and Reaction Buffer to 10 mL Falcon Tube. With gentle vortexing, add 8 equivalents of peptide, slowly. Rotate mixture for 3 hours at room temperature. After 3 hours, add reaction mixture to appropriately sized MW amicon filtration unit (i.e. 3 kDa filter for conjugates derived from 4-arm 2- and 5-kDa PEG, 10-kDa filters for conjugates derived from 4-arm 10- and 20-kDa PEG). Add reaction buffer to 15 mL and concentrate reaction to between 1 and 2 mL. Repeat addition of buffer and concentration for a total of 4 concentration steps. Conjugates are stored at −20° C. until the concentration could be measured via mass spec quantitation assay. Reagents have been stored between 3-25 mg/mL in reaction buffer.

### Thioether Conjugation Reaction (Mal, AcBr):

1. Reduce OPSS- or thiol-functionalized PEG with TCEP
2. Remove TCEP (if necessary) and buffer exchange to reaction buffer (50 mM sodium phosphate, 2 mM EDTA, pH7.5, or similar).
3. Add BrAc-functionalized peptide in excess. Incubate at RT O/N.
4. Purify by RP-HPLC.

### Azide Alkyne Cycloaddition:

Exemplary Preparation of 73136 pE-DGNEELKGK(mPEG)-OH, Linear, 20 kDa, Acetamide

The binding peptide solution was first prepared by dissolving pE-DGNEELKGK(bromoacetyl)-OH in Reaction Buffer 1 (50 mM Phosphate, 2 mM EDTA, pH 7.5) at 5 mg/mL. To a 15-mL conical tube, was added 20 kDa α-mercaptoethyl-ω-methoxy, polyoxyethylene (SUNBRIGHT ME-200SH, NOF America, White Plains, NY). The peptide solution was added to the tube containing the PEG to the final ratio of 2 equivalent of the bromoacetyl-peptide to the thiol. The mixture was pulse-vortexed gently until the PEG was completely dissolved. The pH of the solution was adjusted to 8 by adding a sufficient amount of 1 M HEPES, pH 8.5. The reaction mixture was incubated overnight at an ambient temperature on a rotary incubator. The completed reaction was stored at −20° C. until purification.

Preparation of 79677 pE-DGEEELKGC(PEG)-NH2, 4-Arm, 5 kDa, MAL

The peptide solution was first prepared by dissolving pE-DGEEELKGC-NH₂ at 5 mg/mL in Reaction Buffer 2 (100 mM Sodium phosphate 250 mM NaCl, pH 6.5) in a 50-mL conical tube. The PEG solution was prepared by dissolving 5 kDa 4-Arm PEG-MAL (PSB-453, Creative PEGWorks, Chapel Hill, NC) in the Reaction Buffer 2 at 50 mg/mL. The PEG stock solution was kept on ice during the operation. The PEG solution was added to the peptide solution dropwise at an ambient temperature while gently vortexing intermittently, to the final ratio of 2 equivalent thiol-peptide to maleimide. The completed reaction was stored at −20° C. until purification.

### General Procedure of Purification of TCE Masking Molecules

The binding peptide-PEG conjugates were purified by reverse-phase high-performance liquid chromatography using Agilent HPLC system 1290 Infinity II, IFC equipped with Phenomenex Gemini 5 um NX-C18 110A, 250×30 mm LC column. The compounds were eluted at the flow rate of 30 mL/min in the gradient of 0-60% B over 45 min, with the mobile phase A: 20 mM ammonium bicarbonate, 5% acetonitrile in water, and B: 20 mM ammonium bicarbonate, 75% acetonitrile in water. Fractions containing the desired product were pooled, lyophilized, solubilized in deionized water, and filter-sterilized using 0.22 μm PDVF filter (Steriflip® Vacuum Tube Top Filter, Millipore, Burlington. MA). The solution was re-lyophilized to remove any residual ammonium bicarbonate. The product was re-solubilized in sterile deionized water and the peptide content was determined by chemiluminescence nitrogen detection using Antek MultiTek (PAC, Houston, TX) according to the manufacturer's instructions. The final product was formulated as pre-quantified dry powder by aliquoting the final solution of known peptide content into sterile vials and lyophilizing.

### Binding Assays

SPR Solution Assay of Competition Binding with TCE

### Equilibrium Binding Affinity Measurement by KinExA

1. Preparation of NHS-activated sepharose fast-flow beads:
2. Fast flow sepharose suspension in 100% IPA, washed twice with milliQ H20, mild centrifugation to pool beads in between wash steps.
3. Equilibrated in 0.15 M NaBO3, pH=8.5 buffer
4. Added 80 mM PDEA to activate the resin for thiol exchange, 10 mins, r.t.
5. Equilibrated resin with Hepes Buffered Saline-P (0.01 M HEPES pH 7.4, 0.15 M NaCl, 0.005% v/v Surfactant P2), pH 7.4 binding buffer
6. Added 30 μg/mL of binding peptide, QDGNEEMGC (SEQ ID NO; 297) or pGlu-DGNEEMGKGMEENGDQC (SEQ ID NO: 300), reaction for 1 hr to functionalize resin.
7. Quench with 10 mM L-Cys for 1 hour.
8. Constant Binding Partner was FLT3×CD3 TCE (SEQ ID NO: 93), MW 105720, 3.2 mg/mL 31.21 uM was diluted to 333 pM and the pGlu-DGNEEMGKGMEENGDQC peptide or pGlu-DGNEEMGKGMEENGDQC-PEG (Pep-PEG v2) were titrated from 1 uM or 400 nM down to 2.3 pM concentrations relative to effective peptide molarity.

### Association Kinetics by KinExA

### Results

### In Vitro Binding Affinity and Kinetics

TABLE 5
Tabulated in vitro binding affinity and kinetic parameters for CD3ϵ Peptide and CD3ϵ PepPOL
variant forms.

| | Equilibrium | kon | koff | fold- | fold- | fold- |
|---|---|---|---|---|---|---|
| CD3ϵ Binding Peptide | Kd (nM) | (1/M*s) | (1/s) | kon | koff | Kd |
| QDGNEEMG | 35.8 | 6.92E+05 | 2.48E−02 | | | |
| QDGNEEMG-K | 22.3 | 8.06E+05 | 1.79E−02 | | | |
| QDGNEEMG-GMEENGDQ | 9.88 | 4.76E+05 | 4.71E−03 | 0.69 | 0.19 | 3.62 |
| QDGNEEMG-QDGNEEMG | 11 | 5.33E+05 | 5.86E−03 | 0.77 | 0.24 | 3.25 |
| QDGNEEMG-K-QDGNEEMG | 8.05 | 4.48E+05 | 4.97E−03 | 0.56 | 0.28 | 2.77 |
| QDGNEEMG-K(Nϵ)-GMEENGDQ | 1.03 | 4.34E+06 | 4.47E−03 | 5.38 | 0.25 | 21.65 |
| QDGNEEMG-P(Dap)- | 1.1 | 4.40E+06 | 7.50E−03 | 6.36 | 0.30 | 32.55 |
| GMEENGDQ | | | | | | |
| (QDGNEEMG)3K | 0.104 | 1.90E+06 | 1.60E−04 | 2.75 | 0.01 | 344 |
| (QDGNEEMGKGMEENGDQC)4 - | 22 | 1.99E+05 | 4.70E−03 | 1.14 | — | — |
| PEG(20 kDa) | | | | | | |
| pGlu-DGNEEMGKGMEENGDQC | 0.176 | | | | | 203 |
| (pGlu- | 0.176 | 1.12E+07 | 1.95E−3 | 16.2 | 0.01 | 203 |
| DGNEEMGKGMEENGDQC)4 - | | | | | | |
| PEG(20 kDa) | | | | | | |
| pGlu-DGNEELKGC | 0.142 | | | | | 252 |
| (pGlu-DGNEELKGC)4 -MAL | 0.142 | | | | | |
| PEG(10 kDa) | | | | | | |
| (pGlu-DGNEELKGC)4 -MAL | 0.142 | | | | | |
| PEG(5 kDa) | | | | | | |

As it can be seen from the above results, the functionalization of binding peptide with respect to the original CD3 epitope, in the present case N-terminal CD3ε QDGNEEMG (SEQ ID NO. 262), leads to lower Kd values, i.e. higher affinity which is typically preferred.

Further, Table 6 below shows the results of a surface plasmon resonance competition binding assay that highlights the affinity gains in comparison to the physiologic CD3ε epitope comprising the sequence QDGNEEMG achieved through Q (Gln) to pE (pGlu) substitution and maintained with conservative residue substitutions. As it can be derived from table 6,

TABLE 6
Activity gains by functionalized binding peptide

| | IC₅₀ by SPR | IC₅₀ by SPR |
|---|---|---|
| | (nM) FLT3xCD3 | (nM) STEAP1xCD3 |
| Sequence | TCE | TCE |
| QDGNEEMG | 132.9 | |
| pGlu- | 2.4 | |
| DGNEEMGKGMEENGDQC | | |
| pGlu-DGNEELKGC | 2.7 | 13.4 |
| pGlu-DGNEELKPGC | 5.2 | |
| pGlu-DGNEELPKPGC | 2.7 | |
| pGlu-DGEEELKGC | 14.7 | 28.6 |
| pGlu-DGSEELKGC | 8.2 | 16.7 |
| pGlu-DGTEELKGC | 39.7 | 59.9 |
| pGlu-DGVEELKGC | 9.0 | 26.4 |
| pGlu-DGIEELKGC | 3.6 | 11.6 |

### Example 2: Mouse Pharmacokinetic Study to Evaluate In Vivo PK

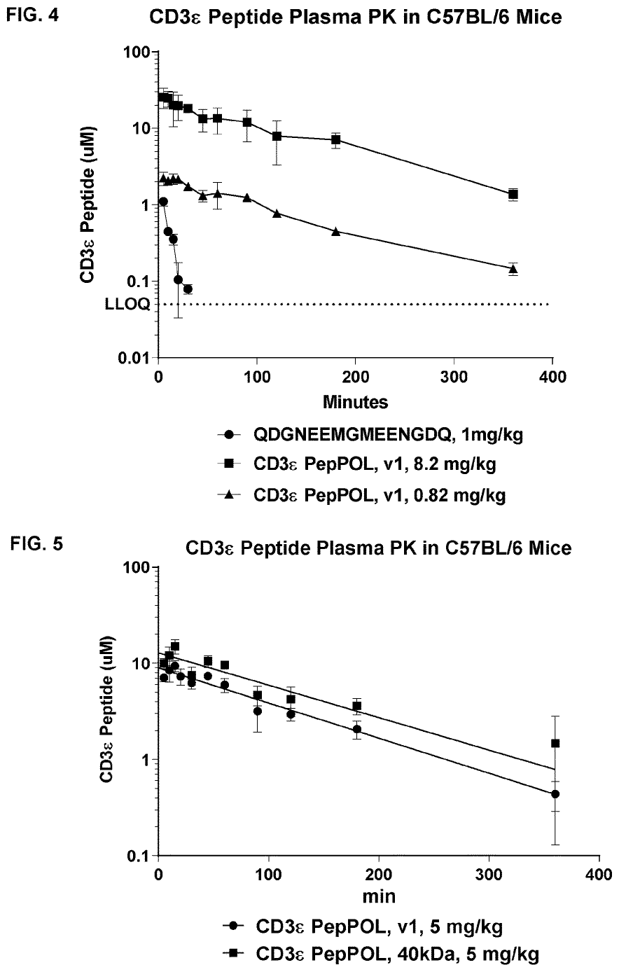

To evaluate the extension of peptide half-life through PEG-peptide conjugation, the dimeric and palindromic cysteine containing peptide, QDGNEEMGKGMEENGDQC, containing lysine engineered in and envisaged as a tryptic cleavable handle for analytical mass spectrometric quantitation, was chosen for initial evaluation in mice. The 4-arm 20 kDa PepPOL conjugate was dosed by IV administration to BL/6 mice at 0.82 or 8.2 mg/kg dose levels. Two non-conjugated parent peptide forms, i.e., QDGNEEMGKGMEENGDQ and QDGNEEMGKGMEENGDQC, were also dosed at 1 mg/kg as control arms to benchmark the half-life extension. The PK profiles for each peptide were evaluated by quantifying plasma concentrations of total intact peptide material over time (see methods). The terminal elimination half-life of the PepPOL conjugate was approximated to be 88 minutes based on curve fitting to the terminal slope in log-linear plotted form. In contrast the unconjugated peptides exhibited a terminal elimination half-life of between 3-10 minutes, consistent with their small size and presumably higher degree of renal clearance (see FIG. 4).

### Example 3: 2^{nd} Mouse PK Study

A second PK study was conducted in BL/6 mice to evaluate the plasma pharmacokinetics of the masking peptide in 8-arm (octavalent), 40 kDa PEG form. Both CD3ε PepPOL, v1 (40 kDa) and CD3ε PepPOL, v1 (20 kDa) (see Table 6 for sequences) were administered to mice by IV bolus at 5 mg/kg dose level and the results are shown in Fig. XX. Interestingly, the plasma PK of the CD3ε PepPOL, v1 (20 kDa) dosed as a control arm in the study and the that of the larger CD3ε PepPOL, v1 (40 kDa) form were similar, which indicates that the rate determining clearance mechanism may be driven by loss of the peptide and not intact CD3ε PepPOL. The terminal elimination half-lives were estimated to be between 90 min and 120 min for the 20 kDa or 40 kDa CD3ε PepPOL, v1 forms, respectively.

### Example 4: TCE Masking Molecule in PK Mouse Study

To further understand the in vivo behavior of the pyro-Glu containing dimeric p-EDGNEEMGKGMEENGDQC PepPOL conjugates, a 4-arm 20 kDa disulfide linked version ((pE-DGNEEMGKGMEENGDQC)4-PEG(20 kDa)) (CD3ε PepPOL, v2) was synthesized and evaluated in a single dose mouse PK study (C57BL/6). The PK profile of CD3ε PepPOL, v2 form is compared to the original CD3ε PepPOL, v1 to illustrate their similar behavior. The elimination half-lives were determined by non-linear one-phase decay fitting and estimated to be 102 minutes.

### Example 5: PK Evaluation of Binding Peptide Maleimide Trials

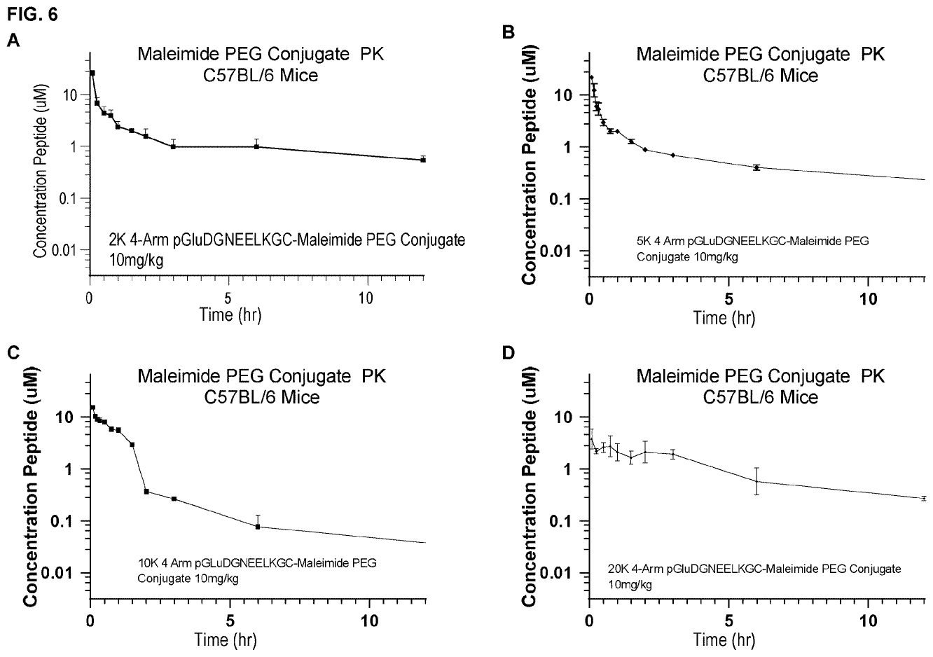

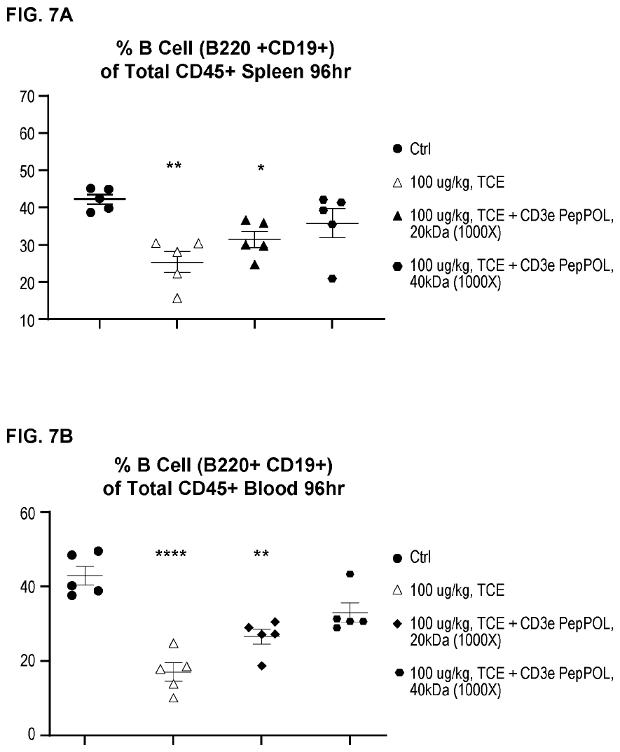

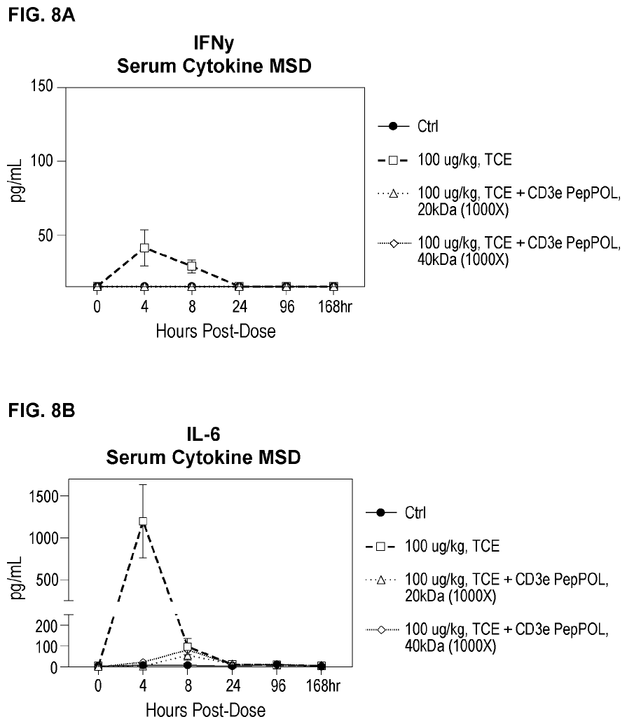

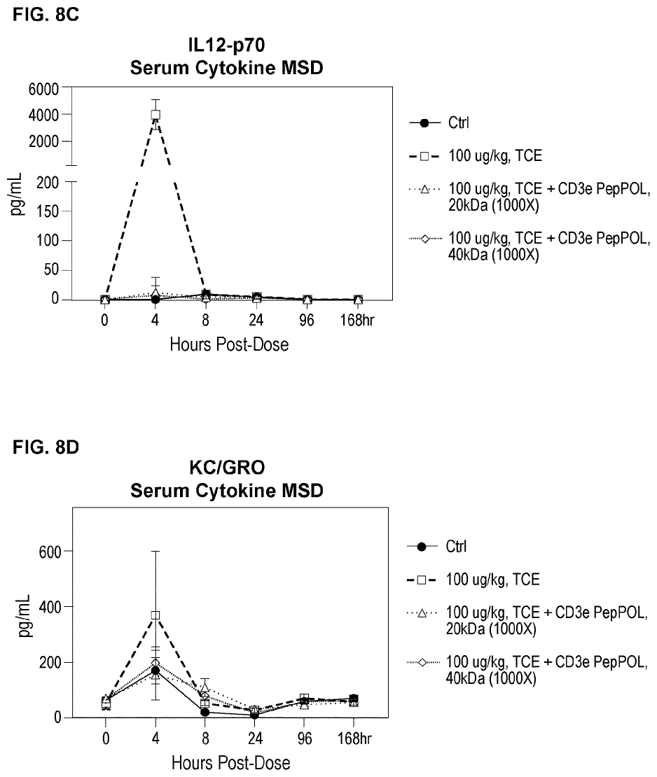

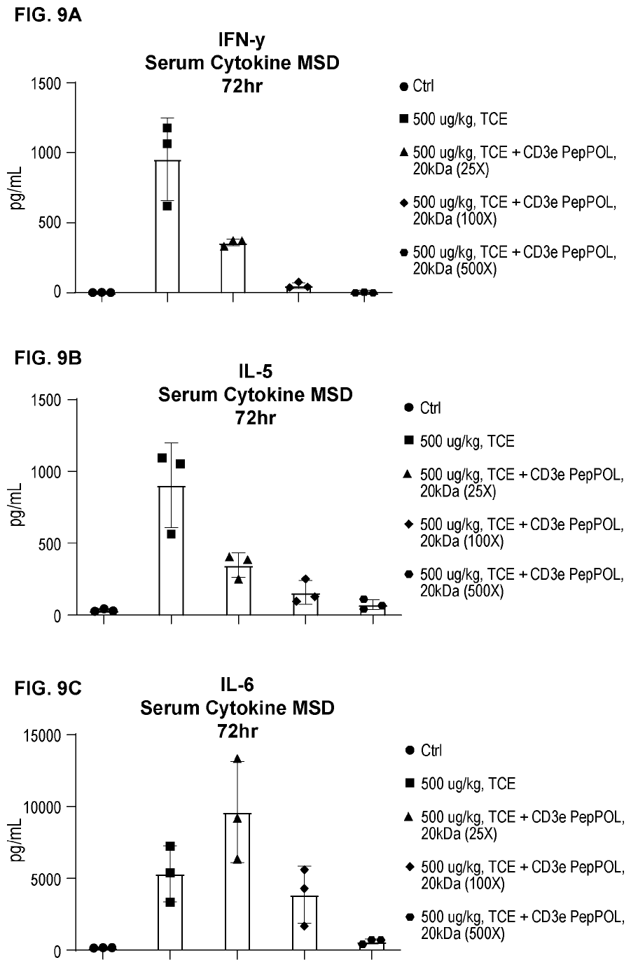

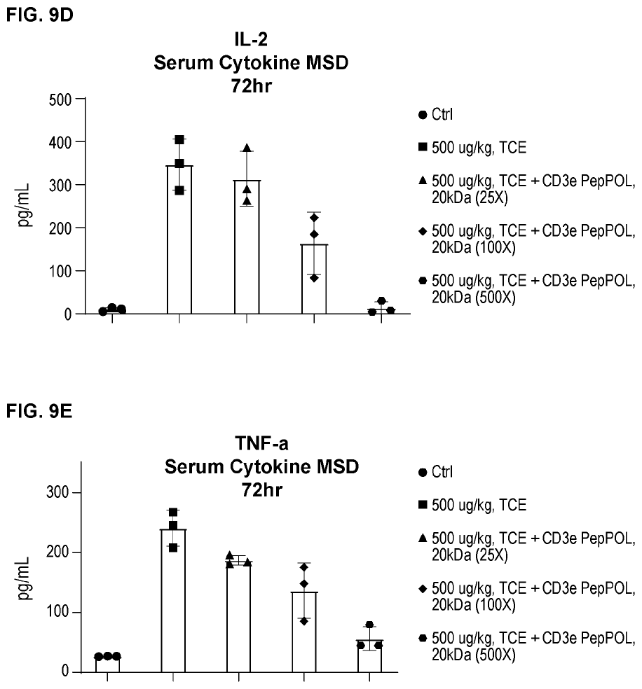

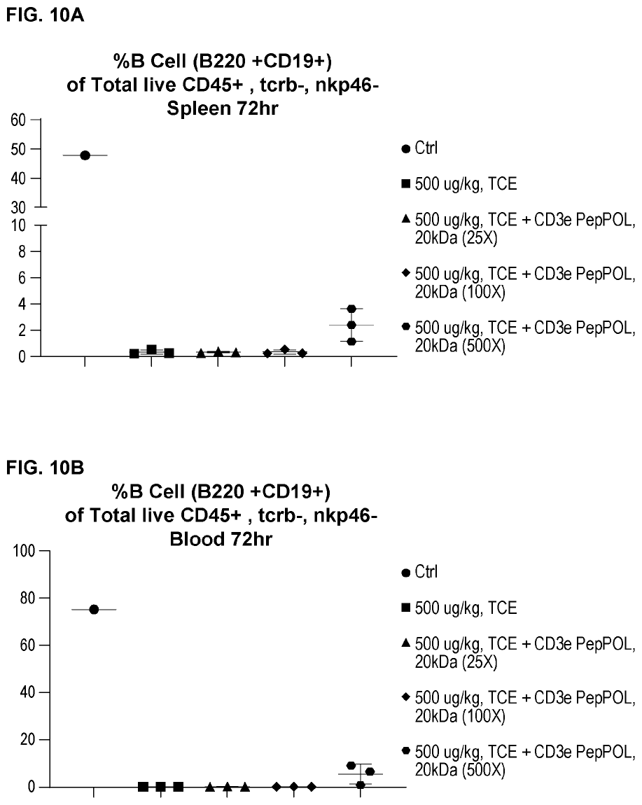

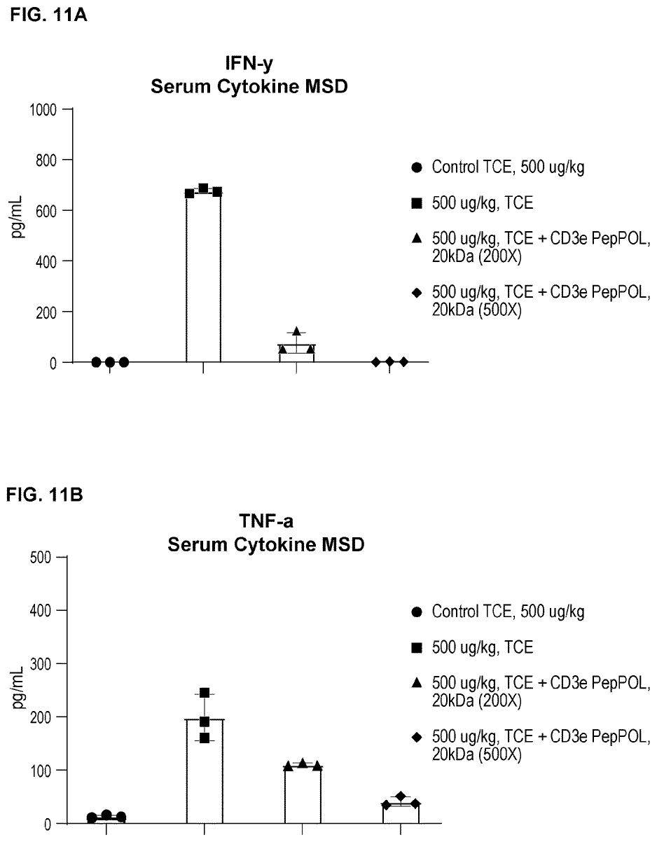

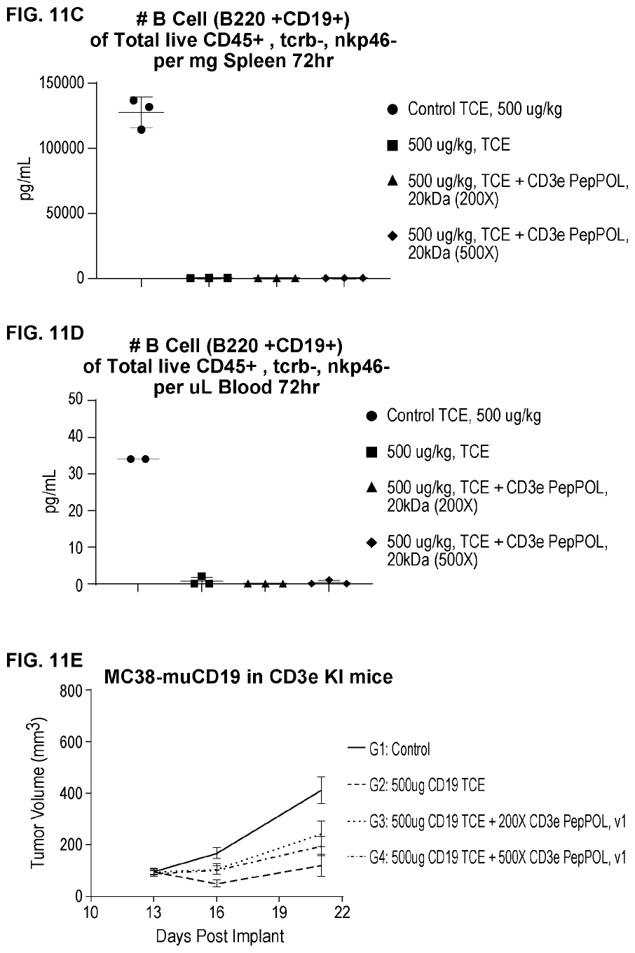

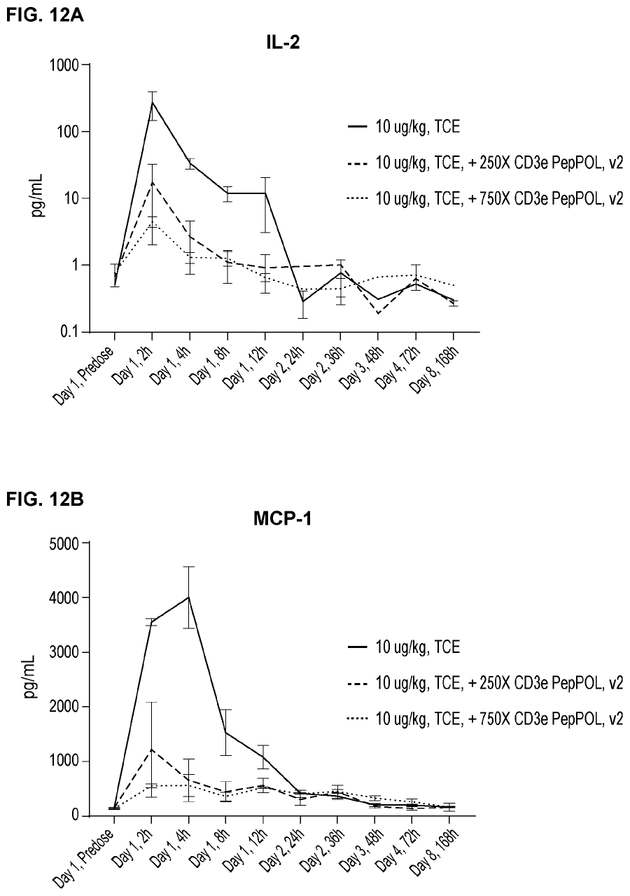

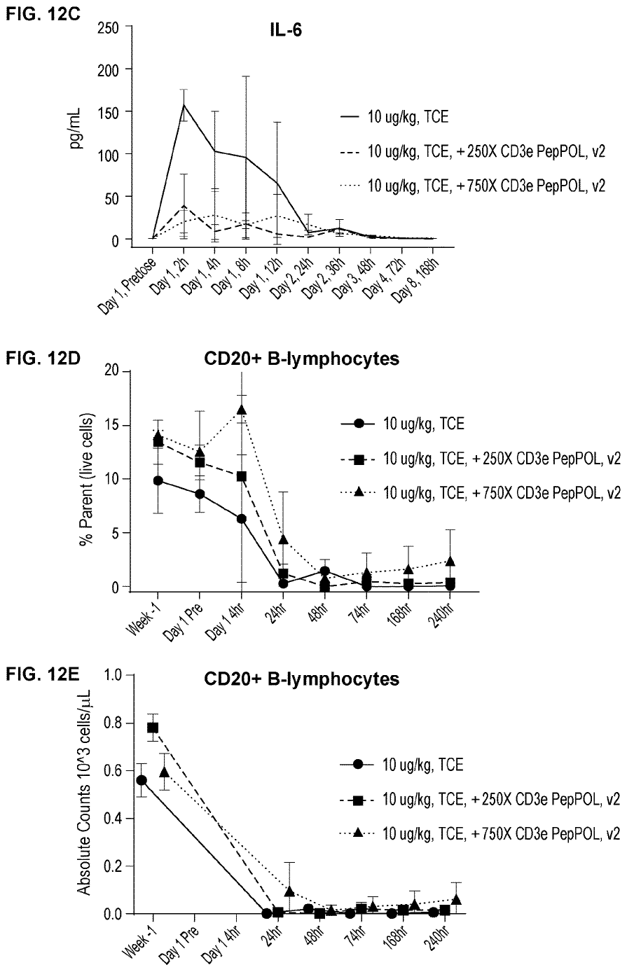

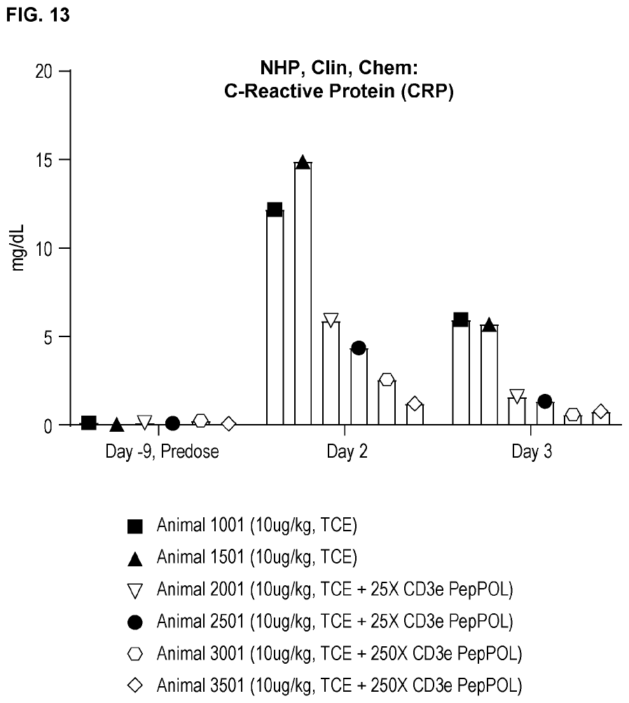

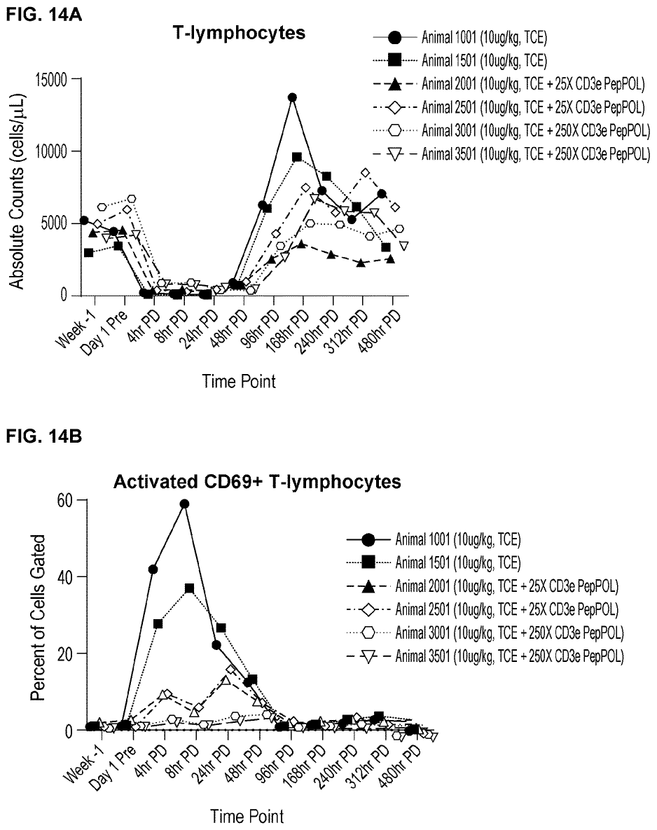

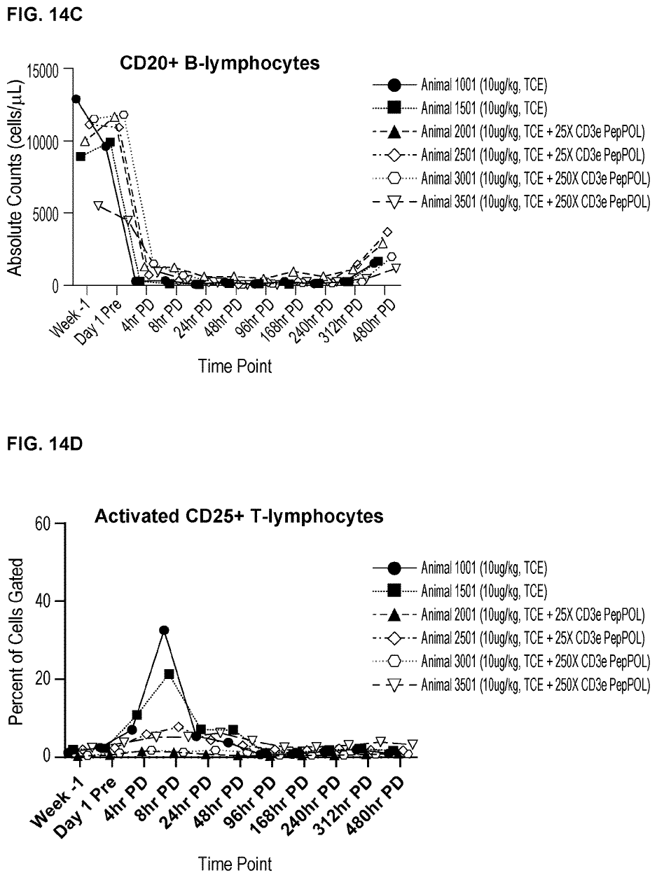

To evaluate the impact of linker chemistry on the plasma PK in rodents, the monomeric p-GluDGNEELGKC peptide was coupled through maleimide conjugation to 4-arm PEG carriers of 2-, 5-, 10-, and 20-kDa sizes, and their single dose plasma PK was evaluated in mice. The data are plotted in FIG. 6, and PK parameters tabulated in Table 3. From these studies, a size-dependence of the apparent distribution kinetics was observed that is illustrated most clearly by comparison of 2-compartment PK models that capture differential extent and duration of the ‘alpha’ or distribution phase of the PK profiles.

### Example 6: Study muCD19 TCE+CD3ε PepPOL, v1 Co-Administration in huCD3ε TCR KI Mouse Model. 100 ug/kg IV Dose Murine Anti-muCD19 TCE Investigated >2300X

An initial pilot study in huCD3ε TCR KI mice was conducted to establish the desired PD effect with CD3ε PepPol v1. Here, CD3ε PepPol, v1 was co-administered at ≥1000× molar overage (peptide:TCE) with 100 μg/kg muCD19 TCE. The molar overage is given as ≥1000× because the PK revealed that the TCE was underdosed, resulting in a higher than desired ratio. Nonetheless, the impact on cytokine reduction can be seen for both 20 kDa and 40 kDa PepPols across the panel, while 20 kDa PepPOL co-admin retained a greater degree of PD effect with respect to CD19+ B cell depletion.

### Example 7: Study muCD19 TCE+CD3ε PepPOL, v1 Co-Administration in huCD3ε TCR KI Mouse Model. 500 ug/kg IV Dose Murine Anti-muCD19 TCE+25×, 100×, and 500× Molar Excess CD3ε PepPOL, v1

This study aimed to characterize CD3ε PepPOL, v1 dose response in huCD3ε TCR KI mice at a projected efficacious muCD19 TCE dose (as determined by prior investigations in a solid tumor MC38-muCD19++ model)

Briefly, CD3ε PepPOL, v1 dose response was characterized in huCD3ε TCR KI mice with co-administration of 500 μg/kg muCD19 TCE delivered via an IV bolus injection. Pharmacodynamic endpoints included: a) CD19+ B cell immunophenotyping of both splenic and peripheral B cell populations by flow cytometry; and b) serum cytokines at 0-96 hr.

Result: Clear B cell depletion across all dose groups in both the peripheral blood and spleen, with no significant difference observed up to 100×molar excess of CD3ε PepPOL, v1. A slight residual B cell population (~2.4-6.5%) was observed in blood and spleen at 72 hrs post dose for the highest CD3ε PepPOL, v1 dosed group (500× molar excess). CD3ε PepPOL, v1 elicited a dose dependent reduction in all serum cytokines evaluated.

### Example 8: muCD19 TCE+CD3ε PepPOL, v1 Co-Administration in huCD3ε TCR KI, MC38-muCD19++Syngeneic Mouse Tumor Model. 500 ug/kg IV Dose Murine Anti-muCD19 TCE+200×, and 500× Molar Excess CD3ε PepPOL, v1

muCD19 TCE+CD3ε PepPOL, v1 co-administration in huCD3ε TCR KI mouse bearing MC38-muCD19++ solid tumors. This study served to extend the results of PD study #1 to include solid tumor growth inhibition after a single muCD19+CD3ε PepPOL, v1 co-administration.

Result: Complete B cell depletion in both spleen and blood by 72 hrs, and a statistically insignificant impact to tumor growth inhibition effect through day 20 for CD3ε PepPOL, v1 combo groups and CD3ε PepPOL, v1 dose dependent reduction in all cytokines measured.

### Example 9: Cynomolgus Study CD20 TCE (10 ug/kg)+CD3ε PepPOL, v1 (+250× (0.160 mg/kg), +750× (0.480 mg/kg) Co-Administration in Cynomolgus Monkeys

This PK/PD study aimed to characterize the ability of CD3ε PepPOL, v1 to uncouple cytokine release from CD20 TCE PD response (CD20+ B cell lysis) across a 3-fold CD3ε PepPOL, v1 dose range in combination with 10 μg/kg CD20 TCE dose. PD response was conserved across all groups with near complete depletion of circulating CD20+ B lymphocyte populations by 24 hrs post dose. There was a modest induction of IL-2, IL-6, and MCP-1 plasma levels following treatment with CD20 TCE alone, peaking at 2 (IL-2 and IL-6) or 4 (MCP-1) hours before returning to baseline by 72 hours. A CD3ε PepPOL, v1 dose response was observed for MCP-1, IL-2, and IL-6 cytokines, with near complete loss of detectable cytokines at all time points at the top CD3ε PepPOL, v1 dose of 0.480 mg/kg (750× molar in peptide).

### Example 10: Cynomolgus Study CD20 TCE (0.010 mg/kg)+CD3ε PepPOL, v2 (+25× (0.016 mg/kg), +250× (0.16 mg/kg) Co-Administration in Cynomolgus Monkeys

PK/PD study 157130 was an exploratory dose-ranging study conducted in cynomolgus monkeys aimed to establish activity of a ‘high affinity’ CD3ε PepPOL, v2 when co-administered with CD20 TCE. Primary study endpoints included: cytokine release, CD20+ B cell lysis, T cell activation, and clinical chemistry end points typically associated with CRS (e.g., CRP). CD20 TCE was dosed at 10 ug/kg alone, or in combination with CD3ε PepPOL, v2 at 0.016 mg/kg (25× molar excess CD3ε peptide), or 0.160 mg/kg (250× molar excess CD3ε peptide). A summary of the results is described below.

### Cytokines

Serum cytokines MCP-1, IL-6, TNFα, IFNγ, and IL-2 were assessed by multiplex sandwich immunoassay at multiple time points from 0-168 hours post dose.

### MCP-1

Intravenous (IV) administration of 0.010 mg/kg CD20 TCE to male (n=1/group) and female (n=1/group) monkeys resulted in increases in serum MCP-1 concentrations with peak serum concentrations observed by 4 hours post-dose for all groups, with MCP-1 concentrations trending to baseline by 48 hrs. For study groups receiving co-administration of CD3ε PepPOL, v2, a clear reduction in peak cytokine production was observed, with the magnitude of reduction being greatest (~98% reduction at 4 hr) for the 250× combo group.

### IL-6

Intravenous (IV) administration of 0.010 mg/kg CD20 TCE to male (n=1/group) and female (n=1/group) monkeys resulted in increases in serum IL-6 concentrations with peak serum concentrations observed by 4 hours post dose (8-12 fold over baseline) and the response trending to baseline by 8-12 hrs. post dose. Co-administration of 0.010 mg/kg CD20 with 0.016 mg/kg (25×) CD3ε PepPOL, v2 resulted in a moderate increase in IL-6 serum concentration for n=1 of 2 animals, with levels returning to baseline by 8-12 hrs. No IL-6 stimulation was observed in animals receiving a co-administration of 0.010 mg/kg CD20 TCE plus 0.16 mg/kg (250×) CD3ε PepPOL, v2 suggesting saturation of CD3ε PepPOL, v2 pharmacodynamic effect at this relative CD3ε PepPOL, v2 dose level.

### TNFα, IFNγ, and IL-2

Intravenous (IV) administration of 0.010 mg/kg CD20 TCE increased serum TNF-α concentrations in both animals that peaked between 2 hr and 4 hr postdose, and an increase in IFN-γ concentration was observed in 1 of 2 animals at 4 hr post dose. All three cytokines returned to baseline by 8 hr post dose. One animal co-administered 0.01 mg/kg CD20 TCE in combination with CD3ε PepPOL, v2 at 0.016 mg/kg had elevated levels of TNF-α and INF-γ at all timepoints (not shown) that did not appear to be treatment related. TNF-α and IFN-γ were not detected above the assay LLOQ at all time points in both animals dosed with CD20 TCE in combination with 0.160 mg/kg CD3ε PepPOL, v2. No detectable IL-2 response was observed in any group.

### T Lymphocyte Activation

CD3ε PepPOL, v2 co-administration had a pronounced effect on T cell activation, as assessed through both early (CD69+) and late (CD25+) markers monitored by flow cytometry. In general, the PD effect of CD3ε PepPOL, v2 co-administration mirrored that of serum cytokines, with clear mitigating CD3ε PepPOL, v2 dose response for T cell activation relative to 0.010 mg/kg CD20 TCE dosed alone, as observed for the duration of the 20 day study.

### Clinical Chemistry

C-reactive protein (CRP) was monitored as a general marker of inflammation in response to CD20 TCE treatment. Similar to CD3ε-PepPOL v2 impact on serum cytokines, a dose-dependent decrease in CRP was observed for CD20 TCE/CD3ε-PepPOL, v2 combo groups relative to CD20 TCE monotherapy at 0.010 mg/kg dosed IV.

### CD20 TCE Cytolytic Activity

Following Intravenous (IV) administration of 0.010 mg/kg CD20 TCE alone, or in combination with CD3ε-PepPOL v2, a complete elimination of circulating CD20+ B lymphocytes was observed. No apparent difference in CD20 TCE cytolytic activity was discernable between CD20 TCE control group dosed at 0.010 mg/kg by IV administration vs. CD3ε-PepPOL v2 dosed groups suggesting that CD3ε-PepPOL v2 successfully uncouples cytokine/inflammatory responses from the CD20 TCE PD effect.

### Example 11: In Silico Mutational Screen of Binding Peptide Binding to CD3ε Domains I2C (SEQ ID NO 26), I2E (SEQ ID NO 382) and CD3 Binder of Heterodimeric Antibody (SEQ ID NO 381)

Computational methods were utilized to identify tolerable mutation combinations for CD3ε peptide binding to I2C. We used the Flex ddG protocol within the ROSETTA macromolecular modeling suite v.3.12 to computationally model and predict changes in binding free energies upon mutation as described in Barlow et al. J. Phys. Chem (2018). This method generates a large ensemble of protein-peptide complexes using the backrub protocol, which samples local backbone and side-chain conformations around a mutation site. The binding free energy difference (ΔΔG) between the wild-type and mutant complexes is then calculated using Rosetta's Talaris energy function. Generalized additive model (GAM) is used to reweigh Rosetta's predicted ΔΔG to experimentally known values. A ΔΔG≥1 indicates a destabilizing mutation, a 1<ΔΔG≤−1 indicates a neutral mutation, and a ΔΔG<−1 indicates a stabilizing mutation. We applied this protocol to the high-resolution crystal structure (1.35 Å) of scFv region of I2C bound to the binding peptide (pE-DGNEELK), keeping the peptide chain's backbone flexible. We first performed single mutational scans of the CD3ε peptide using mutation saturation. We used a lower cut-off score of 1.2 for the ΔΔG as we attempt to increase our sequence diversity in the pool of predicted binders. Then, based on the single mutations with ΔΔG score<1.2, we applied double and quadrupled combinatorial mutations in residues 2, 3, 4 and 6. Mutation of residue 5 to any amino acid is not tolerable, while mutating residue 1, Glu to other residues is not consistent with previous experimental data. Therefore, residues 1 and 5 were not mutated. Residues 7 and 8 can be mutated to any amino acid as they are not in the core binding pocket of I2C. We found that most of the predictions are consistent with the experimental binding data from the previous studies.

A similar exercise was performed with I2E, a closely related variant of I2C with three residues different in the CDRs. A high-resolution crystal structure (2.25 Å) of I2E scFv is available. First, predicted complex structure of I2E with CD3ε peptide was generated by superimposing I2E crystal structure onto I2C/CD3ε peptide co-crystal structure. Then, mutational saturation was performed with residues 2, 6, 7, and 8 using the Flex ddG protocol. From the prediction results, two additional mutations B and F were found to be allowable at residue 2 compared to I2C. Mutation of residue 6 was not tolerable, while any amino acid can replace residues 7 and 8.

For the CD3 binding domain of the heterodimeric antibody, a high resolution structure is not available. Therefore, an scFv model was generated using state-of-the-art DeepAb that applies a deep learning method to predict the antibody Fv structures from amino acid sequences. The predicted structure was then aligned with I2C to generate the complex model with CD3ε peptide, and single mutational scan was performed for residues 2-8. Peptide residues 2, 7, and 8 allow any amino acid substitution, while residues 4 and 6 can be mutated to many other amino acids.

### Example 12: In Vitro Cytotoxicity and Cytokine Release Inhibition by Binding Peptides

In vitro potency of CD3e pepPEG was indirectly assessed by measuring in vitro activity of T cell engager (TCE) using T cell dependent cellular cytotoxicity (TDCC) assay. Either 100 pM or 300 pM of TCE (STEAP1×CD3e heterodimeric TCE, was mixed with increasing concentrations of CD3e pepPEG before the mixture is treated to cells. Peripheral blood mononuclear cells (PBMCs) as effector cells were co-cultured with tumor target cells at an effector cell-to-target cell ratio of 10:1 in the presence of mixture of TCE and CD3e pepPEG for 48 hours at 37° C. Cytotoxicity of TCE was assessed by Steady-Glo (Promega) for luciferase-tagged target cells and CellTiter-Glo (Promega) for non-luciferase-tagged target cells after plates were washed to eliminate T cells. Specific cytotoxicity curves and EC50 values were derived using Prism 7.04 (GraphPad). Cytokine production was assessed in supernatants from TDCC assays using electro-chemiluminescence assays (MesoScale Discovery).

| | | | | | Arm |
|---|---|---|---|---|---|
| | | | PEG | | configu- |
| Identifier | Peptide seq | Affinity | size | Linker | ration |
| 71203-2 | pE-DGNEEL | High | 20K | Thiosuccinimide | 4 arm |
| | KGC (PEG)- | | | | |
| | OH | | | | |
| 79674-1 | pE-DGNEEL | High | 20K | Thiosuccinimide | 4 arm |
| | KGC (PEG)- | | | | |
| | NH2 | | | | |
| 79676-1 | pE-DGEEEL | Inter- | 20K | Thiosuccinimide | 4 arm |
| | KGC (PEG)- | mediate | | | |
| | NH2 | | | | |
| 73135-2 | pE-DGNEEL | High | 20K | Thioether | 4 arm |
| | KGK (PEG)- | | | | |
| | OH | | | | |
| 79921-1 | pE-DGEEEL | Inter- | 20K | Thioether | 4 arm |
| | KGK (PEG)- | mediate | | | |
| | OH | | | | |
| 79675-1 | pE-DGNEEL | High | 5K | Thiosuccinimide | 4 arm |
| | KGC (PEG)- | | | | |
| | NH2 | | | | |
| 79677-1 | pE-DGEEEL | Inter- | 5K | Thiosuccinimide | 4 arm |
| | KGC (PEG)- | mediate | | | |
| | NH2 | | | | |
| 73136-2 | pE-DGNEEL | High | 20K | Thioether | Linear |
| | KGK (mPEG)- | | | | |
| | OH | | | | |
| 82138-1 | pE-DGEEEL | Inter- | 20K | Thioether | Linear |
| | KGK (mPEG)- | mediate | | | |
| | OH | | | | |
| 74769-2 | pE-DGNEEL | High | 10K | Thioether | Linear |
| | KGK (mPEG)- | | | | |
| | OH | | | | |
| 82137-1 | pE-DGEEEL | Inter- | 10K | Thioether | Linear |
| | KGK (mPEG)- | mediate | | | |
| | OH | | | | |

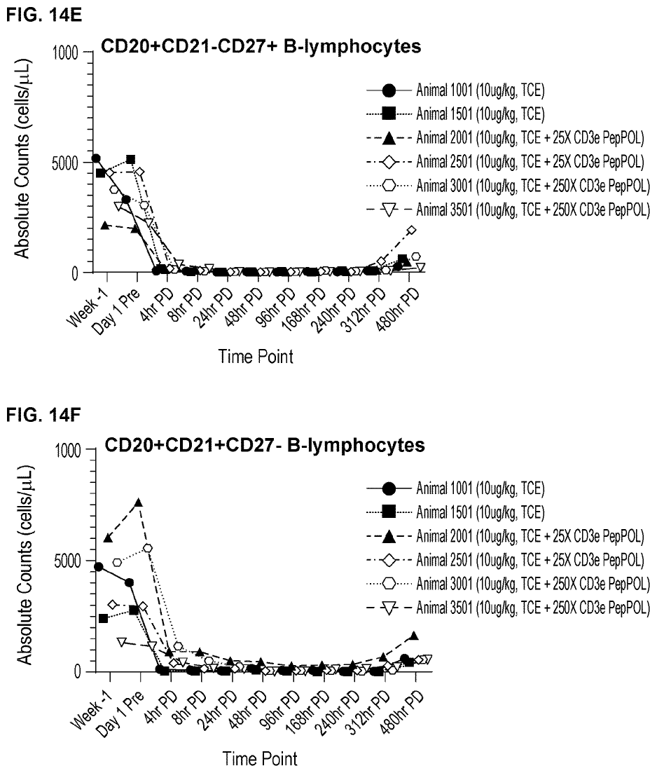

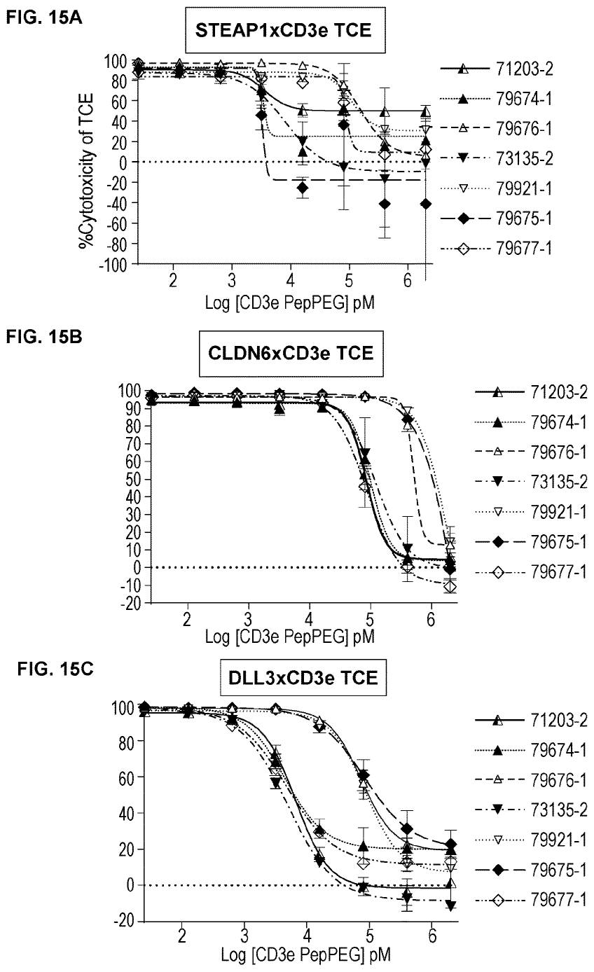

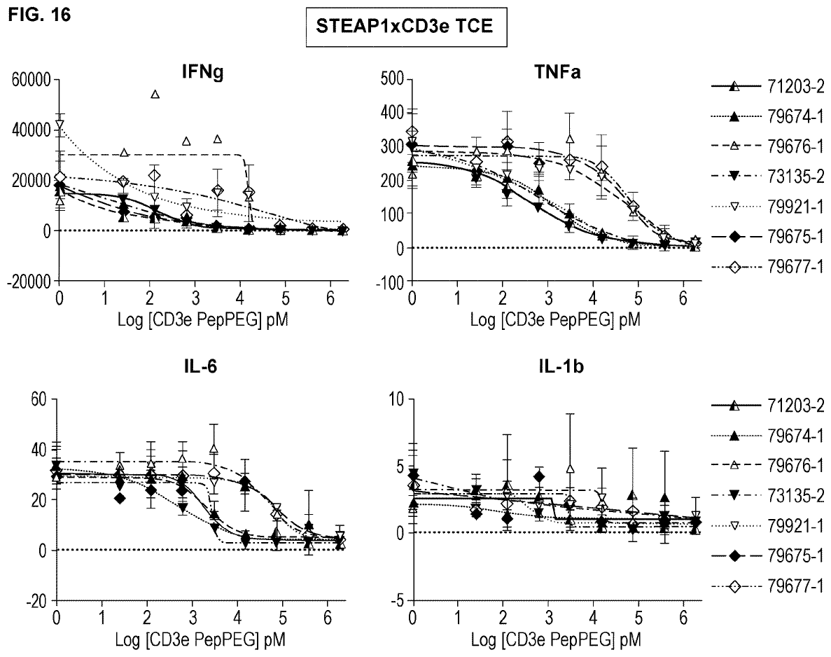

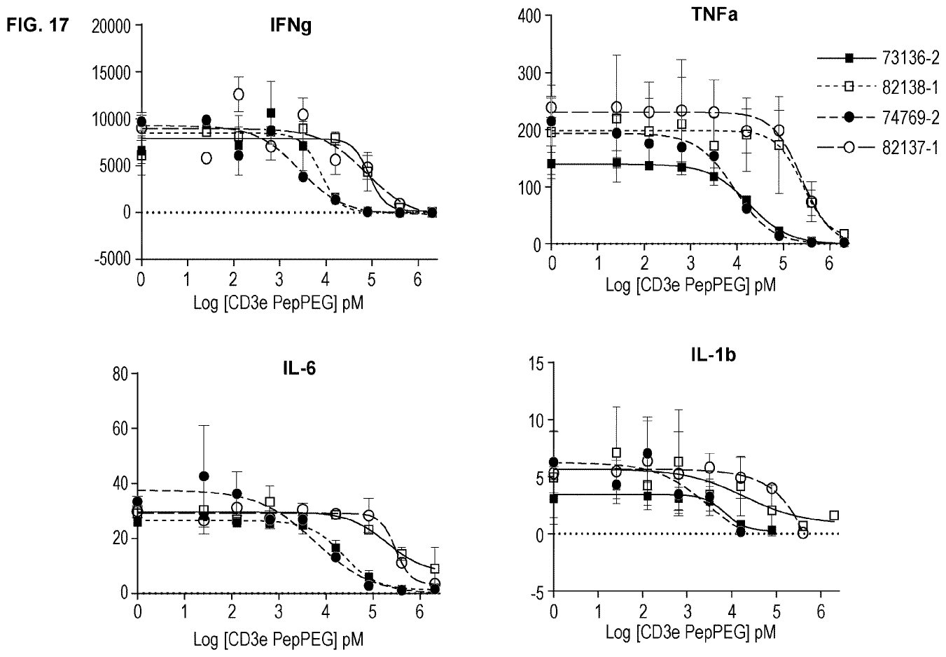

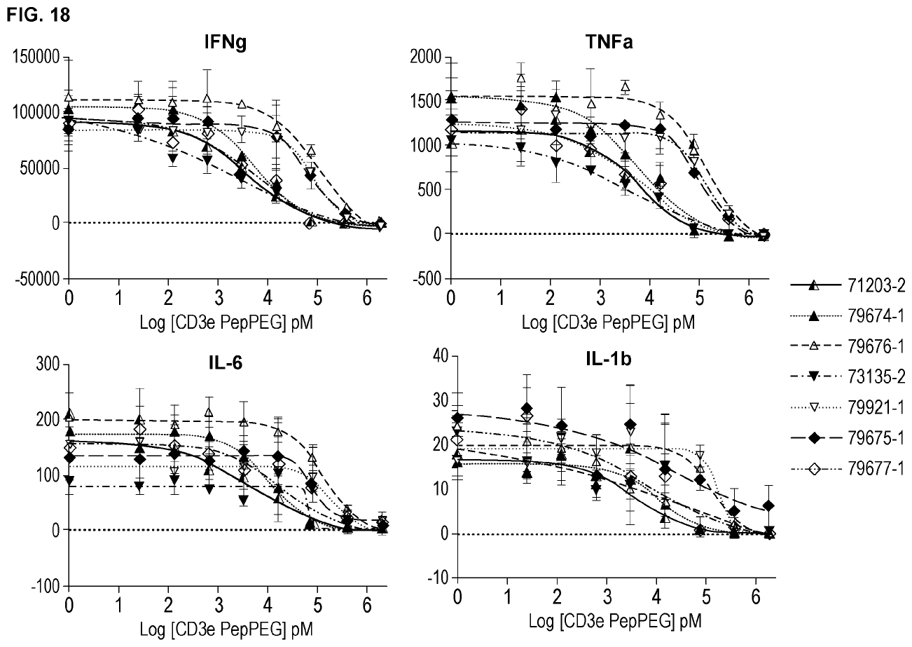

Results are shown in FIG. 15 for cytotoxicity and FIG. 16 for cytokine release and FIG. 17 for DLL3XCD3 TCE

### Example 13

A high affinity SPR ‘capture’ surface was first constructed to facilitate the SPR analysis of pre-equilibrated peptide analyte+TCE solutions. Briefly, a Cytiva CM5 chip was functionalized with PDEA (2-(2-pyridinyldithio)ethaneamine) under standard EDC/NHS coupling conditions where the PDEA is dissolved to 20 mM 2-(2-pyridinyldithio)ethaneamine and 1 M NaCl in 0.1 M sodium acetate pH 4.3, 6 peptide including pEDGNEELKGC (ID 14974, SEQ ID NO 304) were tested:

| Peptide | | Molecular | HPLC | Size |
|---|---|---|---|---|
| ID | Sequence | Weight | Purity | (mg) |
| 14950-1 | pGlu-DGEEELKGC | 1090 | >95% | 30 |
| 14950-2 | pGlu-DGSEELKGC | 1048 | >95% | 30 |
| 14950-3 | pGlu-DGTEELKGC | 1062 | >95% | 30 |
| 14950-4 | pGlu-DGVEELKGC | 1060 | >95% | 30 |
| 14950-5 | pGlu-DGIEELKGC | 1074 | >95% | 30 |

Respective peptide was dissolved to between 50 and 100-ug/mL for injection over the activated CM5 surface in PBST, pH 6.5, and coupling times were optimized to yield the desired surface density (~300RU).

For the binding assessment, Individual binding isotherms were constructed by titrating the respective peptide of interest into a fixed concentration of TCE-containing solution in PBST. At least 4 hrs equilibration time were allotted (r.t.) prior to analysis by SPR. For the analysis, a calibration curve was first constructed of the TCE analyte being assessed. For analysis of the TCE+peptide mixtures, the samples were injected (10 uL/min) over the previously prepared capture surface for 90 seconds, where the SPR response (RU) was recorded at the end of the injection and referenced to the previously prepared standard curve to derive an estimate of the ‘free’ TCE concentration in the presence of peptide analyte. The ‘free’ TCE concentration measured was plotted as a function of log peptide concentration and fit using non-linear regression analysis to a 4 parameter logistic function to derive an IC50.

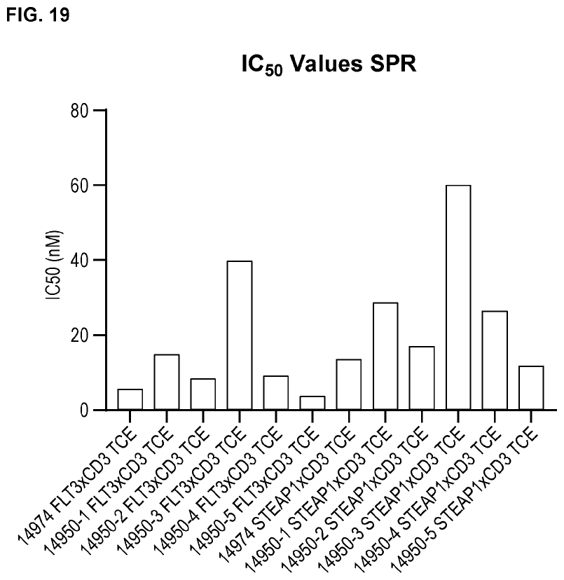

Results are shown in FIG. 19. All binding peptides but negative example 14950-3 showed sufficient reduction in IC50.

### Example 14: SPR Assay of Immobilized Peptides Binding with TCE

A cytiva CM5 chip was first functionalized with PDEA (2-(2pyridinyldithio)ethaneamine) under standard ((1-ethyl-3-(3-dimethylaminopropyl)carbodiimide)/(N-hydroxysuccinimide) EDC/NHS coupling conditions where the PDEA is dissolved to 20 mM and 1 M NaCl in 0.1 M sodium acetate pH 4.3. Binding peptides were dissolved to 50- or 100-ug/mL respectively in PBST, pH 6.5, and coupling times were optimized to yield the desired surface density.

| | | | | Rmax | Chi² | | |
|---|---|---|---|---|---|---|---|
| Sample | ka (M⁻¹s⁻¹) | kd (1/s) | KD (M) | (RU) | (RU²) | Peptide | Model |
| FLT3xCD3 TCE SEQ | 1.88E+06 | 2.40E−03 | 1.28E−09 | 171 | 819 | pEDGNEELKGC | Kinetic 1:1 |
| ID NO 93 | | | | | | | Binding |
| FLT3xCD3 TCE SEQ | 4.04E+06 | 2.95E−02 | 7.30E−09 | 20.3 | 5.53 | pEDGEEELKGC | Kinetic 1:1 |
| ID NO 93 | | | | | | | Binding |
| FLT3xCD3 TCE SEQ | 3.21E+06 | 2.85E−02 | 8.88E−09 | 8.1 | 5.42 | pEDGTEELKGC | Kinetic 1:1 |
| ID NO 93 | | | | | | | Binding |
| DLL3xCD3 TCE SEQ | 2.14E+06 | 2.79E−03 | 1.30E−09 | 58.5 | 3.37 | pEDGNEELKGC | Kinetic 1:1 |
| ID NO 104 | | | | | | | Binding |
| DLL3xCD3 TCE SEQ | 2.64E+06 | 5.42E−02 | 2.05E−08 | 47.5 | 0.124 | pEDGEEELKGC | Kinetic 1:1 |
| ID NO 104 | | | | | | | Binding |
| DLL3xCD3 TCE SEQ | 4.81E+06 | 7.13E−02 | 1.48E−08 | 11 | 0.31 | pEDGTEELKGC | Kinetic 1:1 |
| ID NO 104 | | | | | | | Binding |
| CLDN6xCD3 TCE | 2.72E+06 | 3.80E−03 | 1.40E−09 | 66.9 | 1.52 | pEDGNEELKGC | Kinetic 1:1 |
| SEQ ID NO 340 | | | | | | | Binding |
| CLDN6xCD3 TCE | 3.81E+06 | 6.24E−02 | 1.64E−08 | 47 | 0.998 | pEDGEEELKGC | Kinetic 1:1 |
| SEQ ID NO 340 | | | | | | | Binding |
| Steap 1xCD3 TCE | 2.00E+06 | 5.14E−03 | 2.57E−09 | 395.1 | 139 | pEDGNEELKGC | Kinetic 1:1 |
| SEQ ID NO SEQ ID | | | | | | | Binding |
| NO 373 + 374 + 375 | | | | | | | |
| Steap 1xCD3 TCE | 3.04E+06 | 8.34E−02 | 2.74E−08 | 30.2 | 0.309 | pEDGEEELKGC | Kinetic 1:1 |
| SEQ ID NO SEQ ID | | | | | | | Binding |
| NO 373 + 374 + 375 | | | | | | | |
| Steap 1xCD3 TCE | 2.09E+06 | 7.34E−02 | 3.52E−08 | 15.1 | 0.282 | pEDGTEELKGC | Kinetic 1:1 |
| SEQ ID NO SEQ ID | | | | | | | Binding |
| NO 373 + 374 + 375 | | | | | | | |
| CDH3-CD3-MSLN- | | | 1.33E−08 | | 5.05 | pEDGNEELKGC | SS |
| CD3 TCE (SEQ ID | | | | | | | Affinity |
| NO 255) | | | | | | | |
| CDH3-CD3-MSLN- | | | 7.25E−08 | | 0.574 | pEDGEEELKGC | SS |
| CD3 TCE (SEQ ID | | | | | | | Affinity |
| NO 255) | | | | | | | |

The measured kinetic profiles for the assets evaluated by SPR suggest that the changes in amino acid sequence of the binding peptides are primarily deriving affinity modulation through changes in the off-rates, and demonstrate a similar rank order of affinity changes across each asset evaluated. For CDH3×MSLN dual targeting TCE with two CD3e binders, where the 1:1 kinetic model did not fit the data as well due to high bulk contributions, a bivalent analyte model suggest that the fast component of the on-rate for the high affinity pEDGNEELKGC peptide is 2.42E+06 (M⁻¹s⁻¹), and the fast component for the off-rate is 0.0212 (s⁻¹), while of the affinity modulated pEDGEEELKGC peptide, the fast component of the on-rate is 1.29E+06 (M-'s⁻¹), and the fast component for the off-rate is 0.236 (s⁻¹), consistent with the fitted steady state affinity and the observations for the other assets evaluated where the affinity modulation is derived primarily from changes in the off-rates.

### Equilibrium Binding Affinity by KinExA

In order to understand the equilibrium binding of the conjugates in solution, a kinetic exclusion assay (KinExA) was performed. This method allows for solution equilibrium to be measured on the fully intact conjugates with no modifications to either binding partner and are appropriate for the range of affinities that are observed between the molecules being investigated.
1. Preparation of NHS-activated sepharose fast-flow beads:
2. Fast flow sepharose suspension in 10000 IPA, washed twice with milliQ H20, mild centrifugation to pool beads in between wash steps.
3. Equilibrated in 0.15M NaBO3, pH=8.5 buffer
4. Added 80 mM PDEA to activate the resin for thiol exchange, 10 mins, r.t.
5. Equilibrated resin with Hepes Buffered Saline-P (0.01 M HEPES pH 7.4, 0.15 M NaCl, 0.005% v/v Surfactant P2), pH 7.4 binding buffer
6. Added 30 ug/mL pGlu-DGNEELKC (SEQ ID NO; XXX) binding peptide, reaction for 1 hr to functionalize resin.
7. Quench with 10 mM L-Cys for 1 hour
8. Constant binding partners were diluted to stated concentrations and the peptide or conjugates were 3-fold serially diluted in an appropriate range of concentrations.

| | | | | KD (M) | |
|---|---|---|---|---|---|
| | | | | relative to | |
| | | | CBP | peptide | |
| Asset | Conjugate | Peptide | Concentration | concentration | Conjugate alias |
| CDH3-CD3- | 79674-1 | pEDGNEELKGC- | 250 pM | 6.38E−11 | High affinity 20 k 4-arm |
| MSLN-CD3 | | NH2 | | | Thiosuccinimide-NH2 |
| TCE (SEQ ID | | | | | |
| NO 255) | | | | | |
| CLDN6xCD3 | 79675-1 | pEDGNEELKGC- | 250 pM | 5.25E−11 | High affinity 5 k 4-arm |
| TCE | | NH2 | | | Thiosuccinimide-NH2 |
| Steap1xCD3 | N/A | pEDGNEELKGC- | 333 pM | 1.59E−09 | High affinity peptide |
| TCE SEQ ID | | NH2 | | | |
| NO SEQ ID | | | | | |
| NO | | | | | |
| 373 + 374 + 375 | | | | | |
| DLL3xCD3 | 79676-1 | pEDGEEELKGC- | 250 pM | 1.28E−08 | Intermediate affinity 20 k 4-arm |
| TCE SEQ ID | | NH2 | | | Thiosuccinimide-NH2 |
| NO 104 | | | | | |
| DLL3xCD3 | 79675-1 | pEDGNEELKGC- | 250 pM | 6.23E−11 | High affinity 5 k 4-arm |
| TCE SEQ ID | | NH2 | | | Thiosuccinimide-NH2 |
| NO 104 | | | | | |
| DLL3xCD3 | 79674-1 | pEDGNEELKGC- | 250 pM | 3.72E−11 | High affinity 20 k 4-arm |
| TCE SEQ ID | | NH2 | | | Thiosuccinimide-NH2 |
| NO 104 | | | | | |
| DLL3xCD3 | 79677-1 | pEDGEEELKGC- | 250 pM | 4.69E−09 | Intermediate affinity 5 k 4-arm |
| TCE SEQ ID | | NH2 | | | Thiosuccinimide-NH2 |
| NO 104 | | | | | |
| FLT3xCD3 | 73135-2 | pEDGNEELKGK | 125 pM | 1.22E−11 | High affinity 20 k 4-arm Thioether |
| TCE SEQ ID | | | | | |
| NO 93 | | | | | |
| FLT3xCD3 | 72103-1 | pEDGNEELKGC- | 125 pM | 7.77E−11 | High affinity 5k 4-arm |
| TCE SEQ ID | | OH | | | Thiosuccinimide-OH |
| NO 93 | | | | | |
| FLT3xCD3 | 73136-2 | pEDGNEELKGK | 250 pM | 1.73E−10 | High affinity 20 k Linear Thioether |
| TCE SEQ ID | | | | | |
| NO 93 | | | | | |
| FLT3xCD3 | 73135-2 | pEDGNEELKGK | 250 pM | 1.53E−10 | High affinity 20 k Linear Thioether |
| TCE SEQ ID | | | | | |
| NO 93 | | | | | |

Overall, the solution-based equilibrium data suggests that the TCE masking molecules bind to the representative TCEs despite having four different CD3c binders as disclosed herein, from the surface-based measurements using immobilized peptide, with no negative consequences of linker, polymer-branching or size having been observed.

### Example 15: TCE Masking Molecule Inhibition of Cytotoxicity and Cytokine Release of Representative CDH3×MSLN Dual Targeting TCE with Two CD3e Binders (SEQ ID NO 255)

In vitro potency of CD3e pepPEG was indirectly assessed by measuring in vitro activity of T cell engager (TCE) using T cell dependent cellular cytotoxicity (TDCC) assay. Either 100 pM or 300 pM of TCE was mixed with increasing concentrations of CD3e pepPEG before the mixture is treated to cells. Peripheral blood mononuclear cells (PBMCs) as effector cells were co-cultured with tumor target cells at an effector cell-to-target cell ratio of 10:1 in the presence of mixture of TCE and CD3e pepPEG for 48 hours or 72 hours at 37° C. Cytotoxicity of TCE was assessed by Steady-Glo (Promega) for luciferase-tagged target cells and CellTiter-Glo (Promega) for non-luciferase-tagged target cells after plates were washed to eliminate T cells. Specific cytotoxicity curves and EC50 values were derived using Prism 7.04 (GraphPad). Cytokine production was assessed in supernatants from TDCC assays using electro-chemiluminescence assays (MesoScale Discovery).

TABLE 20
overview of tested TCE masking molecules here with respect to TCE of SEQ ID NO 255

| | | | | | Arm | |
|---|---|---|---|---|---|---|
| Bioreg | Peptide seq | Affinity | PEG size | Linker | configuration | DiscMA |
| 71203-2 | pE−DGNEEL KGC | High | 20 K | Thiosuccinimida | 4 cm | |
| | (PEG)-OH | | | | | |
| 79674-1 | pE−DGNEEL KGC | High | 20 K | Thiosuccinimide | 4 cm | DiscMA |
| | (PEG)-NH2 | | | | | |
| 79676-1 | pE−DGEEEL KGC | Intermediote | 20 K | Thiosuccinimide | 4 cm | DiscMA |
| | (PEG)-NH2 | | | | | |
| 73135-2 | DE−DGNEEL KGK | High | 20 K | Thioether | 4 cm | DiscMA |
| | (PEG)-OH | | | | | |
| 79921-1 | pE−DGEEEL KGK | Intermediate | 20 K | Thioether | 4 cm | DiscMA |
| | (PEG)-OH | | | | | |
| 79675-1 | pE−DGNEEL KGC | High | 5 K | Thiosuccinimide | 4 cm | DiscMA |
| | (PEG)-NH2 | | | | | |
| 79677-1 | pE−DGEEEL KGC | Intermediate | 5 K | Thiosuccinimide | 4 cm | DiscMA |
| | (PEG)-NH2 | | | | | |
| 73136-2 | pE−DGNEEL KGK | High | 20 K | Thioether | Linear | DiscMA |
| | (mPEG)-OH | | | | | |
| 82138-1 | DE−DGEEEL KGK | Intermediate | 20 K | Thioether | Linear | DiscMA |
| | (mPEG)-OH | | | | | |
| 74769-2 | pE−DGNEEL KGK | High | 10 K | Thioether | Linear | DiscMA |
| | (mPEG)-OH | | | | | |
| 82137-1 | pE−DGEEEL KGK | Intermediate | 10 K | Thioether | Linear | DiscMA |
| | imPEGI-OH | | | | | |

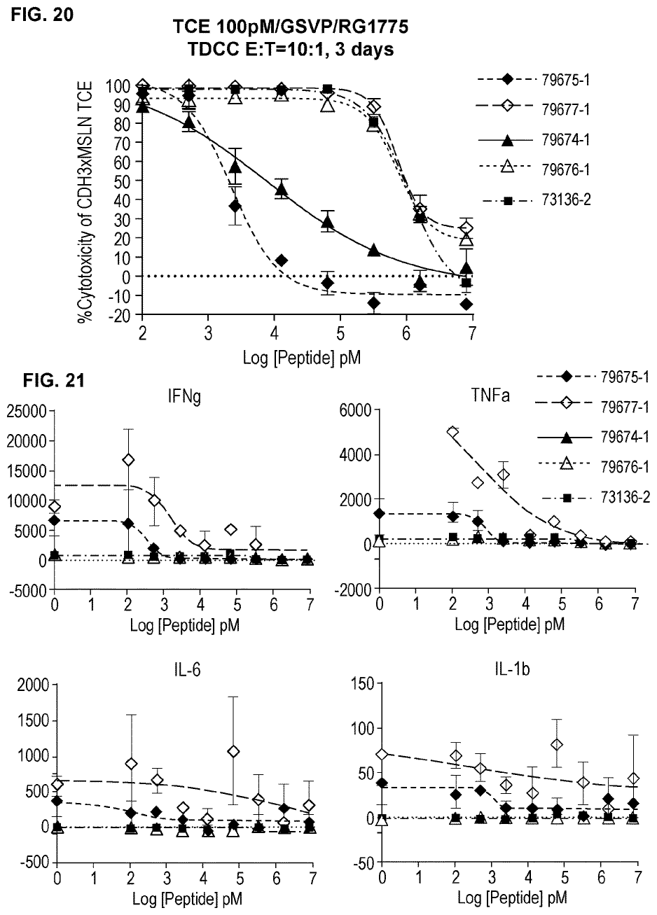

Results are shown in FIG. 20 with respect to inhibition of cytotoxicity and in FIG. 21 with respect to inhibition of cytokine release. The linear PEG was added 4 fold higher as “TCE masking molecule” concentration to match the “Peptide” concentration. Accordingly, x-axis was changed to concentration of “Peptide”. Though high affinity linear molecule was added as same “peptide” concentration as 4-aim molecules, it showed the lower activity than high affinity 4-arm and rather similar activity as low affinity 4-arm against representative dual targeting molecule with two CD3e binding domains (SEQ ID NO 255).

Based on the present in vitro potency assay, peptide 79675-1 would be preferred over 79677-1 and 79676-1=and 73136-2, the latter three yielding comparable results among each other.

TABLE 6
Sequence table

| | | | | |
|---|---|---|---|---|
| 1. | CD19 VL CDR1 | Artificial | Aa | KASQSVDYDGDSYLN |
| 2. | CD19 VL CDR2 | Artificial | aa | DASNLVS |
| 3. | CD19 VL CDR3 | Artificial | aa | QQSTEDPWT |
| 4. | CD19 VH CDR1 | Artificial | aa | SYWMN |
| 5. | CD19 VH CDR2 | Artificial | aa | QIWPGDGDTNYNGKFKG |
| 6. | CD19 VH CDR3 | artificial | aa | RETTTVGRYYYAMDY |
| 7. | CD19 VL | artificial | aa | DIQLTQSPASLAVSLGQRATISCKASQSVDYDG |
| | | | | DSYLNWYQQIPGQPPKLLIYDASNLVSGIPPRFS |
| | | | | GSGSGTDFTLNIHPVEKVDAATYHCQQSTEDP |
| | | | | WTFGGGTKLEIK |
| 8. | CD19 VH | artificial | aa | QVQLQQSGAELVRPGSSVKISCKASGYAFSSY |
| | | | | WMNWVKQRPGQGLEWIGQIWPGDGDTNYNG |
| | | | | KFKGKATLTADESSSTAYMQLSSLASEDSAVY |
| | | | | FCARRETTTVGRYYYAMDYWGQGTTVTVSS |
| 9. | CD3 VH CDR1 | artificial | aa | RYTMH |
| 10. | CD3 VH CDR2 | artificial | aa | YINPSRGYTNYNQKFKD |
| 11. | CD3 VH CDR3 | artificial | aa | YYDDHYCLDY |
| 12. | CD3 VL CDR1 | artificial | aa | RASSSVSYMN |
| 13. | CD3 VL CDR2 | artificial | aa | DTSKVAS |
| 14. | CD3 VL CDR3 | artificial | aa | QQWSSNPLT |
| 15. | CD3 VH | artificial | aa | DIKLQQSGAELARPGASVKMSCKTSGYTFTRY |
| | | | | TMHWVKQRPGQGL |
| | | | | EWIGYINPSRGYTNYNQKFKDKATLTTDKSSST |
| | | | | AYMQLSSLTSEDSAVYYCARYYDDHYCLDYW |
| | | | | GQGTTLTVSS |
| 16. | CD3 VL | artificial | aa | VDDIQLTQSPAIMSASPGEKVTMTCRASSSVSY |
| | | | | MNWYQQKSGTSPKRWIYDTSKVASGVPYRFS |
| | | | | GSGSGTSYSLTISSMEAEDAATYYCQQWSSNP |
| | | | | LTFGAGTKLELK |
| 17. | CD19xCD3 | artificial | aa | DIQLTQSPASLAVSLGQRATISCKASQSVDYDG |
| | scFv incl linker | | | DSYLNWYQQIPGQPPKLLIYDASNLVSGIPPRFS |
| | and his-tag | | | GSGSGTDFTLNIHPVEKVDAATYHCQQSTEDP |
| | | | | WTFGGGTKLEIKGGGGSGGGGSGGGGSQVQL |
| | | | | QQSGAELVRPGSSVKISCKASGYAFSSYWMN |
| | | | | WVKQRPGQGLEWIGQIWPGDGDTNYNGKFKG |
| | | | | KATLTADESSSTAYMQLSSLASEDSAVYFCAR |
| | | | | RETTTVGRYYYAMDYWGQGTTVTVSSGGGGS |
| | | | | DIKLQQSGAELARPGASVKMSCKTSGYTFTRY |
| | | | | TMHWVKQRPGQGLEWIGYINPSRGYTNYNQK |
| | | | | FKDKATLTTDKSSSTAYMQLSSLTSEDSAVYY |
| | | | | CARYYDDHYCLDYWGQGTTLTVSSVEGGSGG |
| | | | | SGGSGGSGGVDDIQLTQSPAIMSASPGEKVTM |
| | | | | TCRASSSVSYMNWYQQKSGTSPKRWIYDTSK |
| | | | | VASGVPYRFSGSGSGTSYSLTISSMEAEDAATY |
| | | | | YCQQWSSNPLTFGAGTKLELKHHHHHH |
| 18. | CDR-LI of I2C | artificial | aa | GSSTGAVTSGNYPN |
| 19. | CDR-L2 of I2C | artificial | aa | GTKFLAP |
| 20. | CDR-L3 of I2C | artificial | aa | VLWYSNRWV |
| 21. | CDR-H1 of I2C | artificial | aa | KYAMN |
| 22. | CDR-H2 of I2C | artificial | aa | RIRSKYNNYATYYADSVKD |
| 23. | CDR-H3 of I2C | artificial | aa | HGNFGNSYISYWAY |
| 24. | VH of I2C | artificial | aa | EVQLVESGGGLVQPGGSLKLSCAASGFTFNK |
| | | | | YAMNWVRQAPGKGLEWVARIRSKYNNYAT |
| | | | | YYADSVKDRFTISRDDSKNTAYLQMNNLKTE |
| | | | | DTAVYYCVRHGNFGNSYISYWAYWGQGTLV |
| | | | | TVSS |
| 25. | VL of I2C | artificial | aa | QTVVTQEPSLTVSPGGTVTLTCGSSTGAVTSG |
| | | | | NYPNWVQQKPGQAPRGLIGGTKFLAPGTPAR |
| | | | | FSGSLLGGKAALTLSGVQPEDEAEYYCVLWY |
| | | | | SNRWVFGGGTKLTVL |
| 26. | VH-VL of I2C | artificial | aa | EVQLVESGGGLVQPGGSLKLSCAASGFTFNK |
| | | | | YAMNWVRQAPGKGLEWVARIRSKYNNYAT |
| | | | | YYADSVKDRFTISRDDSKNTAYLQMNNLKTE |
| | | | | DTAVYYCVRHGNFGNSYISYWAYWGQGTLV |
| | | | | TVSSGGGGSGGGGSGGGGSQTVVTQEPSLTV |
| | | | | SPGGTVTLTCGSSTGAVTSGNYPNWVQQKPG |
| | | | | QAPRGLIGGTKFLAPGTPARFSGSLLGGKAAL |
| | | | | TLSGVQPEDEAEYYCVLWYSNRWVFGGGTK |
| | | | | LTVL |
| 27. | CD33 cc VH of | artificial | aa | QVQLVQSGAEVKKPGESVKVSCKASGYTFTN |
| | E11 | | | YGMNWVKQAPGQCLEWMGWINTYTGEPTY |
| | | | | ADKFQGRVTMTTDTSTSTAYMEIRNLGGDDT |
| | | | | AVYYCARWSWSDGYYVYFDYWGQGTSVTV |
| | | | | SS |
| 28. | CD33 VH of | Artificial | aa | QVQLVQSGAEVKKPGESVKVSCKASGYTFTN |
| | E11 | | | YGMNWVKQAPGQGLEWMGWINTYTGEPTY |
| | | | | ADKFQGRVTMTTDTSTSTAYMEIRNLGGDDT |
| | | | | AVYYCARWSWSDGYYVYFDYWGQGTSVTV |
| | | | | SS |
| 29. | CD33 HCDR1 | artificial | aa | NYGMN |
| | of E11 | | | |
| 30. | CD33 HCDR2 | artificial | aa | WINTYTGEPTYADKFQG |
| | of E11 | | | |
| 31. | CD33 HCDR3 | artificial | aa | WSWSDGYYVYFDY |
| | of E11 | | | |
| 32. | CD33 CC VL of | artificial | aa | DIVMTQSPDSLTVSLGERTTINCKSSQSVLDSS |
| | E11 | | | TNKNSLAWYQQKPGQPPKLLLSWASTRESGI |
| | | | | PDRFSGSGSGTDFTLTIDSPQPEDSATYYCQQS |
| | | | | AHFPITFGCGTRLEIK |
| 33. | CD33 VL of | artificial | aa | DIVMTQSPDSLTVSLGERTTINCKSSQSVLDSS |
| | E11 | | | TNKNSLAWYQQKPGQPPKLLLSWASTRESGI |
| | | | | PDRFSGSGSGTDFTLTIDSPQPEDSATYYCQQS |
| | | | | AHFPITFGQGTRLEIK |
| 34. | CD33 LCDR1 | artificial | aa | KSSQSVLDSSTNKNSLA |
| | of E11 | | | |
| 35. | CD33 LCDR2 | artificial | aa | WASTRES |
| | of E11 | | | |
| 36. | CD33 LCDR3 | artificial | aa | QQSAHFPIT |
| | of E11 | | | |
| 37. | CD33 HL CC of | artificial | aa | QVQLVQSGAEVKKPGESVKVSCKASGYTFTN |
| | E11 | | | YGMNWVKQAPGQCLEWMGWINTYTGEPTY |
| | | | | ADKFQGRVTMTTDTSTSTAYMEIRNLGGDDT |
| | | | | AVYYCARWSWSDGYYVYFDYWGQGTSVTV |
| | | | | SSGGGGSGGGGSGGGGSDIVMTQSPDSLTVS |
| | | | | LGERTTINCKSSQSVLDSSTNKNSLAWYQQKP |
| | | | | GQPPKLLLSWASTRESGIPDRFSGSGSGTDFTL |
| | | | | TIDSPQPEDSATYYCQQSAHFPITFGCGTRLEI |
| | | | | K |
| 38. | CD33 HL of | artificial | aa | QVQLVQSGAEVKKPGESVKVSCKASGYTFTN |
| | E11 | | | YGMNWVKQAPGQGLEWMGWINTYTGEPTY |
| | | | | ADKFQGRVTMTTDTSTSTAYMEIRNLGGDDT |
| | | | | AVYYCARWSWSDGYYVYFDYWGQGTSVTV |
| | | | | SSGGGGSGGGGSGGGGSDIVMTQSPDSLTVS |
| | | | | LGERTTINCKSSQSVLDSSTNKNSLAWYQQKP |
| | | | | GQPPKLLLSWASTRESGIPDRFSGSGSGTDFTL |
| | | | | TIDSPQPEDSATYYCQQSAHFPITFGQGTRLEI |
| | | | | K |
| 39. | CD33 CC E11 | artificial | aa | QVQLVQSGAEVKKPGESVKVSCKASGYTFTN |
| | HL x I2C HL | | | YGMNWVKQAPGQCLEWMGWINTYTGEPTY |
| | Bispecific | | | ADKFQGRVTMTTDTSTSTAYMEIRNLGGDDT |
| | molecule | | | AVYYCARWSWSDGYYVYFDYWGQGTSVTV |
| | | | | SSGGGGSGGGGSGGGGSDIVMTQSPDSLTVS |
| | | | | LGERTTINCKSSQSVLDSSTNKNSLAWYQQKP |
| | | | | GQPPKLLLSWASTRESGIPDRFSGSGSGTDFTL |
| | | | | TIDSPQPEDSATYYCQQSAHFPITFGCGTRLEI |
| | | | | KSGGGGSEVQLVESGGGLVQPGGSLKLSCAA |
| | | | | SGFTFNKYAMNWVRQAPGKGLEWVARIRSK |
| | | | | YNNYATYYADSVKDRFTISRDDSKNTAYLQ |
| | | | | MNNLKTEDTAVYYCVRHGNFGNSYISYWAY |
| | | | | WGQGTLVTVSSGGGGSGGGGGGGGSQTVV |
| | | | | TQEPSLTVSPGGTVTLTCGSSTGAVTSGNYPN |
| | | | | WVQQKPGQAPRGLIGGTKFLAPGTPARFSGS |
| | | | | LLGGKAALTLSGVQPEDEAEYYCVLWYSNR |
| | | | | WVFGGGTKLTVL |
| 40. | CD33 E11 HL x | artificial | aa | MGWSCIILFLVATATGVHSQVQLVQSGAEVK |
| | I2C HL | | | KPGESVKVSCKASGYTFTNYGMNWVKQAPG |
| | | | | QGLEWMGWINTYTGEPTYADKFQGRVTMTT |
| | | | | DTSTSTAYMEIRNLGGDDTAVYYCARWSWS |
| | | | | DGYYVYFDYWGQGTSVTVSSGGGGSGGGGS |
| | | | | GGGGSDIVMTQSPDSLTVSLGERTTINCKSSQ |
| | | | | SVLDSSTNKNSLAWYQQKPGQPPKLLLSWAS |
| | | | | TRESGIPDRFSGSGSGTDFTLTIDSPQPEDSAT |
| | | | | YYCQQSAHFPITFGQGTRLEIKSGGGGSEVQL |
| | | | | VESGGGLVQPGGSLKLSCAASGFTFNKYAMN |
| | | | | WVRQAPGKGLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSG |
| | | | | GGGSGGGGSGGGGSQTVVTQEPSLTVSPGGT |
| | | | | VTLTCGSSTGAVTSGNYPNWVQQKPGQAPR |
| | | | | GLIGGTKFLAPGTPARFSGSLLGGKAALTLSG |
| | | | | VQPEDEAEYYCVLWYSNRWVFGGGTKLTVL |
| | | | | HHHHHH |
| 41. | CD33 CC x I2C- | artificial | aa | QVQLVQSGAEVKKPGESVKVSCKASGYTFTN |
| | scFc Bispecific | | | YGMNWVKQAPGQCLEWMGWINTYTGEPTYA |
| | HLE molecule | | | DKFQGRVTMTTDTSTSTAYMEIRNLGGDDTAV |
| | | | | YYCARWSWSDGYYVYFDYWGQGTSVTVSSG |
| | | | | GGGSGGGGSGGGGSDIVMTQSPDSLTVSLGER |
| | | | | TTINCKSSQSVLDSSTNKNSLAWYQQKPGQPP |
| | | | | KLLLSWASTRESGIPDRFSGSGSGTDFTLTIDSP |
| | | | | QPEDSATYYCQQSAHFPITFGCGTRLEIKSGGG |
| | | | | GSEVQLVESGGGLVQPGGSLKLSCAASGFTFN |
| | | | | KYAMNWVRQAPGKGLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGGGTKLTVLG |
| | | | | GGGDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGK |
| 42. | EGFRVIIIxCD3- | artificial | aa | NYGMH |
| | scFc VH CDR1 | | | |
| 43. | EGFRVIIIxCD3- | artificial | aa | VIWYDGSDKYYADSVRG |
| | scFc VH CDR2 | | | |
| 44. | EGFRVIIIxCD3- | artificial | aa | DGYDILTGNPRDFDY |
| | scFc VH CDR3 | | | |
| 45. | EGFRvIIIxCD3- | artificial | aa | RSSQSLVHSDGNTYLS |
| | scFc VL CDR1 | | | |
| 46. | EGFRVIIIxCD3- | artificial | aa | RISRRFS |
| | scFc VL CDR2 | | | |
| 47. | EGFRvIIIxCD3- | artificial | aa | MQSTHVPRT |
| | scFc VL CDR3 | | | |
| 48. | EGFRVIII_CCx | artificial | aa | QVQLVESGGGVVQSGRSLRLSCAASGFTFRNY |
| | CD3-scFc VH | | | GMHWVRQAPGKCLEWVAVIWYDGSDKYYAD |
| | | | | SVRGRFTISRDNSKNTLYLQMNSLRAEDTAVY |
| | | | | YCARDGYDILTGNPRDFDYWGQGTLVTVSS |
| 49. | EGFRVIII_CCx | artificial | aa | DTVMTQTPLSSHVTLGQPASISCRSSQSLVHSD |
| | CD3-scFc VL | | | GNTYLSWLQQRPGQPPRLLIYRISRRFSGVPDR |
| | | | | FSGSGAGTDFTLEISRVEAEDVGVYYCMQSTH |
| | | | | VPRTFGCGTKVEIK |
| 50. | EGFRVIII_CCx | artificial | aa | QVQLVESGGGVVQSGRSLRLSCAASGFTFRNY |
| | CD3-scFc scFv | | | GMHWVRQAPGKCLEWVAVIWYDGSDKYYAD |
| | | | | SVRGRFTISRDNSKNTLYLQMNSLRAEDTAVY |
| | | | | YCARDGYDILTGNPRDFDYWGQGTLVTVSSG |
| | | | | GGGSGGGGSGGGGSDTVMTQTPLSSHVTLGQP |
| | | | | ASISCRSSQSLVHSDGNTYLSWLQQRPGQPPRL |
| | | | | LIYRISRRFSGVPDRFSGSGAGTDFTLEISRVEA |
| | | | | EDVGVYYCMQSTHVPRTFGCGTKVEIK |
| 51. | EGFRVIII_CCx | artificial | aa | QVQLVESGGGVVQSGRSLRLSCAASGFTFRNY |
| | CD3-scFc | | | GMHWVRQAPGKCLEWVAVIWYDGSDKYYAD |
| | Bispecific | | | SVRGRFTISRDNSKNTLYLQMNSLRAEDTAVY |
| | molecule | | | YCARDGYDILTGNPRDFDYWGQGTLVTVSSG |
| | | | | GGGSGGGGSGGGGSDTVMTQTPLSSHVTLGQP |
| | | | | ASISCRSSQSLVHSDGNTYLSWLQQRPGQPPRL |
| | | | | LIYRISRRFSGVPDRFSGSGAGTDFTLEISRVEA |
| | | | | EDVGVYYCMQSTHVPRTFGCGTKVEIKSGGG |
| | | | | GSEVQLVESGGGLVQPGGSLKLSCAASGFTFN |
| | | | | KYAMNWVRQAPGKGLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGGGTKLTVL |
| 52. | EGFRVIII_CCx | artificial | aa | QVQLVESGGGVVQSGRSLRLSCAASGFTFRNY |
| | CD3-scFc | | | GMHWVRQAPGKCLEWVAVIWYDGSDKYYAD |
| | Bispecific HLE | | | SVRGRFTISRDNSKNTLYLQMNSLRAEDTAVY |
| | molecule | | | YCARDGYDILTGNPRDFDYWGQGTLVTVSSG |
| | | | | GGGSGGGGSGGGGSDTVMTQTPLSSHVTLGQP |
| | | | | ASISCRSSQSLVHSDGNTYLSWLQQRPGQPPRL |
| | | | | LIYRISRRFSGVPDRFSGSGAGTDFTLEISRVEA |
| | | | | EDVGVYYCMQSTHVPRTFGCGTKVEIKSGGG |
| | | | | GSEVQLVESGGGLVQPGGSLKLSCAASGFTFN |
| | | | | KYAMNWVRQAPGKGLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGGGTKLTVLG |
| | | | | GGGDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGK |
| 53. | MSLN_5 VH | artificial | aa | DYYMT |
| | CDR1 | | | |
| 54. | MSLN_5 VH | artificial | aa | YISSSGSTIYYADSVKG |
| | CDR2 | | | |
| 55. | MSLN_5 VH | artificial | aa | DRNSHFDY |
| | CDR3 | | | |
| 56. | MSLN_5 VL | artificial | aa | RASQGINTWLA |
| | CDR1 | | | |
| 57. | MSLN_5 VL | artificial | aa | GASGLQS |
| | CDR2 | | | |
| 58. | MSLN_5 VL | artificial | aa | QQAKSFPRT |
| | CDR3 | | | |
| 59. | MSLN_5 VH | artificial | aa | QVQLVESGGGLVKPGGSLRLSCAASGFTFSD |
| | | | | YYMTWIRQAPGKGLEWLSYISSSGSTIYYADS |
| | | | | VKGRFTISRDNAKNSLFLQMNSLRAEDTAVY |
| | | | | YCARDRNSHFDYWGQGTLVTVSS |
| 60. | MSLN_5 VL | artificial | aa | DIQMTQSPSSVSASVGDRVTITCRASQGINTW |
| | | | | LAWYQQKPGKAPKLLIYGASGLQSGVPSRFS |
| | | | | GSGSGTDFTLTISSLQPEDFATYYCQQAKSFPR |
| | | | | TFGQGTKVEIK |
| 61. | MSLN_5 scFv | artificial | aa | QVQLVESGGGLVKPGGSLRLSCAASGFTFSD |
| | | | | YYMTWIRQAPGKGLEWLSYISSSGSTIYYADS |
| | | | | VKGRFTISRDNAKNSLFLQMNSLRAEDTAVY |
| | | | | YCARDRNSHFDYWGQGTLVTVSSGGGGSGG |
| | | | | GGSGGGGSDIQMTQSPSSVSASVGDRVTITCR |
| | | | | ASQGINTWLAWYQQKPGKAPKLLIYGASGLQ |
| | | | | SGVPSRFSGSGSGTDFTLTISSLQPEDFATYYC |
| | | | | QQAKSFPRTFGQGTKVEIK |
| 62. | MSLN_5xI2C0 | artificial | aa | QVQLVESGGGLVKPGGSLRLSCAASGFTFSD |
| | bispecific | | | YYMTWIRQAPGKGLEWLSYISSSGSTIYYADS |
| | molecule | | | VKGRFTISRDNAKNSLFLQMNSLRAEDTAVY |
| | | | | YCARDRNSHFDYWGQGTLVTVSSGGGGSGG |
| | | | | GGSGGGGSDIQMTQSPSSVSASVGDRVTITCR |
| | | | | ASQGINTWLAWYQQKPGKAPKLLIYGASGLQ |
| | | | | SGVPSRFSGSGSGTDFTLTISSLQPEDFATYYC |
| | | | | QQAKSFPRTFGQGTKVEIKSGGGGSEVQLVES |
| | | | | GGGLVQPGGSLKLSCAASGFTFNKYAMNWV |
| | | | | RQAPGKGLEWVARIRSKYNNYATYYADSVK |
| | | | | DRFTISRDDSKNTAYLQMNNLKTEDTAVYYC |
| | | | | VRHGNFGNSYISYWAYWGQGTLVTVSSGGG |
| | | | | GSGGGGSGGGGSQTVVTQEPSLTVSPGGTVT |
| | | | | LTCGSSTGAVTSGNYPNWVQQKPGQAPRGLI |
| | | | | GGTKFLAPGTPARFSGSLLGGKAALTLSGVQP |
| | | | | EDEAEYYCVLWYSNRWVFGGGTKLTVL |
| 63. | MSLN_5xCD3- | artificial | aa | QVQLVESGGGLVKPGGSLRLSCAASGFTFSD |
| | scFc Bispecific | | | YYMTWIRQAPGKGLEWLSYISSSGSTIYYADS |
| | HLE molecule | | | VKGRFTISRDNAKNSLFLQMNSLRAEDTAVY |
| | | | | YCARDRNSHFDYWGQGTLVTVSSGGGGSGG |
| | | | | GGSGGGGSDIQMTQSPSSVSASVGDRVTITCR |
| | | | | ASQGINTWLAWYQQKPGKAPKLLIYGASGLQ |
| | | | | SGVPSRFSGSGSGTDFTLTISSLQPEDFATYYC |
| | | | | QQAKSFPRTFGQGTKVEIKSGGGGSEVQLVES |
| | | | | GGGLVQPGGSLKLSCAASGFTFNKYAMNWV |
| | | | | RQAPGKGLEWVARIRSKYNNYATYYADSVK |
| | | | | DRFTISRDDSKNTAYLQMNNLKTEDTAVYYC |
| | | | | VRHGNFGNSYISYWAYWGQGTLVTVSSGGG |
| | | | | GSGGGGSGGGGSQTVVTQEPSLTVSPGGTVT |
| | | | | LTCGSSTGAVTSGNYPNWVQQKPGQAPRGLI |
| | | | | GGTKFLAPGTPARFSGSLLGGKAALTLSGVQP |
| | | | | EDEAEYYCVLWYSNRWVFGGGTKLTVLGGG |
| | | | | GDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQD |
| | | | | WLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLY |
| | | | | SKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLF |
| | | | | PPKPKDTLMISRTPEVTCVVVDVSHEDPEVKF |
| | | | | NWYVDGVEVHNAKTKPCEEQYGSTYRCVSV |
| | | | | LTVLHQDWLNGKEYKCKVSNKALPAPIEKTI |
| | | | | SKAKGQPREPQVYTLPPSREEMTKNQVSLTC |
| | | | | LVKGFYPSDIAVEWESNGQPENNYKTTPPVL |
| | | | | DSDGSFFLYSKLTVDKSRWQQGNVFSCSVMH |
| | | | | EALHNHYTQKSLSLSPGK |
| 64. | MSLN_5_CCx | artificial | aa | QVQLVESGGGLVKPGGSLRLSCAASGFTFSD |
| | CD3-scFc | | | HYMSWIRQAPGKCLEWFSYISSSGGIIYYADS |
| | Bispecific HLE | | | VKGRFTISRDNAKNSLYLQMNSLRAEDTAVY |
| | molecule | | | YCARDVGSHFDYWGQGTLVTVSSGGGGSGG |
| | | | | GGSGGGGSDIQMTQSPSSVSASVGDRVTITCR |
| | | | | ASQDISRWLAWYQQKPGKAPKLLISAASRLQ |
| | | | | SGVPSRFSGSGSGTDFTLTISSLQPEDFAIYYC |
| | | | | QQAKSFPRTFGCGTKVEIKSGGGGSEVQLVES |
| | | | | GGGLVQPGGSLKLSCAASGFTFNKYAMNWV |
| | | | | RQAPGKGLEWVARIRSKYNNYATYYADSVK |
| | | | | DRFTISRDDSKNTAYLQMNNLKTEDTAVYYC |
| | | | | VRHGNFGNSYISYWAYWGQGTLVTVSSGGG |
| | | | | GSGGGGSGGGGSQTVVTQEPSLTVSPGGTVT |
| | | | | LTCGSSTGAVTSGNYPNWVQQKPGQAPRGLI |
| | | | | GGTKFLAPGTPARFSGSLLGGKAALTLSGVQP |
| | | | | EDEAEYYCVLWYSNRWVFGGGTKLTVLGGG |
| | | | | GDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQD |
| | | | | WLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLY |
| | | | | SKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLF |
| | | | | PPKPKDTLMISRTPEVTCVVVDVSHEDPEVKF |
| | | | | NWYVDGVEVHNAKTKPCEEQYGSTYRCVSV |
| | | | | LTVLHQDWLNGKEYKCKVSNKALPAPIEKTI |
| | | | | SKAKGQPREPQVYTLPPSREEMTKNQVSLTC |
| | | | | LVKGFYPSDIAVEWESNGQPENNYKTTPPVL |
| | | | | DSDGSFFLYSKLTVDKSRWQQGNVFSCSVMH |
| | | | | EALHNHYTQKSLSLSPGK |
| 65. | CDR-H1 of | artificial | aa | SYGMH |
| | CDH19 | | | |
| | 65254.007 | | | |
| 66. | CDR-H2 of | artificial | aa | FIWYEGSNKYYAESVKD |
| | CDH19 | | | |
| | 65254.007 | | | |
| 67. | CDR-H3 of | artificial | aa | RAGIIGTIGYYYGMDV |
| | CDH19 | | | |
| | 65254.007 | | | |
| 68. | CDR-L1 of | artificial | aa | SGDRLGEKYTS |
| | CDH19 | | | |
| | 65254.007 | | | |
| 69. | CDR-L2 of | artificial | aa | QDTKRPS |
| | CDH19 | | | |
| | 65254.007 | | | |
| 70. | CDR-L3 of | artificial | aa | QAWESSTVV |
| | CDH19 | | | |
| | 65254.007 | | | |
| 71. | VH of CDH19 | artificial | aa | QVQLVESGGGVVQPGGSLRLSCAASGFTFSS |
| | 65254.007 | | | YGMHWVRQAPGKGLEWVAFIWYEGSNKYY |
| | | | | AESVKDRFTISRDNSKNTLYLQMNSLRAEDT |
| | | | | AVYYCARRAGIIGTIGYYYGMDVWGQGTTV |
| | | | | TVSS |
| 72. | VL of CDH19 | artificial | aa | SYELTQPPSVSVSPGQTASITCSGDRLGEKYTS |
| | 65254.007 | | | WYQQRPGQSPLLVIYQDTKRPSGIPERFSGSN |
| | | | | SGNTATLTISGTQAMDEADYYCQAWESSTVV |
| | | | | FGGGTKLTVLS |
| 73. | VH-VL of | artificial | aa | QVQLVESGGGVVQPGGSLRLSCAASGFTFSS |
| | CDH19 | | | YGMHWVRQAPGKGLEWVAFIWYEGSNKYY |
| | 65254.007 | | | AESVKDRFTISRDNSKNTLYLQMNSLRAEDT |
| | | | | AVYYCARRAGIIGTIGYYYGMDVWGQGTTV |
| | | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGGGTKLTVLS |
| 74. | CDH19 | artificial | aa | QVQLVESGGGVVQPGGSLRLSCAASGFTFSS |
| | 65254.007 x I2C | | | YGMHWVRQAPGKGLEWVAFIWYEGSNKYY |
| | | | | AESVKDRFTISRDNSKNTLYLQMNSLRAEDT |
| | | | | AVYYCARRAGIIGTIGYYYGMDVWGQGTTV |
| | | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGGGTKLTVLSG |
| | | | | GGGSEVQLVESGGGLVQPGGSLKLSCAASGF |
| | | | | TFNKYAMNWVRQAPGKGLEWVARIRSKYNN |
| | | | | YATYYADSVKDRFTISRDDSKNTAYLQMNNL |
| | | | | KTEDTAVYYCVRHGNFGNSYISYWAYWGQG |
| | | | | TLVTVSSGGGGSGGGGSGGGGSQTVVTQEPS |
| | | | | LTVSPGGTVTLTCGSSTGAVTSGNYPNWVQQ |
| | | | | KPGQAPRGLIGGTKFLAPGTPARFSGSLLGGK |
| | | | | AALTLSGVQPEDEAEYYCVLWYSNRWVFGG |
| | | | | GTKLTVLHHHHHH |
| 75. | CDH19 | artificial | aa | QVQLVESGGGVVQPGGSLRLSCAASGFTFSS |
| | 65254.007 x I2C- | | | YGMHWVRQAPGKGLEWVAFIWYEGSNKYY |
| | scFc Bispecific | | | AESVKDRFTISRDNSKNTLYLQMNSLRAEDT |
| | HLE molecule | | | AVYYCARRAGIIGTIGYYYGMDVWGQGTTV |
| | | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGGGTKLTVLSG |
| | | | | GGGSEVQLVESGGGLVQPGGSLKLSCAASGF |
| | | | | TFNKYAMNWVRQAPGKGLEWVARIRSKYNN |
| | | | | YATYYADSVKDRFTISRDDSKNTAYLQMNNL |
| | | | | KTEDTAVYYCVRHGNFGNSYISYWAYWGQG |
| | | | | TLVTVSSGGGGSGGGGSGGGGSQTVVTQEPS |
| | | | | LTVSPGGTVTLTCGSSTGAVTSGNYPNWVQQ |
| | | | | KPGQAPRGLIGGTKFLAPGTPARFSGSLLGGK |
| | | | | AALTLSGVQPEDEAEYYCVLWYSNRWVFGG |
| | | | | GTKLTVLGGGGDKTHTCPPCPAPELLGGPSVF |
| | | | | LFPPKPKDTLMISRTPEVTCVVVDVSHEDPEV |
| | | | | KFNWYVDGVEVHNAKTKPCEEQYGSTYRCV |
| | | | | SVLTVLHQDWLNGKEYKCKVSNKALPAPIEK |
| | | | | TISKAKGQPREPQVYTLPPSREEMTKNQVSLT |
| | | | | CLVKGFYPSDIAVEWESNGQPENNYKTTPPV |
| | | | | LDSDGSFFLYSKLTVDKSRWQQGNVFSCSVM |
| | | | | HEALHNHYTQKSLSLSPGKGGGGSGGGGSGG |
| | | | | GGSGGGGSGGGGSGGGGSDKTHTCPPCPAPE |
| | | | | LLGGPSVFLFPPKPKDTLMISRTPEVTCVVVD |
| | | | | VSHEDPEVKFNWYVDGVEVHNAKTKPCEEQ |
| | | | | YGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPEN |
| | | | | NYKTTPPVLDSDGSFFLYSKLTVDKSRWQQG |
| | | | | NVFSCSVMHEALHNHYTQKSLSLSPGK |
| 76. | CDH19 | artificial | aa | QVQLVESGGGVVQPGGSLRLSCAASGFTFSS |
| | 65254.007 x I2C- | | | YGMHWVRQAPGKGLEWVAFIWYEGSNKYY |
| | scFc_delGK | | | AESVKDRFTISRDNSKNTLYLQMNSLRAEDT |
| | Bispecific HLE | | | AVYYCARRAGIIGTIGYYYGMDVWGQGTTV |
| | molecule | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGGGTKLTVLSG |
| | | | | GGGSEVQLVESGGGLVQPGGSLKLSCAASGF |
| | | | | TFNKYAMNWVRQAPGKGLEWVARIRSKYN |
| | | | | NYATYYADSVKDRFTISRDDSKNTAYLQMN |
| | | | | NLKTEDTAVYYCVRHGNFGNSYISYWAYWG |
| | | | | QGTLVTVSSGGGGSGGGGSGGGGSQTVVTQ |
| | | | | EPSLTVSPGGTVTLTCGSSTGAVTSGNYPNW |
| | | | | VQQKPGQAPRGLIGGTKFLAPGTPARFSGSLL |
| | | | | GGKAALTLSGVQPEDEAEYYCVLWYSNRWV |
| | | | | FGGGTKLTVLGGGGDKTHTCPPCPAPELLGG |
| | | | | PSVFLFPPKPKDTLMISRTPEVTCVVVDVSHE |
| | | | | DPEVKFNWYVDGVEVHNAKTKPCEEQYGST |
| | | | | YRCVSVLTVLHQDWLNGKEYKCKVSNKALP |
| | | | | APIEKTISKAKGQPREPQVYTLPPSREEMTKN |
| | | | | QVSLTCLVKGFYPSDIAVEWESNGQPENNYK |
| | | | | TTPPVLDSDGSFFLYSKLTVDKSRWQQGNVF |
| | | | | SCSVMHEALHNHYTQKSLSLSPGGGGSGGGG |
| | | | | SGGGGSGGGGSGGGGSGGGGSDKTHTCPPCP |
| | | | | APELLGGPSVFLFPPKPKDTLMISRTPEVTCVV |
| | | | | VDVSHEDPEVKFNWYVDGVEVHNAKTKPCE |
| | | | | EQYGSTYRCVSVLTVLHQDWLNGKEYKCKV |
| | | | | SNKALPAPIEKTISKAKGQPREPQVYTLPPSRE |
| | | | | EMTKNQVSLTCLVKGFYPSDIAVEWESNGQP |
| | | | | ENNYKTTPPVLDSDGSFFLYSKLTVDKSRWQ |
| | | | | QGNVFSCSVMHEALHNHYTQKSLSLSPGK |
| 77. | CDH19 | artificial | aa | QVQLVESGGGVVQPGGSLRLSCAASGFTFSS |
| | 65254.007_CC | | | YGMHWVRQAPGKCLEWVAFIWYEGSNKYY |
| | x I2C-scFc | | | AESVKDRFTISRDNSKNTLYLQMNSLRAEDT |
| | VH | | | AVYYCARRAGIIGTIGYYYGMDVWGQGTTV |
| | | | | TVSS |
| 78. | CDH19 | artificial | aa | SYELTQPPSVSVSPGQTASITCSGDRLGEKYTS |
| | 65254.007_CC | | | WYQQRPGQSPLLVIYQDTKRPSGIPERFSGSN |
| | x I2C-scFc VL | | | SGNTATLTISGTQAMDEADYYCQAWESSTVV |
| | | | | FGCGTKLTVL |
| 79. | CDH19 | artificial | aa | QVQLVESGGGVVQPGGSLRLSCAASGFTFSS |
| | 65254.007_CC | | | YGMHWVRQAPGKCLEWVAFIWYEGSNKYY |
| | x I2C-scFc | | | AESVKDRFTISRDNSKNTLYLQMNSLRAEDT |
| | scFv | | | AVYYCARRAGIIGTIGYYYGMDVWGQGTTV |
| | | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGCGTKLTVL |
| 80. | CDH19 | artificial | aa | QVQLVESGGGVVQPGGSLRLSCAASGFTFSS |
| | 65254.007_CC | | | YGMHWVRQAPGKCLEWVAFIWYEGSNKYY |
| | x I2C-scFc | | | AESVKDRFTISRDNSKNTLYLQMNSLRAEDT |
| | Bispecific | | | AVYYCARRAGIIGTIGYYYGMDVWGQGTTV |
| | molecule | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGCGTKLTVLSG |
| | | | | GGGSEVQLVESGGGLVQPGGSLKLSCAASGF |
| | | | | TFNKYAMNWVRQAPGKGLEWVARIRSKYN |
| | | | | NYATYYADSVKDRFTISRDDSKNTAYLQMN |
| | | | | NLKTEDTAVYYCVRHGNFGNSYISYWAYWG |
| | | | | QGTLVTVSSGGGGSGGGGSGGGGSQTVVTQ |
| | | | | EPSLTVSPGGTVTLTCGSSTGAVTSGNYPNW |
| | | | | VQQKPGQAPRGLIGGTKFLAPGTPARFSGSLL |
| | | | | GGKAALTLSGVQPEDEAEYYCVLWYSNRWV |
| | | | | FGGGTKLTVL |
| 81. | CDH19 | artificial | aa | QVQLVESGGGVVQPGGSLRLSCAASGFTFSS |
| | 65254.007_CC | | | YGMHWVRQAPGKCLEWVAFIWYEGSNKYY |
| | x I2C-scFc | | | AESVKDRFTISRDNSKNTLYLQMNSLRAEDT |
| | Bispecific HLE | | | AVYYCARRAGIIGTIGYYYGMDVWGQGTTV |
| | molecule | | | TVSSGGGGSGGGGSGGGGSSYELTQPPSVSVS |
| | | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGCGTKLTVLSG |
| | | | | GGGSEVQLVESGGGLVQPGGSLKLSCAASGF |
| | | | | TFNKYAMNWVRQAPGKGLEWVARIRSKYN |
| | | | | NYATYYADSVKDRFTISRDDSKNTAYLQMN |
| | | | | NLKTEDTAVYYCVRHGNFGNSYISYWAYWG |
| | | | | QGTLVTVSSGGGGSGGGGSGGGGSQTVVTQ |
| | | | | EPSLTVSPGGTVTLTCGSSTGAVTSGNYPNW |
| | | | | VQQKPGQAPRGLIGGTKFLAPGTPARFSGSLL |
| | | | | GGKAALTLSGVQPEDEAEYYCVLWYSNRWV |
| | | | | FGGGTKLTVLGGGGDKTHTCPPCPAPELLGG |
| | | | | PSVFLFPPKPKDTLMISRTPEVTCVVVDVSHE |
| | | | | DPEVKFNWYVDGVEVHNAKTKPCEEQYGST |
| | | | | YRCVSVLTVLHQDWLNGKEYKCKVSNKALP |
| | | | | APIEKTISKAKGQPREPQVYTLPPSREEMTKN |
| | | | | QVSLTCLVKGFYPSDIAVEWESNGQPENNYK |
| | | | | TTPPVLDSDGSFFLYSKLTVDKSRWQQGNVF |
| | | | | SCSVMHEALHNHYTQKSLSLSPGKGGGGSGG |
| | | | | GGSGGGGSGGGGSGGGGSGGGGSDKTHTCP |
| | | | | PCPAPELLGGPSVFLFPPKPKDTLMISRTPEVT |
| | | | | CVVVDVSHEDPEVKFNWYVDGVEVHNAKT |
| | | | | KPCEEQYGSTYRCVSVLTVLHQDWLNGKEY |
| | | | | KCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| | | | | GK |
| 82. | CDH19 | artificial | aa | QVQLVESGGGVVQPGGSLRLSCAASGFTFSS |
| | 65254.007_CC | | | YGMHWVRQAPGKCLEWVAFIWYEGSNKYY |
| | x I2C- | | | AESVKDRFTISRDNSKNTLYLQMNSLRAEDT |
| | scFc_delGK | | | AVYYCARRAGIIGTIGYYYGMDVWGQGTTV |
| | Bispecific HLE | | | TVSSGGGGSGGGGSGGGGSSYELTQPPSVSVS |
| | molecule | | | PGQTASITCSGDRLGEKYTSWYQQRPGQSPLL |
| | | | | VIYQDTKRPSGIPERFSGSNSGNTATLTISGTQ |
| | | | | AMDEADYYCQAWESSTVVFGCGTKLTVLSG |
| | | | | GGGSEVQLVESGGGLVQPGGSLKLSCAASGF |
| | | | | TFNKYAMNWVRQAPGKGLEWVARIRSKYN |
| | | | | NYATYYADSVKDRFTISRDDSKNTAYLQMN |
| | | | | NLKTEDTAVYYCVRHGNFGNSYISYWAYWG |
| | | | | QGTLVTVSSGGGGSGGGGSGGGGSQTVVTQ |
| | | | | EPSLTVSPGGTVTLTCGSSTGAVTSGNYPNW |
| | | | | VQQKPGQAPRGLIGGTKFLAPGTPARFSGSLL |
| | | | | GGKAALTLSGVQPEDEAEYYCVLWYSNRWV |
| | | | | FGGGTKLTVLGGGGDKTHTCPPCPAPELLGG |
| | | | | PSVFLFPPKPKDTLMISRTPEVTCVVVDVSHE |
| | | | | DPEVKFNWYVDGVEVHNAKTKPCEEQYGST |
| | | | | YRCVSVLTVLHQDWLNGKEYKCKVSNKALP |
| | | | | APIEKTISKAKGQPREPQVYTLPPSREEMTKN |
| | | | | QVSLTCLVKGFYPSDIAVEWESNGQPENNYK |
| | | | | TTPPVLDSDGSFFLYSKLTVDKSRWQQGNVF |
| | | | | SCSVMHEALHNHYTQKSLSLSPGGGGSGGGG |
| | | | | SGGGGSGGGGSGGGGSGGGGSDKTHTCPPCP |
| | | | | APELLGGPSVFLFPPKPKDTLMISRTPEVTCVV |
| | | | | VDVSHEDPEVKFNWYVDGVEVHNAKTKPCE |
| | | | | EQYGSTYRCVSVLTVLHQDWLNGKEYKCKV |
| | | | | SNKALPAPIEKTISKAKGQPREPQVYTLPPSRE |
| | | | | EMTKNQVSLTCLVKGFYPSDIAVEWESNGQP |
| | | | | ENNYKTTPPVLDSDGSFFLYSKLTVDKSRWQ |
| | | | | QGNVFSCSVMHEALHNHYTQKSLSLSPGK |
| 83. | FLT3_7 | artificial | aa | NARMGVS |
| | A8xCD3-scFc | | | |
| | VH CDR1 | | | |
| 84. | FLT3_7 | artificial | aa | HIFSNDEKSYSTSLKN |
| | A8xCD3-scFc | | | |
| | VH CDR2 | | | |
| 85. | FLT3_7 | artificial | aa | IVGYGSGWYGFFDY |
| | A8xCD3-scFc | | | |
| | VH CDR3 | | | |
| 86 | FLT3_7 | artificial | aa | RASQGIRNDLG |
| | A8xCD3-scFc | | | |
| | VL CDR1 | | | |
| 87. | FLT3_7 | artificial | aa | AASTLQS |
| | A8xCD3-scFc | | | |
| | VL CDR2 | | | |
| 88. | FLT3_7 | artificial | aa | LQHNSYPLT |
| | A8xCD3-scFc | | | |
| | VL CDR3 | | | |
| 89. | FLT3_7 | artificial | aa | QVTLKESGPTLVKPTETLTLTCTLSGFSLNNAR |
| | A8xCD3-scFc | | | MGVSWIRQPPGKCLEWLAHIFSNDEKSYSTSLK |
| | VH | | | NRLTISKDSSKTQVVLTMTNVDPVDTATYYCA |
| | | | | RIVGYGSGWYGFFDYWGQGTLVTVSS |
| 90. | FLT3_A8- | artificial | aa | DIQMTQSPSSLSASVGDRVTITCRASQGIRNDLG |
| | scFc VL | | | WYQQKPGKAPKRLIYAASTLQSGVPSRFSGSGS |
| | | | | GTEFTLTISSLQPEDFATYYCLQHNSYPLTFGCG |
| | | | | TKVEIK |
| 91. | FLT3_7 | artificial | aa | QVTLKESGPTLVKPTETLTLTCTLSGFSLNNAR |
| | A8xCD3- | | | MGVSWIRQPPGKCLEWLAHIFSNDEKSYSTSLK |
| | scFv | | | NRLTISKDSSKTQVVLTMTNVDPVDTATYYCA |
| | | | | RIVGYGSGWYGFFDYWGQGTLVTVSSGGGGS |
| | | | | GGGGSGGGGSDIQMTQSPSSLSASVGDRVTITC |
| | | | | RASQGIRNDLGWYQQKPGKAPKRLIYAASTLQ |
| | | | | SGVPSRFSGSGSGTEFTLTISSLQPEDFATYYCL |
| | | | | QHNSYPLTFGCGTKVEIK |
| 92. | FLT3_7 | artificial | aa | QVTLKESGPTLVKPTETLTLTCTLSGFSLNNAR |
| | A8xCD3 | | | MGVSWIRQPPGKCLEWLAHIFSNDEKSYSTSLK |
| | Bispecific | | | NRLTISKDSSKTQVVLTMTNVDPVDTATYYCA |
| | molecule | | | RIVGYGSGWYGFFDYWGQGTLVTVSSGGGGS |
| | | | | GGGGSGGGGSDIQMTQSPSSLSASVGDRVTITC |
| | | | | RASQGIRNDLGWYQQKPGKAPKRLIYAASTLQ |
| | | | | SGVPSRFSGSGSGTEFTLTISSLQPEDFATYYCL |
| | | | | QHNSYPLTFGCGTKVEIKSGGGGSEVQLVESGG |
| | | | | GLVQPGGSLKLSCAASGFTFNKYAMNWVRQA |
| | | | | PGKGLEWVARIRSKYNNYATYYADSVKDRFTI |
| | | | | SRDDSKNTAYLQMNNLKTEDTAVYYCVRHGN |
| | | | | FGNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVL |
| 93. | FLT3_7 | artificial | aa | QVTLKESGPTLVKPTETLTLTCTLSGFSLNNAR |
| | A8xCD3-scFc | | | MGVSWIRQPPGKCLEWLAHIFSNDEKSYSTSLK |
| | Bispecific | | | NRLTISKDSSKTQVVLTMTNVDPVDTATYYCA |
| | HLE | | | RIVGYGSGWYGFFDYWGQGTLVTVSSGGGGS |
| | molecule | | | GGGGSGGGGSDIQMTQSPSSLSASVGDRVTITC |
| | | | | RASQGIRNDLGWYQQKPGKAPKRLIYAASTLQ |
| | | | | SGVPSRFSGSGSGTEFTLTISSLQPEDFATYYCL |
| | | | | QHNSYPLTFGCGTKVEIKSGGGGSEVQLVESGG |
| | | | | GLVQPGGSLKLSCAASGFTFNKYAMNWVRQA |
| | | | | PGKGLEWVARIRSKYNNYATYYADSVKDRFTI |
| | | | | SRDDSKNTAYLQMNNLKTEDTAVYYCVRHGN |
| | | | | FGNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 94. | VH CDR1 | artificial | aa | SYYWS |
| | DLL3_1_CC_ | | | |
| | delGK | | | |
| 95. | VH CDR2 | artificial | aa | YVYYSGTTNYNPSLKS |
| | DLL3_1_CC_ | | | |
| | delGK | | | |
| 96. | VH CDR3 | artificial | aa | IAVTGFYFDY |
| | DLL3_1_CC_ | | | |
| | delGK | | | |
| 97. | VL CDR1 | artificial | aa | RASQRVNNNYLA |
| | DLL3_1_CC_ | | | |
| | delGK | | | |
| 98. | VL CDR2 | artificial | aa | GASSRAT |
| | DLL3_1_CC_ | | | |
| | delGK | | | |
| 99. | VL CDR3 | artificial | aa | QQYDRSPLT |
| | DLL3_1_CC_ | | | |
| | delGK | | | |
| 100. | VH | artificial | aa | QVQLQESGPGLVKPSETLSLTCTVSGGSISSYY |
| | DLL3_1_CC_ | | | WSWIRQPPGKCLEWIGYVYYSGTTNYNPSLKS |
| | delGK | | | RVTISVDTSKNQFSLKLSSVTAADTAVYYCASI |
| | | | | AVTGFYFDYWGQGTLVTVSS |
| 101. | VL | artificial | aa | EIVLTQSPGTLSLSPGERVTLSCRASQRVNNNYL |
| | DLL3_1_CC_ | | | AWYQQRPGQAPRLLIYGASSRATGIPDRESGSG |
| | delGK | | | SGTDFTLTISRLEPEDFAVYYCQQYDRSPLTFGC |
| | | | | GTKLEIK |
| 102. | DLL3_1_CC_ | artificial | aa | QVQLQESGPGLVKPSETLSLTCTVSGGSISSYY |
| | delGK | | | WSWIRQPPGKCLEWIGYVYYSGTTNYNPSLKS |
| | | | | RVTISVDTSKNQFSLKLSSVTAADTAVYYCASI |
| | | | | AVTGFYFDYWGQGTLVTVSSGGGGSGGGGSG |
| | | | | GGGSEIVLTQSPGTLSLSPGERVTLSCRASQRVN |
| | | | | NNYLAWYQQRPGQAPRLLIYGASSRATGIPDRF |
| | | | | SGSGSGTDFTLTISRLEPEDFAVYYCQQYDRSPL |
| | | | | TFGCGTKLEIK |
| 103. | DLL3_1_CCx | artificial | aa | QVQLQESGPGLVKPSETLSLTCTVSGGSISSYY |
| | CD3_delGK | | | WSWIRQPPGKCLEWIGYVYYSGTTNYNPSLKS |
| | Bispecific | | | RVTISVDTSKNQFSLKLSSVTAADTAVYYCASI |
| | molecule | | | AVTGFYFDYWGQGTLVTVSSGGGGSGGGGSG |
| | | | | GGGSEIVLTQSPGTLSLSPGERVTLSCRASQRVN |
| | | | | NNYLAWYQQRPGQAPRLLIYGASSRATGIPDRF |
| | | | | SGSGSGTDFTLTISRLEPEDFAVYYCQQYDRSPL |
| | | | | TFGCGTKLEIKSGGGGSEVQLVESGGGLVQPGG |
| | | | | SLKLSCAASGFTFNKYAMNWVRQAPGKGLEW |
| | | | | VARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQT |
| | | | | VVTQEPSLTVSPGGTVTLTCGSSTGAVTSGNYP |
| | | | | NWVQQKPGQAPRGLIGGTKFLAPGTPARFSGSL |
| | | | | LGGKAALTLSGVQPEDEAEYYCVLWYSNRWV |
| | | | | FGGGTKLTVL |
| 104. | DLL3_1_CCx | artificial | aa | QVQLQESGPGLVKPSETLSLTCTVSGGSISSYY |
| | CD3- | | | WSWIRQPPGKCLEWIGYVYYSGTTNYNPSLKS |
| | scFc_delGK | | | RVTISVDTSKNQFSLKLSSVTAADTAVYYCASI |
| | Bispecific | | | AVTGFYFDYWGQGTLVTVSSGGGGSGGGGSG |
| | HLE | | | GGGSEIVLTQSPGTLSLSPGERVTLSCRASQRVN |
| | molecule | | | NNYLAWYQQRPGQAPRLLIYGASSRATGIPDRF |
| | | | | SGSGSGTDFTLTISRLEPEDFAVYYCQQYDRSPL |
| | | | | TFGCGTKLEIKSGGGGSEVQLVESGGGLVQPGG |
| | | | | SLKLSCAASGFTFNKYAMNWVRQAPGKGLEW |
| | | | | VARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQT |
| | | | | VVTQEPSLTVSPGGTVTLTCGSSTGAVTSGNYP |
| | | | | NWVQQKPGQAPRGLIGGTKFLAPGTPARFSGSL |
| | | | | LGGKAALTLSGVQPEDEAEYYCVLWYSNRWV |
| | | | | FGGGTKLTVLGGGGDKTHTCPPCPAPELLGGPS |
| | | | | VFLFPPKPKDTLMISRTPEVTCVVVDVSHEDPE |
| | | | | VKFNWYVDGVEVHNAKTKPCEEQYGSTYRCV |
| | | | | SVLTVLHQDWLNGKEYKCKVSNKALPAPIEKTI |
| | | | | SKAKGQPREPQVYTLPPSREEMTKNQVSLTCLV |
| | | | | KGFYPSDIAVEWESNGQPENNYKTTPPVLDSDG |
| | | | | SFFLYSKLTVDKSRWQQGNVFSCSVMHEALHN |
| | | | | HYTQKSLSLSPGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKGF |
| | | | | YPSDIAVEWESNGQPENNYKTTPPVLDSDGSFF |
| | | | | LYSKLTVDKSRWQQGNVFSCSVMHEALHNHY |
| | | | | TQKSLSLSPGK |
| 105. | VH CDR1 | artificial | aa | SYGMH |
| | CD19 97- | | | |
| | G1RE-C2 | | | |
| 106. | VH CDR2 | artificial | aa | VISYEGSNKYYAESVKG |
| | CD19 97- | | | |
| | G1RE-C2 | | | |
| 107. | VH CDR3 | artificial | aa | DRGTIFGNYGLEV |
| | CD19 97- | | | |
| | G1RE-C2 | | | |
| 108. | VH CD19 97- | artificial | aa | QVQLVESGGGVVQPGRSLRLSCAASGFTFSSYG |
| | G1RE-C2 CC | | | MHWVRQAPGKCLEWVAVISYEGSNKYYAESV |
| | | | | KGRFTISRDNSKNTLYLQMNSLRDEDTAVYYC |
| | | | | ARDRGTIFGNYGLEVWGQGTTVTVSS |
| 109 | VL CDR1 | artificial | aa | RSSQSLLHKNAFNYLD |
| | CD19 97- | | | |
| | G1RE-C2 | | | |
| 110. | VL CDR2 | artificial | aa | LGSNRAS |
| | CD19 97- | | | |
| | G1RE-C2 | | | |
| 111. | VL CDR3 | artificial | aa | MQALQTPFT |
| | CD19 97- | | | |
| | G1RE-C2 | | | |
| 112. | VL CD19 97- | artificial | aa | DIVMTQSPLSLPVISGEPASISCRSSQSLLHKNAF |
| | G1RE-C2 CC | | | NYLDWYLQKPGQSPQLLIYLGSNRASGVPDRFS |
| | | | | GSGSGTDFTLKISRVEAEDVGVYYCMQALQTP |
| | | | | FTFGCGTKVDIK |
| 113. | CD19 97- | artificial | aa | MDMRVPAQLLGLLLLWLRGARCDIVMTQSPLS |
| | G1RE-C2 CC | | | LPVISGEPASISCRSSQSLLHKNAFNYLDWYLQ |
| | x I2C0 | | | KPGQSPQLLIYLGSNRASGVPDRFSGSGSGTDFT |
| | | | | LKISRVEAEDVGVYYCMQALQTPFTFGCGTKV |
| | | | | DIKGGGGSGGGGSGGGGSQVQLVESGGGVVQP |
| | | | | GRSLRLSCAASGFTFSSYGMHWVRQAPGKCLE |
| | | | | WVAVISYEGSNKYYAESVKGRFTISRDNSKNTL |
| | | | | YLQMNSLRDEDTAVYYCARDRGTIFGNYGLEV |
| | | | | WGQGTTVTVSSGGGGSEVQLVESGGGLVQPG |
| | | | | GSLKLSCAASGFTFNKYAMNWVRQAPGKGLE |
| | | | | WVARIRSKYNNYATYYADSVKDRFTISRDDSK |
| | | | | NTAYLQMNNLKTEDTAVYYCVRHGNFGNSYIS |
| | | | | YWAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSGS |
| | | | | LLGGKAALTLSGVQPEDEAEYYCVLWYSNRW |
| | | | | VFGGGTKLTVL |
| 114. | CD19 97- | artificial | aa | MDMRVPAQLLGLLLLWLRGARCDIVMTQSPLS |
| | G1RE-C2 CC | | | LPVISGEPASISCRSSQSLLHKNAFNYLDWYLQ |
| | x I2C0-scFc | | | KPGQSPQLLIYLGSNRASGVPDRFSGSGSGTDFT |
| | | | | LKISRVEAEDVGVYYCMQALQTPFTFGCGTKV |
| | | | | DIKGGGGSGGGGSGGGGSQVQLVESGGGVVQP |
| | | | | GRSLRLSCAASGFTFSSYGMHWVRQAPGKCLE |
| | | | | WVAVISYEGSNKYYAESVKGRFTISRDNSKNTL |
| | | | | YLQMNSLRDEDTAVYYCARDRGTIFGNYGLEV |
| | | | | WGQGTTVTVSSGGGGSEVQLVESGGGLVQPG |
| | | | | GSLKLSCAASGFTFNKYAMNWVRQAPGKGLE |
| | | | | WVARIRSKYNNYATYYADSVKDRFTISRDDSK |
| | | | | NTAYLQMNNLKTEDTAVYYCVRHGNFGNSYIS |
| | | | | YWAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSGS |
| | | | | LLGGKAALTLSGVQPEDEAEYYCVLWYSNRW |
| | | | | VFGGGTKLTVLGGGGDKTHTCPPCPAPELLGG |
| | | | | PSVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLDS |
| | | | | DGSFFLYSKLTVDKSRWQQGNVFSCSVMHEAL |
| | | | | HNHYTQKSLSLSPGKGGGGSGGGGSGGGGSGG |
| | | | | GGSGGGGSGGGGSDKTHTCPPCPAPELLGGPSV |
| | | | | FLFPPKPKDTLMISRTPEVTCVVVDVSHEDPEV |
| | | | | KFNWYVDGVEVHNAKTKPCEEQYGSTYRCVS |
| | | | | VLTVLHQDWLNGKEYKCKVSNKALPAPIEKTIS |
| | | | | KAKGQPREPQVYTLPPSREEMTKNQVSLTCLV |
| | | | | KGFYPSDIAVEWESNGQPENNYKTTPPVLDSDG |
| | | | | SFFLYSKLTVDKSRWQQGNVFSCSVMHEALHN |
| | | | | HYTQKSLSLSPGK |
| 115. | VH CDR1 | artificial | aa | SYPIN |
| | CDH3 G8A | | | |
| | 6-B12 | | | |
| 116. | VH CDR2 | artificial | aa | VIWTGGGTNYASSVKG |
| | CDH3 G8A | | | |
| | 6-B12 | | | |
| 117. | VH CDR3 | artificial | aa | SRGVYDFDGRGAMDY |
| | CDH3 G8A | | | |
| | 6-B12 | | | |
| 118. | VL CDR1 | artificial | aa | KSSQSLLYSSNQKNYFA |
| | CDH3 G8A | | | |
| | 6-B12 | | | |
| 119. | VL CDR2 | artificial | aa | WASTRES |
| | CDH3 G8A | | | |
| | 6-B12 | | | |
| 120. | VL CDR3 | artificial | aa | QQYYSYPYT |
| | CDH3 G8A | | | |
| | 6-B12 | | | |
| 121. | VH CDH3 | artificial | aa | EVQLLESGGGLVQPGGSLRLSCAASGFSFSSYPI |
| | G8A 6-B12 | | | NWVRQAPGKGLEWVGVIWTGGGTNYASSVKG |
| | | | | RFTISRDNSKNTVYLQMNSLRAEDTAVYYCAK |
| | | | | SRGVYDFDGRGAMDYWGQGTLVTVSS |
| 122. | VL CDH3 | artificial | aa | DIVMTQSPDSLAVSLGERATINCKSSQSLLYSSN |
| | G8A 6-B12 | | | QKNYFAWYQQKPGQPPKLLIYWASTRESGVPD |
| | | | | RFSGSGSGTDFTLTISSLQAEDVAVYYCQQYYS |
| | | | | YPYTFGQGTKLEIK |
| 123 | CDH3 G8A | artificial | aa | EVQLLESGGGLVQPGGSLRLSCAASGFSFSSYPI |
| | 6-B12 scFv | | | NWVRQAPGKGLEWVGVIWTGGGTNYASSVKG |
| | | | | RFTISRDNSKNTVYLQMNSLRAEDTAVYYCAK |
| | | | | SRGVYDFDGRGAMDYWGQGTLVTVSSGGGGS |
| | | | | GGGGSGGGGSDIVMTQSPDSLAVSLGERATINC |
| | | | | KSSQSLLYSSNQKNYFAWYQQKPGQPPKLLIY |
| | | | | WASTRESGVPDRFSGSGSGTDFTLTISSLQAED |
| | | | | VAVYYCQQYYSYPYTFGQGTKLEIK |
| 124. | CDH3 G8A | artificial | aa | EVQLLESGGGLVQPGGSLRLSCAASGFSFSSYPI |
| | 6-B12 x I2C0 | | | NWVRQAPGKGLEWVGVIWTGGGTNYASSVKG |
| | bispecific | | | RFTISRDNSKNTVYLQMNSLRAEDTAVYYCAK |
| | molecule | | | SRGVYDFDGRGAMDYWGQGTLVTVSSGGGGS |
| | | | | GGGGSGGGGSDIVMTQSPDSLAVSLGERATINC |
| | | | | KSSQSLLYSSNQKNYFAWYQQKPGQPPKLLIY |
| | | | | WASTRESGVPDRFSGSGSGTDFTLTISSLQAED |
| | | | | VAVYYCQQYYSYPYTFGQGTKLEIKSGGGGSE |
| | | | | VQLVESGGGLVQPGGSLKLSCAASGFTFNKYA |
| | | | | MNWVRQAPGKGLEWVARIRSKYNNYATYYA |
| | | | | DSVKDRFTISRDDSKNTAYLQMNNLKTEDTAV |
| | | | | YYCVRHGNFGNSYISYWAYWGQGTLVTVSSG |
| | | | | GGGSGGGGSGGGGSQTVVTQEPSLTVSPGGTV |
| | | | | TLTCGSSTGAVTSGNYPNWVQQKPGQAPRGLI |
| | | | | GGTKFLAPGTPARFSGSLLGGKAALTLSGVQPE |
| | | | | DEAEYYCVLWYSNRWVFGGGTKLTVL |
| 125. | CDH3 G8A | artificial | aa | EVQLLESGGGLVQPGGSLRLSCAASGFSFSSYPI |
| | 6-B12 x I2C0 | | | NWVRQAPGKGLEWVGVIWTGGGTNYASSVKG |
| | bispecific | | | RFTISRDNSKNTVYLQMNSLRAEDTAVYYCAK |
| | molecule | | | SRGVYDFDGRGAMDYWGQGTLVTVSSGGGGS |
| | HLE | | | GGGGSGGGGSDIVMTQSPDSLAVSLGERATINC |
| | | | | KSSQSLLYSSNQKNYFAWYQQKPGQPPKLLIY |
| | | | | WASTRESGVPDRFSGSGSGTDFTLTISSLQAED |
| | | | | VAVYYCQQYYSYPYTFGQGTKLEIKSGGGGSE |
| | | | | VQLVESGGGLVQPGGSLKLSCAASGFTFNKYA |
| | | | | MNWVRQAPGKGLEWVARIRSKYNNYATYYA |
| | | | | DSVKDRFTISRDDSKNTAYLQMNNLKTEDTAV |
| | | | | YYCVRHGNFGNSYISYWAYWGQGTLVTVSSG |
| | | | | GGGSGGGGSGGGGSQTVVTQEPSLTVSPGGTV |
| | | | | TLTCGSSTGAVTSGNYPNWVQQKPGQAPRGLI |
| | | | | GGTKFLAPGTPARFSGSLLGGKAALTLSGVQPE |
| | | | | DEAEYYCVLWYSNRWVFGGGTKLTVLGGGGD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWESN |
| | | | | GQPENNYKTTPPVLDSDGSFFLYSKLTVDKSR |
| | | | | WQQGNVFSCSVMHEALHNHYTQKSLSLSPGK |
| | | | | GGGGSGGGGSGGGGSGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWESN |
| | | | | GQPENNYKTTPPVLDSDGSFFLYSKLTVDKSR |
| | | | | WQQGNVFSCSVMHEALHNHYTQKSLSLSPGK |
| 126. | BCMA A7 | artificial | aa | NHIIH |
| | 27-C4-G7 | | | |
| | CDR1 VH | | | |
| 127. | BCMA A7 | artificial | aa | YINPYPGYHAYNEKFQG |
| | 27-C4-G7 | | | |
| | CDR2 VH | | | |
| 128. | BCMA A7 | artificial | aa | DGYYRDTDVLDY |
| | 27-C4-G7 | | | |
| | CDR3 VH | | | |
| 129 | BCMA A7 | artificial | aa | QASQDISNYLN |
| | 27-C4-G7 | | | |
| | CDR1 VL | | | |
| 130. | BCMA A7 | artificial | aa | YTSRLHT |
| | 27-C4-G7 | | | |
| | CDR2 VL | | | |
| 131. | BCMA A7 | artificial | aa | QQGNTLPWT |
| | 27-C4-G7 | | | |
| | CDR3 VL | | | |
| 132. | BCMA A7 | artificial | aa | QVQLVQSGAEVKKPGASVKVSCKASGYTFTNH |
| | 27-C4-G7 CC | | | IIHWVRQAPGQCLEWMGYINPYPGYHAYNEKF |
| | (44/100) VH | | | QGRATMTSDTSTSTVYMELSSLRSEDTAVYYC |
| | | | | ARDGYYRDTDVLDYWGQGTLVTVSS |
| 133. | BCMA A7 | artificial | aa | DIQMTQSPSSLSASVGDRVTITCQASQDISNYLN |
| | 27-C4-G7 CC | | | WYQQKPGKAPKLLIYYTSRLHTGVPSRFSGSGS |
| | (44/100) VL | | | GTDFTFTISSLEPEDIATYYCQQGNTLPWTFGCG |
| | | | | TKLEIK |
| 134. | BCMA A7 | artificial | aa | QVQLVQSGAEVKKPGASVKVSCKASGYTFTNH |
| | 27-C4-G7 CC | | | IIHWVRQAPGQCLEWMGYINPYPGYHAYNEKF |
| | (44/100) scFv | | | QGRATMTSDTSTSTVYMELSSLRSEDTAVYYC |
| | | | | ARDGYYRDTDVLDYWGQGTLVTVSSGGGGSG |
| | | | | GGGSGGGGSDIQMTQSPSSLSASVGDRVTITCQ |
| | | | | ASQDISNYLNWYQQKPGKAPKLLIYYTSRLHT |
| | | | | GVPSRFSGSGSGTDFTFTISSLEPEDIATYYCQQ |
| | | | | GNTLPWTFGCGTKLEIK |
| 135. | BCMA A7 | artificial | aa | QVQLVQSGAEVKKPGASVKVSCKASGYTFTNH |
| | 27-C4-G7 CC | | | IIHWVRQAPGQCLEWMGYINPYPGYHAYNEKF |
| | (44/100) x | | | QGRATMTSDTSTSTVYMELSSLRSEDTAVYYC |
| | I2C0 | | | ARDGYYRDTDVLDYWGQGTLVTVSSGGGGSG |
| | bispecific | | | GGGSGGGGSDIQMTQSPSSLSASVGDRVTITCQ |
| | molecule | | | ASQDISNYLNWYQQKPGKAPKLLIYYTSRLHT |
| | | | | GVPSRFSGSGSGTDFTFTISSLEPEDIATYYCQQ |
| | | | | GNTLPWTFGCGTKLEIKSGGGGSEVQLVESGG |
| | | | | GLVQPGGSLKLSCAASGFTFNKYAMNWVRQA |
| | | | | PGKGLEWVARIRSKYNNYATYYADSVKDRFTI |
| | | | | SRDDSKNTAYLQMNNLKTEDTAVYYCVRHGN |
| | | | | FGNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVL |
| 136. | BCMA A7 | artificial | aa | QVQLVQSGAEVKKPGASVKVSCKASGYTFTNH |
| | 27-C4-G7 CC | | | IIHWVRQAPGQCLEWMGYINPYPGYHAYNEKF |
| | (44/100) x | | | QGRATMTSDTSTSTVYMELSSLRSEDTAVYYC |
| | I2C0-scFc | | | ARDGYYRDTDVLDYWGQGTLVTVSSGGGGSG |
| | bispecific | | | GGGSGGGGSDIQMTQSPSSLSASVGDRVTITCQ |
| | molecule | | | ASQDISNYLNWYQQKPGKAPKLLIYYTSRLHT |
| | HLE | | | GVPSRFSGSGSGTDFTFTISSLEPEDIATYYCQQ |
| | | | | GNTLPWTFGCGTKVEIKSGGGGSEVQLVESGG |
| | | | | GLVQPGGSLKLSCAASGFTFNKYAMNWVRQA |
| | | | | PGKGLEWVARIRSKYNNYATYYADSVKDRFTI |
| | | | | SRDDSKNTAYLQMNNLKTEDTAVYYCVRHGN |
| | | | | FGNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 137. | PM 76- | artificial | aa | DYYMY |
| | B10.17 CC | | | |
| | VH CDR1 | | | |
| 138. | PM 76- | artificial | aa | IISDAGYYTYYSDIIKG |
| | B10.17 CC | | | |
| | VH CDR2 | | | |
| 139. | PM 76- | artificial | aa | GFPLLRHGAMDY |
| | B10.17 CC | | | |
| | VH CDR3 | | | |
| 140. | PM 76- | artificial | aa | KASQNVDANVA |
| | B10.17 CC | | | |
| | VL CDR1 | | | |
| 141. | PM 76- | artificial | aa | SASYVYW |
| | B10.17 CC | | | |
| | VL CDR2 | | | |
| 142. | PM 76- | artificial | aa | QQYDQQLIT |
| | B10.17 CC | | | |
| | VL CDR3 | | | |
| 143. | PM 76- | artificial | aa | QVQLVESGGGLVKPGESLRLSCAASGFTFSDYY |
| | B10.17 CC | | | MYWVRQAPGKCLEWVAIISDAGYYTYYSDIIK |
| | VH | | | GRFTISRDNAKNSLYLQMNSLKAEDTAVYYCA |
| | | | | RGFPLLRHGAMDYWGQGTLVTVSS |
| 144. | PM 76- | artificial | aa | DIQMTQSPSSLSASVGDRVTITCKASQNVDANV |
| | B10.17 CC | | | AWYQQKPGQAPKSLIYSASYVYWDVPSRFSGS |
| | VL | | | ASGTDFTLTISSVQSEDFATYYCQQYDQQLITFG |
| | | | | CGTKLEIK |
| 145. | PM 76- | artificial | aa | QVQLVESGGGLVKPGESLRLSCAASGFTFSDYY |
| | B10.17 CC | | | MYWVRQAPGKCLEWVAIISDAGYYTYYSDIIK |
| | scFv | | | GRFTISRDNAKNSLYLQMNSLKAEDTAVYYCA |
| | | | | RGFPLLRHGAMDYWGQGTLVTVSSGGGGSGG |
| | | | | GGSGGGGSDIQMTQSPSSLSASVGDRVTITCKA |
| | | | | SQNVDANVAWYQQKPGQAPKSLIYSASYVYW |
| | | | | DVPSRFSGSASGTDFTLTISSVQSEDFATYYCQQ |
| | | | | YDQQLITFGCGTKLEIK |
| 146. | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0 | | | GRFTISRDNAKNSLYLQMNSLKAEDTAVYYCA |
| | bispecific | | | RGFPLLRHGAMDYWGQGTLVTVSSGGGGSGG |
| | molecule | | | GGSGGGGSDIQMTQSPSSLSASVGDRVTITCKA |
| | | | | SQNVDANVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVL |
| 147. | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0-scFc | | | GRFTISRDNAKNSLYLQMNSLKAEDTAVYYCA |
| | bispecific | | | RGFPLLRHGAMDYWGQGTLVTVSSGGGGSGG |
| | HLE | | | GGSGGGGSDIQMTQSPSSLSASVGDRVTITCKA |
| | molecule | | | SQNVDANVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 148. | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0- | | | GRFTISRDNAKNSLYLQMNSLKAEDTAVYYCA |
| | scFc_delGK | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDANVAWYQQKPGQAPKSLIYSASYVYW |
| | molecule | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 149 | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | molecule | | | SQNVDANVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVL |
| 150. | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDANVAWYQQKPGQAPKSLIYSASYVYW |
| | molecule | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 151. | PM 76- | artificial | aa | QVQLVESGGGLVKPGESLRLSCAASGFTFSDYY |
| | B10.17 CC x | | | MYWVRQAPGKCLEWVAIISDAGYYTYYSDIIK |
| | I2C0 CC | | | GRFTISRDNAKNSLYLQMNSLKAEDTAVYYCA |
| | (103/43)- | | | RGFPLLRHGAMDYWGQGTLVTVSSGGGGSGG |
| | scFc_delGK | | | GGSGGGGSDIQMTQSPSSLSASVGDRVTITCKA |
| | bispecific | | | SQNVDANVAWYQQKPGQAPKSLIYSASYVYW |
| | HLE | | | DVPSRFSGSASGTDFTLTISSVQSEDFATYYCQQ |
| | molecule | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 152. | PM 76- | artificial | aa | DYYMY |
| | B10.11 CC | | | |
| | VH CDR1 | | | |
| 153. | PM 76- | artificial | aa | IISDGGYYTYYSDIIKG |
| | B10.11 CC | | | |
| | VH CDR2 | | | |
| 154. | PM 76- | artificial | aa | GFPLLRHGAMDY |
| | B10.11 CC | | | |
| | VH CDR3 | | | |
| 155. | PM 76- | artificial | aa | KASQNVDTNVA |
| | B10.11 CC | | | |
| | VL CDR1 | | | |
| 156. | PM 76- | artificial | aa | SASYVYW |
| | B10.11 CC | | | |
| | VL CDR2 | | | |
| 157. | PM 76- | artificial | aa | QQYDQQLIT |
| | B10.11 CC | | | |
| | VL CDR3 | | | |
| 158. | PM 76- | artificial | aa | QVQLVESGGGLVKPGESLRLSCAASGFTFSDYY |
| | B10.11 CC | | | MYWVRQAPGKGLEWVAIISDGGYYTYYSDIIK |
| | VH | | | GRFTISRDNAKNSLYLQMNSLKAEDTAVYYCA |
| | | | | RGFPLLRHGAMDYWGQGTLVTVSS |
| 159. | PM 76- | artificial | aa | DIQMTQSPSSLSASVGDRVTITCKASQNVDTNV |
| | B10.11 CC | | | AWYQQKPGQAPKSLIYSASYVYWDVPSRFSGS |
| | Vl | | | ASGTDFTLTISSVQSEDFATYYCQQYDQQLITFG |
| | | | | GGTKLEIK |
| 160. | PM 76- | artificial | aa | QVQLVESGGGLVKPGESLRLSCAASGFTFSDYY |
| | B10.11 CC | | | MYWVRQAPGKGLEWVAIISDGGYYTYYSDIIK |
| | scFv | | | GRFTISRDNAKNSLYLQMNSLKAEDTAVYYCA |
| | | | | RGFPLLRHGAMDYWGQGTLVTVSSGGGGSGG |
| | | | | GGSGGGGSDIQMTQSPSSLSASVGDRVTITCKA |
| | | | | SQNVDTNVAWYQQKPGQAPKSLIYSASYVYW |
| | | | | DVPSRFSGSASGTDFTLTISSVQSEDFATYYCQQ |
| | | | | YDQQLITFGGGTKLEIK |
| 161. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0 | | | GRFTISRDNAKNSLYLQMNSLKAEDTAVYYCA |
| | bispecific | | | RGFPLLRHGAMDYWGQGTLVTVSSGGGGSGG |
| | molecule | | | GGSGGGGSDIQMTQSPSSLSASVGDRVTITCKA |
| | | | | SQNVDTNVAWYQQKPGQAPKSLIYSASYVYW |
| | | | | DVPSRFSGSASGTDFTLTISSVQSEDFATYYCQQ |
| | | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVL |
| 162. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0-scFc | | | GRFTISRDNAKNSLYLQMNSLKAEDTAVYYCA |
| | bispecific | | | RGFPLLRHGAMDYWGQGTLVTVSSGGGGSGG |
| | HLE | | | GGSGGGGSDIQMTQSPSSLSASVGDRVTITCKA |
| | molecule | | | SQNVDTNVAWYQQKPGQAPKSLIYSASYVYW |
| | | | | DVPSRFSGSASGTDFTLTISSVQSEDFATYYCQQ |
| | | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 163. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0- | | | GRFTISRDNAKNSLYLQMNSLKAEDTAVYYCA |
| | scFc_delGK | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDTNVAWYQQKPGQAPKSLIYSASYVYW |
| | molecule | | | DVPSRFSGSASGTDFTLTISSVQSEDFATYYCQQ |
| | | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 164. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | molecule | | | SQNVDTNVAWYQQKPGQAPKSLIYSASYVYW |
| | | | | DVPSRFSGSASGTDFTLTISSVQSEDFATYYCQQ |
| | | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVL |
| 165. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDTNVAWYQQKPGQAPKSLIYSASYVYW |
| | molecule | | | DVPSRFSGSASGTDFTLTISSVQSEDFATYYCQQ |
| | | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 166. | PM 76- | artificial | aa | QVQLVESGGGLVKPGESLRLSCAASGFTFSDYY |
| | B10.11 CC x | | | MYWVRQAPGKGLEWVAIISDGGYYTYYSDIIK |
| | I2C0 CC | | | GRFTISRDNAKNSLYLQMNSLKAEDTAVYYCA |
| | (103/43)- | | | RGFPLLRHGAMDYWGQGTLVTVSSGGGGSGG |
| | scFc_delGK | | | GGSGGGGSDIQMTQSPSSLSASVGDRVTITCKA |
| | bispecific | | | SQNVDTNVAWYQQKPGQAPKSLIYSASYVYW |
| | HLE | | | DVPSRFSGSASGTDFTLTISSVQSEDFATYYCQQ |
| | molecule | | | YDQQLITFGGGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 167. | PM 76- | artificial | aa | DYYMY |
| | B10.11 CC x | | | |
| | I2C0-scFc | | | |
| | VH CDR1 | | | |
| 168. | PM 76- | artificial | aa | IISDGGYYTYYSDIIKG |
| | B10.11 CC x | | | |
| | I2C0-scFc | | | |
| | VH CDR2 | | | |
| 169. | PM 76- | artificial | aa | GFPLLRHGAMDY |
| | B10.11 CC x | | | |
| | I2C0-scFc | | | |
| | VH CDR3 | | | |
| 170. | PM 76- | artificial | aa | KASQNVDTNVA |
| | B10.11 CC x | | | |
| | I2C0-scFc VL | | | |
| | CDR1 | | | |
| 171. | PM 76- | artificial | aa | SASYVYW |
| | B10.11 CC x | | | |
| | I2C0-scFc VL | | | |
| | CDR2 | | | |
| 172. | PM 76- | artificial | aa | QQYDQQLIT |
| | B10.11 CC x | | | |
| | I2C0-scFc VL | | | |
| | CDR3 | | | |
| 173. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0-scFc | | | GRFTISRDNAKNSLYLQMNSLKAEDTAVYYCA |
| | VH | | | RGFPLLRHGAMDYWGQGTLVTVSS |
| 174. | PM 76- | artificial | aa | DIQMTQSPSSLSASVGDRVTITCKASQNVDTNV |
| | B10.11 CC x | | | AWYQQKPGQAPKSLIYSASYVYWDVPSRFSGS |
| | I2C0-scFc VL | | | ASGTDFTLTISSVQSEDFATYYCQQYDQQLITFG |
| | | | | CGTKLEIK |
| 175. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0-scFc | | | GRFTISRDNAKNSLYLQMNSLKAEDTAVYYCA |
| | scFv | | | RGFPLLRHGAMDYWGQGTLVTVSSGGGGSGG |
| | | | | GGSGGGGSDIQMTQSPSSLSASVGDRVTITCKA |
| | | | | SQNVDTNVAWYQQKPGQAPKSLIYSASYVYW |
| | | | | DVPSRFSGSASGTDFTLTISSVQSEDFATYYCQQ |
| | | | | YDQQLITFGCGTKLEIK |
| 176. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0-scFc | | | GRFTISRDNAKNSLYLQMNSLKAEDTAVYYCA |
| | bispecific | | | RGFPLLRHGAMDYWGQGTLVTVSSGGGGSGG |
| | molecule | | | GGSGGGGSDIQMTQSPSSLSASVGDRVTITCKA |
| | | | | SQNVDTNVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVL |
| 177. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0-scFc | | | GRFTISRDNAKNSLYLQMNSLKAEDTAVYYCA |
| | bispecific | | | RGFPLLRHGAMDYWGQGTLVTVSSGGGGSGG |
| | HLE | | | GGSGGGGSDIQMTQSPSSLSASVGDRVTITCKA |
| | molecule | | | SQNVDTNVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 178. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0- | | | GRFTISRDNAKNSLYLQMNSLKAEDTAVYYCA |
| | scFc_delGK | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDTNVAWYQQKPGQAPKSLIYSASYVYW |
| | molecule | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGGGTKLTVLGGGGDKTHTCPPCP |
| | | | | APELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFSC |
| | | | | SVMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 179. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | molecule | | | SQNVDTNVAWYQQKPGQAPKSLIYSASYVYW |
| | | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVL |
| 180. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0 CC | | | GRFTISRDNAKNSLYLQMNSLKAEDTAVYYCA |
| | (103/43)-scFc | | | RGFPLLRHGAMDYWGQGTLVTVSSGGGGSGG |
| | bispecific | | | GGSGGGGSDIQMTQSPSSLSASVGDRVTITCKA |
| | HLE | | | SQNVDTNVAWYQQKPGQAPKSLIYSASYVYW |
| | molecule | | | DVPSRFSGSASGTDFTLTISSVQSEDFATYYCQQ |
| | | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGK |
| 181. | PM 76- | artificial | aa | QVQLVESGGGLVKPGESLRLSCAASGFTFSDYY |
| | B10.11 CC x | | | MYWVRQAPGKCLEWVAIISDGGYYTYYSDIIK |
| | I2C0 CC | | | GRFTISRDNAKNSLYLQMNSLKAEDTAVYYCA |
| | (103/43)- | | | RGFPLLRHGAMDYWGQGTLVTVSSGGGGSGG |
| | scFc delGK | | | GGSGGGGSDIQMTQSPSSLSASVGDRVTITCKA |
| | bispecific | | | SQNVDTNVAWYQQKPGQAPKSLIYSASYVYW |
| | HLE | | | DVPSRFSGSASGTDFTLTISSVQSEDFATYYCQQ |
| | molecule | | | YDQQLITFGCGTKLEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYCGQGTLVTVSSGGGGSGGGGSG |
| | | | | GGGSQTVVTQEPSLTVSPGGTVTLTCGSSTGAV |
| | | | | TSGNYPNWVQQKPGQCPRGLIGGTKFLAPGTP |
| | | | | ARFSGSLLGGKAALTLSGVQPEDEAEYYCVLW |
| | | | | YSNRWVFGGGTKLTVLGGGGDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSCS |
| | | | | VMHEALHNHYTQKSLSLSPGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTTP |
| | | | | PVLDSDGSFFLYSKLTVDKSRWQQGNVFSCSV |
| | | | | MHEALHNHYTQKSLSLSPGK |
| 182. | IgG1 hinge | artificial | aa | DKTHTCPPCP |
| 183. | IgG2 subtype | artificial | aa | ERKCCVECPPCP |
| | hinge | | | |
| 184. | IgG3 subtype | artificial | aa | ELKTPLDTTHTCPRCP |
| | hinge | | | |
| 185. | IgG3 subtype | artificial | aa | ELKTPLGDTTHTCPRCP |
| | hinge | | | |
| 186. | IgG4 subtype | artificial | aa | ESKYGPPCPSCP |
| | hinge | | | |
| 187. | G4S linker | artificial | aa | GGGGS |
| 188. | (G4S)2 linker | artificial | aa | GGGGSGGGGS |
| 189. | (G4S)3 linker | artificial | aa | GGGGSGGGGSGGGGS |
| 190. | (G4S)4 linker | artificial | aa | GGGGSGGGGSGGGGSGGGGS |
| 191. | (G4S)5 linker | artificial | aa | GGGGSGGGGSGGGGSGGGGSGGGGS |
| 192. | (G4S)6 linker | artificial | aa | GGGGSGGGGSGGGGSGGGGSGGGGSGGGGS |
| 193. | (G4S)7 linker | artificial | aa | GGGGSGGGGSGGGGSGGGGSGGGGSGGGGSG |
| | | | | GGGS |
| 194. | (G4S)8 linker | artificial | aa | GGGGSGGGGSGGGGSGGGGSGGGGSGGGGSG |
| | | | | GGGSGGGGS |
| 195. | Peptide linker | artificial | aa | PGGGGS |
| 196. | Peptide linker | artificial | aa | PGGDGS |
| 197. | Peptide linker | artificial | aa | SGGGGS |
| 198. | Peptide linker | artificial | aa | GGGG |
| 199. | hexa-histidine | artificial | aa | HHHHHH |
| | tag | | | |
| 200. | CD3ϵ binder | artificial | aa | QTVVTQEPSLTVSPGGTVTLTCGSSTGAVTSGN |
| | VL | | | YPNWVQQKPGQAPRGLIGGTKFLAPGTPARFS |
| | | | | GSLLGGKAALTLSGVQPEDEAEYYCVLWYSN |
| | | | | RWVFGGGTKLTVL |
| 201. | CD3ϵ binder | artificial | aa | EVQLVESGGGLVQPGGSLRLSCAASGFTFNSY |
| | VH | | | AMNWVRQAPGKGLEWVARIRSKYNNYATYY |
| | | | | ADSVKGRFTISRDDSKNTAYLQMNSLKTEDTA |
| | | | | VYYCVRHGNFGNSYVSWWAYWGQGTLVTVS |
| | | | | S |
| 202. | CD3ϵ binder | artificial | aa | EVQLVESGGGLVQPGGSLRLSCAASGFTFNSY |
| | scFv | | | AMNWVRQAPGKGLEWVARIRSKYNNYATYY |
| | | | | ADSVKGRFTISRDDSKNTAYLQMNSLKTEDTA |
| | | | | VYYCVRHGNFGNSYVSWWAYWGQGTLVTVS |
| | | | | SGGGGSGGGGSGGGGSQTVVTQEPSLTVSPGG |
| | | | | TVTLTCGSSTGAVTSGNYPNWVQQKPGQAPR |
| | | | | GLIGGTKFLAPGTPARFSGSLLGGKAALTLSGV |
| | | | | QPEDEAEYYCVLWYSNRWVFGGGTKLTVL |
| 203. | MU 8-B7 CC x | artificial | aa | GYYWS |
| | I2C0-scFc VH | | | |
| | CDR1 | | | |
| 204. | MU 8-B7 CC x | artificial | aa | DIDASGSTKYNPSLKS |
| | I2C0-scFc VH | | | |
| | CDR2 | | | |
| 205. | MU 8-B7 CC x | artificial | aa | KKYSTVWSYFDN |
| | I2C0-scFc VH | | | |
| | CDR3 | | | |
| 206. | MU 8-B7 CC x | artificial | aa | SGDKLGDKYAS |
| | I2C0-scFc VL | | | |
| | CDR1 | | | |
| 207. | MU 8-B7 CC x | artificial | aa | QDRKRPS |
| | I2C0-scFc VL | | | |
| | CDR2 | | | |
| 208. | MU 8-B7 CC x | artificial | aa | QAWGSSTAV |
| | I2C0-scFc VL | | | |
| | CDR3 | | | |
| 209. | MU 8-B7 CC x | artificial | aa | QVQLQQWGAGLLKPSETLSLTCAVYGGSFSGYYWS |
| | I2C0-scFc VH | | | WIRQPPGKCLEWIGDIDASGSTKYNPSLKSRVTISLD |
| | | | | TSKNQFSLKLNSVTAADTAVYFCARKKYSTVWSYF |
| | | | | DNWGQGTLVTVSS |
| 210. | MU 8-B7 CC x | artificial | aa | SYELTQPSSVSVPPGQTASITCSGDKLGDKYASWYQ |
| | I2C0-scFc VL | | | QKPGQSPVLVIYQDRKRPSGVPERFSGSNSGNTATL |
| | | | | TISGTQAMDEADYYCQAWGSSTAVFGCGTKLTVL |
| 211. | MU 8-B7 CC x | artificial | aa | QVQLQQWGAGLLKPSETLSLTCAVYGGSFSGYYWS |
| | I2C0-scFc scFv | | | WIRQPPGKCLEWIGDIDASGSTKYNPSLKSRVTISLD |
| | | | | TSKNQFSLKLNSVTAADTAVYFCARKKYSTVWSYF |
| | | | | DNWGQGTLVTVSSGGGGSGGGGSGGGGSSYELTQP |
| | | | | SSVSVPPGQTASITCSGDKLGDKYASWYQQKPGQSP |
| | | | | VLVIYQDRKRPSGVPERFSGSNSGNTATLTISGTQA |
| | | | | MDEADYYCQAWGSSTAVFGCGTKLTVL |
| 212. | MU 8-B7 CC x | artificial | aa | QVQLQQWGAGLLKPSETLSLTCAVYGGSFSGYYWS |
| | I2C0-scFc | | | WIRQPPGKCLEWIGDIDASGSTKYNPSLKSRVTISLD |
| | Bispecific | | | TSKNQFSLKLNSVTAADTAVYFCARKKYSTVWSYF |
| | molecule | | | DNWGQGTLVTVSSGGGGSGGGGSGGGGSSYELTQP |
| | | | | SSVSVPPGQTASITCSGDKLGDKYASWYQQKPGQSP |
| | | | | VLVIYQDRKRPSGVPERFSGSNSGNTATLTISGTQA |
| | | | | MDEADYYCQAWGSSTAVFGCGTKLTVLSGGGGSE |
| | | | | VQLVESGGGLVQPGGSLKLSCAASGFTENKYAMN |
| | | | | WVRQAPGKGLEWVARIRSKYNNYATYYADSVKDR |
| | | | | FTISRDDSKNTAYLQMNNLKTEDTAVYYCVRHGNF |
| | | | | GNSYISYWAYWGQGTLVTVSSGGGGSGGGGSGGG |
| | | | | GSQTVVTQEPSLTVSPGGTVTLTCGSSTGAVTSGNY |
| | | | | PNWVQQKPGQAPRGLIGGTKFLAPGTPARFSGSLLG |
| | | | | GKAALTLSGVQPEDEAEYYCVLWYSNRWVFGGGT |
| | | | | KLTVL |
| 213. | MU 8-B7 CC x | artificial | aa | QVQLQQWGAGLLKPSETLSLTCAVYGGSFSGYYWS |
| | I2C0-scFc | | | WIRQPPGKCLEWIGDIDASGSTKYNPSLKSRVTISLD |
| | Bispecific HLE | | | TSKNQFSLKLNSVTAADTAVYFCARKKYSTVWSYF |
| | molecule | | | DNWGQGTLVTVSSGGGGGGGGSGGGGSSYELTQP |
| | | | | SSVSVPPGQTASITCSGDKLGDKYASWYQQKPGQSP |
| | | | | VlVIYQDRKRPSGVPERFSGSNSGNTATITISGTQA |
| | | | | MDEADYYCQAWGSSTAVFGCGTKLTVLSGGGGSE |
| | | | | VQLVESGGGLVQPGGSLKLSCAASGFTFNKYAMN |
| | | | | WVRQAPGKGLEWVARIRSKYNNYATYYADSVKDR |
| | | | | FTISRDDSKNTAYLQMNNLKTEDTAVYYCVRHGNF |
| | | | | GNSYISYWAYWGQGTLVTVSSGGGGSGGGGSGGG |
| | | | | GSQTVVTQEPSLTVSPGGTVTLTCGSSTGAVTSGNY |
| | | | | PNWVQQKPGQAPRGLIGGTKFLAPGTPARFSGSLLG |
| | | | | GKAALTLSGVQPEDEAEYYCVLWYSNRWVFGGGT |
| | | | | KLTVLGGGGDKTHTCPPCPAPELLGGPSVFLFPPKP |
| | | | | KDTLMISRTPEVTCVVVDVSHEDPEVKFNWYVDGV |
| | | | | EVHNAKTKPCEEQYGSTYRCVSVLTVLHQDWLNG |
| | | | | KEYKCKVSNKALPAPIEKTISKAKGQPREPQVYTLP |
| | | | | PSREEMTKNQVSLTCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSPGKGGGGSGGGGSG |
| | | | | GGGSGGGGSGGGGSGGGGSDKTHTCPPCPAPELLG |
| | | | | GPSVFLFPPKPKDTLMISRTPEVTCVVVDVSHEDPEV |
| | | | | KFNWYVDGVEVHNAKTKPCEEQYGSTYRCVSVLT |
| | | | | VLHQDWLNGKEYKCKVSNKALPAPIEKTISKAKGQ |
| | | | | PREPQVYTLPPSREEMTKNQVSLTCLVKGFYPSDIA |
| | | | | VEWESNGQPENNYKTTPPVLDSDGSFFLYSKLTVD |
| | | | | KSRWQQGNVFSCSVMHEALHNHYTQKSLSLSPGK |
| 214. | CL-1 VH | artificial | aa | GYYMH |
| | CDR1 | | | |
| 215. | CL-1 VH | artificial | aa | WINPNSGGTKYAQKFQG |
| | CDR2 | | | |
| 216. | CL-1 VH | artificial | aa | DRITVAGTYYYYGMDV |
| | CDR3 | | | |
| 217. | CL-1 VL | artificial | aa | RASQGVNNWLA |
| | CDR1 | | | |
| 218. | CL-1 VL | artificial | aa | TASSLQS |
| | CDR2 | | | |
| 219. | CL-1 VL | artificial | aa | QQANSFPIT |
| | CDR3 | | | |
| 220. | CL-1 VH | artificial | aa | QVQLVQSGAEVKKPGASVKVSCKASGYTFTGYYM |
| | | | | HWVRQAPGQCLEWMGWINPNSGGTKYAQKFQGR |
| | | | | VTMTRDTSISTAYMELSRLRSDDTAVYYCARDRITV |
| | | | | AGTYYYYGMDVWGQGTTVTVSS |
| 221. | CL-1 VL | artificial | aa | DIQMTQSPSSVSASVGDRVTITCRASQGVNNWLAW |
| | | | | YQQKPGKAPKLLIYTASSLQSGVPSRFSGSGSGTDFT |
| | | | | LTIRSLQPEDFATYYCQQANSFPITFGCGTRLEIK |
| 222. | CL-1 scFv | artificial | aa | QVQLVQSGAEVKKPGASVKVSCKASGYTFTGYYM |
| | | | | HWVRQAPGQCLEWMGWINPNSGGTKYAQKFQGR |
| | | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIK |
| 223. | CL-1 x I2C | | | |
| | bispecific | | | |
| 224. | molecule | artificial | aa | QVQLVQSGAEVKKPGASVKVSCKASGYTFTGYYM |
| | | | | HWVRQAPGQCLEWMGWINPNSGGTKYAQKFQGR |
| | | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKILIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVL |
| 225. | CL-1 x I2C- | artificial | aa | QVQLVQSGAEVKKPGASVKVSCKASGYTFTGYYM |
| | 6His bispecific | | | HWVRQAPGQCLEWMGWINPNSGGTKYAQKFQGR |
| | molecule-his | | | VTMTRDTSISTAYMELSRLRSDDTAVYYCARDRITV |
| | tag | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVLHHHHHH |
| 226. | CL-1 x I2C- | artificial | aa | QVQLVQSGAEVKKPGASVKVSCKASGYTFTGYYM |
| | scFc bispecific | | | HWVRQAPGQCLEWMGWINPNSGGTKYAQKFQGR |
| | scFc molecule | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVLGGGGDKTHTCPPCPAPELLGGP |
| | | | | SVFLFPPKPKDTLMISRTPEVTCVVVDVSHEDPEVKF |
| | | | | NWYVDGVEVHNAKTKPCEEQYGSTYRCVSVLTVL |
| | | | | HQDWLNGKEYKCKVSNKALPAPIEKTISKAKGQPR |
| | | | | EPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGG |
| | | | | GSGGGGSGGGGSGGGGSGGGGSGGGGSDKTHTCPP |
| | | | | CPAPELLGGPSVFLFPPKPKDTLMISRTPEVTCVVVD |
| | | | | VSHEDPEVKFNWYVDGVEVHNAKTKPCEEQYGST |
| | | | | YRCVSVLTVLHQDWLNGKEYKCKVSNKALPAPIEK |
| | | | | TISKAKGQPREPQVYTLPPSREEMTKNQVSLTCLVK |
| | | | | GFYPSDIAVEWESNGQPENNYKTTPPVLDSDGSFFL |
| | | | | YSKLTVDKSRWQQGNVFSCSVMIIEALIINIIYTQKS |
| | | | | LSLSPGK |
| 227. | CL-2 VH | artificial | aa | GYYMH |
| | CDR1 | | | |
| 228. | CL-2 VH | artificial | aa | WINPNSGGTKYAQKFQG |
| | CDR2 | | | |
| 229. | CL-2 VH | artificial | aa | DRITVAGTYYYYGMDV |
| | CDR3 | | | |
| 230. | CL-2 VL | artificial | aa | RASQGVNNWLA |
| | CDR1 | | | |
| 231. | CL-2VL CDR2 | artificial | aa | TASSLQS |
| 232. | CL-2 VL | artificial | aa | QQANSFPIT |
| | CDR3 | | | |
| 233. | CL-2 VH | artificial | aa | QVQMVQSGAEVKKHGASVKVSCKASGYTFTGYYM |
| | | | | HWVRQAPGQCLEWMGWINPNSGGTKYAQKFQGR |
| | | | | VTMTRDTSISTAYMELSRLRSDDTAVYYCARDRITV |
| | | | | AGTYYYYGMDVWGQGTTVTVSS |
| 234. | CL-2 VL | artificial | aa | DIQMTQSPSSVSASVGDRVTITCRASQGVNNWLAW |
| | | | | YQQKPGKAPKLLIYTASSLQSGVPSRFSGSGSGTDFT |
| | | | | LTIRSLQPEDFATYYCQQANSFPITFGCGTRLEIK |
| 235. | CL-2 scFv | artificial | aa | QVQMVQSGAEVKKHGASVKVSCKASGYTFTGYYM |
| | | | | HWVRQAPGQCLEWMGWINPNSGGTKYAQKFQGR |
| | | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIK |
| 236. | CL-2 x I2C | artificial | aa | QVQMVQSGAEVKKHGASVKVSCKASGYTFTGYYM |
| | bispecific | | | HWVRQAPGQCLEWMGWINPNSGGTKYAQKFQGR |
| | molecule | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVL |
| 237. | CL-2 x I2C- | artificial | aa | QVQMVQSGAEVKKHGASVKVSCKASGYTFTGYYM |
| | 6His bispecific | | | HWVRQAPGQCLEWMGWINPNSGGTKYAQKFQGR |
| | molecule-his | | | VTMTRDTSISTAYMELSRLRSDDTAVYYCARDRITV |
| | tag | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVLHHHHHH |
| 238. | CL-2 x I2C- | artificial | aa | QVQMVQSGAEVKKHGASVKVSCKASGYTFTGYYM |
| | scFc bispecific | | | HWVRQAPGQCLEWMGWINPNSGGTKYAQKFQGR |
| | scFc molecule | | | VTMTRDTSISTAYMELSRLRSDDTAVYYCARDRITV |
| | | | | AGTYYYYGMDVWGQGTTVTVSSGGGGSGGGGSG |
| | | | | GGGSDIQMTQSPSSVSASVGDRVTITCRASQGVNN |
| | | | | WLAWYQQKPGKAPKLLIYTASSLQSGVPSRFSGSGS |
| | | | | GTDFTLTIRSLQPEDFATYYCQQANSFPITFGCGTRL |
| | | | | EIKSGGGGSEVQLVESGGGLVQPGGSLKLSCAASGF |
| | | | | TFNKYAMNWVRQAPGKGLEWVARIRSKYNNYATY |
| | | | | YADSVKDRFTISRDDSKNTAYLQMNNLKTEDTAVY |
| | | | | YCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSN |
| | | | | RWVFGGGTKLTVLGGGGDKTHTCPPCPAPELLGGP |
| | | | | SVFLFPPKPKDTLMISRTPEVTCVVVDVSHEDPEVKF |
| | | | | NWYVDGVEVHNAKTKPCEEQYGSTYRCVSVLTVL |
| | | | | HQDWLNGKEYKCKVSNKALPAPIEKTISKAKGQPR |
| | | | | EPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGG |
| | | | | GSGGGGSGGGGSGGGGSGGGGSGGGGSDKTHTCPP |
| | | | | CPAPELLGGPSVFLFPPKPKDTLMISRTPEVTCVVVD |
| | | | | VSHEDPEVKFNWYVDGVEVHNAKTKPCEEQYGST |
| | | | | YRCVSVLTVLHQDWLNGKEYKCKVSNKALPAPIEK |
| | | | | TISKAKGQPREPQVYTLPPSREEMTKNQVSLTCLVK |
| | | | | GFYPSDIAVEWESNGQPENNYKTTPPVLDSDGSFFL |
| | | | | YSKLTVDKSRWQQGNVFSCSVMHEALHNHYTQKS |
| | | | | LSLSPGK |
| 239. | CD70 HCDR1 | artificial | aa | TYAMS |
| 240. | CD70 HCDR2 | artificial | aa | AISGSGGRTFYAESVEG |
| 241. | CD70 HCDR3 | artificial | aa | HDYSNYPYFDY |
| 242. | CD70 LCDR1 | artificial | aa | RASQSVRSTYLA |
| 243. | CD70 LCDR2 | artificial | aa | GASSRAT |
| 244. | CD70 LCDR3 | artificial | aa | QQYGDLPFT |
| 245. | CD70 VH | artificial | aa | EVQLLESGGGMVQPGGSLRLSCAASGFTFSTYAMS |
| | | | | WVRQAPGKCLEWVSAISGSGGRTFYAESVEGRFTIS |
| | | | | RDNSKNTLYLQMNSLRAEDTAVYYCAKHDYSNYP |
| | | | | YFDYWGQGTLVTVSS |
| 246. | CD70 VL | artificial | aa | EIVLTQSPGTLSLSPGERATLSCRASQSVRSTYLAWY |
| | | | | QQKPGQAPRLLIYGASSRATGIPDRFSGSGSGTDFTL |
| | | | | TISRLEPEDFAVYSCQQYGDLPFTFGCGTKLEIK |
| 247. | CD70 VHVL | artificial | aa | EVQLLESGGGMVQPGGSLRLSCAASGFTFSTYAMS |
| | | | | WVRQAPGKCLEWVSAISGSGGRTFYAESVEGRFTIS |
| | | | | RDNSKNTLYLQMNSLRAEDTAVYYCAKHDYSNYP |
| | | | | YFDYWGQGTLVTVSSGGGGSGGGGSGGGGSEIVLT |
| | | | | QSPGTLSLSPGERATLSCRASQSVRSTYLAWYQQKP |
| | | | | GQAPRLLIYGASSRATGIPDRFSGSGSGTDFTLTISRL |
| | | | | EPEDFAVYSCQQYGDLPFTFGCGTKLEI |
| 248. | CD70 x I2C | artificial | aa | EVQLLESGGGMVQPGGSLRLSCAASGFTFSTYAMS |
| | | | | WVRQAPGKCLEWVSAISGSGGRTFYAESVEGRFTIS |
| | | | | RDNSKNTLYLQMNSLRAEDTAVYYCAKHDYSNYP |
| | | | | YFDYWGQGTLVTVSSGGGGSGGGGSGGGGSEIVLT |
| | | | | QSPGTLSLSPGERATLSCRASQSVRSTYLAWYQQKP |
| | | | | GQAPRLLIYGASSRATGIPDRFSGSGSGTDFTLTISRL |
| | | | | EPEDFAVYSCQQYGDLPFTFGCGTKLEIKSGGGGSE |
| | | | | VQLVESGGGLVQPGGSLKLSCAASGFTFNKYAMN |
| | | | | WVRQAPGKGLEWVARIRSKYNNYATYYADSVKDR |
| | | | | FTISRDDSKNTAYLQMNNLKTEDTAVYYCVRHGNF |
| | | | | GNSYISYWAYWGQGTLVTVSSGGGGSGGGGSGGG |
| | | | | GSQTVVTQEPSLTVSPGGTVTLTCGSSTGAVTSGNY |
| | | | | PNWVQQKPGQAPRGLIGGTKFLAPGTPARFSGSLLG |
| | | | | GKAALTLSGVQPEDEAEYYCVLWYSNRWVFGGGT |
| | | | | KLTV |
| 249. | MSLN 15-B12 | artificial | aa | QVQLQESGPGLVKPSETLSLTCTVSGGSISSSSYFWG |
| | CC-VH | | | WIRQPPGKCLEWIGNIYYSGSSNYNPSLKSRVTISVD |
| | | | | TSKNQFSLKLSSVTAADTAVYYCARLPRGDRDAFDI |
| | | | | WGQGTMVTVSS |
| 250. | MSLN 15-B12 | artificial | aa | DIVMTQSPSSLSASVGDRVTITCRASQGISNYLAWY |
| | CC-VL | | | QQKPGKVPKLLIYAASTLQSGVPSRFSGSGSGTDFTL |
| | | | | TISSLQPEDFATYYCQQSYSTPFTFGCGTKVEIK |
| 251. | MSLN 15-B12 | artificial | aa | EIVMTQSPSSLSASVGDRVTITCRASQGISNYLAWY |
| | CC EI-VL | | | QQKPGKVPKLLIYAASTLQSGVPSRFSGSGSGTDFTL |
| | | | | TISSLQPEDFATYYCQQSYSTPFTFGCGTKVEIK |
| 252. | CDH3 15-E11 | artificial | aa | QVQLVQSGAEVKKPGASVKVSCKASGYTFTNYWM |
| | CC-VH | | | NWVRQAPGQCLEWMGNIAYGVKGTNYNQKFQGR |
| | | | | VTMTVDTSSSTAYMELSRLRSDDTAVYYCATRYFY |
| | | | | VMDYWGQGTLVTVSS |
| 253. | CDH3 15-E11 | artificial | Aa | EIQMTQSPSSLSASVGDRVTITCRASQDISNYLNWY |
| | CC EI-VL | | | QQKPGKVPKLLIYYTSRLHSGVPSRFSGSGSGTDFTL |
| | | | | TISSLQPEDVATYYCVQYAQFPLTFGCGTKVEIK |
| 254. | CDH3 15- | artificial | Aa | QVQLVQSGAEVKKPGASVKVSCKASGYTFTNYWM |
| | E11_1_VAG_ | | | NWVRQAPGQCLEWMGNIAYGVAGTNYNQKFQGR |
| | CC-VH | | | VTMTVDTSSSTAYMELSRLRSDDTAVYYCATRYFY |
| | | | | VMDYWGQGTLVTVSS |
| 255. | CDH3 15- | artificial | Aa | QVQLVQSGAEVKKPGASVKVSCKASGYTFTNYWM |
| | E11_1_VAG_ | | | NWVRQAPGQCLEWMGNIAYGVAGTNYNQKFQGR |
| | CC x I2L x G4 | | | VTMTVDTSSSTAYMELSRLRSDDTAVYYCATRYFY |
| | x scFc x G4 x | | | VMDYWGQGTLVTVSSGGGGQGGGGQGGGGQDIQ |
| | MSLN 15-B12 | | | MTQSPSSLSASVGDRVTITCRASQDISNYLNWYQQK |
| | CC x I2L | | | PGKVPKLLIYYTSRLHSGVPSRFSGSGSGTDFTLTISS |
| | clipopt_DI | | | LQPEDVATYYCVQYAQFPLTFGCGTKVEIKSGGGG |
| | | | | QEVQLVESGGGLVQPGGSLKLSCAASGFTFNKYAM |
| | | | | NWVRQAPGKGMEWVARIRSKYNNYATYYADAVK |
| | | | | DRFTISRDDSKNTLYLQMNNLKTEDTAVYYCVRAG |
| | | | | NFGSSYISYFAYWGQGTLVTVSSGGGGQGGGGQGG |
| | | | | GGQQTVVTQEPSLTVSPGGTVTITCGSSTGAVTSGN |
| | | | | YPNWIQKKPGQAPRGLIGGTKFLAPGTPARFSGSLE |
| | | | | GGKAALTLSGVQPEDEAEYYCVLYYSNRWVFGSGT |
| | | | | KLTVLGGGGCPPCPAPELLGGPSVFLFPPKPKDTLMI |
| | | | | SRTPEVTCVVVDVSHEEPEVKFNWYVDGVEVHNA |
| | | | | KTKPCEEQYGSTYRCVSVLTVLHQDWLNGKEYKC |
| | | | | KVSNKALPAPIEKTISKAKGQPREPQVYTLPPSREEM |
| | | | | TKNQVSLTCLVKGFYPSDIAVEWESNGQPENNYKT |
| | | | | TPPVLDSDGSFFLYSKLTVDKSRWQQGNVFSCSVM |
| | | | | HEALHNHYTQKSLSLSPGKGGGGQGGGGQGGGGQ |
| | | | | GGGGQGGGGQGGGGQCPPCPAPELLGGPSVFLFPP |
| | | | | KPKDTLMISRTPEVTCVVVDVSHEEPEVKFNWYVD |
| | | | | GVEVHNAKTKPCEEQYGSTYRCVSVLTVLHQDWL |
| | | | | NGKEYKCKVSNKALPAPIEKTISKAKGQPREPQVYT |
| | | | | LPPSREEMTKNQVSLTCLVKGFYPSDIAVEWESNGQ |
| | | | | PENNYKTTPPVLDSDGSFFLYSKLTVDKSRWQQGN |
| | | | | VFSCSVMIIEALIINIIYTQKSLSLSPGKGGGGQVQLQ |
| | | | | ESGPGLVKPSETLSLTCTVSGGSISSSSYFWGWIRQP |
| | | | | PGKCLEWIGNIYYSGSSNYNPSLKSRVTISVDTSKNQ |
| | | | | FSLKLSSVTAADTAVYYCARLPRGDRDAFDIWGQG |
| | | | | TMVTVSSGGGGQGGGGQGGGGQDIVMTQSPSSLSA |
| | | | | SVGDRVTITCRASQGISNYLAWYQQKPGKVPKLLIY |
| | | | | AASTLQSGVPSRFSGSGSGTDFTLTISSLQPEDFATY |
| | | | | YCQQSYSTPFTFGCGTKVEIKSGGGGQEVQLVESGG |
| | | | | GLVQPGGSLKLSCAASGFTFNKYAMNWVRQAPGK |
| | | | | GMEWVARIRSKYNNYATYYADAVKDRFTISRDDSK |
| | | | | NTLYLQMNNLKTEDTAVYYCVRAGNFGSSYISYFA |
| | | | | YWGQGTLVTVSSGGGGQGGGGQGGGGQQTVVTQ |
| | | | | EPSLTVSPGGTVTITCGSSTGAVTSGNYPNWIQKKP |
| | | | | GQAPRGLIGGTKFLAPGTPARFSGSLEGGKAALTLS |
| | | | | GVQPEDEAEYYCVLYYSNRWVFGSGTKLTVL |
| 256. | CD3δ | human | aa | MEHSTFLSGLVLATLLSQVSPFKIPIEELEDRVFVNC |
| | | | | NTSITWVEGTVGTLLSDITRLDLGKRILDPRGIYRCN |
| | | | | GTDIYKDKESTVQVHYRMCQSCVELDPATVAGIIVT |
| | | | | DVIATLLLALGVFCFAGHETGRLSGAADTQALLRN |
| | | | | DQVYQPLRDRDDAQYSHLGGNWARNK |
| 257. | CD3ϵ | human | aa | MQSGTHWRVLGLCLLSVGVWGQDGNEEMAYKVSI |
| | | | | SGTTVILTCPQYPGSEILWQHNDKNIGGDEDDKNIG |
| | | | | SDEDHLSLKEFSELEQSGYYVCYPRGSKPEDANFYL |
| | | | | YLRARVCENCMEMDVMSVATIVIVDICITGGLLLLV |
| | | | | YYWSKNRKAKAKPVTRGAGAGGRQRGQNKERPPP |
| | | | | VPNPDYEPIRKGQRDLYSGLNQRRI |
| 258. | CD3ϵ epitope 1 | human | aa | QDGNEEMGGITQTPYKVSISGTTVILT |
| 259. | CD3ϵ epitope 2 | human | aa | QDGNE |
| 260. | Anti-CD3ϵ | artificial | aa | XDGXE |
| | paratope | | | |
| | binding peptide | | | |
| | consensus 5mer | | | |
| 261. | Anti-CD3ϵ | artificial | aa | XDGXEE |
| | paratope | | | |
| | binding peptide | | | |
| | consensus 6mer | | | |
| 262. | Anti-CD3ϵ | artificial | aa | QDGNEEMG |
| | paratope | | | |
| | binding peptide | | | |
| 263. | Anti-CD3ϵ | artificial | aa | QDGNEEM |
| | paratope | | | |
| | binding peptide | | | |
| 264. | Anti-CD3ϵ | artificial | aa | QDGNEE |
| | paratope | | | |
| | binding peptide | | | |
| 265. | Anti-CD3ϵ | artificial | aa | QDGNDE |
| | paratope | | | |
| | binding peptide | | | |
| 266. | Anti-CD3ϵ | artificial | aa | SDGNEEMG |
| | paratope | | | |
| | binding peptide | | | |
| 267. | Anti-CD3ϵ | artificial | aa | QDGDEEMG |
| | paratope | | | |
| | binding peptide | | | |
| 268. | Anti-CD3ϵ | artificial | aa | QDGEEEMG |
| | paratope | | | |
| | binding peptide | | | |
| 269 | Anti-CD3ϵ | artificial | aa | QDGSEEMG |
| | paratope | | | |
| | binding peptide | | | |
| 270. | Anti-CD3ϵ | artificial | aa | QDGTEEMG |
| | paratope | | | |
| | binding peptide | | | |
| 271. | Anti-CD3ϵ | artificial | aa | QDGVEEMG |
| | paratope | | | |
| | binding peptide | | | |
| 272. | Anti-CD3ϵ | artificial | aa | QDGIEEMG |
| | paratope | | | |
| | binding peptide | | | |
| 273. | Anti-CD3ϵ | artificial | aa | pEDGNEEMG |
| | paratope | | | |
| | binding peptide | | | |
| 274. | Anti-CD3ϵ | artificial | aa | pEDGNEEM |
| | paratope | | | |
| | binding peptide | | | |
| 275. | Anti-CD3ϵ | artificial | aa | pEDGNEE |
| | paratope | | | |
| | binding peptide | | | |
| 276. | Anti-CD3ϵ | artificial | aa | pEDGDEEMG |
| | paratope | | | |
| | binding peptide | | | |
| 277. | Anti-CD3ϵ | artificial | aa | pEDGEEEMG |
| | paratope | | | |
| | binding peptide | | | |
| 278. | Anti-CD3ϵ | artificial | aa | pEDGSEEMG |
| | paratope | | | |
| | binding peptide | | | |
| 279. | Anti-CD3ϵ | artificial | aa | pEDGTEEMG |
| | paratope | | | |
| | binding peptide | | | |
| 280. | Anti-CD3ϵ | artificial | aa | pEDGVEEMG |
| | paratope | | | |
| | binding peptide | | | |
| 281. | Anti-CD3ϵ | artificial | aa | pEDGIEEMG |
| | paratope | | | |
| | binding peptide | | | |
| 282. | Anti-CD3ϵ | artificial | aa | pEDGDEELG |
| | paratope | | | |
| | binding peptide | | | |
| 283. | Anti-CD3ϵ | artificial | aa | pEDGEEELG |
| | paratope | | | |
| | binding peptide | | | |
| 284. | Anti-CD3ϵ | artificial | aa | pEDGSEELG |
| | paratope | | | |
| | binding peptide | | | |
| 285. | Anti-CD3ϵ | artificial | aa | pEDGTEELG |
| | paratope | | | |
| | binding peptide | | | |
| 286. | Anti-CD3ϵ | artificial | aa | pEDGVEELG |
| | paratope | | | |
| | binding peptide | | | |
| 287. | Anti-CD3ϵ | artificial | aa | pEDGIEELG |
| | paratope | | | |
| | binding peptide | | | |
| 288. | Anti-CD3ϵ | artificial | aa | pEDGNEELKG |
| | paratope | | | |
| | binding peptide | | | |
| 289. | Anti-CD3ϵ | artificial | aa | pEDGNEELRG |
| | paratope | | | |
| | binding peptide | | | |
| 290. | Anti-CD3ϵ | artificial | aa | pEDGNEEMRG |
| | paratope | | | |
| | binding peptide | | | |
| 291. | Anti-CD3ϵ | artificial | aa | pEDGDEELKG |
| | paratope | | | |
| | binding peptide | | | |
| 292. | Anti-CD3ϵ | artificial | aa | pEDGEEELKG |
| | paratope | | | |
| | binding peptide | | | |
| 293. | Anti-CD3ϵ | artificial | aa | pEDGSEELKG |
| | paratope | | | |
| | binding peptide | | | |
| 294. | Anti-CD3ϵ | artificial | aa | pEDGTEELKG |
| | paratope | | | |
| | binding peptide | | | |
| 295. | Anti-CD3ϵ | artificial | aa | pEDGVEELKG |
| | paratope | | | |
| | binding peptide | | | |
| 296. | Anti-CD3ϵ | artificial | aa | pEDGIEELKG |
| | paratope | | | |
| | binding peptide | | | |
| 297. | CD3ϵ PEP v1 | artificial | aa | QDGNEEMGKGMEENGDQ |
| 298. | Anti-CD3ϵ | artificial | aa | QDGNEEMGKQDGNEEMG |
| | paratope | | | |
| | binding peptide | | | |
| 299 | Anti-CD3ϵ | artificial | aa | QDGNEEMGRQDGNEEMG |
| | paratope | | | |
| | binding peptide | | | |
| 300. | CD3&ϵ PEP v2 | artificial | aa | pEDGNEEMGKGMEENGDQ |
| 301. | Anti-CD3ϵ | artificial | aa | pEDGNEELKGLEENGDQ |
| | paratope | | | |
| | binding peptide | | | |
| 302. | Anti-CD3ϵ | artificial | aa | pEDGNEEMGRGMEENGDQ |
| | paratope | | | |
| | binding peptide | | | |
| 303. | Anti-CD3ϵ | artificial | aa | pEDGNEELRGLEENGDQ |
| | paratope | | | |
| | binding peptide | | | |
| 304. | Anti-CD3ϵ | artificial | aa | pEDGNEELKGC |
| | paratope | | | |
| | binding peptide | | | |
| 305. | Anti-CD3ϵ | artificial | aa | pEDGNEELKGK |
| | paratope | | | |
| | binding peptide | | | |
| 306. | Anti-CD3ϵ | artificial | aa | pEDGEEELKGC |
| | paratope | | | |
| | binding peptide | | | |
| 307. | Anti-CD3ϵ | artificial | aa | pEDGEEELKGK |
| | paratope | | | |
| | binding peptide | | | |
| 308. | Anti-CD3ϵ | artificial | aa | pEHGNEELK |
| | paratope | | | |
| | binding peptide | | | |
| 309. | Anti-CD3ϵ | artificial | aa | pENGNEELK |
| | paratope | | | |
| | binding peptide | | | |
| 310. | Anti-CD3ϵ | artificial | aa | pEDFNEELK |
| | paratope | | | |
| | binding peptide | | | |
| 311. | Anti-CD3ϵ | artificial | aa | pEDYNEELK |
| | paratope | | | |
| | binding peptide | | | |
| 312. | Anti-CD3ϵ | artificial | aa | pEDGHEELK |
| | paratope | | | |
| | binding peptide | | | |
| 313. | Anti-CD3ϵ | artificial | aa | pEDGQEELK |
| | paratope | | | |
| | binding peptide | | | |
| 314. | Anti-CD3ϵ | artificial | aa | pEDGREELK |
| | paratope | | | |
| | binding peptide | | | |
| 315. | Anti-CD3ϵ | artificial | aa | pEDGNELLK |
| | paratope | | | |
| | binding peptide | | | |
| 316. | Anti-CD3ϵ | artificial | aa | pEDGNEPLK |
| | paratope | | | |
| | binding peptide | | | |
| 317. | Anti-CD3ϵ | artificial | aa | pEDGNEWLK |
| | paratope | | | |
| | binding peptide | | | |
| 318. | Anti-CD3ϵ | artificial | aa | pEDGHELLK |
| | paratope | | | |
| | binding peptide | | | |
| 319. | Anti-CD3ϵ | artificial | aa | pEDGREWLK |
| | paratope | | | |
| | binding peptide | | | |
| 320. | Anti-CD3ϵ | artificial | aa | pENFNEELK |
| | paratope | | | |
| | binding peptide | | | |
| 321. | Anti-CD3ϵ | artificial | aa | pEHFNEELK |
| | paratope | | | |
| | binding peptide | | | |
| 322. | Anti-CD3ϵ | artificial | aa | pENFHELLK |
| | paratope | | | |
| | binding peptide | | | |
| 323. | Anti-CD3ϵ | artificial | aa | pENYHELLK |
| | paratope | | | |
| | binding peptide | | | |
| 324. | Anti-CD3ϵ | artificial | aa | pEHFHELLK |
| | paratope | | | |
| | binding peptide | | | |
| 325. | Anti-CD3ϵ | artificial | aa | pENFREWLK |
| | paratope | | | |
| | binding peptide | | | |
| 326. | Anti-CD3ϵ | artificial | aa | pENFEELLK |
| | paratope | | | |
| | binding peptide | | | |
| 327. | Anti-CD3ϵ | artificial | aa | pENYEELLK |
| | paratope | | | |
| | binding peptide | | | |
| 328. | Anti-CD3ϵ | artificial | aa | pENFEEWLK |
| | paratope | | | |
| | binding peptide | | | |
| 329. | Anti-CD3ϵ | artificial | aa | pENFEEPLK |
| | paratope | | | |
| | binding peptide | | | |
| 330. | Anti-CD3ϵ | artificial | aa | pENFQELLK |
| | paratope | | | |
| | binding peptide | | | |
| 331. | Anti-CD3ϵ | artificial | aa | pENFREPLK |
| | paratope | | | |
| | binding peptide | | | |
| 332. | Anti-CD3ϵ | artificial | aa | PENFRELLK |
| | paratope | | | |
| | binding peptide | | | |
| 333. | Anti-CD3ϵ | artificial | aa | pENFHELLK |
| | paratope | | | |
| | binding peptide | | | |
| 334. | Anti-CD3ϵ | artificial | aa | pENYQELLK |
| | paratope | | | |
| | binding peptide | | | |
| 335. | Anti-CD3ϵ | artificial | aa | pENYEEWLK |
| | paratope | | | |
| | binding peptide | | | |
| 336. | Anti-CD3ϵ | artificial | aa | pEHFEELLK |
| | paratope | | | |
| | binding peptide | | | |
| 337. | Anti-CD38 | artificial | aa | pEHFQELLK |
| | paratope | | | |
| | binding peptide | | | |
| 338. | Anti-CD38 | artificial | aa | pEHFREPLK |
| | paratope | | | |
| | binding peptide | | | |
| 339. | I2L-HCDR1 | artificial | aa | KYAMN |
| 340. | I2L-HCDR2 | artificial | aa | RIRSKYNNYATYYADAVKD |
| 341. | I2L-HCDR3 | artificial | aa | AGNFGSSYISYFAY |
| 342. | I2L-LCDR1 | artificial | aa | GSSTGAVTSGNYPN |
| 343. | I2L-LCDR2 | artificial | aa | GTKFLAP |
| 344. | I2L-LCDR3 | artificial | aa | VLYYSNRWV |
| 345. | I2L-VH | artificial | aa | EVQLVESGGGIVQPGGSLKISCAASGFTFNKYAMN |
| | | | | WVRQAPGKGMEWVARIRSKYNNYATYYADAVKD |
| | | | | RFTISRDDSKNTLYLQMNNLKTEDTAVYYCVRAGN |
| | | | | FGSSYISYFAYWGQGTLVTVSS |
| 346. | I2L-VL | artificial | aa | QTVVTQEPSLTVSPGGTVTITCGSSTGAVTSGNYPN |
| | | | | WIQKKPGQAPRGLIGGTKFLAPGTPARFSGSLEGGK |
| | | | | AALTLSGVQPEDEAEYYCVLYYSNRWVFGSGTKLT |
| | | | | VL |
| 347. | I2E-HCDR1 | artificial | aa | KYAIN |
| 348. | I2E-HCDR2 | artificial | aa | RIRSKYNNYATYYADAVKD |
| 349. | I2E-HCDR3 | artificial | aa | AGNFGSSYISYWAY |
| 350. | I2E-LCDR1 | artificial | aa | GSSTGAVTSGNYPN |
| 351. | I2E-LCDR2 | artificial | aa | GTKFLAP |
| 352. | I2E-LCDR3 | artificial | aa | VLWYSNRWV |
| 353. | I2E-VH | artificial | aa | EVQLVESGGGLVQPGGSLKLSCAASGFTFNKYAIN |
| | | | | WVRQAPGKGLEWVARIRSKYNNYATYYADAVKD |
| | | | | RFTISRDDSKNTVYLQMNNLKTEDTAVYYCARAGN |
| | | | | FGSSYISYWAYWGQGTLVTVSS |
| 354. | I2E-VL | artificial | aa | QTVVTQEPSLTVSPGGTVTITCGSSTGAVTSGNYPN |
| | | | | WVQKKPGQAPRGLIGGTKFLAPGTPARFSGSLSGGK |
| | | | | AALTLSGVQPEDEAEYYCVLWYSNRWVFGSGTKLT |
| | | | | VL |
| 355. | MS 15-B12 CC- | artificial | aa | SSSYFWG |
| | HCDR1 | | | |
| 356. | MS 15-B12 CC- | artificial | aa | NIYYSGSSNYNPSLKS |
| | HCDR2 | | | |
| 357. | MS 15-B12 CC- | artificial | aa | LPRGDRDAFDI |
| | HCDR3 | | | |
| 358 | MS 15-B12 CC- | artificial | aa | RASQGISNYLA |
| | LCDR1 | | | |
| 359. | MS 15-B12 CC- | artificial | aa | AASTLQS |
| | LCDR2 | | | |
| 360. | MS 15-B12 CC- | artificial | aa | QQSYSTPFT |
| | LCDR3 | | | |
| 361. | CH3 15-E11 | artificial | aa | NYWMN |
| | CC-HCDR1 | | | |
| 362. | CH3 15- | artificial | Aa | NIAYGVAGTNYNQKFQG |
| | E11_1_VAG_ | | | |
| | CC-HCDR2 | | | |
| 363. | CH3 15-E11 | artificial | aa | RYFYVMDY |
| | CC-HCDR3 | | | |
| 364. | CH3 15-E11 | artificial | aa | RASQDISNYLN |
| | CC-LCDR1 | | | |
| 365. | CH3 15-E11 | artificial | aa | YTSRLHS |
| | CC-LCDR2 | | | |
| 366. | CH3 15-E11 | artificial | aa | VQYAQFPLT |
| | CC-LCDR3 | | | |
| 367. | Ab-A1 | artificial | aa | TYAMN |
| | XmAb2 + 1, Ab- | | | |
| | A2 | | | |
| | (N670) | | | |
| | XmAb2 + 1, Ab- | | | |
| | B1 | | | |
| | XmAb2 + 1 | | | |
| | Anti-CD3, | | | |
| | HCDR1 | | | |
| 368. | Ab-A1 | artificial | aa | RIRSKYNNYATYYADSVKG |
| | XmAb2 + 1, Ab- | | | |
| | A2 | | | |
| | (N670) | | | |
| | XmAb2 + 1, Ab- | | | |
| | B1 | | | |
| | XmAb2 + 1 | | | |
| | Anti-CD3, | | | |
| | HCDR2 | | | |
| 369. | Ab-A1 | artificial | aa | HGNFGDSYVSWFAY |
| | XmAb2 + 1, Ab- | | | |
| | A2 | | | |
| | (N67Q) | | | |
| | XmAb2 + 1, Ab- | | | |
| | B1 | | | |
| | XmAb2 + 1 | | | |
| | Anti-CD3, | | | |
| | HCDR3 | | | |
| 370. | Ab-A1 | artificial | aa | GSSTGAVTTSNYAN |
| | XmAb2 + 1, Ab- | | | |
| | A2 | | | |
| | (N670) | | | |
| | XmAb2 + 1, Ab- | | | |
| | B1 | | | |
| | XmAb2 + 1 | | | |
| | Anti-CD3, | | | |
| | LCDR1 | | | |
| 371. | Ab-A1 | artificial | aa | GTNKRAP |
| | XmAb2 + 1, Ab- | | | |
| | A2 | | | |
| | (N670) | | | |
| | XmAb2 + 1, Ab- | | | |
| | B1 | | | |
| | XmAb2 + 1 | | | |
| | Anti-CD3, | | | |
| | LCDR2 | | | |
| 372. | Ab-A1 | artificial | aa | ALWYSNIIWV |
| | XmAb2 + 1, Ab- | | | |
| | A2 | | | |
| | (N670) | | | |
| | XmAb2 + 1, Ab- | | | |
| | B1 | | | |
| | XmAb2 + 1 | | | |
| | Anti-CD3, | | | |
| | LCDR3 | | | |
| 373. | Ab-A1/A2 | artificial | aa | EIVLTQSPATLSLSPGERATLSCRASSSVSYMHWFQ |
| | (N670) | | | QKPGQAPRLLIYSTSNLASGIPARFSGSGSGTDYTLTI |
| | Xmab2 + 1 | | | SSLEPEDFAVYYCQQRRSFPYTFGQGTKLEIKRTVA |
| | light chain | | | APSVFIFPPSDEQLKSGTASVVCLLNNFYPREAKVQ |
| | minus signal | | | WKVDNALQSGNSQESVTEQDSKDSTYSLSSTLTLSK |
| | sequence | | | ADYEKHKVYACEVTHQGLSSPVTKSFNRGEC |
| 374. | Ab-A1 | artificial | aa | QVQLVQSGAEVKKPGASVKVSCKASGYTFSTYWI |
| | Xmab2 + 1 | | | EWVRQAPGQRLEWMGEILPGSGNTDFNEKFQG |
| | heavy chain | | | RVTFTADTSSDTAYMELSSLRSEDTAVYYCTRWGY |
| | minus the | | | YGTRGYFNVWGQGTLVTVSSASTKGPSVFPLAPSSK |
| | signal sequence | | | STSGGTAALGCLVKDYFPEPVTVSWNSGALTSGVH |
| | | | | TFPAVLQSSGLYSLSSVVTVPSSSLGTQTYICNVNHK |
| | | | | PSDTKVDKKVEPKSCDKTHTCPPCPAPPVAGPSVFL |
| | | | | FPPKPKDTLMISRTPEVTCVVVDVKHEDPEVKFNW |
| | | | | YVDGVEVHNAKTKPREEEYNSTYRVVSVLTVLHQD |
| | | | | WLNGKEYKCKVSNKALPAPIEKTISKAKGQPREPQV |
| | | | | YTLPPSREEMTKNQVSLTCDVSGFYPSDIAVEWESD |
| | | | | GQPENNYKTTPPVLDSDGSFFLYSKLTVDKSRWEQ |
| | | | | GDVFSCSVMHEALHNHYTQKSLSLSPGK |
| 375. | Ab-A1 | artificial | aa | QVQLVQSGAEVKKPGASVKVSCKASGYTFSTYWI |
| | Xmab2 + 1 | | | EWVRQAPGQRLEWMGEILPGSGNTDENEKFQG |
| | XVHMx | | | RVTFTADTSSDTAYMELSSLRSEDTAVYYCTRWGY |
| | minus the | | | YGTRGYFNVWGQGTLVTVSSASTKGPSVFPLAPSSK |
| | signal | | | STSGGTAALGCLVKDYFPEPVTVSWNSGALTSGVH |
| | sequence | | | TFPAVLQSSGLYSLSSVVTVPSSSLGTQTYICNVNH |
| | | | | KPSNTKVDKKVEPKSCGGGGSGGGGSEVQLVESGG |
| | | | | GLVQPGGSLRLSCAASGFTFSTYAMNWVRQAPG |
| | | | | KGLEWVGRIRSKYNNYATYYADSVKGRFTISRDDS |
| | | | | KNTLYLQMNSLRAEDTAVYYCVRHGNFGDSYVSW |
| | | | | FAYWGQGTLVTVSSGKPGSGKPGSGKPGSGKPGSQ |
| | | | | AVVTQEPSLTVSPGGTVTLTCGSSTGAVTTSNYAN |
| | | | | WVQQKPGKSPRGLIGGTNKRAPGVPARFSGSLLGG |
| | | | | KAALTISGAQPEDEADYYCALWYSNHWVFGGGTK |
| | | | | LTVLGGGGSGGGGSKTHTCPPCPAPVAGPSVFLFPP |
| | | | | KPKDTLMISRTPEVTCVVVDVKHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVYT |
| | | | | LPPSREQMTKNQVKTCLVKGFYPSDIAVEWESNGQ |
| | | | | PENNYKTTPPVLDSDGSFFLYSKLTVDKSRWQQGN |
| | | | | VFSCSVMHEALHNHYTQKSSLSPGK |
| 376 | Ab-A2 (N67Q) | artificial | aa | QVQLVQSGAEVKKPGASVKVSCKASGYTFSTYWIE |
| | Xmab2 + 1 | | | WVRQAPGQRLEWMGEILPGSGQTDFNEKFQGRVTF |
| | heavy chain | | | TADTSSDTAYMELSSLRSEDTAVYYCTRWGYYGTR |
| | minus | | | GYFNVWGQGTLVTVSSASTKGPSVFPLAPSSKSTSG |
| | signal sequence | | | GTAALGCLVKDYFPEPVTVSWNSGALTSGVHTFPA |
| | | | | VLQSSGLYSLSSVVTVPSSSLGTQTYICNVNHKPSDT |
| | | | | KVDKKVEPKSCDKTHTCPPCPAPPVAGPSVFLFPPK |
| | | | | PKDTLMISRTPEVTCVVVDVKHEDPEVKFNWYVDG |
| | | | | VEVHNAKTKPCEEEYGSTYRCVS |
| 377. | Ab-B1 | artificial | aa | DIVLTQTPLSLSVTPGQPASISCKASQSVDYDGDSFM |
| | Xmab2 + 1 | | | NWYLQKPGQPPQLLIYVASNLESGVPDRFSGSGSGT |
| | light chain | | | DFTLKISRVEAEDVGVYYCQQSNEEPPTFGQGTKLE |
| | minus signal | | | IKRTVAAPSVFIFPPSDEQLKSGTASVVCLLNNFYPR |
| | sequence | | | EAKVQWKVDNALQSGNSQESVTEQDSKDSTYSLSS |
| | | | | TLTLSKADYEKHKVYACEVTHQGLSSPVTKSFNRG |
| | | | | EC |
| 378. | Ab-B1 | artificial | aa | QLVQSGAEVKKPGATVKISCKASGYTFTNYGMNW |
| | Xmab2 + 1 | | | VQQAPGQGLEWMGWMNTYTGEPTYADKFQGRVT |
| | light chain | | | FTLDTSARTVYMELSSLRSEDTAVYFCARAGGQLRP |
| | minus signal | | | GAMDYWGQGTMVTVSSASTKGPSVFPLAPSSKSTS |
| | sequence | | | GGTAALGCLVKDYFPEPVTVSWNSGALTSGVHTFP |
| | | | | AVLQSSGLYSLSSVVTVPSSSLGTQTYICNVNHKPSD |
| | | | | TKVDKKVEPKSCDKTHTCPPCPAPPVAGPSVFLFPP |
| | | | | KPKDTLMISRTPEVTCVVVDVKHEDPEVKFNWYVD |
| | | | | GVEVHNAKTKPREEEYNSTYRVVSVLTVLHQDWL |
| | | | | NGKEYKCKVSNKALPAPIEKTISKAKGQPREPQVYT |
| | | | | LPPSREEMTKNQVSLTCDVSGFYPSDIAVEWESDGQ |
| | | | | PENNYKTIPPVLDSDGSFFLYSKLTVDKSRWEQGDV |
| | | | | FSCSVMHEALHNHYTQKSLSLSPGK |
| 379. | Ab-B1 | artificial | aa | QLVQSGAEVKKPGATVKISCKASGYTFTNYGMNW |
| | Xmab2 + 1 | | | VQQAPGQGLEWMGWMNTYTGEPTYADKFQGRVT |
| | XMVHx | | | FTLDTSARTVYMELSSLRSEDTAVYFCARAGGQLRP |
| | minus signal | | | GAMDYWGQGTMVTVSSASTKGPSVFPLAPSSKSTS |
| | sequence | | | GGTAALGCLVKDYFPEPVTVSWNSGALTSGVHTFP |
| | | | | AVLQSSGLYSLSSVVTVPSSSLGTQTYICNVNIIKPSN |
| | | | | TKVDKKVEPKSCGGGGSGGGGSEVQLVESGGGLV |
| | | | | QPGGSLRLSCAASGFTFSTYAMNWVRQAPGKGLE |
| | | | | WVGRIRSKYNNYATYYADSVKGRFTISRDDSKNTL |
| | | | | YLQMNSLRAEDTAVYYCVRHGNFGDSYVSWFAY |
| | | | | WGQGTLVTVSSGKPGSGKPGSGKPGSGKPGSQAVV |
| | | | | TQEPSLTVSPGGTVTLTCGSSTGAVTTSNYANWVQ |
| | | | | QKPGKSPRGLIGGTNKRAPGVPARFSGSLLGGKAAL |
| | | | | TISGAQPEDEADYYCALWYSNHWVFGGGTKLTVL |
| | | | | GGGGSGGGGSKTHTCPPCPAPPVAGPSVFLFPPKPK |
| | | | | DTLMISRTPEVTCVVVDVKHEDPEVKFNWYVDGVE |
| | | | | VHNAKTKPREEQYNSTYRVVSVLTVLHQDWLNGK |
| | | | | EYKCKVSNKALPAPIEKTISKAKGQPREPQVYTLPPS |
| | | | | REQMTKNQVKLTCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSPGK |
| 380. | Ab-A2 (N670) | artificial | aa | QVQLVQSGAEVKKPGASVKVSCKASGYTFSTYWIE |
| | Xmab2 + 1 | | | WVRQAPGQRLEWMGEILPGSGQTDFNEKFQGRVTF |
| | XVHMx minus | | | TADTSSDTAYMELSSLRSEDTAVYYCTRWGYYGTR |
| | signal sequence | | | GYFNVWGQGTLVTVSSASTKGPSVFPLAPSSKSTSG |
| | | | | GTAALGCLVKDYFPEPVTVSWNSGALTSGVHTFPA |
| | | | | VLUSSGLYSLSSVVTVPSSSLGTQTYICNVNHKPSNT |
| | | | | KVDKKVEPKSCGGGGSGGGGSEVQLVESGGGLVQP |
| | | | | GGSLRLSCAASGFTFSTYAMNWVRQAPGKGLEWV |
| | | | | GRIRSKYNNYATYYADSVKGRFTISRDDSKNTLYLQ |
| | | | | MNSLRAEDTAVYYCVRHGNFGDSYVSWFAYWGQ |
| | | | | GTLVTVSSGKPGSGKPGSGKPGSGKPGSQAVVTQEP |
| | | | | SLTVSPGGTVTLTCGSSTGAVTTSNYANWVQQKPG |
| | | | | KSPRGLIGGTNKRAPGVPARFSGSLLGGKAALTISG |
| | | | | AQPEDEADYYCALWYSNHWVFGGGTKLTVLGGGG |
| | | | | SGGGGSKTHTCPPCPAPPVAGPSVFLFPPKPKDTLMI |
| | | | | SRTPEVTCVVVDVKHEDPEVKFNWYVDGVEVHNA |
| | | | | KTKPCEEQYGSTYRCVSVLTVLHQDWLNGKEYKC |
| | | | | KVSNKALPAPIEKTISKAKGQPREPQVYTLPPSREQ |
| | | | | MTKNQVKLTCLVKGFYPSDIAVEWESNGQPENNYK |
| | | | | TTPPVLDSDGSFFLYSKLTVDKSRWQQGNVFSCSV |
| | | | | MHEALHNHYTQKSLSLSPGK |
| 381. | CD3ϵ binding | artificial | aa | EVQLVESGGGLVQPGGSLRLSCAASGFTFSTYAMN |
| | domain of Ab- | | | WVRQAPGKGLEWVGRIRSKYNNYATYYADSVKGR |
| | B1 and Ab-A2 | | | FTISRDDSKNTLYLQMNSLRAEDTAVYYCVRHGNF |
| | Xmab2 + 1 | | | GDSYVSWFAYWGQGTLVTVSSGKPGSGKPGSGKP |
| | | | | GSGKPGSQAVVTQEPSLTVSPGGTVTLTCGSSTGAV |
| | | | | TTSNYANWVQQKPGKSPRGLIGGTNKRAPGVPARF |
| | | | | SGSLLGGKAALTISGAQPEDEADYYCALWYSNHWV |
| | | | | FGGGTKLTVL |
| 382. | I2E VHVL | artificial | aa | EVQLVESGGGLVQPGGSLKLSCAASGFTFNKYAIN |
| | CD3ϵ binding | | | WVRQAPGKGLEWVARIRSKYNNYATYYADAVKD |
| | domain | | | RFTISRDDSKNTVYLQMNNLKTEDTAVYYCARAGN |
| | | | | FGSSYISYWAYWGQGTLVTVSSGGGGSGGGGSGGG |
| | | | | GSQTVVTQEPSLTVSPGGTVTITCGSSTGAVTSGNYP |
| | | | | NWVQKKPGQAPRGLIGGTKFLAPGTPARFSGSLSGG |
| | | | | KAALTLSGVQPEDEAEYYCVLWYSNRWVFGSGTK |
| | | | | LTVL |
| 383. | I2L VHVL | artificial | aa | EVQLVESGGGLVQPGGSLKLSCAASGFTFNKYAMN |
| | CD3ϵ binding | | | WVRQAPGKGMEWVARIRSKYNNYATYYADAVKD |
| | domain | | | RFTISRDDSKNTLYLQMNNLKTEDTAVYYCVRAGN |
| | | | | FGSSYISYFAYWGQGTLVTVSSGGGGSGGGGSGGG |
| | | | | GSQTVVTQEPSLTVSPGGTVTITCGSSTGAVTSGNYP |
| | | | | NWIQKKPGQAPRGLIGGTKFLAPGTPARFSGSLEGG |
| | | | | KAALTLSGVQPEDEAEYYCVLYYSNRWVFGSGTKL |
| | | | | TVL |
| 384. | Anti-CD3ϵ | artificial | aa | pEEGNEELKGC |
| | paratope | | | |
| | binding peptide | | | |
| 385. | Anti-CD3ϵ | artificial | aa | pEEGNEE |
| | paratope | | | |
| | binding peptide | | | |
| 386. | Anti-CD3ϵ | artificial | aa | pEEGNEEL |
| | paratope | | | |
| | binding peptide | | | |
| 387. | Anti-CD3ϵ | artificial | aa | pEEGNEELK |
| | paratope | | | |
| | binding peptide | | | |
| 388. | Anti-CD3ϵ | artificial | aa | pEEGNEELKG |
| | paratope | | | |
| | binding peptide | | | |
| 389. | Anti-CD3ϵ | artificial | aa | pEEGEEELKGC |
| | paratope | | | |
| | binding peptide | | | |
| 390. | Anti-CD3ϵ | artificial | aa | pEFGNEE |
| | paratope | | | |
| | binding peptide | | | |
| 391. | Anti-CD3ϵ | artificial | aa | pEFGNEEK |
| | paratope | | | |
| | binding peptide | | | |
| 392. | Anti-CD3ϵ | artificial | aa | X1X2X3X4EX5 |
| | paratope | | | |
| | binding peptide | | | |
| | Consensus | | | |
| | 6 mer | | | |
| 393. | Anti-CD3ϵ | artificial | aa | X1X2X3X4EX5X6X7 |
| | paratope | | | |
| | binding peptide | | | |
| | Consensus | | | |
| | 8 mer | | | |
| 394. | Anti-CD3ϵ | artificial | aa | pEX1X2X3EX4 |
| | paratope | | | |
| | binding peptide | | | |
| | Consensus | | | |
| | 6 mer | | | |
| 395. | Anti-CD3ϵ | artificial | aa | pEX1X2X3EX4X5X6 |
| | paratope | | | |
| | binding peptide | | | |
| | Consensus | | | |
| | 8 mer | | | |
| 396. | Anti-CD3ϵ | artificial | aa | pEX1X2X3EX4X5LK |
| | paratope | | | |
| | binding peptide | | | |
| | Consensus | | | |
| | 8 mer | | | |
| 397. | Anti-CD3ϵ | artificial | aa | pEDGCEE |
| | paratope | | | |
| | binding peptide | | | |
| 398. | Anti-CD3ϵ | artificial | aa | pEDGDEE |
| | paratope | | | |
| | binding peptide | | | |
| 399. | Anti-CD3ϵ | artificial | aa | pEDGEEE |
| | paratope | | | |
| | binding peptide | | | |
| 400. | Anti-CD3ϵ | artificial | aa | pEDGFEE |
| | paratope | | | |
| | binding peptide | | | |
| 401. | Anti-CD3ϵ | artificial | aa | pEDGKEE |
| | paratope | | | |
| | binding peptide | | | |
| 402. | Anti-CD3ϵ | artificial | aa | pEDGLEE |
| | paratope | | | |
| | binding peptide | | | |
| 403. | Anti-CD3ϵ | artificial | aa | pEDGMEE |
| | paratope | | | |
| | binding peptide | | | |
| 404. | Anti-CD3ϵ | artificial | aa | pEDGWEE |
| | paratope | | | |
| | binding peptide | | | |
| 405. | Anti-CD3ϵ | artificial | aa | pEDGYEE |
| | paratope | | | |
| | binding peptide | | | |
| 406. | Anti-CD3ϵ | artificial | aa | pEDGNEC |
| | paratope | | | |
| | binding peptide | | | |
| 407. | Anti-CD3ϵ | artificial | aa | pEDGNED |
| | paratope | | | |
| | binding peptide | | | |
| 408. | Anti-CD3ϵ | artificial | aa | pEDGNEF |
| | paratope | | | |
| | binding peptide | | | |
| 409. | Anti-CD3ϵ | artificial | aa | pEDGNEG |
| | paratope | | | |
| | binding peptide | | | |
| 410. | Anti-CD3ϵ | artificial | aa | pEDGNEH |
| | paratope | | | |
| | binding peptide | | | |
| 411. | Anti-CD3ϵ | artificial | aa | pEDGNEK |
| | paratope | | | |
| | binding peptide | | | |
| 412. | Anti-CD3ϵ | artificial | aa | pEDGNEL |
| | paratope | | | |
| | binding peptide | | | |
| 413. | Anti-CD3ϵ | artificial | aa | pEDGNEM |
| | paratope | | | |
| | binding peptide | | | |
| 414. | Anti-CD3ϵ | artificial | aa | pEDGNEQ |
| | paratope | | | |
| | binding peptide | | | |
| 415. | Anti-CD3ϵ | artificial | aa | pEDGNER |
| | paratope | | | |
| | binding peptide | | | |
| 416. | Anti-CD3ϵ | artificial | aa | pEDGNES |
| | paratope | | | |
| | binding peptide | | | |
| 417. | Anti-CD3ϵ | artificial | aa | pEDGNET |
| | paratope | | | |
| | binding peptide | | | |
| 418. | Anti-CD3ϵ | artificial | aa | pEDGNEV |
| | paratope | | | |
| | binding peptide | | | |
| 419. | Anti-CD3ϵ | artificial | aa | PEDGNEY |
| | paratope | | | |
| | binding peptide | | | |
| 420. | CLDN6/CDR- | artificial | aa | GYYMH |
| | H1 | | | |
| 421. | CLDN6/CDR- | artificial | aa | WINPNSGETNYAQKFQG |
| | H2 | | | |
| 422. | CLDN6/CDR- | artificial | aa | DALIVVAPVTRDYYYYGMDV |
| | H3 | | | |
| 423. | CLDN6/CDR- | artificial | aa | RASQSVSSSYLA |
| | L1 | | | |
| 424. | CLDN6/CDR- | artificial | aa | GASSRAT |
| | L2 | | | |
| 425. | CLDN6/CDR- | artificial | aa | QQYGSSPLT |
| | L3 | | | |
| 426. | CLDN6/VH | artificial | aa | QVQLVQSGAEVKKPGASVKVSCKASGYTFTGYYM |
| | | | | HWVRQAPGQCLEWMGWINPNSGETNYAQKFQGR |
| | | | | VTMTRDTSISTAYMELSRLRSDDTAVYYCARDALIV |
| | | | | VAPVTRDYYYYGMDVWGQGTTVTVSS |
| 427. | CLDN6/VL | artificial | aa | EIVLTQSPGTLSLSPGERATLSCRASQSVSSSYLAWY |
| | | | | QQKPGQAPRLLIYGASSRATGIPDRFSGSGSGTDFTL |
| | | | | TISRLEPEDFAVYYCQQYGSSPLTFGCGTKLEIK |
| 428. | CLDN6/SCFV | artificial | aa | EIVLTQSPGTLSLSPGERATLSCRASQSVSSSYLAWY |
| | I2E | | | QQKPGQAPRLLIYGASSRATGIPDRFSGSGSGTDFTL |
| | | | | TISRLEPEDFAVYYCQQYGSSPLTFGCGTKLEIKGGG |
| | | | | GSGGGGSGGGGSQVQLVQSGAEVKKPGASVKVSC |
| | | | | KASGYTFTGYYMHWVRQAPGQCLEWMGWINPNS |
| | | | | GETNYAQKFQGRVTMTRDTSISTAYMELSRLRSDD |
| | | | | TAVYYCARDALIVVAPVTRDYYYYGMDVWGQGTT |
| | | | | VTVSS |
| 429. | CLDN6/ | artificial | aa | EIVLTQSPGTLSLSPGERATLSCRASQSVSSSYLAWY |
| | BISPECIFIC | | | QQKPGQAPRLLIYGASSRATGIPDRFSGSGSGTDFTL |
| | MOL I2E | | | TISRLEPEDFAVYYCQQYGSSPLTFGCGTKLEIKGGG |
| | | | | GSGGGGSGGGGSQVQLVQSGAEVKKPGASVKVSC |
| | | | | KASGYTFTGYYMHWVRQAPGQCLEWMGWINPNS |
| | | | | GETNYAQKFQGRVTMTRDTSISTAYMELSRLRSDD |
| | | | | TAVYYCARDALIVVAPVTRDYYYYGMDVWGQGTT |
| | | | | VTVSSSGGGGSEVQLVESGGGLVQPGGSLKLSCAAS |
| | | | | GFTFNKYAINWVRQAPGKGLEWVARIRSKYNNYAT |
| | | | | YYADAVKDRFTISRDDSKNTVYLQMNNLKTEDTAV |
| | | | | YYCARAGNFGSSYISYWAYWGQGTLVTVSSGGGG |
| | | | | SGGGGSGGGGSQTVVTQEPSLTVSPGGTVTITCGSS |
| | | | | TGAVTSGNYPNWVQKKPGQAPRGLIGGTKFLAPGT |
| | | | | PARFSGSLSGGKAALTLSGVQPEDEAEYYCVLWYS |
| | | | | NRWVFGSGTKLTVL |
| 430. | CLDN6/HLE- | artificial | aa | EIVLTQSPGTLSLSPGERATLSCRASQSVSSSYLAWY |
| | BITE I2E | | | QQKPGQAPRLLIYGASSRATGIPDRFSGSGSGTDFTL |
| | | | | TISRLEPEDFAVYYCQQYGSSPLTFGCGTKLEIKGGG |
| | | | | GSGGGGSGGGGSQVQLVQSGAEVKKPGASVKVSC |
| | | | | KASGYTFTGYYMHWVRQAPGQCLEWMGWINPNS |
| | | | | GETNYAQKFQGRVTMTRDTSISTAYMELSRLRSDD |
| | | | | TAVYYCARDALIVVAPVTRDYYYYGMDVWGQGTT |
| | | | | VTVSSSGGGGSLVQPGGSLKLSCAASGFTFNKYAIN |
| | | | | WVRQAPGKGLEWVARIRSKYNNYATYYADAVKD |
| | | | | RFTISRDDSKNTVYLQMNNLKTEDTAVYYCARAGN |
| | | | | FGSSYISYWAYWGQGTLVTVSSGGGGSGGGGSGGG |
| | | | | GSQTVVTQEPSLTVSPGGTVTITCGSSTGAVTSGNYP |
| | | | | NWVQKKPGQAPRGLIGGTKFLAPGTPARFSGSLSGG |
| | | | | KAALTLSGVQPEDEAEYYCVLWYSNRWVFGSGTK |
| | | | | LTVLGGGGDKTHTCPPCPAPELLGGPSVFLFPPKPK |
| | | | | DTLMISeRTPEVTCVVVDVSHEDPEVKFNWYVDGV |
| | | | | EVHNAKTKPCEEQYGSTYRCVSVLTVLHQDWLNG |
| | | | | KEYKCKVSNKALPAPIEKTISKAKGQPREPQVYTLP |
| | | | | PSREEMTKNQVSLTCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSPGKGGGGSGGGGSG |
| | | | | GGGSGGGGSGGGGSGGGGSDKTIITCPPCPAPELLG |
| | | | | GPSVFLFPPKPKDTLMISRTPEVTCVVVDVSHEDPEV |
| | | | | KFNWYVDGVEVHNAKTKPCEEQYGSTYRCVSVLT |
| | | | | VLHQDWLNGKEYKCKVSNKALPAPIEKTISKAKGQ |
| | | | | PREPQVYTLPPSREEMTKNQVSLTCLVKGFYPSDIA |
| | | | | VEWESNGQPENNYKTTPPVLDSDGSFFLYSKLTVD |
| | | | | KSRWQQGNVFSCSVMHEALHNHYTQKSLSLSPGK |
| 431. | Anti-CD3ϵ | artificial | aa | pEDGNEELK |
| | paratope | | | |
| | binding peptide | | | |
| 432 | Anti-CD3ϵ | artificial | aa | pEX1X2X3EX4X5X6X7X8 |
| | paratope | | | |
| | binding peptide | | | |
| | consensus | | | |
| | 10 mer | | | |
| 433. | Anti-CD3ϵ | artificial | aa | pEX1X2X3EX4LKX5X6 |
| | paratope | | | |
| | binding peptide | | | |
| | consensus | | | |
| | 10 mer | | | |
| 434. | Anti-CD3ϵ | artificial | aa | pEX1X2X3EELKGX4 |
| | paratope | | | |
| | binding peptide | | | |
| | consensus | | | |
| | 10 mer | | | |
| 435. | Anti-CD3ϵ | artificial | aa | pEX1GX2EELKGX3 |
| | paratope | | | |
| | binding peptide | | | |
| | consensus | | | |
| | 10 mer | | | |
| 436. | Anti-CD3ϵ | artificial | aa | pEDGX1EELKGX2 |
| | paratope | | | |
| | binding peptide | | | |
| | consensus | | | |
| | 10 mer | | | |
| 437. | Fc monomer-1 | artificial | aa | DKTHTCPPCPAPELLGGPSVFLFPPKPKDTLMISRTP |
| | +c/−g | | | EVTCVVVDVSHEDPEVKFNWYVDGVEVHNAKTKP |
| | | | | CEEQYGSTYRCVSVLTVLIIQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 438. | Fc monomer-2 | artificial | aa | DKTHTCPPCPAPELLGGPSVFLFPPKPKDTLMISRTP |
| | +c/−g/delGK | | | EVTCVVVDVSHEDPEVKFNWYVDGVEVHNAKTKP |
| | | | | CEEQYGSTYRCVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSP |
| 439. | Fc monomer-3 | artificial | aa | DKTHTCPPCPAPELLGGPSVFLFPPKPKDTLMISRTP |
| | −c/+g | | | EVTCVVVDVSHEDPEVKFNWYVDGVEVHNAKTKP |
| | | | | REEQYNSTYRVVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 440 | Fc monomer-4 | artificial | aa | DKTHTCPPCPAPELLGGPSVFLFPPKPKDTLMISRTP |
| | −c/+g/delGK | | | EVTCVVVDVSHEDPEVKFNWYVDGVEVHNAKTKP |
| | | | | REEQYNSTYRVVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSP |
| 441. | Fc monomer-5 | artificial | aa | DKTHTCPPCPAPELLGGPSVFLFPPKPKDTLMISRTP |
| | −c/−g | | | EVTCVVVDVSHEDPEVKFNWYVDGVEVHNAKTKP |
| | | | | REEQYGSTYRVVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMIIEALII |
| | | | | NHYTQKSLSLSPGK |
| 442. | Fc monomer-6 | artificial | aa | DKTHTCPPCPAPELLGGPSVFLFPPKPKDTLMISRTP |
| | −c/−g/delGK | | | EVTCVVVDVSHEDPEVKFNWYVDGVEVIINAKTKP |
| | | | | REEQYGSTYRVVSVLTVLHQDWLNGKEYKCKVSN |
| | | | | KALPAPIEKTISKAKGQPREPQVYTIPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSP |
| 443. | Fc monomer-7 | artificial | aa | DKTHTCPPCPAPELLGGPSVFLFPPKPKDTLMISRTP |
| | +c/+g | | | EVTCVVVDVSHEDPEVKFNWYVDGVEVHNAKTKP |
| | | | | CEEQYNSTYRCVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 444. | Fc monomer-8 | artificial | aa | DKTHTCPPCPAPELLGGPSVFLFPPKPKDTLMISRTP |
| | +c/+g/delGK | | | EVTCVVVDVSHEDPEVKFNWYVDGVEVHNAKTKP |
| | | | | CEEQYNSTYRCVSVLTVLHQDWLNGKEYKCKVSN |
| | | | | KALPAPIEKTISKAKGQPREPQVYTLPPSREEMTKNQ |
| | | | | VSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSP |

1. A T cell engager (TCE) masking molecule comprising:
(i) a binding peptide that binds to an anti-CD3 epsilon (CD3ε) paratope of a CD3ε binding domain of a TCE, wherein the anti-CD3ε paratope is directed against an epitope located within CD3ε, wherein CD3ε comprises the amino acid sequence of SEQ ID NO: 257;
(ii) a linker covalently linked to the C-terminus of the binding peptide; and
(iii) a half-life extending polymer covalently linked to the linker.

2. The TCE masking molecule of claim 1, wherein the anti-CD3ε paratope binds to an epitope comprising (i.) at least one amino acid residue of K67 or S77 of SEQ ID NO: 257; or (ii.) at least the amino acid residues K67, N68, I69, G70, S71, D72, E73, D74, H75, L76, and S77 of SEQ ID NO: 257,

3. The TCE masking molecule of claim 1, wherein the anti-CD3ε paratope is directed to a CD3ε epitope consisting of the amino acid sequence of SEQ ID NO: 258 or consisting of the amino acid sequence of SEQ ID NO: 259.

4. The TCE masking molecule of any one of the preceding claims, wherein the binding peptide comprises 5, 6, 7, 8, 9, 10, 11, 12, 13, 14, 15, 16, 17 or 18 amino acids, preferably 5, 6, 7, 8, 9 or 10 amino acids, more preferably 10 amino acids.

5. The TCE masking molecule of any one of the preceding claims, wherein the binding peptide binds to an anti-CD3ε paratope and comprises at the N-terminus at least the sequence of X1X2X3X4EX5 (SEQ ID NO: 392), wherein X1 is Q, pyroglutamic acid (pE), or S, and wherein X2 is D, H or N, and wherein X3 is G, F or Y, and wherein X4 is N, E, S, T, V, or I, and wherein X5 is E, L, P, or W.

6. The TCE masking molecule of any one of the preceding claims, wherein the binding peptide binds to an anti-CD3ε paratope and comprises at the N-terminus at least the sequence of X1DGX2EE (SEQ ID NO: 261), wherein X1 is Q, pE, or S, and wherein X2 is N, E, S, T, V, or I.

7. The TCE masking molecule of any one of the preceding claims, wherein the binding peptide binds to an anti-CD3ε paratope and comprises at the N-terminus at least the sequence of X1X2X3X4EX5X6X7 (SEQ ID NO: 393), wherein X1 is Q, pyroglutamic acid (pE), or S, and wherein X2 is D, H or N, and wherein X3 is G, F or Y, and wherein X4 is N, E, S, T, V, or I, and wherein X5 is E, L, P, or W, and wherein X6 is A, C, D, E, F, G, H, I, K, L, M, N, P, Q, R, S, T, V, W, or Y, and wherein X7 is A, C, D, E, F, G, H, I, K, L, M, N, P, Q, R, S, T, V, W, or Y.

8. The TCE masking molecule of any one of the preceding claims, wherein the binding peptide binds to an anti-CD3ε paratope and comprises at the N-terminus at least the sequence of pEX1X2X3EX4LK (SEQ ID NO: 396), wherein X1 is D, H or N, and wherein X2 is G, F or Y, and wherein X3 is N, E, S, T, V, or I, and wherein X4 is E, L, P, or W.

9. The TCE masking molecule of any one of the preceding claims, wherein the anti-CD3ε binding peptide comprises a palindrome.

10. The TCE masking molecule of any one of the preceding claims, wherein the palindrome has a K at the position between the mirrored amino acids.

11. The TCE masking molecule of any one of the preceding claims, wherein the binding peptide binds to an anti-CD3ε paratope and comprises at the N-terminus at least the amino acid sequence of any one of SEQ ID NO: 263 to 284, 286 to 293, 295 to 303, 308 to 338, 385 to 388, 397 to 419 and 431.

12. The TCE masking molecule of any one of the preceding claims, wherein the binding peptide binds to an anti-CD3ε paratope and comprises at the C-terminus a cysteine.

13. The TCE masking molecule of any one of the preceding claims, wherein the binding peptide at its C-terminus further comprises the amino acids G and C or G and K.

14. The TCE masking molecule of any one of claims 1 to 8, wherein the binding peptide binds to an anti-CD3ε paratope and comprises the sequence of pEX1X2X3EX4X5X6GX7 (SEQ ID NO: 433), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, and wherein X4 is E, L, P, or W, and wherein X5 is A, C, D, E, F, G, H, I, K, L, M, N, P, Q, R, S, T, V, W, or Y, and wherein X5 is A, C, D, E, F, G, H, I, K, L, M, N, P, Q, R, S, T, V, W, or Y, and wherein X7 is C or K.

15. The TCE masking molecule of any one of claims 1 to 8 and 14, wherein the binding peptide binds to an anti-CD3ε paratope and comprises the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, most preferably N or E, and wherein and wherein X4 is C or K.

16. The TCE masking molecule of any one of claims 1 to 8 and 14 to 15, wherein the binding peptide binds to an anti-CD3ε paratope and comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably E, N, H, Q or R, most preferably N or E, and wherein and wherein X3 is C or K.

17. The TCE masking molecule of any one of claims 1 to 8 and 14 to 16, wherein the binding peptide binds to an anti-CD3ε paratope and comprises the sequence of pEDGX1EELKGX2 (SEQ ID NO: 436), wherein X1 is N, H, Q, R, E, S, T, V, C, D, F, K L, M, W, Y or I, preferably N, E, H, Q or R, most preferably N or E, and wherein and wherein X2 is C or K.

18. The TCE masking molecule of any one of the preceding claims, wherein the binding peptide binds to an anti-CD3ε paratope and comprises the sequence of SEQ ID NO: 297, 300, 303, 304, 305, 306, 307, 384 or 389.

19. The TCE masking molecule of any one of the preceding claims, wherein the linker is
(a) a disulfide linker comprising disulfide (R—S—S—R′), wherein R is a half-life extending polymer and R′ is a binding peptide;
(b) a free thiol containing linear or branched PEGs, an OPSS derivative, a maleimide, a nor-bornene, an acrylimide, an acrylate, a vinyl sulfone, or an amine; or
(c) a carboxyl linker comprising an amine, an amide, or an epsilon derivatized acetyl bromide.

20. The TCE masking molecule of any one of the preceding claims, wherein the linker is selected from any one of the following moieties: 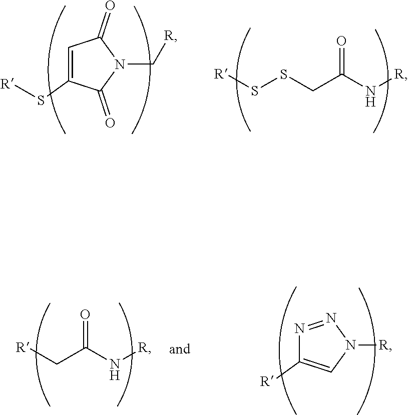
and or wherein the linker is selected from a maleimide-thiosuccinimide linker having the formula 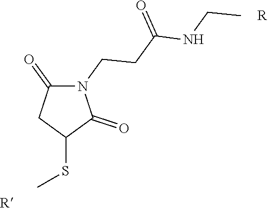
when the C-terminal amino acid of the binding peptide is K; and
an acetamide-thioether having the formula 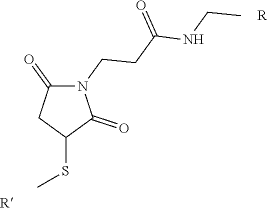
when the C-terminal amino acid of the binding peptide is C, and a disulfide having the formula R′—S—S—R when the C-terminal amino acid of the binding peptide is C,
wherein R is a half-life extending polymer and R′ is a binding peptide.

21. The TCE masking molecule of any one of the preceding claims, wherein the half-life extending polymer is mono-methoxy polyethylene glycol (mPEG), linear, 2-arm, 4-arm, 8-arm polyethylene glycol (PEG).; PLGA; peptide acrylate, polyglycerols, polyoxazolines, polyvinylpyrrolidone, polyacrylamides, poly(N-acryloylmorpholine), poly(N,N-dimethylacrylamide), ply(2-hydroxypropylmethacrylamide), polysarcosine, poly(2-hydroxyethylmethacrylamide), hyaluronic acids, sialic acid, poly[(organo)phosphazenes], or heparin.

22. The TCE masking molecule of any one of the preceding claims, wherein the half-life extending polymer is: 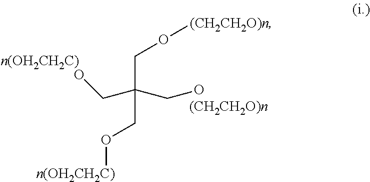
wherein n equals an integer of about 20 to 200, preferably 20 to 169, more preferably 20, 103 or 113 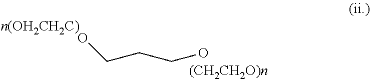
wherein n equals an integer of about 40 to 400. preferably about 40 or about 208, or
(iii.) (CH₂CH₂O)n, wherein n equals a number of about 80 to about 700, preferably about 80 to about 675, more preferably about 83, about 166 or about 417.

23. The TCE masking molecule of any of the preceding claims, wherein the half-life extending polymer is a non-branched linear, or a branched 2-arm, 4-arm, or 8-arm PEG, preferably linear or 4-arm, with a molecular weight of about 2 kDa to about 60 kDa, preferably about 4 kDa to about 30 kDa or 10 kDa to about 30 kDa, or more preferably about 5 or about 20 kDa.

24. The TCE masking molecule of any one of the preceding claims, wherein the half-life extending polymer is a branched polymer, preferably a 2-arm, 4-arm or 8-arm PEG with a molecular weight of about 2 kDa to about 60 kDa to which more than one binding peptide is linked via a linker each, preferably 2, 4 or 8 binding peptides to one half-life extending polymer.

25. The TCE masking molecule of any one of claims 1 to 8, 14 to 17, and 19 to 24, comprising the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E H Q or R, most preferably N or E, and
(a.) wherein and wherein X4 is C; a linker chosen from maleimide-thiosuccinimide linker having the formula 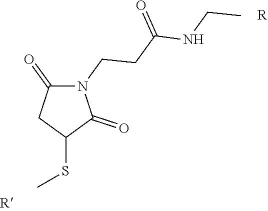
and a disulfide having the formula R′—S—S—R; and a half-life extending polymer that is a PEG of the following formula 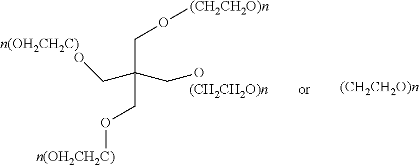
having a molecular weight of about 5 to 20 kDa, preferably about 5 kDa or about 10 kDa or about 20 kDa; or (b.) wherein and wherein X4 is K; an acetamide-thioether having the formula 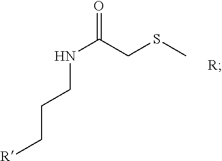
and a half-life extending polymer that is a PEG of the following formula 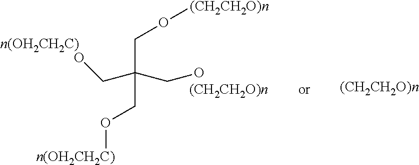
having a molecular weight of about 5 to 20 kDa, preferably about 5 kDa or about 10 kDa or about 20 kDa.

26. The TCE masking molecule of any one of claims 1 to 8, 14 to 17, and 19 to 25 comprising the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably E, N, H, Q or R, most preferably N or E, and
(a.) wherein and wherein X3 is C; a linker chosen from maleimide-thiosuccinimide linker having the formula 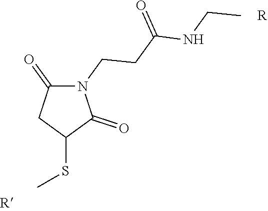
and a disulfide having the formula R′—S—S—R; and a half-life extending polymer that is a PEG of the following formula 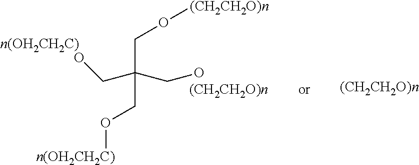
having a molecular weight of about 5 to 20 kDa, preferably about 5 kDa or about 10 kDa or about 20 kDa; or (b.) wherein and wherein X3 is K; an acetamide-thioether having the formula 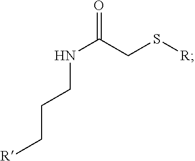
and a half-life extending polymer that is a PEG of the following formula 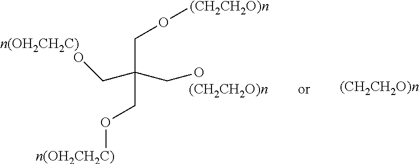
having a molecular weight of about 5 kDa to about 20 kDa, preferably about 5 kDa or about 10 kDa, or about 20 kDa.

27. The TCE masking molecule of any one of claims 1 to 8, 14 to 17 and 19 to 26 comprising the sequence of pEDGXIEELKGX2 (SEQ ID NO: 436), wherein X1 is N or E, and wherein (a.) X2 is C; a linker chosen from maleimide-thiosuccinimide linker having the formula 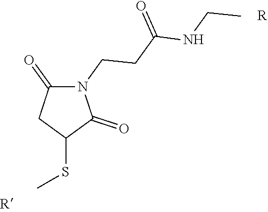
and a disulfide having the formula R′—S—S—R; and a half-life extending polymer that is a PEG of the following formula 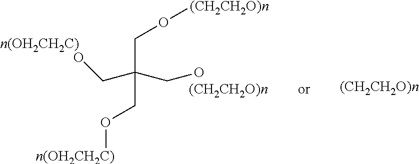
having a molecular weight of about 5 kDa or about 20 kDa; or (b) the sequence of pEDGXIEELKGX2 (SEQ ID NO: 436), wherein X1 is N or E, and wherein and wherein X2 is K; an acetamide-thioether having the formula 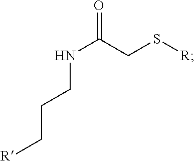
and a half-life extending polymer that is a PEG of the following formula 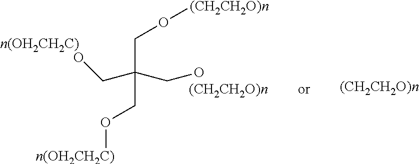
having a molecular weight of about 5 kDa to about 20 kDa, preferably about 5 kDa or about 10 kDa or about 20 kDa.

28. The TCE masking molecule of any one of claims 1 to 8, 14 to 17 and 19 to 27, comprising
(a) the amino acid sequence of SEQ ID 304, 306, 384 or 389, and
a linker chosen from maleimide thiosuccinimide linker having the formula 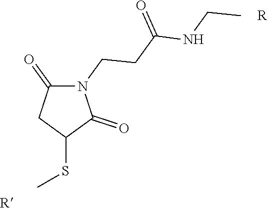
and a disulfide having the formula R′—S—S—R;
and a half-life extending polymer that is a PEG of the following formula 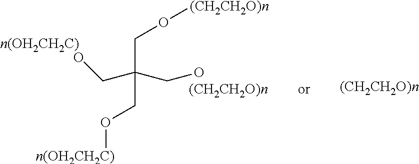
having a molecular weight of about 5 kDa to about 20 kDa, preferably about 5 kDa or about 10 kDa or about 20 kDa or
(b) the amino acid sequence of SEQ ID 305 or 307;
an acetamide-thioether having the formula 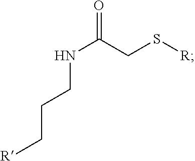
and a half-life extending polymer that is a PEG of the following formula 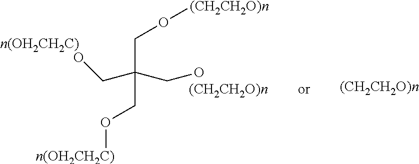
having a molecular weight of about 5 kDa to about 20 kDa preferably about 5 kDa or about 10 kDa or about 20 kDa.

29. The TCE masking molecule of any one of the preceding claims comprising any of the combinations (a.) to (k.) of a binding peptide, a linker and a half-life extending PEG polymer, wherein R′ stands for the binding peptide and R stands for the half-life extending PEG polymer:

| | Peptide seq | Linker | PEG configuration |
|---|---|---|---|
| (a.) | pEDGNEELKGC (SEQ ID NO 304) | 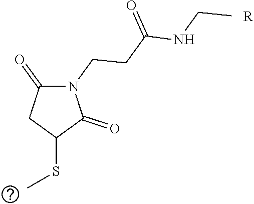 | 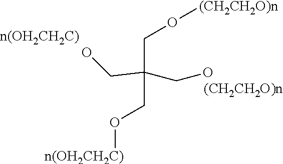 4 arm, 20 kDa |
| | | Thiosuccinimide | |
| (b.) | pEDGNEELKGC (SEQ ID NO 304) | 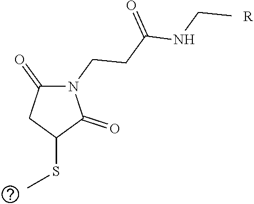 | 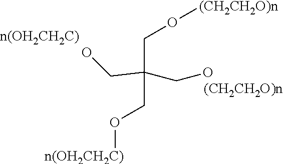 4 arm, 20 kDa |
| | | Thiosuccinimide | |
| (c.) | pEDGEEELKGC (SEQ ID NO 306) | 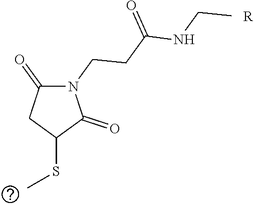 | 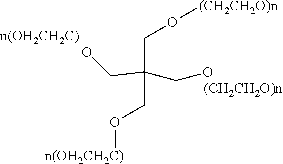 4 arm, 20 kDa |
| | | Thiosuccinimide | |
| (d.) | pEDGNEELKGK (SEQ ID NO 305) | 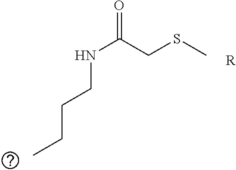 | 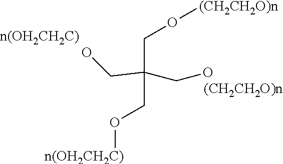 |
| | | Thioether | 4 arm, 20 kDA |
| (e.) | pEDGEEELKGK (SEQ ID NO 307) | 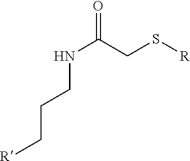 | 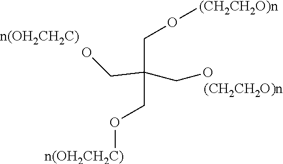 |
| | | Thioether | 4 arm, 20 kDA |
| (f.) | pEDGNEELKGC (SEQ ID NO 306) | 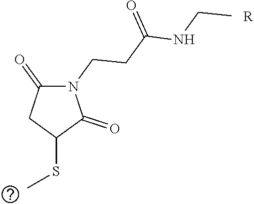 | 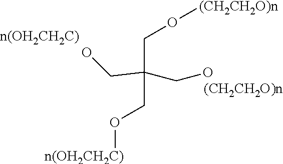 4 arm, 5 kDa |
| | | Thiosuccinimide | |
| (g.) | pEDGEEELKGC (SEQ ID NO 306) | 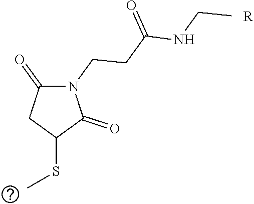 | 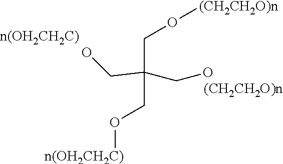 4 arm, 5 kDa |
| | | Thiosuccinimide | |
| (h.) | pEDGNEELKGK (SEQ ID NO 305) | 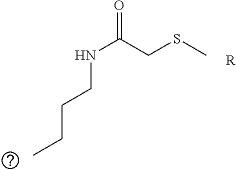 | (CH₂CH₂O)n Linear, 20 kDa |
| (i.) | pEDGEEELKGK (SEQ ID NO 307) | 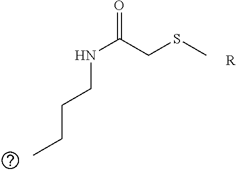 | (CH₂CH₂O)n Linear, 20 kDa |
| | | Thioether | |
| (j.) | pEDGNEELKGK (SEQ ID NO 305) | 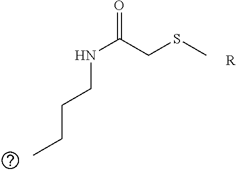 | (CH₂CH₂O)n Linear, 10 kDa |
| | | Thioether | |
| (k.) | pEDGEEELKGK (SEQ ID NO 307) | 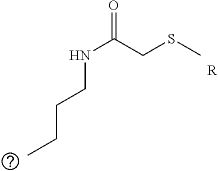 | (CH₂CH₂O)n Linear, 10 kDa |
| | | Thioether | |

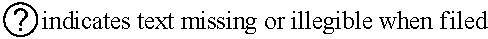

30. The TCE masking molecule of any of the one preceding claims, wherein the molecule has a half-life of about 1 hour to about 48 hours, preferably about 1.5 hours to about 24 hours, more preferably about 1.5 hours to about 4 hours, or more preferably about 2 hours.

31. A method for reducing the severity of cytokine release syndrome in a human subject undergoing treatment with a TCE, the method comprising administering to the human subject an effective dose of a T cell engager masking molecule, wherein the TCE masking molecule comprises:
(i.) a binding peptide that binds an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii.) a linker, wherein the linker is covalently linked to the C-terminus of the binding peptide; and
(iii.) at least one half-life extending polymer of preferably at least 2 kDa, wherein the polymer is covalently linked to the linker.

32. The method of claim 31, wherein the T cell engager masking molecule comprises:
a binding peptide which binds to an anti-CD3ε paratope and comprises the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, and (a.) wherein and wherein X4 is C; a linker chosen from maleimide-thiosuccinimide linker having the formula 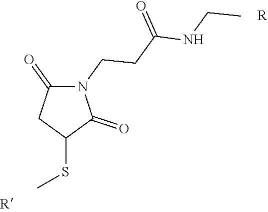
and a disulfide having the formula R′—S—S—R; and a half-life extending polymer that is a PEG of the following formula 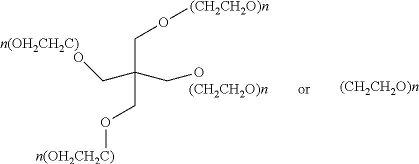
having a molecular weight of about 5 to 20 kDa, preferably about 5 kDa or about 10 kDa or about 20 kDa; or (b.) wherein and wherein X4 is K: an acetamide-thioether having the formula 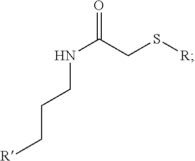
and a half-life extending polymer that is a PEG of the following formula 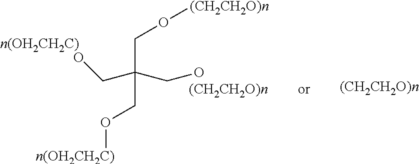
having a molecular weight of about 5 to 20 kDa, preferably about 5 kDa or about 10 kDa or about 20 kDa, wherein R′ stands for the binding peptide and R stands for the half-life extending PEG polymer.

33. The method of claim 31 or 32, wherein the T cell engager masking molecule comprises:
a binding peptide which binds to an anti-CD3ε paratope and comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably N, H, Q or R, and (a.) wherein and wherein X3 is C; a linker chosen from maleimide-thiosuccinimide linker having the formula 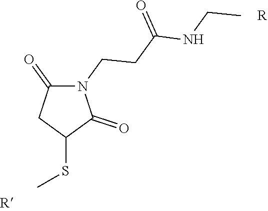
and a disulfide having the formula R′—S—S—R; and a half-life extending polymer that is a PEG of the following formula 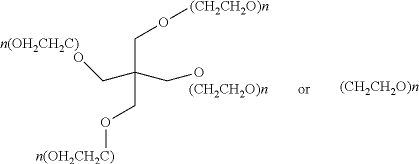
having a molecular weight of about 5 to 20 kDa, preferably about 5 kDa or about 10 kDa or about 20 kDa; or (b.) wherein and wherein X3 is K; an acetamide-thioether having the formula 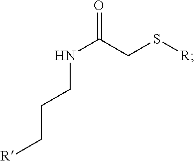
and a half-life extending polymer that is a PEG of the following formula 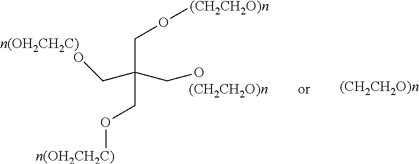
having a molecular weight of about 5 to 20 kDa, preferably about 5 kDa or about 10 kDa or about 20 kDa, wherein R′ stands for the binding peptide and R stands for the half-life extending PEG polymer.

34. The method of claims 31 to 33, wherein the T cell engager masking molecule comprises:
a binding peptide which binds to an anti-CD3ε paratope and comprises the sequence of pEDGX1EELKGX2 (SEQ ID NO: 436), wherein X1 is D, H or N, and wherein X1 is N, H, Q, R, E, S, T, V, C, D, F, K L, M, W, Y or I, preferably N, E, H, Q or R, most preferably N or E, and (a.) wherein and wherein X2 is C; a linker chosen from maleimide-thiosuccinimide linker having the formula 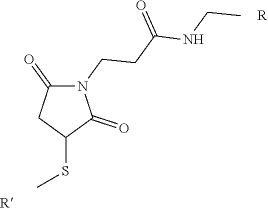
and a disulfide having the formula R′—S—S—R; and a half-life extending polymer that is a PEG of the following formula 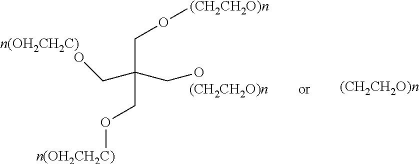
having a molecular weight of about 5 to 20 kDa, preferably about 5 kDa or about 10 kDa or about 20 kDa; or (b.) wherein and wherein X2 is K; an acetamide-thioether having the formula 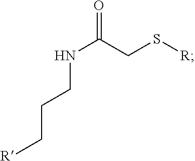
and a half-life extending polymer that is a PEG of the following formula 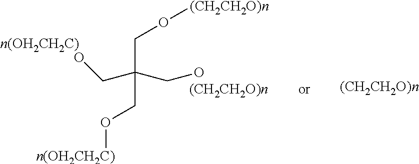
having a molecular weight of about 5 to 20 kDa, preferably about 5 kDa or about 10 kDa or about 20 kDa, wherein R′ stands for the binding peptide and R stands for the half-life extending PEG polymer.

35. The method of claims 31 to 34, wherein the T cell engager masking molecule comprises:
or preferably consists of
(a) the amino acid sequence of SEQ ID 304, 306, 384 or 389, and
a linker chosen from maleimide-thiosuccinimide linker having the formula 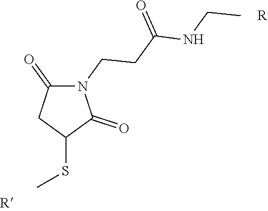
and a disulfide having the formula R′—S—S—R;
and a half-life extending polymer that is a PEG of the following formula 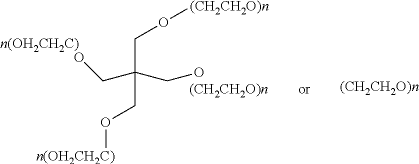
having a molecular weight of about 5 kDa to about 20 kDa, preferably about 5 kDa or about 10 kDa or about 20 kDa; or
(b) the amino acid sequence of SEQ ID 305 or 307:
an acetamide-thioether having the formula 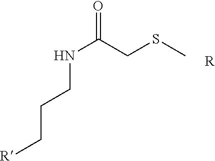
and a half-life extending polymer that is a PEG of the following formula 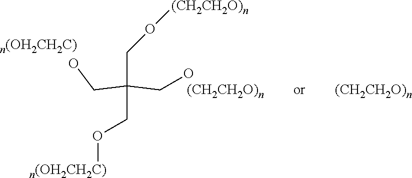
having a molecular weight of about 5 kDa to about 20 kDa preferably about 5 kDa or about 10 kDa or about 20 kDa, wherein R′ stands for the binding peptide and R stands for the half-life extending PEG polymer.

36. The method of any one of claims 31 to 35, wherein the immunotherapy comprises administering a T cell engager.

37. The method of any one of claims 31-36, wherein the molar ratio of the TCE masking molecule to the T cell engager is in the range of about 1000 about 1 kDa 250:1 to about 10:1, or about 100:1 to about 25:1.

38. The method of any of claims 31 to 37, wherein the TCE masking molecule is administered before, during or after administration of a TCE.

39. The method of claim 31 to 38, wherein the TCE is:
(i) A TCE comprising at least a first, a second, and a third domain in an amino to carboxyl order, wherein:
the first domain binds to a target cell surface antigen, wherein the antigen is preferably a tumor antigen:
the second domain binds to an epitope of CD3ε; and
the third domain comprises two polypeptide monomers, each comprising a hinge, a CH2, and a CH3 domain, wherein the two polypeptide monomers are fused to each other via a peptide linker, and wherein the third domain comprises in an amino to carboxyl order: hinge-CH2-CH3-linker-hinge-CH2-CH3;
(ii) A TCE comprising:
a first binding domain, wherein the first binding domain comprises an scFv, scFab, or Fab which binds to a first target cell surface antigen (TAA1);
a second binding domain wherein the second binding domain comprises an scFv. scFab, or Fab which binds to an extracellular epitope on the human CD3ε chain;
a spacer, wherein said spacer is a single chain fragment crystallizable (scFe), human serum albumin (HSA), programmed death receptor 1 (PD1), or a hetero fragment crystallizable (hetero FC):
a third binding domain wherein the third binding domain comprises an scFv, scFab, or Fab which binds to a second target cell surface antigen (TAA2); and
a fourth binding domain wherein the fourth binding domain comprises an scFv, scFab, or Fab which binds to an epitope of CD3ε,
wherein the first binding domain binds to the first target cell surface antigen and the third binding domain binds to the second target cell surface antigen simultaneously, wherein the first target cell surface antigen and the second target cell surface antigen are on the same target cell,
wherein the TCE is preferably a single polypeptide chain,
wherein the first target cell surface antigen and the second target cell surface antigen are not identical, and
wherein the first binding domain and the second binding domain form a first bispecific entity and the third and the fourth binding domain form a second bispecific entity, and
wherein the spacer entity is positioned between the first and the second bispecific entities:
(iii) an IgG-based bispecific antibody; or (iv) a heterodimeric antibody.

40. The method of claim 39, wherein the TCE under (i.) of claim 39 is a single chain molecule.

41. The method of claim 39 or 40, wherein a glycosylation site at Kabat position 314 of the CH2 domains in the third domain of the TCE under (i.) and (ii.) of claim 39 is removed by a N314X substitution, wherein X is any amino acid excluding Q.

42. The method of any one of claims 39 to 41, wherein each of the polypeptide monomers of the third domain comprises an amino acid sequence that is at least 90% identical to an amino acid sequence selected from the group consisting of: SEQ ID NOs: 437 to 444, or comprises the amino acid sequence selected from the group consisting of SEQ ID NOs: 437 to 444.

43. The method of any one of claims 39 to 42, wherein the CH2 domain comprises an intra domain cysteine disulfide bridge.

44. The method of any one of claims 39 to 43, wherein the tumor antigen is CDH19, CDH3, MSLN, DLL3, FLT3, EGFRvIII, BCMA, PSMA, CD33, CD19, CD20, CLDN18.2, CLDN6, MUC17, EpCAM, STEAP1, or CD70.

45. The method of any one of claims 39-44, wherein the TCE under (i.) in claim 39 comprises in an amino to carboxyl order:
(a) a first domain;
(b) a peptide linker having an amino acid sequence of any one of SEQ ID NOs: 187-189;
(c) a second domain;
(d) a peptide linker having an amino acid sequence of any one of SEQ ID NOs: 187, 188, 189, 195, 196, 197 and 198;
(e) a first peptide monomer of the third domain having an amino acid sequence of any of SEQ ID NOs 437 to 444;
(f) a peptide linker having an amino acid sequence of any one of SEQ ID NOs: 191, 192, 193 and 194; and
(g) a second peptide monomer of the third domain having an amino acid sequence of any of SEQ ID NOs 437 to 444.

46. The method of any of claims 39 to 45, wherein the first and/or third binding domain of the TCE under (ii.) in claim 39 comprises a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 1, CDR-L2 as depicted in SEQ ID NO: 2 and CDR-L3 as depicted in SEQ ID NO: 3;
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36;
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47;
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58;
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70;
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88;
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99;
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111;
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120;
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131;
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142;
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157;
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172,;
(n) CDR-H1 as depicted in SEQ ID NO: 203, CDR-H2 as depicted in SEQ ID NO: 204, CDR-H3 as depicted in SEQ ID NO: 205, CDR-L1 as depicted in SEQ ID NO: 206, CDR-L2 as depicted in SEQ ID NO: 207 and CDR-L3 as depicted in SEQ ID NO: 208;
(o) CDR-H1 as depicted in SEQ ID NO: 214, CDR-H2 as depicted in SEQ ID NO: 215, CDR-H3 as depicted in SEQ ID NO: 216, CDR-L1 as depicted in SEQ ID NO: 217, CDR-L2 as depicted in SEQ ID NO: 218 and CDR-L3 as depicted in SEQ ID NO: 219;
(p) CDR-H1 as depicted in SEQ ID NO: 226, CDR-H2 as depicted in SEQ ID NO: 227, CDR-H3 as depicted in SEQ ID NO: 228, CDR-L1 as depicted in SEQ ID NO: 229, CDR-L2 as depicted in SEQ ID NO: 230 and CDR-L3 as depicted in SEQ ID NO: 231;
(q) CDR-H1 as depicted in SEQ ID NO: 238, CDR-H2 as depicted in SEQ ID NO: 239, CDR-H3 as depicted in SEQ ID NO: 240, CDR-L1 as depicted in SEQ ID NO: 241, CDR-L2 as depicted in SEQ ID NO: 242 and CDR-L3 as depicted in SEQ ID NO: 243; and
(r) CDR-H1 as depicted in SEQ ID NO: 420, CDR-H2 as depicted in SEQ ID NO: 421, CDR-H3 as depicted in SEQ ID NO: 422, CDR-L1 as depicted in SEQ ID NO: 423, CDR-L2 as depicted in SEQ ID NO: 424 and CDR-L3 as depicted in SEQ ID NO: 425.

47. The method of any one of claims 39 to 46, wherein the TCE comprises the amino acid sequence of SEQ ID NO: 17, 52, 63, 81, 93, 104, 114, 125, 136, 147, 162, 177, 213, 226, 238, 248, 255 or 430, preferably 104 or 255.

48. The method of claim 39, wherein the heterodimeric antibody comprises:
(a) a first monomer comprising a first heavy chain comprising:
(i) a first variable heavy domain;
(ii) a first constant heavy chain comprising a first CH1 domain and a first Fc domain; (iii) a scFv that binds CD3ε and comprises a scFv variable light domain, an scFv linker, and a scFv variable heavy domain wherein the scFv is covalently attached between the C-terminus of the CH1 domain and the N-terminus of the first Fc domain using domain linker(s);
(b) a second monomer comprising a second heavy chain comprising a second variable heavy domain and a second constant heavy chain comprising a second Fc domain; and
(c) a common light chain comprising a variable light domain and a constant light domain. wherein the first variable heavy domain and the variable light domain bind human STEAP1, and
wherein the second variable heavy domain and the variable light domain bind human STEAP1, and wherein
(1) the first monomer comprises the amino acid sequence of SEQ ID NO: 375, the second monomer comprises the sequence of SEQ ID NO: 374, and the common light chain comprises the sequence of SEQ ID NO: 373;
(2) the first monomer comprises the sequence of SEQ ID NO: 380, the second monomer comprises the sequence of SEQ ID NO: 376, and the common light chain comprises the sequence of SEQ ID NO: 373; or
(3) the first monomer comprises the sequence of SEQ ID NO: 379, the second monomer comprises the sequence of SEQ ID NO: 378, and the common light chain comprises the sequence of SEQ ID NO: 377.

49. The TCE masking molecule or the method of any of the preceding claims, wherein the CD3ε binding domain has 80, 90, 95 or 99% sequence identity with the sequence of SEQ ID NO 26, 381, 382 or 383 or has the sequence of SEQ ID NO 26, 381, 382 or 383

50. A TCE masking molecule for use in reducing the severity of cytokine release in a human subject undergoing treatment with a TCE, wherein the TCE masking molecule comprises:
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii) a linker covalently linked to the C-terminus of the binding peptide; and
(iii) a half-life extending polymer covalently linked to the linker.

51. A method of attenuating in vivo exposure of a TCE after administration of the TCE, wherein the administration is preferably via the i.v. or s.c. route, the method comprising the steps of
(a) providing a TCE masking molecule, comprising
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257,
(ii) a linker covalently linked to the C-terminus of the binding peptide; and
(iii) a half-life extending polymer covalently linked to the linker, wherein the binding peptide binds to an anti-CD3ε paratope with a Kd which is at least 1.5-fold lower than the Kd for the amino acid sequence of SEQ ID NO: 262 binding to an anti-CD3ε paratope, preferably, at least 2-fold lower, more preferably 3-fold, lower, and
(b) administering the TCE masking molecule at a molar excess of about 10 to about 1000, or about 25 to about 250 with respect to the TCE.

52. The method of claim 51, wherein the TCE masking molecule is administered before, during or after the administration of the TCE.

53. The method of claim 51 or 52, wherein the T cell engager masking molecule comprises:
a binding peptide which binds to an anti-CD3ε paratope and comprises the sequence of pEX1X2X3EELKGX4 (SEQ ID NO: 434), wherein X1 is D, H or N, and wherein X2 is G, F or Y, preferably G or F, and wherein X3 is N, H, Q, R, E, S, T, V, C, D, F, K L, M, W, Y or I, preferably N, E, H, Q or R, and (a.) wherein and wherein X4 is C; a linker chosen from maleimide-thiosuccinimide linker having the formula 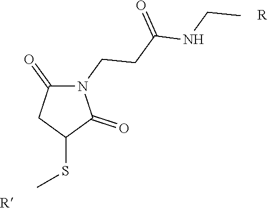
and a disulfide having the formula R′—S—S—R; and a half-life extending polymer that is a PEG of the following formula 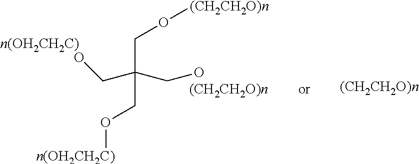
having a molecular weight of about 5 to 20 kDa, preferably about 5 kDa or about 10 kDa or about 20 kDa; or (b.) wherein and wherein X4 is K; an acetamide-thioether having the formula 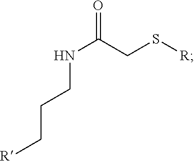
and a half-life extending polymer that is a PEG of the following formula 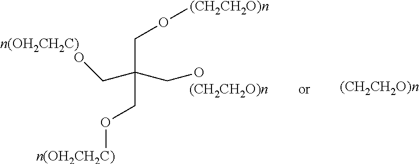
having a molecular weight of about 5 to 20 kDa, preferably about 5 kDa or about 10 kDa or about 20 kDa, wherein R′ stands for the binding peptide and R stands for the half-life extending PEG polymer.

54. The method of claims 51 to 53, wherein the T cell engager masking molecule comprises:
a binding peptide which binds to an anti-CD3ε paratope and comprises the sequence of pEX1GX2EELKGX3 (SEQ ID NO: 435), wherein X1 is D, H or N, and wherein X2 is N, H, Q, R, E, S, T, V, C, D, F, K L, M, W, Y or I, preferably N, H, Q or R, and (a.) wherein and wherein X3 is C; a linker chosen from maleimide-thiosuccinimide linker having the formula 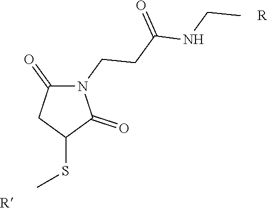
and a disulfide having the formula R′—S—S—R; and a half-life extending polymer that is a PEG of the following formula 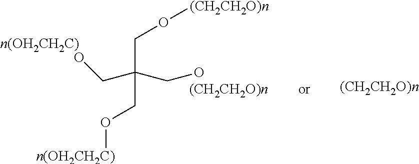
having a molecular weight of about 5 to 20 kDa, preferably about 5 kDa or about 10 kDa or about 20 kDa; or (b.) wherein and wherein X3 is K; an acetamide-thioether having the formula 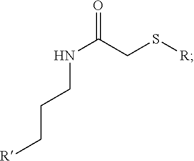
and a half-life extending polymer that is a PEG of the following formula 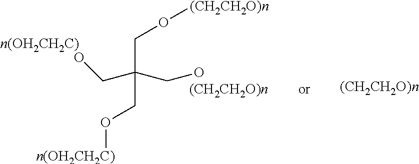
having a molecular weight of about 5 to 20 kDa, preferably about 5 kDa or about 10 kDa or about 20 kDa, wherein R′ stands for the binding peptide and R stands for the half-life extending PEG polymer.

55. The method of claims 51 to 54, wherein the T cell engager masking molecule comprises:
a binding peptide which binds to an anti-CD3ε paratope and comprises the sequence of pEDGX1EELKGX2 (SEQ ID NO: 436), wherein X1 is D, H or N, and wherein X1 is N, H, Q, R, E, S, T, V, C, D, F, K L, M, W, Y or I, preferably N, E, H, Q or R, most preferably N or E, and (a.) wherein and wherein X2 is C; a linker chosen from maleimide-thiosuccinimide linker having the formula 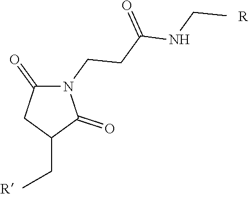
and a disulfide having the formula R′—S—S—R; and a half-life extending polymer that is a PEG of the following formula 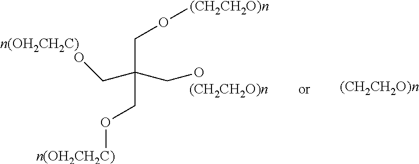
having a molecular weight of about 5 to 20 kDa, preferably about 5 kDa or about 10 kDa or about 20 kDa; or (b.) wherein and wherein X2 is K; an acetamide-thioether having the formula 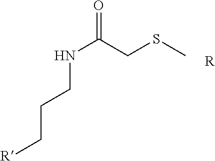
and a half-life extending polymer that is a PEG of the following formula 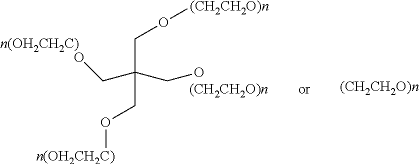
having a molecular weight of about 5 to 20 kDa, preferably about 5 kDa or about 10 kDa or about 20 kDa, wherein R′ stands for the binding peptide and R stands for the half-life extending PEG polymer.

56. The method of claims 51 to 55, wherein the T cell engager masking molecule comprises:
(a) the amino acid sequence of SEQ ID 304, 306, 384 or 389, and
a linker chosen from maleimide-thiosuccinimide linker having the formula 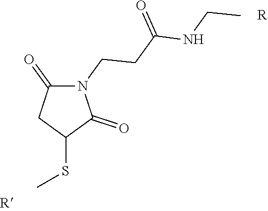
and a disulfide having the formula R′—S—S—R;
and a half-life extending polymer that is a PEG of the following formula 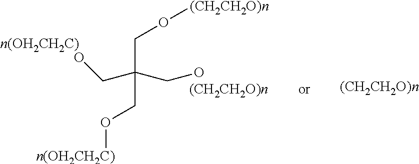
having a molecular weight of about 5 kDa to about 20 kDa, preferably about 5 kDa or about 10 kDa or about 20 kDa; or
(b) the amino acid sequence of SEQ ID 305 or 307;
an acetamide-thioether having the formula 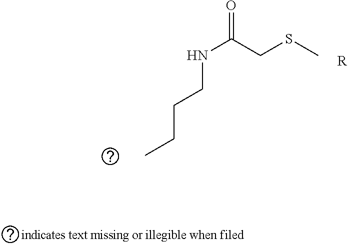
and a half-life extending polymer that is a PEG of the following formula 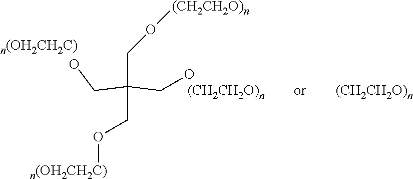
having a molecular weight of about 5 kDa to about 20 kDa, preferably about 5 kDa or about 10 kDa or about 20 kDa, wherein R′ stands for the binding peptide and R stands for the half-life extending PEG polymer.

57. The method of any one of claims 51 to 56, wherein the immunotherapy comprises administering a T cell engager.

58. The method of any one of claims 51-57, wherein the molar ratio of the TCE masking molecule to the TCE is in the range of about 1000 to about 1, or about 250:1 to about 10:1, or about 100:1 to about 25:1.

59. The method of any of claims 51 to 58, wherein the TCE masking molecule is administered before, during or after administration of a TCE.

60. The method of claim 51 to 58, wherein the TCE is:
(i) A TCE comprising at least a first, a second, and a third domain in an amino to carboxyl order, wherein:
the first domain binds to a target cell surface antigen, wherein the antigen is preferably a tumor antigen;
the second domain binds to an epitope of CD3ε; and
the third domain comprises two polypeptide monomers, each comprising a hinge, a CH2, and a CH3 domain, wherein the two polypeptide monomers are fused to each other via a peptide linker, and wherein the third domain comprises in an amino to carboxyl order: hinge-CH2-CH3-linker-hinge-CH2-CH3;
(ii) A TCE comprising:
a first binding domain, wherein the first binding domain comprises an scFv, scFab, or Fab which binds to a first target cell surface antigen (TAA1):
a second binding domain wherein the second binding domain comprises an scFv. scFab, or Fab which binds to an extracellular epitope on the human CD3ε chain;
a spacer, wherein said spacer is a single chain fragment crystallizable (scFc), human serum albumin (HSA), programmed death receptor 1 (PD1), or a hetero fragment crystallizable (hetero FC);
a third binding domain wherein the third binding domain comprises an scFv, scFab, or Fab which binds to a second target cell surface antigen (TAA2); and
a fourth binding domain wherein the fourth binding domain comprises an scFv, scFab, or Fab which binds to an epitope of CD3ε,
wherein the first binding domain binds to the first target cell surface antigen and the third binding domain binds to the second target cell surface antigen simultaneously, wherein the first target cell surface antigen and the second target cell surface antigen are on the same target cell,
wherein the TCE is a preferably a single polypeptide chain;
wherein the first target cell surface antigen and the second target cell surface antigen are not identical, and
wherein the first binding domain and the second binding domain form a first bispecific entity and the third and the fourth binding domain form a second bispecific entity, and
wherein
the spacer entity is positioned between the first and the second bispecific entities;
(iii) an IgG-based bispecific antibody; or
(iv) a heterodimeric antibody.

61. The method of claim 60, wherein the TCE (i.) of claim 60 is a single chain molecule.

62. The method of claim 60 or 61, wherein a glycosylation site at Kabat position 314 of the CH2 domains in the third domain of the TCE is removed by a N314X substitution, wherein X is any amino acid excluding Q.

63. The method of any one of claims 60 to 62, wherein each of the polypeptide monomers of the third domain comprises an amino acid sequence that is at least 90% identical to an amino acid sequence selected from the group consisting of: SEQ ID NOs: 437 to 444, or comprises the amino acid sequence selected from the group consisting of SEQ ID NOs: 437 to 444.

64. The method of any one of claims 60 to 63, wherein the CH2 domain comprises an intra domain cysteine disulfide bridge.

65. The method of any one of claims 60 to 64, wherein the tumor antigen is CDH19, CDH3, MSLN, DLL3, FLT3, EGFRvIII, BCMA, PSMA, CD33, CD19, CD20, CLDN18.2, CLDN6, MUC17, EpCAM, STEAP1, or CD70.

66. The method of any one of claims 60 to 65, wherein the TCE under (i.) in claim 59 comprises in an amino to carboxyl order:
(a) a first domain:
(b) a peptide linker having an amino acid sequence of any one of SEQ ID NOs: 187-189;
€ a second domain;
(d) a peptide linker having an amino acid sequence of any one of SEQ ID NOs: 187, 188, 189, 195, 196, 197 and 198;
(e) a first peptide monomer of the third domain having an amino acid sequence of any of SEQ ID NOs 437 to 444;
(f) a peptide linker having an amino acid sequence of any one of SEQ ID NOs: 191, 192, 193 and 194; and
(g) a second peptide monomer of the third domain having an amino acid sequence of any of SEQ ID NOs 437 to 444.

67. The method of any of claims 60 to 66, wherein the first and/or third binding domain of the TCE under (ii.) in claim 39 comprises a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 1, CDR-L2 as depicted in SEQ ID NO: 2 and CDR-L3 as depicted in SEQ ID NO: 3;
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36;
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47;
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58;
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70;
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88;
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99;
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111;
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120;
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131;
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142;
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157;
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172;
(n) CDR-H1 as depicted in SEQ ID NO: 203, CDR-H2 as depicted in SEQ ID NO: 204, CDR-H3 as depicted in SEQ ID NO: 205, CDR-L1 as depicted in SEQ ID NO: 206, CDR-L2 as depicted in SEQ ID NO: 207 and CDR-L3 as depicted in SEQ ID NO: 208;
(o) CDR-H1 as depicted in SEQ ID NO: 214, CDR-H2 as depicted in SEQ ID NO: 215, CDR-H3 as depicted in SEQ ID NO: 216, CDR-L1 as depicted in SEQ ID NO: 217, CDR-L2 as depicted in SEQ ID NO: 218 and CDR-L3 as depicted in SEQ ID NO: 219;
(p) CDR-H1 as depicted in SEQ ID NO: 226, CDR-H2 as depicted in SEQ ID NO: 227, CDR-H3 as depicted in SEQ ID NO: 228, CDR-L1 as depicted in SEQ ID NO: 229, CDR-L2 as depicted in SEQ ID NO: 230 and CDR-L3 as depicted in SEQ ID NO: 231;
(q) CDR-H1 as depicted in SEQ ID NO: 238, CDR-H2 as depicted in SEQ ID NO: 239, CDR-H3 as depicted in SEQ ID NO: 240, CDR-L1 as depicted in SEQ ID NO: 241, CDR-L2 as depicted in SEQ ID NO: 242 and CDR-L3 as depicted in SEQ ID NO: 243; and
(r) CDR-H1 as depicted in SEQ ID NO: 420, CDR-H2 as depicted in SEQ ID NO: 421, CDR-H3 as depicted in SEQ ID NO: 422, CDR-L1 as depicted in SEQ ID NO: 423, CDR-L2 as depicted in SEQ ID NO: 424 and CDR-L3 as depicted in SEQ ID NO: 425.

68. The method of any one of claims 60 to 67, wherein the TCE comprises the amino acid sequence of SEQ ID NO: 17, 52, 63, 81, 93, 104, 114, 125, 136, 147, 162, 177, 213, 226, 238, 248, 255 or 430, preferably 104 or 255.

69. The method of claim 60, wherein the heterodimeric antibody comprises:
(a) a first monomer comprising a first heavy chain comprising:
(i) a first variable heavy domain;
(ii) a first constant heavy chain comprising a first CH1 domain and a first Fc domain; (iii) a scFv that binds CD3ε and comprises a scFv variable light domain, an scFv linker, and a scFv variable heavy domain
wherein the scFv is covalently attached between the C-terminus of the CH1 domain and the N-terminus of the first Fc domain using domain linker(s);
(b) a second monomer comprising a second heavy chain comprising a second variable heavy domain and a second constant heavy chain comprising a second Fc domain; and
(c) a common light chain comprising a variable light domain and a constant light domain. wherein the first variable heavy domain and the variable light domain bind human STEAP1, and
wherein the second variable heavy domain and the variable light domain bind human STEAP1, and wherein
(1) the first monomer comprises the amino acid sequence of SEQ ID NO: 375, the second monomer comprises the sequence of SEQ ID NO: 374, and the common light chain
comprises the sequence of SEQ ID NO: 373;
(2) the first monomer comprises the sequence of SEQ ID NO: 380, the second monomer comprises the sequence of SEQ ID NO: 376, and the common light chain comprises the sequence of SEQ ID NO: 373; or
(3) the first monomer comprises the sequence of SEQ ID NO: 379, the second monomer comprises the sequence of SEQ ID NO: 378, and the common light chain comprises the sequence of SEQ ID NO: 377.

70. The TCE masking molecule or the method of any of the preceding claims, wherein the CD3ε binding domain has 80, 90, 95 or 99% sequence identity with the sequence of SEQ ID NO 26, 381, 382 or 383 or has the sequence of SEQ ID NO 26, 381, 382 or 383.

71. A TCE masking molecule for use in the attenuation of in vivo exposure of a TCE after administration of the TCE molecule, wherein the administration is preferably via the i.v. or s.c. route, the method comprising the steps of
(a) providing a TCE masking molecule, comprising
(i.) a binding peptide that binds to an anti-CD3ε paratope of a CD3ε binding domain of a TCE, wherein the anti-CD3ε paratope is directed against an epitope located within CD3ε, and wherein CD3ε comprises the amino acid sequence of SEQ ID NO: 257;
(ii) a linker covalently linked to the C-terminus of the binding peptide; and
(iii) a half-life extending polymer covalently linked to the linker,
wherein the binding peptide binds to an anti-CD3ε paratope with a Kd which is at least 1.5-fold lower than the Kd for the amino acid sequence of SEQ ID NO: 262 binding to an anti-CD3ε paratope, preferably, at least 2-fold lower, more preferably 3-fold lower, and
(b) administering the TCE masking molecule before, during or after the administration of the TCE at a molar excess of about 10 to about 1000, or about 25 to about 250 with respect to the TCE.


SEQUENCE LISTING (444 sequences)

SEQ ID NO: 1 - protein, 15 aa, synthetic construct
KASQSVDYDGDSYLN

SEQ ID NO: 2 - protein, 7 aa, synthetic construct
DASNLVS

SEQ ID NO: 3 - protein, 9 aa, synthetic construct
QQSTEDPWT

SEQ ID NO: 4 - protein, 5 aa, synthetic construct
SYWMN

SEQ ID NO: 5 - protein, 17 aa, synthetic construct
QIWPGDGDTNYNGKFKG

SEQ ID NO: 6 - protein, 15 aa, synthetic construct
RETTTVGRYYYAMDY

SEQ ID NO: 7 - protein, 111 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 8 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 9 - protein, 5 aa, synthetic construct
RYTMH

SEQ ID NO: 10 - protein, 17 aa, synthetic construct
YINPSRGYTNYNQKFKD

SEQ ID NO: 11 - protein, 10 aa, synthetic construct
YYDDHYCLDY

SEQ ID NO: 12 - protein, 10 aa, synthetic construct
RASSSVSYMN

SEQ ID NO: 13 - protein, 7 aa, synthetic construct
DTSKVAS

SEQ ID NO: 14 - protein, 9 aa, synthetic construct
QQWSSNPLT

SEQ ID NO: 15 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 16 - protein, 108 aa, synthetic construct
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 504 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 18 - protein, 14 aa, synthetic construct
GSSTGAVTSGNYPN

SEQ ID NO: 19 - protein, 7 aa, synthetic construct
GTKFLAP

SEQ ID NO: 20 - protein, 9 aa, synthetic construct
VLWYSNRWV

SEQ ID NO: 21 - protein, 5 aa, synthetic construct
KYAMN

SEQ ID NO: 22 - protein, 19 aa, synthetic construct
RIRSKYNNYATYYADSVKD

SEQ ID NO: 23 - protein, 14 aa, synthetic construct
HGNFGNSYISYWAY

SEQ ID NO: 24 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 25 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 26 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 27 - protein, 122 aa, synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 28 - protein, 122 aa, synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 29 - protein, 5 aa, synthetic construct
NYGMN

SEQ ID NO: 30 - protein, 17 aa, synthetic construct
WINTYTGEPTYADKFQG

SEQ ID NO: 31 - protein, 13 aa, synthetic construct
WSWSDGYYVYFDY

SEQ ID NO: 32 - protein, 113 aa, synthetic construct
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 33 - protein, 113 aa, synthetic construct
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 34 - protein, 17 aa, synthetic construct
KSSQSVLDSSTNKNSLA

SEQ ID NO: 35 - protein, 7 aa, synthetic construct
WASTRES

SEQ ID NO: 36 - protein, 9 aa, synthetic construct
QQSAHFPIT

SEQ ID NO: 37 - protein, 250 aa, synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 38 - protein, 250 aa, synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 39 - protein, 505 aa, synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 530 aa, synthetic construct
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 41 - protein, 993 aa, synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 5 aa, synthetic construct
NYGMH

SEQ ID NO: 43 - protein, 17 aa, synthetic construct
VIWYDGSDKYYADSVRG

SEQ ID NO: 44 - protein, 15 aa, synthetic construct
DGYDILTGNPRDFDY

SEQ ID NO: 45 - protein, 16 aa, synthetic construct
RSSQSLVHSDGNTYLS

SEQ ID NO: 46 - protein, 7 aa, synthetic construct
RISRRFS

SEQ ID NO: 47 - protein, 9 aa, synthetic construct
MQSTHVPRT

SEQ ID NO: 48 - protein, 124 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 49 - protein, 112 aa, synthetic construct
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 50 - protein, 251 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 51 - protein, 506 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 994 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 5 aa, synthetic construct
DYYMT

SEQ ID NO: 54 - protein, 17 aa, synthetic construct
YISSSGSTIYYADSVKG

SEQ ID NO: 55 - protein, 8 aa, synthetic construct
DRNSHFDY

SEQ ID NO: 56 - protein, 11 aa, synthetic construct
RASQGINTWLA

SEQ ID NO: 57 - protein, 7 aa, synthetic construct
GASGLQS

SEQ ID NO: 58 - protein, 9 aa, synthetic construct
QQAKSFPRT

SEQ ID NO: 59 - protein, 117 aa, synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 60 - protein, 107 aa, synthetic construct
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 61 - protein, 239 aa, synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 62 - protein, 494 aa, synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 63 - protein, 982 aa, synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 982 aa, synthetic construct
QVQLVESGGGLVKPGGSLRLSCAASGFTFSDHYMSWIRQAPGKCLEWFSYISSSGGIIYYADSVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 5 aa, synthetic construct
SYGMH

SEQ ID NO: 66 - protein, 17 aa, synthetic construct
FIWYEGSNKYYAESVKD

SEQ ID NO: 67 - protein, 16 aa, synthetic construct
RAGIIGTIGYYYGMDV

SEQ ID NO: 68 - protein, 11 aa, synthetic construct
SGDRLGEKYTS

SEQ ID NO: 69 - protein, 7 aa, synthetic construct
QDTKRPS

SEQ ID NO: 70 - protein, 9 aa, synthetic construct
QAWESSTVV

SEQ ID NO: 71 - protein, 125 aa, synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 72 - protein, 107 aa, synthetic construct
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 73 - protein, 247 aa, synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 74 - protein, 507 aa, synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 75 - protein, 989 aa, synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 987 aa, synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 125 aa, synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSS

SEQ ID NO: 78 - protein, 106 aa, synthetic construct
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 79 - protein, 246 aa, synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 80 - protein, 501 aa, synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 989 aa, synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 987 aa, synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 7 aa, synthetic construct
NARMGVS

SEQ ID NO: 84 - protein, 16 aa, synthetic construct
HIFSNDEKSYSTSLKN

SEQ ID NO: 85 - protein, 14 aa, synthetic construct
IVGYGSGWYGFFDY

SEQ ID NO: 86 - protein, 11 aa, synthetic construct
RASQGIRNDLG

SEQ ID NO: 87 - protein, 7 aa, synthetic construct
AASTLQS

SEQ ID NO: 88 - protein, 9 aa, synthetic construct
LQHNSYPLT

SEQ ID NO: 89 - protein, 124 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 90 - protein, 107 aa, synthetic construct
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 91 - protein, 246 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 92 - protein, 501 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 989 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 5 aa, synthetic construct
SYYWS

SEQ ID NO: 95 - protein, 16 aa, synthetic construct
YVYYSGTTNYNPSLKS

SEQ ID NO: 96 - protein, 10 aa, synthetic construct
IAVTGFYFDY

SEQ ID NO: 97 - protein, 12 aa, synthetic construct
RASQRVNNNYLA

SEQ ID NO: 98 - protein, 7 aa, synthetic construct
GASSRAT

SEQ ID NO: 99 - protein, 9 aa, synthetic construct
QQYDRSPLT

SEQ ID NO: 100 - protein, 118 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 101 - protein, 108 aa, synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 102 - protein, 241 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 103 - protein, 496 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 982 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 5 aa, synthetic construct
SYGMH

SEQ ID NO: 106 - protein, 17 aa, synthetic construct
VISYEGSNKYYAESVKG

SEQ ID NO: 107 - protein, 13 aa, synthetic construct
DRGTIFGNYGLEV

SEQ ID NO: 108 - protein, 122 aa, synthetic construct
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 109 - protein, 16 aa, synthetic construct
RSSQSLLHKNAFNYLD

SEQ ID NO: 110 - protein, 7 aa, synthetic construct
LGSNRAS

SEQ ID NO: 111 - protein, 9 aa, synthetic construct
MQALQTPFT

SEQ ID NO: 112 - protein, 112 aa, synthetic construct
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 113 - protein, 525 aa, synthetic construct
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 1013 aa, synthetic construct
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 5 aa, synthetic construct
SYPIN

SEQ ID NO: 116 - protein, 16 aa, synthetic construct
VIWTGGGTNYASSVKG

SEQ ID NO: 117 - protein, 15 aa, synthetic construct
SRGVYDFDGRGAMDY

SEQ ID NO: 118 - protein, 17 aa, synthetic construct
KSSQSLLYSSNQKNYFA

SEQ ID NO: 119 - protein, 7 aa, synthetic construct
WASTRES

SEQ ID NO: 120 - protein, 9 aa, synthetic construct
QQYYSYPYT

SEQ ID NO: 121 - protein, 123 aa, synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSS

SEQ ID NO: 122 - protein, 113 aa, synthetic construct
DIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 123 - protein, 251 aa, synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 124 - protein, 506 aa, synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 125 - protein, 994 aa, synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 5 aa, synthetic construct
NHIIH

SEQ ID NO: 127 - protein, 17 aa, synthetic construct
YINPYPGYHAYNEKFQG

SEQ ID NO: 128 - protein, 12 aa, synthetic construct
DGYYRDTDVLDY

SEQ ID NO: 129 - protein, 11 aa, synthetic construct
QASQDISNYLN

SEQ ID NO: 130 - protein, 7 aa, synthetic construct
YTSRLHT

SEQ ID NO: 131 - protein, 9 aa, synthetic construct
QQGNTLPWT

SEQ ID NO: 132 - protein, 121 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 133 - protein, 107 aa, synthetic construct
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 134 - protein, 243 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 135 - protein, 498 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 986 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 5 aa, synthetic construct
DYYMY

SEQ ID NO: 138 - protein, 17 aa, synthetic construct
IISDAGYYTYYSDIIKG

SEQ ID NO: 139 - protein, 12 aa, synthetic construct
GFPLLRHGAMDY

SEQ ID NO: 140 - protein, 11 aa, synthetic construct
KASQNVDANVA

SEQ ID NO: 141 - protein, 7 aa, synthetic construct
SASYVYW

SEQ ID NO: 142 - protein, 9 aa, synthetic construct
QQYDQQLIT

SEQ ID NO: 143 - protein, 121 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 144 - protein, 107 aa, synthetic construct
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 145 - protein, 243 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 146 - protein, 498 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 986 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 984 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 498 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 150 - protein, 986 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 984 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 5 aa, synthetic construct
DYYMY

SEQ ID NO: 153 - protein, 17 aa, synthetic construct
IISDGGYYTYYSDIIKG

SEQ ID NO: 154 - protein, 12 aa, synthetic construct
GFPLLRHGAMDY

SEQ ID NO: 155 - protein, 11 aa, synthetic construct
KASQNVDTNVA

SEQ ID NO: 156 - protein, 7 aa, synthetic construct
SASYVYW

SEQ ID NO: 157 - protein, 9 aa, synthetic construct
QQYDQQLIT

SEQ ID NO: 158 - protein, 121 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 159 - protein, 107 aa, synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 160 - protein, 243 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 161 - protein, 498 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 162 - protein, 986 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 984 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 498 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 165 - protein, 986 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 984 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 5 aa, synthetic construct
DYYMY

SEQ ID NO: 168 - protein, 17 aa, synthetic construct
IISDGGYYTYYSDIIKG

SEQ ID NO: 169 - protein, 12 aa, synthetic construct
GFPLLRHGAMDY

SEQ ID NO: 170 - protein, 11 aa, synthetic construct
KASQNVDTNVA

SEQ ID NO: 171 - protein, 7 aa, synthetic construct
SASYVYW

SEQ ID NO: 172 - protein, 9 aa, synthetic construct
QQYDQQLIT

SEQ ID NO: 173 - protein, 121 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 174 - protein, 107 aa, synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 175 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 176 - protein, 498 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 177 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 984 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 498 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 180 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 984 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 10 aa, synthetic construct
DKTHTCPPCP

SEQ ID NO: 183 - protein, 12 aa, synthetic construct
ERKCCVECPPCP

SEQ ID NO: 184 - protein, 16 aa, synthetic construct
ELKTPLDTTHTCPRCP

SEQ ID NO: 185 - protein, 17 aa, synthetic construct
ELKTPLGDTTHTCPRCP

SEQ ID NO: 186 - protein, 12 aa, synthetic construct
ESKYGPPCPSCP

SEQ ID NO: 187 - protein, 5 aa, synthetic construct
GGGGS

SEQ ID NO: 188 - protein, 10 aa, synthetic construct
GGGGSGGGGS

SEQ ID NO: 189 - protein, 15 aa, synthetic construct
GGGGSGGGGSGGGGS

SEQ ID NO: 190 - protein, 20 aa, synthetic construct
GGGGSGGGGSGGGGSGGGGS

SEQ ID NO: 191 - protein, 25 aa, synthetic construct
GGGGSGGGGSGGGGSGGGGSGGGGS

SEQ ID NO: 192 - protein, 30 aa, synthetic construct
GGGGSGGGGSGGGGSGGGGSGGGGSGGGGS

SEQ ID NO: 193 - protein, 35 aa, synthetic construct
GGGGSGGGGSGGGGSGGGGSGGGGSGGGGSGGGGS

SEQ ID NO: 194 - protein, 40 aa, synthetic construct
GGGGSGGGGSGGGGSGGGGSGGGGSGGGGSGGGGSGGGGS

SEQ ID NO: 195 - protein, 6 aa, synthetic construct
PGGGGS

SEQ ID NO: 196 - protein, 6 aa, synthetic construct
PGGDGS

SEQ ID NO: 197 - protein, 6 aa, synthetic construct
SGGGGS

SEQ ID NO: 198 - protein, 4 aa, synthetic construct
GGGG

SEQ ID NO: 199 - protein, 6 aa, synthetic construct
HHHHHH

SEQ ID NO: 200 - protein, 109 aa, synthetic construct; identical to SEQ ID NO: 25

SEQ ID NO: 201 - protein, 125 aa, synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 202 - protein, 249 aa, synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 203 - protein, 5 aa, synthetic construct
GYYWS

SEQ ID NO: 204 - protein, 16 aa, synthetic construct
DIDASGSTKYNPSLKS

SEQ ID NO: 205 - protein, 12 aa, synthetic construct
KKYSTVWSYFDN

SEQ ID NO: 206 - protein, 11 aa, synthetic construct
SGDKLGDKYAS

SEQ ID NO: 207 - protein, 7 aa, synthetic construct
QDRKRPS

SEQ ID NO: 208 - protein, 9 aa, synthetic construct
QAWGSSTAV

SEQ ID NO: 209 - protein, 120 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 210 - protein, 106 aa, synthetic construct
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 211 - protein, 241 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 212 - protein, 496 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 213 - protein, 984 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 5 aa, synthetic construct
GYYMH

SEQ ID NO: 215 - protein, 17 aa, synthetic construct
WINPNSGGTKYAQKFQG

SEQ ID NO: 216 - protein, 16 aa, synthetic construct
DRITVAGTYYYYGMDV

SEQ ID NO: 217 - protein, 11 aa, synthetic construct
RASQGVNNWLA

SEQ ID NO: 218 - protein, 7 aa, synthetic construct
TASSLQS

SEQ ID NO: 219 - protein, 9 aa, synthetic construct
QQANSFPIT

SEQ ID NO: 220 - protein, 125 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 221 - protein, 107 aa, synthetic construct
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 222 - protein, 247 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 223 - skipped

SEQ ID NO: 224 - protein, 502 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 225 - protein, 508 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 226 - protein, 990 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 5 aa, synthetic construct
GYYMH

SEQ ID NO: 228 - protein, 17 aa, synthetic construct
WINPNSGGTKYAQKFQG

SEQ ID NO: 229 - protein, 16 aa, synthetic construct
DRITVAGTYYYYGMDV

SEQ ID NO: 230 - protein, 11 aa, synthetic construct
RASQGVNNWLA

SEQ ID NO: 231 - protein, 7 aa, synthetic construct
TASSLQS

SEQ ID NO: 232 - protein, 9 aa, synthetic construct
QQANSFPIT

SEQ ID NO: 233 - protein, 125 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 234 - protein, 107 aa, synthetic construct; identical to SEQ ID NO: 221

SEQ ID NO: 235 - protein, 247 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 236 - protein, 502 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 237 - protein, 508 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 238 - protein, 990 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 5 aa, synthetic construct
TYAMS

SEQ ID NO: 240 - protein, 17 aa, synthetic construct
AISGSGGRTFYAESVEG

SEQ ID NO: 241 - protein, 11 aa, synthetic construct
HDYSNYPYFDY

SEQ ID NO: 242 - protein, 12 aa, synthetic construct
RASQSVRSTYLA

SEQ ID NO: 243 - protein, 7 aa, synthetic construct
GASSRAT

SEQ ID NO: 244 - protein, 9 aa, synthetic construct
QQYGDLPFT

SEQ ID NO: 245 - protein, 120 aa, synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 246 - protein, 108 aa, synthetic construct
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 247 - protein, 242 aa, synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEI

SEQ ID NO: 248 - protein, 497 aa, synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTV

SEQ ID NO: 249 - protein, 121 aa, synthetic construct
QVQLQESGPGLVKPSETLSLTCTVSGGSISSSSYFWGWIRQPPGKCLEWIGNIYYSGSSNYNPSLKSRVTISVDTSKNQFSLKLSSVTAADTAVYYCARLPRGDRDAFDIWGQGTMVTVSS

SEQ ID NO: 250 - protein, 107 aa, synthetic construct
DIVMTQSPSSLSASVGDRVTITCRASQGISNYLAWYQQKPGKVPKLLIYAASTLQSGVPSRFSGSGSGTDFTLTISSLQPEDFATYYCQQSYSTPFTFGCGTKVEIK

SEQ ID NO: 251 - protein, 107 aa, synthetic construct
EIVMTQSPSSLSASVGDRVTITCRASQGISNYLAWYQQKPGKVPKLLIYAASTLQSGVPSRFSGSGSGTDFTLTISSLQPEDFATYYCQQSYSTPFTFGCGTKVEIK

SEQ ID NO: 252 - protein, 117 aa, synthetic construct
QVQLVQSGAEVKKPGASVKVSCKASGYTFTNYWMNWVRQAPGQCLEWMGNIAYGVKGTNYNQKFQGRVTMTVDTSSSTAYMELSRLRSDDTAVYYCATRYFYVMDYWGQGTLVTVSS

SEQ ID NO: 253 - protein, 107 aa, synthetic construct
EIQMTQSPSSLSASVGDRVTITCRASQDISNYLNWYQQKPGKVPKLLIYYTSRLHSGVPSRFSGSGSGTDFTLTISSLQPEDVATYYCVQYAQFPLTFGCGTKVEIK

SEQ ID NO: 254 - protein, 117 aa, synthetic construct
QVQLVQSGAEVKKPGASVKVSCKASGYTFTNYWMNWVRQAPGQCLEWMGNIAYGVAGTNYNQKFQGRVTMTVDTSSSTAYMELSRLRSDDTAVYYCATRYFYVMDYWGQGTLVTVSS

SEQ ID NO: 255 - protein, 1474 aa, synthetic construct
QVQLVQSGAEVKKPGASVKVSCKASGYTFTNYWMNWVRQAPGQCLEWMGNIAYGVAGTNYNQKFQGRVTMTVDTSSSTAYMELSRLRSDDTAVYYCATRYFYVMDYWGQGTLVTVSSGGGGQGGGGQGGGGQDIQMTQSPSSLSASVGDRVTITCRASQDISNYLNWYQQKPGKVPKLLIYYTSRLHSGVPSRFSGSGSGTDFTLTISSLQPEDVATYYCVQYAQFPLTFGCGTKVEIKSGGGGQEVQLVESGGGLVQPGGSLKLSCAASGFTFNKYAMNWVRQAPGKGMEWVARIRSKYNNYATYYADAVKDRFTISRDDSKNTLYLQMNNLKTEDTAVYYCVRAGNFGSSYISYFAYWGQGTLVTVSSGGGGQGGGGQGGGGQQTVVTQEPSLTVSPGGTVTITCGSSTGAVTSGNYPNWIQKKPGQAPRGLIGGTKFLAPGTPARFSGSLEGGKAALTLSGVQPEDEAEYYCVLYYSNRWVFGSGTKLTVLGGGGCPPCPAPELLGGPSVFLFPPKPKDTLMISRTPEVTCVVVDVSHEEPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGQGGGGQGGGGQGGGGQGGGGQGGGGQCPPCPAPELLGGPSVFLFPPKPKDTLMISRTPEVTCVVVDVSHEEPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGQVQLQESGPGLVKPSETLSLTCTVSGGSISSSSYFWGWIRQPPGKCLEWIGNIYYSGSSNYNPSLKSRVTISVDTSKNQFSLKLSSVTAADTAVYYCARLPRGDRDAFDIWGQGTMVTVSSGGGGQGGGGQGGGGQDIVMTQSPSSLSASVGDRVTITCRASQGISNYLAWYQQKPGKVPKLLIYAASTLQSGVPSRFSGSGSGTDFTLTISSLQPEDFATYYCQQSYSTPFTFGCGTKVEIKSGGGGQEVQLVESGGGLVQPGGSLKLSCAASGFTFNKYAMNWVRQAPGKGMEWVARIRSKYNNYATYYADAVKDRFTISRDDSKNTLYLQMNNLKTEDTAVYYCVRAGNFGSSYISYFAYWGQGTLVTVSSGGGGQGGGGQGGGGQQTVVTQEPSLTVSPGGTVTITCGSSTGAVTSGNYPNWIQKKPGQAPRGLIGGTKFLAPGTPARFSGSLEGGKAALTLSGVQPEDEAEYYCVLYYSNRWVFGSGTKLTVL

SEQ ID NO: 256 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 257 - protein, 201 aa, synthetic construct
MQSGTHWRVLGLCLLSVGVWGQDGNEEMAYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 258 - protein, 27 aa, synthetic construct
QDGNEEMGGITQTPYKVSISGTTVILT

SEQ ID NO: 259 - protein, 5 aa, synthetic construct
QDGNE

SEQ ID NO: 260 - skipped

SEQ ID NO: 261 - protein, 6 aa, synthetic construct
XDGXEE

SEQ ID NO: 262 - protein, 8 aa, synthetic construct
QDGNEEMG

SEQ ID NO: 263 - protein, 7 aa, synthetic construct
QDGNEEM

SEQ ID NO: 264 - protein, 6 aa, synthetic construct
QDGNEE

SEQ ID NO: 265 - protein, 6 aa, synthetic construct
QDGNDE

SEQ ID NO: 266 - protein, 8 aa, synthetic construct
SDGNEEMG

SEQ ID NO: 267 - protein, 8 aa, synthetic construct
QDGDEEMG

SEQ ID NO: 268 - protein, 8 aa, synthetic construct
QDGEEEMG

SEQ ID NO: 269 - protein, 8 aa, synthetic construct
QDGSEEMG

SEQ ID NO: 270 - protein, 8 aa, synthetic construct
QDGTEEMG

SEQ ID NO: 271 - protein, 8 aa, synthetic construct
QDGVEEMG

SEQ ID NO: 272 - protein, 8 aa, synthetic construct
QDGIEEMG

SEQ ID NO: 273 - protein, 7 aa, synthetic construct
  VARIANT 1: note = Pyroglutamic acid
DGNEEMG

SEQ ID NO: 274 - protein, 6 aa, synthetic construct
  VARIANT 1: note = Pyroglutamic acid
DGNEEM

SEQ ID NO: 275 - protein, 5 aa, synthetic construct
  VARIANT 1: note = Pyroglutamic acid
DGNEE

SEQ ID NO: 276 - protein, 7 aa, synthetic construct
  VARIANT 1: note = Pyroglutamic acid
DGDEEMG

SEQ ID NO: 277 - protein, 7 aa, synthetic construct
  VARIANT 1: note = Pyroglutamic acid
DGEEEMG

SEQ ID NO: 278 - protein, 7 aa, synthetic construct
  VARIANT 1: note = Pyroglutamic acid
DGSEEMG

SEQ ID NO: 279 - protein, 7 aa, synthetic construct
  VARIANT 1: note = Pyroglutamic acid
DGTEEMG

SEQ ID NO: 280 - protein, 7 aa, synthetic construct
  VARIANT 1: note = Pyroglutamic acid
DGVEEMG

SEQ ID NO: 281 - protein, 7 aa, synthetic construct
  VARIANT 1: note = Pyroglutamic acid
DGIEEMG

SEQ ID NO: 282 - protein, 7 aa, synthetic construct
  VARIANT 1: note = Pyroglutamic acid
DGDEELG

SEQ ID NO: 283 - protein, 7 aa, synthetic construct
  VARIANT 1: note = Pyroglutamic acid
DGEEELG

SEQ ID NO: 284 - protein, 7 aa, synthetic construct
  VARIANT 1: note = Pyroglutamic acid
DGSEELG

SEQ ID NO: 285 - protein, 7 aa, synthetic construct
  VARIANT 1: note = Pyroglutamic acid
DGTEELG

SEQ ID NO: 286 - protein, 7 aa, synthetic construct
  VARIANT 1: note = Pyroglutamic acid
DGVEELG

SEQ ID NO: 287 - protein, 7 aa, synthetic construct
  VARIANT 1: note = Pyroglutamic acid
DGIEELG

SEQ ID NO: 288 - protein, 8 aa, synthetic construct
  VARIANT 1: note = Pyroglutamic acid
DGNEELKG

SEQ ID NO: 289 - protein, 8 aa, synthetic construct
  VARIANT 1: note = Pyroglutamic acid
DGNEELRG

SEQ ID NO: 290 - protein, 8 aa, synthetic construct
  VARIANT 1: note = Pyroglutamic acid
DGNEEMRG

SEQ ID NO: 291 - protein, 8 aa, synthetic construct
  VARIANT 1: note = Pyroglutamic acid
DGDEELKG

SEQ ID NO: 292 - protein, 8 aa, synthetic construct
  VARIANT 1: note = Pyroglutamic acid
DGEEELKG

SEQ ID NO: 293 - protein, 8 aa, synthetic construct
  VARIANT 1: note = Pyroglutamic acid
DGSEELKG

SEQ ID NO: 294 - protein, 8 aa, synthetic construct
  VARIANT 1: note = Pyroglutamic acid
DGTEELKG

SEQ ID NO: 295 - protein, 8 aa, synthetic construct
  VARIANT 1: note = Pyroglutamic acid
DGVEELKG

SEQ ID NO: 296 - protein, 8 aa, synthetic construct
  VARIANT 1: note = Pyroglutamic acid
DGIEELKG

SEQ ID NO: 297 - protein, 17 aa, synthetic construct
QDGNEEMGKGMEENGDQ

SEQ ID NO: 298 - protein, 17 aa, synthetic construct
QDGNEEMGKQDGNEEMG

SEQ ID NO: 299 - protein, 17 aa, synthetic construct
QDGNEEMGRQDGNEEMG

SEQ ID NO: 300 - protein, 16 aa, synthetic construct
  VARIANT 1: note = Pyroglutamic acid
DGNEEMGKGMEENGDQ

SEQ ID NO: 301 - protein, 15 aa, synthetic construct
  VARIANT 1: note = Pyroglutamic acid
DGNEELKGLEENGDQ

SEQ ID NO: 302 - protein, 16 aa, synthetic construct
  VARIANT 1: note = Pyroglutamic acid
DGNEEMGRGMEENGDQ

SEQ ID NO: 303 - protein, 15 aa, synthetic construct
  VARIANT 1: note = Pyroglutamic acid
DGNEELRGLEENGDQ

SEQ ID NO: 304 - protein, 9 aa, synthetic construct
  VARIANT 1: note = Pyroglutamic acid
DGNEELKGC

SEQ ID NO: 305 - protein, 9 aa, synthetic construct
  VARIANT 1: note = Pyroglutamic acid
DGNEELKGK

SEQ ID NO: 306 - protein, 9 aa, synthetic construct
  VARIANT 1: note = Pyroglutamic acid
DGEEELKGC

SEQ ID NO: 307 - protein, 9 aa, synthetic construct
  VARIANT 1: note = Pyroglutamic acid
DGEEELKGK

SEQ ID NO: 308 - protein, 7 aa, synthetic construct
  VARIANT 1: note = Pyroglutamic acid
HGNEELK

SEQ ID NO: 309 - protein, 7 aa, synthetic construct
  VARIANT 1: note = Pyroglutamic acid
NGNEELK

SEQ ID NO: 310 - protein, 7 aa, synthetic construct
  VARIANT 1: note = Pyroglutamic acid
DFNEELK

SEQ ID NO: 311 - protein, 7 aa, synthetic construct
  VARIANT 1: note = Pyroglutamic acid
DYNEELK

SEQ ID NO: 312 - protein, 7 aa, synthetic construct
  VARIANT 1: note = Pyroglutamic acid
DGHEELK

SEQ ID NO: 313 - protein, 7 aa, synthetic construct
  VARIANT 1: note = Pyroglutamic acid
DGQEELK

SEQ ID NO: 314 - protein, 7 aa, synthetic construct
  VARIANT 1: note = Pyroglutamic acid
DGREELK

SEQ ID NO: 315 - protein, 7 aa, synthetic construct
  VARIANT 1: note = Pyroglutamic acid
DGNELLK

SEQ ID NO: 316 - protein, 7 aa, synthetic construct
  VARIANT 1: note = Pyroglutamic acid
DGNEPLK

SEQ ID NO: 317 - protein, 7 aa, synthetic construct
  VARIANT 1: note = Pyroglutamic acid
DGNEWLK

SEQ ID NO: 318 - protein, 7 aa, synthetic construct
  VARIANT 1: note = Pyroglutamic acid
DGHELLK

SEQ ID NO: 319 - protein, 7 aa, synthetic construct
  VARIANT 1: note = Pyroglutamic acid
DGREWLK

SEQ ID NO: 320 - protein, 7 aa, synthetic construct
  VARIANT 1: note = Pyroglutamic acid
NFNEELK

SEQ ID NO: 321 - protein, 7 aa, synthetic construct
  VARIANT 1: note = Pyroglutamic acid
HFNEELK

SEQ ID NO: 322 - protein, 7 aa, synthetic construct
  VARIANT 1: note = Pyroglutamic acid
NFHELLK

SEQ ID NO: 323 - protein, 7 aa, synthetic construct
  VARIANT 1: note = Pyroglutamic acid
NYHELLK

SEQ ID NO: 324 - protein, 7 aa, synthetic construct
  VARIANT 1: note = Pyroglutamic acid
HFHELLK

SEQ ID NO: 325 - protein, 7 aa, synthetic construct
  VARIANT 1: note = Pyroglutamic acid
NFREWLK

SEQ ID NO: 326 - protein, 7 aa, synthetic construct
  VARIANT 1: note = Pyroglutamic acid
NFEELLK

SEQ ID NO: 327 - protein, 7 aa, synthetic construct
  VARIANT 1: note = Pyroglutamic acid
NYEELLK

SEQ ID NO: 328 - protein, 7 aa, synthetic construct
  VARIANT 1: note = Pyroglutamic acid
NFEEWLK

SEQ ID NO: 329 - protein, 7 aa, synthetic construct
  VARIANT 1: note = Pyroglutamic acid
NFEEPLK

SEQ ID NO: 330 - protein, 7 aa, synthetic construct
  VARIANT 1: note = Pyroglutamic acid
NFQELLK

SEQ ID NO: 331 - protein, 7 aa, synthetic construct
  VARIANT 1: note = Pyroglutamic acid
NFREPLK

SEQ ID NO: 332 - protein, 7 aa, synthetic construct
  VARIANT 1: note = Pyroglutamic acid
NFRELLK

SEQ ID NO: 333 - protein, 7 aa, synthetic construct
  VARIANT 1: note = Pyroglutamic acid
NFHELLK

SEQ ID NO: 334 - protein, 7 aa, synthetic construct
  VARIANT 1: note = Pyroglutamic acid
NYQELLK

SEQ ID NO: 335 - protein, 7 aa, synthetic construct
  VARIANT 1: note = Pyroglutamic acid
NYEEWLK

SEQ ID NO: 336 - protein, 7 aa, synthetic construct
  VARIANT 1: note = Pyroglutamic acid
HFEELLK

SEQ ID NO: 337 - protein, 7 aa, synthetic construct
  VARIANT 1: note = Pyroglutamic acid
HFQELLK

SEQ ID NO: 338 - protein, 7 aa, synthetic construct
  VARIANT 1: note = Pyroglutamic acid
HFREPLK

SEQ ID NO: 339 - protein, 5 aa, synthetic construct
KYAMN

SEQ ID NO: 340 - protein, 19 aa, synthetic construct
RIRSKYNNYATYYADAVKD

SEQ ID NO: 341 - protein, 14 aa, synthetic construct
AGNFGSSYISYFAY

SEQ ID NO: 342 - protein, 14 aa, synthetic construct
GSSTGAVTSGNYPN

SEQ ID NO: 343 - protein, 7 aa, synthetic construct
GTKFLAP

SEQ ID NO: 344 - protein, 9 aa, synthetic construct
VLYYSNRWV

SEQ ID NO: 345 - protein, 125 aa, synthetic construct
EVQLVESGGGLVQPGGSLKLSCAASGFTFNKYAMNWVRQAPGKGMEWVARIRSKYNNYATYYADAVKDRFTISRDDSKNTLYLQMNNLKTEDTAVYYCVRAGNFGSSYISYFAYWGQGTLVTVSS

SEQ ID NO: 346 - protein, 109 aa, synthetic construct
QTVVTQEPSLTVSPGGTVTITCGSSTGAVTSGNYPNWIQKKPGQAPRGLIGGTKFLAPGTPARFSGSLEGGKAALTLSGVQPEDEAEYYCVLYYSNRWVFGSGTKLTVL

SEQ ID NO: 347 - protein, 5 aa, synthetic construct
KYAIN

SEQ ID NO: 348 - protein, 19 aa, synthetic construct
RIRSKYNNYATYYADAVKD

SEQ ID NO: 349 - protein, 14 aa, synthetic construct
AGNFGSSYISYWAY

SEQ ID NO: 350 - protein, 14 aa, synthetic construct
GSSTGAVTSGNYPN

SEQ ID NO: 351 - protein, 7 aa, synthetic construct
GTKFLAP

SEQ ID NO: 352 - protein, 9 aa, synthetic construct
VLWYSNRWV

SEQ ID NO: 353 - protein, 125 aa, synthetic construct
EVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSS

SEQ ID NO: 354 - protein, 109 aa, synthetic construct
QTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVL

SEQ ID NO: 355 - protein, 7 aa, synthetic construct
SSSYFWG

SEQ ID NO: 356 - protein, 16 aa, synthetic construct
NIYYSGSSNYNPSLKS

SEQ ID NO: 357 - protein, 11 aa, synthetic construct
LPRGDRDAFDI

SEQ ID NO: 358 - protein, 11 aa, synthetic construct
RASQGISNYLA

SEQ ID NO: 359 - protein, 7 aa, synthetic construct
AASTLQS

SEQ ID NO: 360 - protein, 9 aa, synthetic construct
QQSYSTPFT

SEQ ID NO: 361 - protein, 5 aa, synthetic construct
NYWMN

SEQ ID NO: 362 - protein, 17 aa, synthetic construct
NIAYGVAGTNYNQKFQG

SEQ ID NO: 363 - protein, 8 aa, synthetic construct
RYFYVMDY

SEQ ID NO: 364 - protein, 11 aa, synthetic construct
RASQDISNYLN

SEQ ID NO: 365 - protein, 7 aa, synthetic construct
YTSRLHS

SEQ ID NO: 366 - protein, 9 aa, synthetic construct
VQYAQFPLT

SEQ ID NO: 367 - protein, 5 aa, synthetic construct
TYAMN

SEQ ID NO: 368 - protein, 19 aa, synthetic construct
RIRSKYNNYATYYADSVKG

SEQ ID NO: 369 - protein, 14 aa, synthetic construct
HGNFGDSYVSWFAY

SEQ ID NO: 370 - protein, 14 aa, synthetic construct
GSSTGAVTTSNYAN

SEQ ID NO: 371 - protein, 7 aa, synthetic construct
GTNKRAP

SEQ ID NO: 372 - protein, 9 aa, synthetic construct
ALWYSNHWV

SEQ ID NO: 373 - protein, 199 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQG

SEQ ID NO: 374 - protein, 450 aa, synthetic construct
QVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLOSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 375 - protein, 720 aa, synthetic construct
QVQLVQSGAEVKKPGASVKVSCKASGYTFSTYWIEWVRQAPGQRLEWMGEILPGSGNTDENEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLOSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKTCLVKGFYPSDIAVEWESNGQPENNYKTTPPVLDSDGSFFLYSKLTVDKSRWQQGNVFSCSVMHEALHNHYTQKSSLSPGK

SEQ ID NO: 376 - protein, 307 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPCEEEYGSTYRCVS

SEQ ID NO: 377 - protein, 218 aa, synthetic construct
DIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 378 - protein, 448 aa, synthetic construct
QLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTIPPVLDSDGSFFLYSKLTVDKSRWEQGDVFSCSVMHEALHNHYTQKSLSLSPGK

SEQ ID NO: 379 - protein, 721 aa, synthetic construct
QLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 723 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLUSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPCEEQYGSTYRCVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 382 - protein, 249 aa, synthetic construct
EVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSSGGGGSGGGGSGGGGSQTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVL

SEQ ID NO: 383 - protein, 249 aa, synthetic construct
EVQLVESGGGLVQPGGSLKLSCAASGFTFNKYAMNWVRQAPGKGMEWVARIRSKYNNYATYYADAVKDRFTISRDDSKNTLYLQMNNLKTEDTAVYYCVRAGNFGSSYISYFAYWGQGTLVTVSSGGGGSGGGGSGGGGSQTVVTQEPSLTVSPGGTVTITCGSSTGAVTSGNYPNWIQKKPGQAPRGLIGGTKFLAPGTPARFSGSLEGGKAALTLSGVQPEDEAEYYCVLYYSNRWVFGSGTKLTVL

SEQ ID NO: 384 - protein, 9 aa, synthetic construct
  VARIANT 1: note = Pyroglutamic acid
EGNEELKGC

SEQ ID NO: 385 - protein, 5 aa, synthetic construct
  VARIANT 1: note = Pyroglutamic acid
EGNEE

SEQ ID NO: 386 - protein, 6 aa, synthetic construct
  VARIANT 1: note = Pyroglutamic acid
EGNEEL

SEQ ID NO: 387 - protein, 7 aa, synthetic construct
  VARIANT 1: note = Pyroglutamic acid
EGNEELK

SEQ ID NO: 388 - protein, 8 aa, synthetic construct
  VARIANT 1: note = Pyroglutamic acid
EGNEELKG

SEQ ID NO: 389 - protein, 9 aa, synthetic construct
  VARIANT 1: note = Pyroglutamic acid
EGEEELKGC

SEQ ID NO: 390 - protein, 5 aa, synthetic construct
  VARIANT 1: note = Pyroglutamic acid
FGNEE

SEQ ID NO: 391 - protein, 6 aa, synthetic construct
  VARIANT 1: note = Pyroglutamic acid
FGNEEK

SEQ ID NO: 392-396 - skipped (5 entries)

SEQ ID NO: 397 - protein, 5 aa, synthetic construct
  VARIANT 1: note = Pyroglutamic acid
DGCEE

SEQ ID NO: 398 - protein, 5 aa, synthetic construct
  VARIANT 1: note = Pyroglutamic acid
DGDEE

SEQ ID NO: 399 - protein, 5 aa, synthetic construct
  VARIANT 1: note = Pyroglutamic acid
DGEEE

SEQ ID NO: 400 - protein, 5 aa, synthetic construct
  VARIANT 1: note = Pyroglutamic acid
DGFEE

SEQ ID NO: 401 - protein, 5 aa, synthetic construct
  VARIANT 1: note = Pyroglutamic acid
DGKEE

SEQ ID NO: 402 - protein, 5 aa, synthetic construct
  VARIANT 1: note = Pyroglutamic acid
DGLEE

SEQ ID NO: 403 - protein, 5 aa, synthetic construct
  VARIANT 1: note = Pyroglutamic acid
DGMEE

SEQ ID NO: 404 - protein, 5 aa, synthetic construct
  VARIANT 1: note = Pyroglutamic acid
DGWEE

SEQ ID NO: 405 - protein, 5 aa, synthetic construct
  VARIANT 1: note = Pyroglutamic acid
DGYEE

SEQ ID NO: 406 - protein, 5 aa, synthetic construct
  VARIANT 1: note = Pyroglutamic acid
DGNEC

SEQ ID NO: 407 - protein, 5 aa, synthetic construct
  VARIANT 1: note = Pyroglutamic acid
DGNED

SEQ ID NO: 408 - protein, 5 aa, synthetic construct
  VARIANT 1: note = Pyroglutamic acid
DGNEF

SEQ ID NO: 409 - protein, 5 aa, synthetic construct
  VARIANT 1: note = Pyroglutamic acid
DGNEG

SEQ ID NO: 410 - protein, 5 aa, synthetic construct
  VARIANT 1: note = Pyroglutamic acid
DGNEH

SEQ ID NO: 411 - protein, 5 aa, synthetic construct
  VARIANT 1: note = Pyroglutamic acid
DGNEK

SEQ ID NO: 412 - protein, 5 aa, synthetic construct
  VARIANT 1: note = Pyroglutamic acid
DGNEL

SEQ ID NO: 413 - protein, 5 aa, synthetic construct
  VARIANT 1: note = Pyroglutamic acid
DGNEM

SEQ ID NO: 414 - protein, 5 aa, synthetic construct
  VARIANT 1: note = Pyroglutamic acid
DGNEQ

SEQ ID NO: 415 - protein, 5 aa, synthetic construct
  VARIANT 1: note = Pyroglutamic acid
DGNER

SEQ ID NO: 416 - protein, 5 aa, synthetic construct
  VARIANT 1: note = Pyroglutamic acid
DGNES

SEQ ID NO: 417 - protein, 5 aa, synthetic construct
  VARIANT 1: note = Pyroglutamic acid
DGNET

SEQ ID NO: 418 - protein, 5 aa, synthetic construct
  VARIANT 1: note = Pyroglutamic acid
DGNEV

SEQ ID NO: 419 - protein, 5 aa, synthetic construct
  VARIANT 1: note = Pyroglutamic acid
DGNEY

SEQ ID NO: 420 - protein, 5 aa, synthetic construct
GYYMH

SEQ ID NO: 421 - protein, 17 aa, synthetic construct
WINPNSGETNYAQKFQG

SEQ ID NO: 422 - protein, 20 aa, synthetic construct
DALIVVAPVTRDYYYYGMDV

SEQ ID NO: 423 - protein, 12 aa, synthetic construct
RASQSVSSSYLA

SEQ ID NO: 424 - protein, 7 aa, synthetic construct
GASSRAT

SEQ ID NO: 425 - protein, 9 aa, synthetic construct
QQYGSSPLT

SEQ ID NO: 426 - protein, 129 aa, synthetic construct
QVQLVQSGAEVKKPGASVKVSCKASGYTFTGYYMHWVRQAPGQCLEWMGWINPNSGETNYAQKFQGRVTMTRDTSISTAYMELSRLRSDDTAVYYCARDALIVVAPVTRDYYYYGMDVWGQGTTVTVSS

SEQ ID NO: 427 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGCGTKLEIK

SEQ ID NO: 428 - protein, 252 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGCGTKLEIKGGGGSGGGGSGGGGSQVQLVQSGAEVKKPGASVKVSCKASGYTFTGYYMHWVRQAPGQCLEWMGWINPNSGETNYAQKFQGRVTMTRDTSISTAYMELSRLRSDDTAVYYCARDALIVVAPVTRDYYYYGMDVWGQGTTVTVSS

SEQ ID NO: 429 - protein, 507 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGCGTKLEIKGGGGSGGGGSGGGGSQVQLVQSGAEVKKPGASVKVSCKASGYTFTGYYMHWVRQAPGQCLEWMGWINPNSGETNYAQKFQGRVTMTRDTSISTAYMELSRLRSDDTAVYYCARDALIVVAPVTRDYYYYGMDVWGQGTTVTVSSSGGGGSEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSSGGGGSGGGGSGGGGSQTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVL

SEQ ID NO: 430 - protein, 985 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGCGTKLEIKGGGGSGGGGSGGGGSQVQLVQSGAEVKKPGASVKVSCKASGYTFTGYYMHWVRQAPGQCLEWMGWINPNSGETNYAQKFQGRVTMTRDTSISTAYMELSRLRSDDTAVYYCARDALIVVAPVTRDYYYYGMDVWGQGTTVTVSSSGGGGSLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSSGGGGSGGGGSGGGGSQTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 7 aa, synthetic construct
  VARIANT 1: note = Pyroglutamic acid
DGNEELK

SEQ ID NO: 432-433 - skipped (2 entries)

SEQ ID NO: 434 - protein, 9 aa, synthetic construct
  VARIANT 1: note = Pyroglutamic acid
XXXEELKGX

SEQ ID NO: 435 - protein, 9 aa, synthetic construct
  VARIANT 1: note = Pyroglutamic acid
XGXEELKGX

SEQ ID NO: 436 - protein, 11 aa, synthetic construct
PEDGXEELKGX

SEQ ID NO: 437 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 439 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 440 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 441 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 443 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 444 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP